(19) United States
(12) Patent Application Publication
(10) Pub. No.: US 2008/0176240 A1
(43) Pub. Date: Jul. 24, 2008

(54) GENES ASSOCIATED WITH SCHIZOPHRENIA IDENTIFIED USING A WHOLE GENOME SCAN

(75) Inventor: Pamela St. Jean, Morrisville, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 11/970,611

(22) Filed: Jan. 8, 2008

Related U.S. Application Data
(60) Provisional application No. 60/885,654, filed on Jan. 19, 2007.

(51) Int. Cl. C12Q 1/68

(52) U.S. Cl. 435/6

(57) ABSTRACT

Genes associated with schizophrenia are identified. Methods of screening small molecule compounds for use in treating schizophrenia are described.

### CROSS-REFERENCE TO RELATED APPLICATIONS

This application is based on and claims priority to U.S. provisional patent application No. 60/885,654 filed on Jan. 19, 2007 and is incorporated herein in its entirety.

### FIELD OF THE INVENTION

The present invention relates to identification of genes that are associated with schizophrenia and to screening methods to identify chemical compounds that act on those targets for the treatment of Schizophrenia or its associated pathologies.

### BACKGROUND OF THE INVENTION

The purpose of the present study was to identify Single Nucleotide Polymorphisms (SNPs) that are associated with schizophrenia using whole genome scan panels, to develop methods of screening compounds to identify those that act on such targets, and to develop such compounds as medicines to treat Schizophrenia and its associated pathologies.

Schizophrenia is a common disorder, affecting 1% of the population, independent of regional origin or cultural background (Thaker, 2001). The socio-economic impact of the disease is heavy, since direct costs of schizophrenia in western countries range between 1.6% to 2.6% of total health care expenditures. About 75% of these costs are due to inpatient or residential care, while drug therapy represents less than 5% (Barbato, 1998). Therefore, any new medicine could reduce the cost of care for schizophrenia while targeting the most disabled patients in order to improve their independent living skill. Further understanding of schizophrenia is therefore a valuable strategy for the development of innovative drugs to treat the disease and reduce both its social and economical burden.

Although the aetiology of schizophrenia is still unexplained, diverse evidence favours a genetic component. The lifetime morbidity risk for schizophrenia in the general population is close to 1%. This risk increases to about 3-5% in second degree relatives or in half-siblings, and up to 9-12% in siblings and dizygotic twins. Monozygotic twins share a 50% relative risk, as well as the children of two schizophrenic parents. Together these data suggest a heritability of 80%, emphasizing the strong genetic basis of this disorder (Cardno, 1999; Cardno, 2000 and references herein). However, in addition, epigenetic factors clearly play a role in the disease given the lack of complete sharing of the risk in monozygotic twins, and the lack of any major gene identified or reproducibly localised to date in genetic studies (McGue, 1989; Cardno, 2000; Riley, 2000 and references herein). Thus, schizophrenia is a multifactorial disorder, with a high likelihood of involving many genes of mild to moderate contribution in conjunction with environmental risk factors (Risch, 1990; Cardno, 2000). The identification of these genes, even of small and combined effects, will be of considerable therapeutic importance, and will help establish a complete picture of the pathophysiology of the disease. Ultimately, a better understanding of the underlying pathophysiology of the disease would permit more rational drug development.

### SUMMARY OF THE INVENTION

A first aspect of the present invention is a method for screening small molecule compounds for use in treating Schizophrenia by screening a test compound against a target selected from the group consisting of the products of genes with Schizophrenia associated SNPs listed in tables 2-4. Activity against said target indicates the test compound has potential use in treating Schizophrenia.

### DETAILED DESCRIPTION

The present inventors tested SNPs within whole genome scan panels of 300,000 to 550,000 SNPs in two collections of Schizophrenia case/control collections from Munich, Germany and from Aberdeen, Scotland. Each cohort comprised roughly 450 cases and 450 controls.

Biological molecules known to be responsive to manipulation by small molecule chemical compounds, e.g., can be activated or inhibited by small molecule chemical compounds, are known as ‘tractable’ or ‘druggable’ targets. Classes of ‘tractable targets’ include, but are not limited to, 7-transmembrane receptors (7™ receptors), ion channels, nuclear receptors, kinases, proteases and integrins. Genes that encode such molecules, and the molecules so produced, are preferred targets in the present methods.

### EXAMPLE 1

### Subjects and Methods

### Sample Set

The sample set consisted of Caucasian cases and Caucasian controls collected in Munich (Klinikum der Universitat München—Innenstadt, Munich, Germany, Principal Investigator (PI) Hans-Juergen Moeller & Dan Rujescu; The Max Planck Psychiatry Institute, Munich, Germany (PI Florian Holsboer); and the University of Aberdeen, Aberdeen, Scotland (PI David St Clair). All subjects gave informed consent for the use of their DNA in this study. Caucasian is defined as having 3 of 4 grandparents self-reported as Caucasian. The Munich sample set consisted of 438 cases and 414 controls. The Aberdeen sample consisted of 440 cases and 453 controls.

An individual was eligible for inclusion in this study only if all the following criteria applied: was at least 18 years of age at the time of entering the study; was of Caucasian background; and gave voluntary written consent to participate in the study. In addition to the general inclusion criteria, each schizophrenia case (patient) must have had the diagnosis of schizophrenia as defined by the Diagnostic and Statistical Manual of Mental Disorders (DSM-IV) and/or International Classification of Diseases ICD-10, American Psychiatric Association 1994). A schizophrenia case was not eligible for inclusion in this study if any of the following criteria applied: Have been diagnosed/or presented with schizoaffective disorder, or a mood disorder unrelated to schizophrenia; was a monozygotic twin of a case already included in the study and/or had been diagnosed as an intravenous drug user with a lifetime diagnosis of dependency.

### Whole Genome Scan Panels: Sample Preparation and Genotyping

DNA was isolated from whole blood using a basic salting-out procedure. Samples were arrayed and normalized in water to a standard concentration of 100 ng/ul. Fifty ul aliquots of the DNA samples were arrayed into 96-well PCR plates. For purposes of quality control, 6% of the samples were duplicated on the plates. The plates were stored at −20° C. until use. Samples were genotyped using the Illumina HumanHAP 300 and HumanHAP 550 genotyping platforms at the Center for Population Genomics and Pharmacogenetics within Duke University's Institute for Genome Science and Policy. The genotyptic data was then returned to GlaxoSmithKline (GSK) where it was loaded into the GSK database of record, SubjectLand, and then analyzed at GSK for association to schizophrenia.

### Data Handling

The GSK database of record for analysis-ready data is called SubjectLand. This database contains all genotypes, phenotypes (i.e. clinical data), and pedigree information, where applicable, on all subjects used in the analysis of data for these studies. SubjectLand does not maintain information regarding DNA samples, but is closely integrated with the sample tracking system to maintain the connection between subjects and their samples and phenotypic data at all times. All subjects gave informed consent for the use of their DNA and phenotypic data in this study. The analytical tools used in the analysis process described below interface directly with subject data in SubjectLand. This interface also archives the files used in analysis as well as the results.

### Analysis

Only subjects with a subject type (SBTY) of case or control were analyzed. Subjects with a SBTY of affected family member or other SBTY values were excluded from analysis. Subjects were also excluded if he/she, either parent, or more than one grandparent were non-Caucasian as indicated by self-report. In addition, subjects were excluded if their putative gender was inconsistent with SNP genotypes on the X chromosome.

Each marker was examined for Hardy-Weinberg equilibrium (HWE) and minor allele frequency (MAF). Monomorphic markers were not analyzed. Markers with a Hardy-Weinberg p-value<(0.05/number of markers in whole genome scan) in controls were not analyzed. The HWE thresholds for exclusion for analysis were 3E-6 and 1.8E-6 for the HumanHAP 300 panel and HumanHAP 550, respectively. Markers that returned genotypes on <80% of subjects plated were not analyzed. Genotypic and allelic associations test were then performed using a Fishers Exact Test, followed by identification of the risk allele and risk genotype using chi-square tests. An odds ratio and confidence interval of greater than 95% was calculated for the risk allele and risk genotype. Next, population stratification was evaluated by determining if the number of allelic and genotypic tests observed to be significant at a given threshold was inflated with respect to what would be expected under the null hypothesis of no association. If population stratification was observed, a genomic-control adjustment was applied to the Fast Fishers Exact Test results.

The two collections (Munich and Aberdeen) were genotyped and analyzed separately. In each collection, statistically significant SNPs were identified as those with an allelic or genotypic Fishers Exact P-value ≦0.05. SNPs significant in both the Munich and Aberdeen collections were then identified. This set of SNPs was then evaluated to determine if test direction was concordant in each of the collections. For example, if a SNP had a significant allelic test in the Munich collection and a significant allelic test in the Aberdeen collection, then test direction would be concordant if each collection had the same risk allele and discordant if the risk alleles differed. To determine test concordancy, the Munich and Aberdeen subjects were combined into a single set and analyzed in the same manner as the individual collections (i.e., genotypic and allelic associations test were then performed using a Fishers Exact Test, followed by identification of the risk allele and risk genotype using chi-square tests). SNPs that were significantly associated in this combined set were defined as being concordant.

### Hardy Weinberg Equilibrium

Hardy Weinberg equilibrium (HWE) is a measure of the association between two alleles at an individual locus. A bi-allelic marker is in HWE if the genotype frequencies are p2, 2pq and q2 for the genotypes 1, 1; 1, 2; and 2, 2 where p and q are the frequencies of the 1 and 2 alleles, respectively. The departure from HWE was tested using a Chi square test, by testing the difference between the expected (calculated from the allele frequencies) and observed genotype frequencies. A HWE permutation test was performed when the HWE chi-square p-value <0.05 and when at least one genotype cell had an expected count less than five (Zaykin et al, 1995). When these conditions exist, the HWE chi-square test may not be valid and a permutation test to assess departure from HWE is warranted. Markers failing HWE at p≦0.001 in controls were removed from the pooled analysis marker cluster used in association analyses. HWE failure may indicate a non-robust assay.

### Minor Allele Frequency

For minor allele frequency, markers which were monomorphic were removed from the analysis marker cluster used in association analyses.

### Allelic and Genotypic Test of Association

Testing for association in the study data was carried out using the ‘PROC FREQ’ fast Fisher's exact test (FET) procedure in the statistical software package SASv8.2. An exact test is warranted in situations when asymptotic assumptions are not met such as when the sample size is not large or when the distribution is sparse or skewed. Such situations occur for SNPs with rare minor allele frequencies where the number of expected cases and/or controls for the rare homozygote are less than five. Under these conditions, the asymptotic results many not be valid and the asymptotic p-value may differ substantially from the exact p-value. The classic Fisher's Exact Test computes exact p-values by enumerating all tables as extreme as, or more extreme than, that observed. This direct enumeration approach is very time-consuming and only feasible for small problems. The fast Fisher's Exact test computes exact p-values for general R×C tables using the network algorithm developed by Mehta and Patel (1983). The network algorithm provides substantial advantage over direct enumeration and is rapid and accurate.

Tables I and II show the structure of the genotype and allele contingency tables, respectively.

TABLE I
Generic disease status by genotype contingency table.
Disease Status

| | | Case | Control | Total |
|---|---|---|---|---|
| Genotype | AA | n11 | n12 | n1. |
| | Aa | n21 | n22 | n2. |
| | aa | n31 | n32 | n3. |

| | | | |
|---|---|---|---|
| Total | n.1 | n.2 | N |

TABLE II
Generic disease status by allele contingency table.
Disease Status

| | | Case | Control | Total |
|---|---|---|---|---|
| Allele | A | 2n11 + n21 | 2n12 + n22 | 2n1. + n2. |
| | a | 2n31 + n21 | 2n32 + n22 | 2n3. + n2. |

| | | | |
|---|---|---|---|
| Total | 2n.1 | 2n.2 | 2N |

### Risk Allele and Risk Genotype

The “risk allele” refers to the allele that appeared more frequently in cases than controls. The “risk genotype” was determined after identifying the genotype that had the largest chi-square value when compared against the other two genotypes combined in the genotypic association test. For example, if a SNP had genotypes AA, AG and GG, three chi-square tests were performed contrasting cases and controls: 1) AA vs AG+GG, 2) AG vs AA+GG and 3) GG vs AA+AG. An odds ratio was then calculated for the test with the largest chi-square statistic. If the odds ratio was >1, this genotype was reported as the risk genotype. If the odds ratio was <1, then 1) the risk genotype was reported as “!” (“!” means “not”) this genotype and 2) a new odds ratio was calculated as the inverse of the original odds ratio. This new odds ratio was reported.

### Odds Ratios and Confidence Intervals

An odds ratio was constructed for the risk allele and risk genotype.

Odds ratio(OR)=(n11*n22)/(n12*n21)

where
n11=cases with risk genotype
n21=cases without risk genotype
n12=controls with risk genotype
n22=controls without risk genotype

In order to avoid division or multiplication by zero, 0.5 was added to each cell in the contingency table (as recommended in “Statistical Methods for Rates and Proportions” by Fleiss, Ch 5.3 p. 64)

A 95% confidence interval for the odds ratio was also calculated as follows:

where
z=97.5th percentile of the standard normal distribution

v=[1/(n11)]+[1/(n12)]+[1/(n21)]+[1/(n22)]

### Evaluation of Population Stratification

In this assessment, cases and control frequencies were compared across a subset of relatively independent markers (markers in low LD) selected from the set of all markers analyzed. Since the vast majority of genes on the gene list are not associated with a specific disease, this constitutes a null data set. If the cases and controls are from the same underlying population, the expectation is to see 5% of the tests significant at the 5% level, 1% significant at the 1% level, etc. If, on the other hand, the cases and controls are from different populations, (for example, cases from Finland and controls from Japan), there would be an inflation in the proportion of tests significant across thresholds due to genetic differences between the two populations that are unrelated to disease. Inflation in the number of observed significant tests over a range of cut-points suggests that the case and control groups are not well matched. Consequently, the inflated number of positive tests may be due to population stratification rather than to association between the associated SNPs and disease.

The probability of ≧m observed number of significant tests out of n total tests at a cut-point p was calculated using the binomial probability as implemented in SAS v8.2 as PROBNML (p,n,m) computes the probability that an observation from a binomial (n,p) distribution will be less than or equal to m.

### Adjusting for Population Stratification

Using a Genomic Control approach can correct for overdispersion of test statistics caused by population substructure. Devlin et al (2002) propose dividing the tests for association by \hat{λ}

The simplest implementation suggested is to estimate \hat{λ} as follows

\hat{λ}={median (S₁, S₂ . . . , Sₙ)/X} where Sᵢ refers to the chi-square statistic for loci i=1 to n. X is the median of the chi-square distribution given degrees of freedom (X=0.456 for 1 df test and 1.39 for 2 df test). When evidence of population stratification existed, we adjusted the FET allelic or genotypic p-values. The p-values were 1^{st} converted to a chi-square statistics. \hat{λ} was estimated using the derived chi-square statistics. Next, the derived chi-square statistic was multiplied by to yield an adjusted chi-square statistic. The adjusted chi-square statistic then was then used to calculate an adjusted FET p-value.

### Linkage Disequilibrium

The Linkage Disequilibrium (LD) between two markers is given by DAB=pAB-pApB, where pA is the allele frequency of A allele of the first marker, pB is the allele frequency of B allele of the second marker, and pAB is the joint frequency of alleles A and B on the same haplotype. LD tends to decline with distance between markers and generally exists for markers that are less than 100 kb apart

The SAS procedure PROC CORR was used to calculate r using the Pearson product-moment correlation. To determine whether significant LD existed between a pair of markers we made use of the fact that nr2 has an approximate chi square distribution with 1 df for biallelic markers. The significance level of pairwise LD was computed in SAS.

### EXAMPLE 2

### Results

The Munich collection of 856 subjects was genotyped on the Illumina HumanHAP 300 panel of 317,503 SNPs. Four collected subjects were excluded from the study based on sample set quality control (QC) measures. Two were excluded for subject type and two for gender inconsistency. The analysis set of 438 cases and 414 controls was reasonably well-matched with respect to gender with the proportion of males being slightly higher among cases.

The Aberdeen collection of 913 subjects was genotyped on the Illumina HumanHAP 550 panel of 555,352 SNPs. Six collected subjects were excluded from the study based on sample set quality control (QC) measures. Three were excluded as they belonged to the Munich collection, and three for gender inconsistency. The analysis set of 447 cases and 460 controls was well-matched with respect to age and gender. Key demographic characteristics of the Munich and Aberdeen sets are detailed in Table 1.

During SNP marker quality control of the 317,503 SNPs for the Munich collection, 13 SNPs were excluded due to Hardy-Weinberg Equilibrium (HWE); 72 SNPs were excluded because SNPs were monomorphic in cases and controls; and 9,686 SNPs were excluded as the returned genotypes on fewer than 80% of samples. As a result, 307,732 SNPs were analyzed for association with schizophrenia in the Munich collection.

During SNP marker quality control of the 555,352 SNPs for the Aberdeen collection, 69 SNPs were excluded due to Hardy-Weinberg Equilibrium (HWE); 10,452 SNPs were excluded because SNPs were monomorphic in cases and controls; 8,714 SNPs were excluded as the returned genotypes on fewer than 80% of samples. As a result, 536,117 SNPs were analyzed for association with schizophrenia in the Aberdeen collection.

Out of the 307,732 SNPs analyzed in the Munich collection, 23,466 SNPs had a genotypic or allelic Fisher's Exact p-value of ≦0.05 (data not shown). Since there was no evidence of population stratification in the Munich sample, a genomic control adjustment was not applied to the p-values. Across all SNPs, the number of cases genotyped was between 434 and 438 while the number of controls was between 409 and 414.

Of the 536,117 SNPs analyzed for association with Schizophrenia in the Aberdeen collection, 42,502 SNPs had a genotypic or allelic Fisher's Exact p-value of ≦0.05 (data not shown). Since there was no evidence of population stratification in the Aberdeen sample, a genomic control adjustment was not applied to the p-values. Across all SNPs, the number of cases genotyped was between 442 and 447 while the number of controls was between 456 and 460.

An assessment of the results from the Munich and the Aberdeen collections, revealed that 1,814 SNPs showed statistical evidence in both individual collections. These SNPs had p-values ≦0.05 from the genotypic and/or allelic exact tests in the Munich collection and also in the Aberdeen collection. In order to assess concordance of test direction, these 1,814 SNPs were then analyzed for association in a combined set of all Munich and Aberdeen subjects. Among these 1,814 SNPs, 1,069 SNPs showed concordant test direction between the two collections (i.e., had a genotypic or allelic Fisher's Exact p-value of ≦0.05 in the combined set). These 1,069 SNPs with concordant test direction demonstrate the strongest evidence of replication. It is possible, albeit not likely, for a gene have a discordant test direction yet still be a risk factor in two different collections, if the gene has undergone a divergent evolutionary history in the two collections. However concordance of test direction provides stronger evidence of replication than does discordance of test direction. The association results for the two individual collections for the 1,069 concordant SNPs are displayed in Table 2. Due to space limitations, the results from the combined Munich+Aberdeen set could not be displayed in Table 2. Instead the association results for these 1,069 SNPs in the combined Munich+Aberdeen set are displayed in Table 3. For convenience the genotyping and allelic p-values for the individual Munich and Aberdeen collections are also shown in Table 3 (as well as in Table 2).

It is possible that some of the associations are false positives. Statistical association between a polymorphic marker and disease may occur for several reasons. The marker may be a mutation that influences disease susceptibility directly or may be correlated with a mutation that influences disease susceptibility because the marker and disease susceptibility mutation are physically close to one another. Spurious association may result from issues such as confounding or bias although the study design attempts to remove or minimize these factors. The association between a marker and disease may also be due to chance.

For the assessment, 1,069 SNPs were significant at p<0.05 in two separate collections, Munich and Aberdeen and showed concordant test direction between the two collections. These SNPs and their genes (Tables 2-4) have the strongest statistical evidence for association with Schizophrenia.

TABLE 1
Collections analysed
Schizophrenia CASES

| | Sites | | | |
|---|---|---|---|---|
| | Munich (Germany) | | Aberdeen (UK) | |
| | n = 438 | | n = 447 | |
| Male(%):Female(%) | 282 (64.4%):156 (35.6%) | | 326 (72.9%):121 (27.1%) | |
| Mean age (+/−sd) | 38.6 | (11.5) | 42.8 | (12.9) |
| DSM score: | | | | |
| Disorganised type | 67 | (15.7%) | 9 | (2%) |
| Catatonic type | 6 | (1.4%) | 1 | (0.2%) |
| Paranoid type | 330 | (77.1%) | 357 | (79.9%) |
| Residual type | 3 | (0.7%) | 46 | (10.3%) |
| Undifferentiated | 22 | (5.1%) | 34 | (7.6%) |
| type | | | | |

CONTROL

| | Munich (Germany) | | Aberdeen (UK) | |
|---|---|---|---|---|
| | n = 414 | | n = 460 | |
| Male(%):Female | 203 (49.0%):211 (51.0%) | | 304 (66.1%):156 (33.9%) | |
| (%) | | | | |
| Mean age (+/−sd) | 48.8 | (14.7) | 45.6 | (12.5) |

TABLE 2
Schizophrenia SNPs Significantly Associated and Concordant in Both Collections - Results
from the Individual Munich and Aberdeen Collections¹.

| | | | Munich | | Munich | Munich 300K | Aberdeen | | Aberdeen | Aberdeen 550K |
|---|---|---|---|---|---|---|---|---|---|---|
| | | | 300K | Munich 300K Risk | 300K | Risk Allele & | 550K | Aberdeen 550K Risk | 550K | Risk Allele & |
| | NCBI 36 | Gene | Genotypic | Genotype & Odds | Allelic | Odds Ratio | Genotypic | Genotype & Odds | Allelic | Odds Ratio |
| Poly Name² | position | Symbols³ | Exact P⁴ | Ratio (95% CI)⁵ | Exact P⁴ | (95% CI) | Exact P⁴ | Ratio (95% CI)⁵ | Exact P⁴ | (95% CI) |
| RS12026226 | 01: 004787770 | | 0.0093 | C,C: 10.52(0.58, 190.81) | 0.3112 | A: 1.19(0.86, 1.66) | 0.0529 | A,A: 1.44(1, 2.09) | 0.0414 | A: 1.44(1.01, 2.05) |
| RS2011197 | 01: 005942458 | NPHP4 | 0.0595 | A,G: 1.43(1.06, 1.94) | 0.0425 | G: 1.31(1.01, 1.69) | 0.0255 | G,G: 2.44(1.24, 4.8) | 0.0552 | G: 1.27(1, 1.6) |
| RS2047811 | 01: 005954440 | NPHP4 | 0.0595 | A,C: 1.43(1.06, 1.94) | 0.0425 | A: 1.31(1.01, 1.69) | 0.0255 | A,A: 2.44(1.24, 4.8) | 0.0552 | A: 1.27(1, 1.6) |
| RS1294028 | 01: 009287221 | SPSB1 | 0.0738 | !C,C: 1.38(1.05, 1.82) | 0.0468 | T: 1.22(1.01, 1.49) | 0.0223 | T,T: 1.56(1.08, 2.27) | 0.0082 | T: 1.29(1.07, 1.56) |
| RS1133398 | 01: 011737218 | | 0.0038 | G,G: 2.12(1.28, 3.5) | 0.0026 | G: 1.38(1.12, 1.7) | 0.0135 | A,G: 1.48(1.14, 1.93) | 0.2029 | G: 1.14(0.94, 1.4) |
| RS3765379 | 01: 015063006 | KIAA1026 | 0.0439 | G,G: 2.07(1.02, 4.23) | 1.0000 | G: 1.01(0.8, 1.27) | 0.0838 | T,T: 1.35(1.03, 1.77) | 0.0329 | T: 1.28(1.02, 1.61) |
| RS12740975 | 01: 019310510 | ZUBR1 | 0.0317 | !A,A: 1.43(1.09, 1.87) | 0.0139 | G: 1.3(1.06, 1.61) | 0.0612 | G,G: 1.62(1.01, 2.59) | 0.0295 | G: 1.25(1.02, 1.52) |
| RS1009806 | 01: 019373138 | ZUBR1 | 0.0317 | !C,C: 1.43(1.09, 1.87) | 0.0139 | T: 1.3(1.06, 1.61) | 0.0825 | T,T: 1.56(0.98, 2.49) | 0.0381 | T: 1.23(1.01, 1.51) |
| RS2273046 | 01: 019438883 | KIAA0090 | 0.0476 | !A,A: 1.41(1.07, 1.85) | 0.0179 | G: 1.32(1.05, 1.65) | 0.0483 | G,G: 1.87(1.04, 3.36) | 0.0253 | G: 1.28(1.03, 1.59) |
| RS7523192 | 01: 019835634 | | 0.0413 | !C,T: 1.45(1.07, 1.95) | 0.1789 | T: 1.19(0.93, 1.53) | 0.0364 | C,C: 1.94(1.01, 3.72) | 1.0000 | C: 1.01(0.79, 1.28) |
| RS2473277 | 01: 022234432 | | 0.0172 | T,T: 1.51(1.11, 2.06) | 0.0057 | T: 1.31(1.08, 1.59) | 0.0591 | !C,C: 1.46(1.06, 2) | 0.0382 | T: 1.22(1.01, 1.47) |
| RS3737807 | 01: 028134489 | SMPDL3B | 0.0416 | G,T: 1.39(1.06, 1.82) | 0.8070 | T: 1.03(0.85, 1.25) | 0.0117 | G,T: 1.48(1.14, 1.92) | 0.4220 | G: 1.08(0.9, 1.3) |
| RS4949294 | 01: 031008523 | | 0.0326 | !G,T: 1.43(1.09, 1.88) | 0.8079 | G: 1.03(0.85, 1.24) | 0.0079 | G,G: 1.54(1.15, 2.08) | 0.0907 | G: 1.17(0.98, 1.41) |
| RS10914202 | 01: 031024793 | | 0.0262 | !C,T: 1.45(1.11, 1.9) | 0.9226 | C: 1.01(0.84, 1.22) | 0.0109 | C,C: 1.55(1.15, 2.09) | 0.0602 | C: 1.2(0.99, 1.44) |
| RS747020 | 01: 032470521 | LOC339483, | 0.0559 | G,G: 1.6(1.05, 2.44) | 0.0323 | G: 1.57(1.05, 2.35) | 0.0285 | G,G: 1.61(1.1, 2.38) | 0.0130 | G: 1.59(1.1, 2.3) |
| | | EIF3S2 | | | | | | | | |
| RS2135592 | 01: 036930532 | | 0.0275 | C,T: 1.47(1.05, 2.07) | 0.1164 | T: 1.28(0.94, 1.75) | 0.0820 | !C,C: 1.36(1, 1.84) | 0.0340 | T: 1.35(1.02, 1.79) |
| RS12734651 | 01: 042103172 | HIVEP3 | 0.0789 | A,A: 1.35(1.03, 1.76) | 0.0252 | A: 1.26(1.03, 1.55) | 0.1029 | A,A: 1.28(0.99, 1.67) | 0.0318 | A: 1.25(1.02, 1.52) |
| RS557435 | 01: 055293452 | PCSK9, | 0.0600 | !A,A: 1.39(1.05, 1.84) | 0.0265 | G: 1.32(1.04, 1.69) | 0.0829 | !A,A: 1.35(1.03, 1.77) | 0.0288 | G: 1.29(1.03, 1.62) |
| | | TIG44321 | | | | | | | | |
| RS12144887 | 01: 068116230 | | 0.0549 | !T,T: 1.62(1.04, 2.51) | 0.0223 | C: 1.27(1.04, 1.56) | 0.0739 | C,C: 1.33(1.02, 1.73) | 0.0264 | C: 1.25(1.03, 1.52) |
| RS4287129 | 01: 074788735 | | 0.0188 | G,G: 5.24(1.35, 20.32) | 0.3176 | G: 1.15(0.88, 1.49) | 0.0962 | !T,T: 1.38(1.03, 1.86) | 0.0419 | G: 1.32(1.01, 1.73) |
| RS6703099 | 01: 075306907 | | 0.0375 | C,C: 1.45(1.06, 1.96) | 0.1598 | C: 1.15(0.95, 1.39) | 0.0398 | C,C: 1.38(1.04, 1.81) | 0.0118 | C: 1.28(1.06, 1.54) |
| RS2734700 | 01: 086722908 | CLCA1 | 0.0472 | !G,G: 1.42(1.08, 1.88) | 0.0170 | A: 1.33(1.05, 1.69) | 0.1033 | !G,G: 1.34(1.02, 1.75) | 0.0427 | A: 1.27(1.01, 1.59) |
| RS6693438 | 01: 091935762 | TGFBR3 | 0.0096 | A,A: 1.55(1.17, 2.05) | 0.0042 | A: 1.42(1.12, 1.81) | 0.0240 | !G,G: 2.38(1.21, 4.68) | 0.0272 | A: 1.3(1.03, 1.65) |
| RS2046736 | 01: 091947596 | TGFBR3 | 0.0195 | T,T: 1.47(1.12, 1.94) | 0.0076 | T: 1.37(1.09, 1.72) | 0.0429 | !C,C: 1.85(1.06, 3.22) | 0.0152 | T: 1.32(1.06, 1.65) |
| RS17513917 | 01: 091977785 | TGFBR3 | 0.0374 | T,T: 1.34(1.02, 1.76) | 0.0110 | T: 1.3(1.06, 1.58) | 0.0054 | !C,C: 1.85(1.25, 2.76) | 0.0029 | T: 1.35(1.11, 1.64) |
| RS2029356 | 01: 091981899 | TGFBR3 | 0.1231 | C,C: 1.31(1, 1.72) | 0.0472 | C: 1.25(1.01, 1.55) | 0.0007 | !T,T: 2.69(1.53, 4.75) | 0.0009 | C: 1.44(1.16, 1.79) |
| RS309058 | 01: 099174812 | PAP2D | 0.0030 | !A,A: 1.83(1.29, 2.61) | 0.0104 | G: 1.29(1.06, 1.57) | 0.1458 | G,G: 1.28(0.97, 1.67) | 0.0492 | G: 1.21(1, 1.46) |
| RS1376367 | 01: 105251544 | | 0.0157 | !A,G: 1.6(1.15, 2.22) | 0.0384 | A: 1.36(1.02, 1.82) | 0.0253 | !G,G: 3.66(1.28, 10.46) | 0.2778 | A: 1.18(0.89, 1.57) |
| RS10158153 | 01: 106780559 | | 0.0118 | !C,C: 2.48(1.32, 4.68) | 0.0275 | T: 1.29(1.03, 1.63) | 0.0140 | C,T: 1.52(1.14, 2.03) | 0.0861 | C: 1.23(0.97, 1.55) |
| RS1444038 | 01: 107782839 | NTNG1 | 0.0959 | G,G: 1.37(1.02, 1.83) | 0.0284 | G: 1.33(1.03, 1.71) | 0.1086 | !A,A: 1.87(0.93, 3.76) | 0.0479 | G: 1.28(1.01, 1.64) |
| RS1146335 | 01: 118804322 | | 0.0185 | C,C: 1.53(1.13, 2.06) | 0.0327 | C: 1.23(1.02, 1.49) | 0.0007 | !T,T: 1.77(1.31, 2.39) | 0.0036 | C: 1.32(1.1, 1.59) |
| RS1146324 | 01: 118814926 | | 0.0351 | A,A: 1.47(1.09, 1.98) | 0.0522 | A: 1.21(1, 1.47) | 0.0005 | !G,G: 1.8(1.33, 2.42) | 0.0022 | A: 1.34(1.11, 1.61) |
| RS1146320 | 01: 118817763 | | 0.0240 | T,T: 1.5(1.12, 2.02) | 0.0414 | T: 1.22(1.01, 1.48) | 0.0005 | !G,G: 1.8(1.33, 2.42) | 0.0026 | T: 1.33(1.11, 1.6) |
| RS1229119 | 01: 118825163 | | 0.0156 | T,T: 1.49(1.13, 1.96) | 0.0067 | T: 1.32(1.08, 1.6) | 0.0010 | !C,C: 1.9(1.34, 2.71) | 0.0012 | T: 1.37(1.13, 1.65) |
| RS1146313 | 01: 118827613 | | 0.0133 | A,A: 1.52(1.14, 2.04) | 0.0404 | A: 1.22(1.01, 1.48) | 0.0002 | !G,G: 1.92(1.4, 2.63) | 0.0006 | A: 1.38(1.15, 1.66) |
| RS12408596 | 01: 118836057 | | 0.1172 | T,T: 1.34(1.01, 1.79) | 0.0409 | T: 1.29(1.01, 1.64) | 0.0032 | !C,C: 3.16(1.5, 6.63) | 0.0130 | T: 1.33(1.06, 1.66) |
| RS17257972 | 01: 118908135 | | 0.0022 | !A,G: 1.55(1.18, 2.03) | 0.1459 | A: 1.16(0.96, 1.4) | 0.0002 | !G,G: 1.86(1.38, 2.5) | 0.0008 | A: 1.37(1.14, 1.65) |
| RS2798445 | 01: 118931337 | | 0.0590 | T,T: 1.39(1.06, 1.83) | 0.0438 | T: 1.23(1.01, 1.5) | 0.0209 | !C,C: 1.57(1.07, 2.29) | 0.0066 | T: 1.3(1.08, 1.58) |
| RS4329498 | 01: 118935132 | | 0.0164 | C,C: 1.54(1.13, 2.09) | 0.0655 | C: 1.2(0.99, 1.45) | 4.68E−05 | !T,T: 1.96(1.45, 2.65) | 0.0004 | C: 1.4(1.16, 1.68) |
| RS1009458 | 01: 118959432 | LOC644094 | 0.0595 | C,C: 1.39(1.06, 1.83) | 0.0343 | C: 1.24(1.02, 1.51) | 0.0236 | !T,T: 1.57(1.07, 2.29) | 0.0076 | C: 1.3(1.07, 1.57) |
| RS10923658 | 01: 119074302 | | 0.0386 | G,G: 1.53(1.1, 2.13) | 0.0322 | G: 1.23(1.02, 1.49) | 0.0021 | !A,A: 1.63(1.24, 2.15) | 0.0057 | G: 1.31(1.08, 1.57) |
| RS1766807 | 01: 119208283 | | 0.0603 | A,A: 1.43(1.05, 1.94) | 0.0181 | A: 1.4(1.06, 1.84) | 0.0514 | A,A: 1.36(1.03, 1.79) | 0.0176 | A: 1.34(1.05, 1.7) |
| RS4845771 | 01: 150697304 | | 0.0938 | C,C: 1.34(1.02, 1.75) | 0.0300 | C: 1.26(1.03, 1.54) | 0.0113 | !T,T: 1.79(1.14, 2.81) | 0.5829 | C: 1.06(0.87, 1.29) |
| RS4656888 | 01: 158395295 | ATP1A4 | 0.0428 | !A,G: 1.37(1.05, 1.79) | 0.2067 | A: 1.13(0.94, 1.37) | 0.0096 | !A,G: 1.46(1.12, 1.9) | 0.2220 | G: 1.12(0.93, 1.35) |
| RS10494387 | 01: 161524157 | | 0.0040 | !A,A: 1.69(1.23, 2.32) | 0.0259 | G: 1.24(1.03, 1.5) | 0.0457 | A,G: 1.39(1.07, 1.81) | 0.7774 | A: 1.03(0.86, 1.24) |
| RS10800048 | 01: 163014032 | PBX1 | 0.0589 | !C,C: 1.81(1.06, 3.08) | 0.0319 | A: 1.28(1.03, 1.6) | 0.0436 | !A,C: 1.41(1.08, 1.86) | 0.1247 | A: 1.19(0.96, 1.47) |
| RS545833 | 01: 166956564 | DPT | 0.0046 | !C,C: 1.57(1.2, 2.06) | 0.0022 | T: 1.42(1.14, 1.77) | 0.0464 | C,T: 1.38(1.05, 1.8) | 0.5580 | T: 1.07(0.87, 1.32) |
| RS859398 | 01: 173642425 | TNR | 0.0229 | G,G: 1.62(1.08, 2.42) | 0.4183 | G: 1.09(0.89, 1.33) | 0.1073 | !A,A: 1.29(0.99, 1.69) | 0.0372 | G: 1.23(1.02, 1.49) |
| RS10913008 | 01: 173798257 | | 0.0312 | A,G: 1.6(1.12, 2.27) | 0.0291 | G: 1.43(1.04, 1.97) | 0.0261 | A,G: 1.56(1.12, 2.17) | 0.0181 | G: 1.45(1.07, 1.95) |
| RS1908645 | 01: 185539963 | | 2.56E−05 | A,A: 2(1.47, 2.72) | 0.0019 | A: 1.36(1.12, 1.64) | 0.0229 | A,A: 1.47(1.08, 2) | 0.2402 | A: 1.12(0.93, 1.35) |
| RS1170919 | 01: 188882260 | LOC440704 | 0.0477 | C,T: 1.41(1.07, 1.85) | 0.2920 | T: 1.12(0.91, 1.38) | 0.0300 | !C,C: 1.36(1.05, 1.77) | 0.0086 | T: 1.31(1.07, 1.59) |
| RS6428282 | 01: 193533695 | | 0.0172 | A,C: 1.48(1.13, 1.94) | 0.4010 | A: 1.09(0.9, 1.32) | 0.0345 | !C,C: 1.45(1.09, 1.95) | 0.0891 | A: 1.18(0.98, 1.42) |
| RS11584286 | 01: 198686624 | | 0.0364 | G,G: 6.1(1.08, 34.3) | 0.6412 | G: 1.08(0.8, 1.47) | 0.0339 | !A,G: 1.57(1.11, 2.24) | 0.0367 | A: 1.41(1.02, 1.93) |
| RS2887480 | 01: 203147961 | NFASC | 0.1081 | !C,C: 1.37(0.98, 1.91) | 0.0410 | T: 1.23(1.01, 1.48) | 0.1248 | !C,C: 1.32(0.96, 1.82) | 0.0479 | T: 1.21(1.01, 1.46) |
| RS1567190 | 01: 205702412 | CR2 | 0.0241 | C,T: 1.4(1.07, 1.84) | 0.2073 | T: 1.13(0.94, 1.37) | 0.0141 | C,T: 1.46(1.12, 1.89) | 0.3978 | C: 1.09(0.9, 1.31) |
| RS2785622 | 01: 206281127 | PLXNA2 | 0.1110 | T,T: 1.36(1.02, 1.81) | 0.0500 | T: 1.29(1, 1.65) | 0.0225 | T,T: 1.4(1.06, 1.84) | 0.0087 | T: 1.39(1.09, 1.77) |
| RS841865 | 01: 206292532 | PLXNA2 | 0.0372 | A,A: 1.45(1.09, 1.94) | 0.0136 | A: 1.37(1.07, 1.76) | 0.0881 | A,A: 1.28(0.97, 1.69) | 0.0374 | A: 1.29(1.02, 1.63) |
| RS2921129 | 01: 210563370 | PPP2R5A | 0.0052 | C,C: 3.21(1.4, 7.36) | 0.0062 | C: 1.41(1.1, 1.8) | 0.0366 | C,T: 1.4(1.07, 1.85) | 0.2741 | C: 1.14(0.91, 1.43) |
| RS3121291 | 01: 210572483 | PPP2R5A | 0.0052 | T,T: 3.21(1.4, 7.36) | 0.0062 | T: 1.41(1.1, 1.8) | 0.0366 | G,T: 1.4(1.07, 1.85) | 0.2741 | T: 1.14(0.91, 1.43) |
| RS701907 | 01: 210588027 | PPP2R5A | 0.0056 | C,C: 3.21(1.4, 7.36) | 0.0075 | C: 1.4(1.09, 1.79) | 0.0366 | C,T: 1.4(1.07, 1.85) | 0.2741 | C: 1.14(0.91, 1.43) |
| RS792442 | 01: 210592911 | PPP2R5A | 0.0024 | G,G: 3.18(1.52, 6.67) | 0.0059 | G: 1.4(1.1, 1.77) | 0.0438 | A,G: 1.4(1.07, 1.84) | 0.1656 | G: 1.17(0.94, 1.45) |
| RS1655996 | 01: 211558022 | | 0.1236 | T,T: 1.37(0.96, 1.96) | 0.0496 | T: 1.22(1, 1.48) | 0.0096 | T,T: 1.68(1.18, 2.39) | 0.0067 | T: 1.3(1.08, 1.57) |
| RS4615815 | 01: 212670859 | PTPN14 | 0.0711 | A,A: 1.48(1.03, 2.14) | 0.0304 | A: 1.24(1.02, 1.5) | 0.0142 | !G,G: 1.48(1.14, 1.93) | 0.0105 | A: 1.29(1.06, 1.56) |
| RS4390149 | 01: 214057466 | USH2A | 0.0462 | !A,G: 1.35(1.03, 1.76) | 0.5547 | A: 1.06(0.88, 1.29) | 0.1268 | !G,G: 1.27(0.96, 1.67) | 0.0415 | A: 1.21(1.01, 1.46) |
| RS10495071 | 01: 215958401 | SPATA17 | 0.0864 | A,A: 1.31(0.98, 1.76) | 0.0448 | A: 1.31(1.01, 1.71) | 0.0052 | !A,G: 1.6(1.2, 2.14) | 0.0128 | A: 1.36(1.07, 1.72) |
| RS7546839 | 01: 216133915 | | 0.0573 | G,T: 1.47(1.07, 2.03) | 0.0441 | G: 1.35(1.01, 1.79) | 0.0604 | !T,T: 1.44(1.06, 1.96) | 0.0203 | G: 1.39(1.05, 1.83) |
| RS7536586 | 01: 217417479 | LYPLAL1 | 0.0969 | G,G: 1.47(0.96, 2.23) | 0.0293 | G: 1.26(1.03, 1.54) | 0.0348 | !A,G: 1.35(1.03, 1.76) | 1.0000 | G: 1(0.82, 1.23) |
| RS1890758 | 01: 220315446 | | 0.0325 | G,G: 1.56(1.1, 2.19) | 0.0192 | G: 1.26(1.04, 1.53) | 0.0087 | !A,G: 1.49(1.15, 1.94) | 0.9620 | G: 1.01(0.84, 1.22) |
| RS1591912 | 01: 220322763 | | 0.0405 | G,G: 1.53(1.09, 2.14) | 0.0283 | G: 1.24(1.02, 1.5) | 0.0379 | !A,G: 1.38(1.06, 1.79) | 0.7032 | G: 1.04(0.86, 1.25) |
| RS17163737 | 01: 221369774 | TLR5 | 0.0203 | G,G: 10.52(0.58, 190.81) | 0.0273 | G: 1.47(1.05, 2.06) | 0.0639 | !T,T: 1.51(1.06, 2.14) | 0.0218 | G: 1.46(1.06, 2.02) |
| RS10916197 | 01: 222296930 | | 0.0461 | T,T: 1.45(1.04, 2.02) | 0.0174 | T: 1.26(1.04, 1.53) | 0.0956 | !C,C: 1.34(1.01, 1.78) | 0.0372 | T: 1.22(1.01, 1.47) |
| RS7517868 | 01: 222318289 | | 0.0138 | G,G: 1.41(1.06, 1.88) | 0.0038 | G: 1.33(1.1, 1.61) | 0.0715 | !A,A: 1.45(1.04, 2.01) | 0.0333 | G: 1.23(1.02, 1.48) |
| RS9435806 | 01: 227522499 | C1ORF96 | 0.0827 | !C,C: 1.39(0.99, 1.96) | 0.0281 | T: 1.24(1.02, 1.5) | 0.0720 | T,T: 1.35(1.02, 1.78) | 0.0233 | T: 1.24(1.03, 1.5) |
| RS1890350 | 01: 228960988 | CAPN9 | 0.0333 | !A,A: 1.52(1.1, 2.11) | 0.1202 | G: 1.17(0.96, 1.41) | 0.0184 | !A,A: 1.46(1.08, 1.98) | 0.0048 | G: 1.3(1.08, 1.57) |
| RS4847011 | 01: 228966634 | CAPN9 | 0.0450 | !C,C: 1.74(1.12, 2.7) | 0.0822 | T: 1.2(0.98, 1.46) | 0.0438 | !C,C: 1.61(1.1, 2.38) | 0.0307 | T: 1.24(1.02, 1.51) |
| RS3828128 | 01: 228995933 | CAPN9 | 0.0388 | !G,G: 1.97(1.13, 3.44) | 0.3792 | A: 1.11(0.89, 1.37) | 0.0084 | !G,G: 2.23(1.28, 3.88) | 0.0107 | A: 1.31(1.07, 1.62) |
| RS1202524 | 01: 229017795 | | 0.0133 | !A,A: 2.02(1.24, 3.29) | 0.1292 | G: 1.17(0.96, 1.44) | 0.0075 | !A,A: 1.82(1.18, 2.81) | 0.0032 | G: 1.35(1.11, 1.64) |
| RS722732 | 01: 229027992 | | 0.0461 | !A,A: 1.91(1.13, 3.24) | 0.2176 | G: 1.15(0.93, 1.41) | 0.0064 | !A,A: 1.91(1.21, 3.03) | 0.0026 | G: 1.36(1.11, 1.66) |
| RS946131 | 01: 230505719 | | 0.0141 | C,T: 1.55(1.14, 2.1) | 0.0815 | T: 1.26(0.98, 1.63) | 0.0140 | !T,T: 2.24(1.07, 4.69) | 0.8097 | T: 1.04(0.82, 1.31) |
| RS16835237 | 01: 235653007 | RYR2 | 0.0162 | T,T: 12.46(0.7, 221.86) | 0.0331 | T: 1.43(1.03, 1.98) | 0.0539 | C,T: 1.52(1.07, 2.16) | 0.0470 | T: 1.4(1.01, 1.93) |
| RS790900 | 01: 236023932 | RYR2 | 0.0218 | !T,T: 1.39(1.06, 1.82) | 0.0053 | G: 1.34(1.09, 1.64) | 0.1154 | !T,T: 1.31(1.01, 1.7) | 0.0466 | G: 1.22(1.01, 1.49) |
| RS12732104 | 01: 236455086 | | 0.0134 | C,T: 1.47(1.12, 1.93) | 0.7951 | T: 1.03(0.84, 1.27) | 0.0215 | !T,T: 1.69(1.14, 2.52) | 0.0121 | C: 1.29(1.06, 1.56) |
| RS6682403 | 01: 236483906 | | 0.0006 | A,G: 1.65(1.26, 2.17) | 0.0440 | A: 1.22(1.01, 1.48) | 0.0057 | !A,A: 1.74(1.23, 2.46) | 0.0059 | G: 1.3(1.08, 1.57) |
| RS4658504 | 01: 241107670 | | 0.0052 | C,C: 1.57(1.19, 2.08) | 0.0176 | C: 1.27(1.04, 1.54) | 0.0084 | C,C: 1.51(1.16, 1.97) | 0.0137 | C: 1.28(1.06, 1.55) |
| RS1173656 | 01: 243689684 | | 0.0198 | G,G: 2.06(1.09, 3.91) | 0.9080 | G: 1.01(0.81, 1.27) | 0.0320 | G,G: 1.76(1.01, 3.06) | 0.9557 | G: 1.01(0.81, 1.25) |
| RS7561317 | 02: 000634953 | | 0.0275 | !A,G: 1.52(1.12, 2.06) | 0.0532 | G: 1.28(1, 1.65) | 0.0483 | !A,G: 1.42(1.07, 1.89) | 0.0763 | G: 1.26(0.98, 1.61) |
| RS357958 | 02: 003761591 | | 0.0350 | G,G: 1.54(1.09, 2.18) | 0.1570 | G: 1.15(0.95, 1.4) | 0.0112 | A,G: 1.47(1.13, 1.9) | 0.1851 | G: 1.14(0.94, 1.37) |
| RS2249380 | 02: 003767258 | | 0.0654 | C,C: 1.37(1.03, 1.82) | 0.0211 | C: 1.26(1.04, 1.52) | 0.0274 | !A,A: 1.57(1.12, 2.19) | 0.0227 | C: 1.25(1.03, 1.5) |
| RS1564332 | 02: 004592098 | | 0.0382 | !A,A: 1.53(1.1, 2.12) | 0.0326 | G: 1.23(1.02, 1.49) | 0.0057 | !A,G: 1.47(1.13, 1.91) | 0.0901 | G: 1.17(0.98, 1.41) |
| RS2163027 | 02: 005890517 | | 0.0606 | G,G: 1.68(1.04, 2.73) | 0.0296 | G: 1.27(1.03, 1.56) | 0.0320 | !T,T: 1.4(1.08, 1.82) | 0.0109 | G: 1.3(1.06, 1.58) |
| RS2709460 | 02: 005896443 | | 0.1000 | !C,C: 1.34(1.02, 1.75) | 0.0378 | T: 1.25(1.01, 1.54) | 0.0442 | !C,C: 1.37(1.06, 1.78) | 0.0129 | T: 1.29(1.06, 1.57) |
| RS2357889 | 02: 011117422 | | 0.0028 | !T,T: 1.63(1.23, 2.16) | 0.0019 | C: 1.36(1.12, 1.65) | 0.1053 | C,C: 1.43(0.99, 2.07) | 0.0420 | C: 1.22(1.01, 1.48) |
| RS983037 | 02: 012997671 | | 0.0059 | A,A: 1.51(1.15, 1.99) | 0.0012 | A: 1.39(1.14, 1.69) | 0.0032 | !C,C: 1.97(1.31, 2.96) | 0.0052 | A: 1.32(1.09, 1.6) |
| RS12692488 | 02: 013052254 | | 0.0081 | A,A: 1.54(1.17, 2.03) | 0.0079 | A: 1.31(1.08, 1.6) | 0.0018 | !G,G: 2.01(1.31, 3.07) | 0.1630 | A: 1.15(0.95, 1.4) |
| RS1818595 | 02: 013085683 | | 0.0322 | !G,G: 1.57(1.11, 2.22) | 0.0969 | A: 1.18(0.97, 1.43) | 0.0042 | A,A: 1.55(1.18, 2.05) | 0.0014 | A: 1.36(1.13, 1.64) |
| RS1976809 | 02: 017329493 | LOC645521 | 0.0573 | G,G: 1.58(1.02, 2.43) | 0.0254 | G: 1.26(1.03, 1.54) | 0.0244 | !A,G: 1.44(1.11, 1.87) | 0.7034 | A: 1.04(0.86, 1.25) |
| RS7583409 | 02: 025344560 | DNMT3A | 0.0311 | G,G: 1.76(1.11, 2.79) | 0.0195 | G: 1.28(1.04, 1.57) | 0.0322 | !A,G: 1.41(1.09, 1.84) | 0.3871 | A: 1.1(0.9, 1.34) |
| RS12621279 | 02: 031195804 | GALNT14 | 0.1066 | T,T: 1.29(0.98, 1.69) | 0.0267 | T: 1.25(1.03, 1.53) | 0.0164 | !C,T: 1.45(1.12, 1.89) | 0.6971 | T: 1.04(0.86, 1.26) |
| RS2568465 | 02: 032715454 | TTC27 | 0.0560 | C,C: 1.38(1.05, 1.81) | 0.0188 | C: 1.27(1.04, 1.56) | 0.0334 | C,C: 1.42(1.09, 1.84) | 0.0207 | C: 1.27(1.04, 1.56) |
| RS1000951 | 02: 033115912 | LTBP1 | 0.0263 | G,G: 1.52(1.12, 2.07) | 0.0115 | G: 1.43(1.09, 1.89) | 0.0663 | !A,G: 1.42(1.06, 1.92) | 0.0494 | G: 1.3(1, 1.68) |
| RS10490450 | 02: 033186442 | LTBP1 | 0.0666 | T,T: 1.4(1.04, 1.88) | 0.0206 | T: 1.36(1.05, 1.77) | 0.0107 | T,T: 1.54(1.16, 2.04) | 0.0051 | T: 1.43(1.11, 1.82) |
| RS1446297 | 02: 038061737 | FAM82A | 0.0106 | A,G: 1.46(1.11, 1.92) | 0.7131 | G: 1.04(0.85, 1.28) | 0.0307 | !A,A: 1.39(1.07, 1.81) | 0.0093 | G: 1.31(1.07, 1.61) |
| RS2373327 | 02: 038075590 | FAM82A | 0.0483 | A,G: 1.38(1.05, 1.81) | 0.6379 | G: 1.05(0.86, 1.29) | 0.0482 | !A,A: 1.34(1.03, 1.74) | 0.0147 | G: 1.29(1.05, 1.58) |
| RS6749927 | 02: 043338060 | THADA | 0.0118 | !A,G: 1.49(1.14, 1.96) | 0.8041 | A: 1.03(0.85, 1.25) | 0.0076 | G,G: 1.82(1.24, 2.67) | 0.0171 | G: 1.26(1.04, 1.53) |
| RS9309199 | 02: 050867151 | NRXN1 | 0.0168 | !G,T: 1.45(1.11, 1.9) | 0.4377 | G: 1.08(0.9, 1.31) | 0.1110 | !T,T: 1.33(0.98, 1.79) | 0.0433 | G: 1.21(1.01, 1.46) |
| RS10164725 | 02: 053559728 | | 0.0533 | !T,T: 1.48(1.07, 2.04) | 0.0258 | C: 1.4(1.04, 1.88) | 0.0007 | !C,C: 8.98(1.65, 48.76) | 0.9426 | C: 1.01(0.76, 1.34) |
| RS13405071 | 02: 057946258 | LOC647021, | 0.0703 | !C,T: 1.65(1.05, 2.58) | 0.0444 | T: 1.55(1.02, 2.36) | 0.0249 | !C,T: 1.5(1.04, 2.18) | 0.2253 | T: 1.25(0.89, 1.76) |
| | | LOC652202 | | | | | | | | |
| RS11887238 | 02: 057996771 | | 0.0117 | !A,C: 1.88(1.17, 3.03) | 0.0126 | A: 1.78(1.13, 2.8) | 0.0207 | !A,C: 1.67(1.08, 2.58) | 0.1316 | A: 1.37(0.92, 2.03) |
| RS6758100 | 02: 058968243 | FLJ30838, | 0.0391 | !T,T: 1.93(1.15, 3.24) | 0.1037 | C: 1.19(0.97, 1.46) | 0.0113 | C,T: 1.49(1.15, 1.94) | 0.2509 | T: 1.12(0.92, 1.37) |
| | | LOC647029, | | | | | | | | |
| | | LOC652214 | | | | | | | | |
| RS2422008 | 02: 063634109 | | 0.0318 | G,G: 1.56(1.09, 2.22) | 0.0142 | G: 1.28(1.05, 1.55) | 0.0455 | G,T: 1.39(1.07, 1.81) | 0.7048 | G: 1.04(0.86, 1.25) |
| RS1477054 | 02: 065758368 | FLJ16124 | 0.0472 | A,G: 1.67(1.1, 2.54) | 0.0560 | A: 1.46(1, 2.14) | 0.0414 | A,A: 13.53(0.76, 240.91) | 0.1522 | A: 1.35(0.9, 2.02) |
| RS2872075 | 02: 069922139 | GMCL1 | 0.0077 | !T,T: 1.52(1.14, 2.04) | 0.0797 | G: 1.19(0.98, 1.44) | 0.0314 | G,G: 1.53(1.11, 2.11) | 0.0237 | G: 1.24(1.03, 1.5) |
| RS10496175 | 02: 070007352 | MXD1 | 0.0471 | C,T: 1.37(1.04, 1.81) | 0.4912 | C: 1.09(0.87, 1.36) | 0.1041 | !T,T: 1.32(1.01, 1.71) | 0.0369 | C: 1.26(1.02, 1.56) |
| RS11893500 | 02: 070007691 | MXD1 | 0.0252 | C,T: 1.44(1.1, 1.89) | 0.3031 | C: 1.11(0.92, 1.34) | 0.0446 | C,C: 1.53(1.09, 2.16) | 0.0416 | C: 1.21(1.01, 1.46) |
| RS6741449 | 02: 070010093 | MXD1 | 0.0234 | A,G: 1.44(1.1, 1.89) | 0.2596 | G: 1.12(0.92, 1.36) | 0.0476 | G,G: 1.53(1.09, 2.15) | 0.0418 | G: 1.21(1.01, 1.46) |
| RS7580273 | 02: 071130206 | | 0.0197 | !A,A: 1.47(1.12, 1.92) | 0.0176 | G: 1.29(1.05, 1.6) | 0.0375 | !G,G: 1.87(1.1, 3.18) | 0.4932 | A: 1.08(0.88, 1.33) |
| RS11126315 | 02: 071144566 | | 0.0220 | !T,T: 1.46(1.11, 1.91) | 0.0204 | C: 1.29(1.04, 1.59) | 0.0376 | !C,C: 1.87(1.1, 3.18) | 0.4937 | T: 1.08(0.88, 1.33) |
| RS898238 | 02: 071867166 | | 0.0156 | !A,G: 1.55(1.14, 2.11) | 0.0146 | A: 1.37(1.07, 1.77) | 0.0305 | A,A: 1.47(1.1, 1.96) | 0.0085 | A: 1.41(1.1, 1.82) |
| RS10496214 | 02: 079271451 | | 0.0116 | !G,T: 1.65(1.08, 2.52) | 0.1303 | T: 1.38(0.93, 2.05) | 0.0025 | T,T: 1.93(1.26, 2.96) | 0.0017 | T: 1.92(1.27, 2.9) |
| RS17016549 | 02: 079286292 | | 0.0081 | G,G: 10.52(0.58, 190.81) | 0.3316 | A: 1.21(0.83, 1.77) | 0.0022 | A,A: 1.92(1.27, 2.91) | 0.0016 | A: 1.9(1.27, 2.84) |
| RS1549765 | 02: 079670651 | CTNNA2 | 0.0511 | !C,C: 2.54(1.07, 6.04) | 0.0441 | T: 1.3(1.01, 1.68) | 0.0366 | C,T: 1.42(1.07, 1.89) | 0.1384 | C: 1.21(0.95, 1.54) |
| RS2902106 | 02: 079960523 | CTNNA2 | 0.0210 | !G,G: 2.52(1.25, 5.11) | 0.0257 | A: 1.34(1.04, 1.72) | 0.0245 | !G,G: 3.14(1.29, 7.66) | 0.2526 | A: 1.16(0.91, 1.46) |
| RS333228 | 02: 107997483 | SLC5A7 | 0.0535 | A,A: 1.34(1.02, 1.76) | 0.0156 | A: 1.32(1.05, 1.64) | 0.0208 | A,A: 1.42(1.09, 1.85) | 0.0072 | A: 1.35(1.09, 1.67) |
| RS1384795 | 02: 111488118 | ACOXL | 0.0916 | !G,G: 1.44(1.02, 2.04) | 0.0361 | A: 1.41(1.02, 1.94) | 0.0327 | !G,G: 1.52(1.1, 2.09) | 0.0096 | A: 1.48(1.11, 1.98) |
| RS2062374 | 02: 114612733 | | 0.0250 | !A,A: 2.56(1.23, 5.34) | 0.3285 | G: 1.13(0.89, 1.44) | 0.0098 | !A,A: 2.64(1.28, 5.45) | 0.5577 | G: 1.08(0.86, 1.35) |
| RS10864971 | 02: 118717122 | | 0.0806 | !C,C: 1.57(1.02, 2.43) | 0.0474 | T: 1.23(1.01, 1.5) | 0.0131 | T,T: 1.49(1.14, 1.96) | 0.0343 | T: 1.23(1.02, 1.49) |
| RS1371562 | 02: 119449418 | MARCO | 0.0426 | !G,G: 1.46(1.04, 2.05) | 0.3284 | T: 1.11(0.91, 1.34) | 0.0495 | !G,G: 1.46(1.04, 2.03) | 0.2763 | T: 1.11(0.92, 1.34) |
| RS2422392 | 02: 119514743 | | 0.0300 | !C,C: 1.42(1.08, 1.86) | 0.0084 | T: 1.35(1.08, 1.68) | 0.0421 | !C,C: 1.39(1.07, 1.81) | 0.0377 | T: 1.24(1.01, 1.53) |
| RS4662817 | 02: 128908256 | | 0.0134 | A,G: 1.6(1.16, 2.19) | 0.0465 | G: 1.31(1.01, 1.7) | 0.0609 | !A,A: 1.37(1.04, 1.81) | 0.0198 | G: 1.33(1.05, 1.68) |
| RS714527 | 02: 128920942 | | 0.0261 | A,G: 1.57(1.1, 2.23) | 0.1425 | G: 1.26(0.93, 1.71) | 0.0308 | !A,A: 1.49(1.11, 2.01) | 0.0099 | G: 1.42(1.09, 1.86) |
| RS12611515 | 02: 133258045 | NAP5 | 0.0391 | T,T: 1.39(1.06, 1.82) | 0.0125 | T: 1.3(1.06, 1.59) | 0.0218 | C,T: 1.35(1.03, 1.76) | 0.9579 | T: 1.01(0.82, 1.24) |
| RS579076 | 02: 134983030 | TMEM163 | 0.1241 | C,C: 1.39(0.95, 2.04) | 0.0464 | C: 1.22(1, 1.49) | 0.0332 | C,C: 1.64(1.1, 2.43) | 0.2391 | C: 1.12(0.93, 1.36) |
| RS6734966 | 02: 135262217 | | 0.0262 | !T,T: 1.41(1.05, 1.88) | 0.0101 | C: 1.4(1.09, 1.81) | 0.0861 | !T,T: 1.39(1.02, 1.89) | 0.0321 | C: 1.37(1.04, 1.82) |
| RS2176717 | 02: 136616329 | | 0.0027 | !T,T: 1.82(1.27, 2.62) | 0.0013 | C: 1.38(1.13, 1.67) | 0.0108 | C,C: 1.46(1.09, 1.97) | 0.0031 | C: 1.32(1.1, 1.59) |
| RS882300 | 02: 136692725 | | 0.0042 | !A,A: 1.8(1.25, 2.59) | 0.0026 | G: 1.35(1.11, 1.64) | 0.0106 | G,G: 1.48(1.1, 1.98) | 0.0030 | G: 1.33(1.1, 1.6) |
| RS10496767 | 02: 137581892 | KIAA1679 | 0.0201 | T,T: 2.63(1.23, 5.61) | 0.4749 | T: 1.1(0.87, 1.38) | 0.0715 | !C,C: 1.3(1, 1.68) | 0.0252 | T: 1.26(1.03, 1.55) |
| RS1370327 | 02: 142185062 | LRP1B | 0.0349 | !G,G: 1.5(1.1, 2.04) | 0.0371 | A: 1.23(1.01, 1.48) | 0.0145 | !G,G: 1.52(1.13, 2.05) | 0.0602 | A: 1.2(0.99, 1.44) |
| RS6744928 | 02: 142549797 | LRP1B | 0.0401 | !C,C: 1.44(1.09, 1.9) | 0.0257 | T: 1.25(1.03, 1.52) | 0.1288 | T,T: 1.36(0.95, 1.96) | 0.0493 | T: 1.21(1, 1.46) |
| RS354690 | 02: 143534188 | | 0.0115 | T,T: 1.74(1.2, 2.52) | 0.0116 | T: 1.29(1.06, 1.57) | 0.0207 | T,T: 1.59(1.15, 2.21) | 0.0296 | T: 1.23(1.02, 1.48) |
| RS3768655 | 02: 152422269 | CACNB4 | 0.0564 | !A,A: 1.57(1.06, 2.32) | 0.0175 | C: 1.56(1.08, 2.25) | 0.0076 | C,C: 15.68(0.89, 275.38) | 0.0234 | C: 1.47(1.05, 2.05) |
| RS3791626 | 02: 153336153 | | 0.0370 | C,T: 1.41(1.05, 1.9) | 0.3765 | C: 1.12(0.88, 1.44) | 0.0225 | T,T: 1.39(1.04, 1.85) | 0.0114 | T: 1.4(1.08, 1.81) |
| RS10179554 | 02: 158778702 | LOC130940 | 0.0157 | !T,T: 2.01(1.16, 3.47) | 0.0094 | C: 2.01(1.18, 3.43) | 0.0394 | C,T: 1.81(1.05, 3.14) | 0.0845 | C: 1.62(0.96, 2.75) |
| RS888629 | 02: 159931293 | BAZ2B | 0.0600 | !A,A: 1.4(0.99, 1.98) | 0.0282 | G: 1.44(1.04, 1.98) | 0.0051 | !A,A: 1.76(1.24, 2.5) | 0.0020 | G: 1.66(1.2, 2.3) |
| RS2042772 | 02: 160370304 | LY75 | 0.1190 | !T,T: 1.26(0.96, 1.66) | 0.0455 | C: 1.26(1.01, 1.58) | 0.0595 | !T,T: 1.34(1.03, 1.74) | 0.0216 | C: 1.28(1.04, 1.58) |
| RS1990842 | 02: 169916281 | LRP2 | 0.0223 | A,A: 1.36(1.04, 1.79) | 0.0067 | A: 1.33(1.08, 1.63) | 0.0214 | A,A: 1.42(1.09, 1.85) | 0.0528 | A: 1.22(1, 1.48) |
| RS4145305 | 02: 174694824 | PTD004 | 0.0935 | !A,A: 1.36(1, 1.85) | 0.0267 | G: 1.36(1.04, 1.79) | 0.0152 | A,G: 1.54(1.14, 2.06) | 0.0215 | G: 1.34(1.05, 1.71) |
| RS2119137 | 02: 174725960 | PTD004 | 0.0115 | G,G: 1.8(1.21, 2.66) | 0.0160 | G: 1.28(1.05, 1.55) | 0.0194 | !A,A: 1.39(1.07, 1.82) | 0.0046 | G: 1.32(1.09, 1.6) |
| RS2588868 | 02: 177709231 | | 0.0538 | !C,C: 1.42(1.04, 1.92) | 0.0155 | T: 1.4(1.07, 1.84) | 0.1189 | !C,C: 1.42(1.02, 1.98) | 0.0478 | T: 1.38(1.01, 1.88) |
| RS4894058 | 02: 179446750 | FLJ39502 | 0.0531 | C,C: 1.67(1.06, 2.63) | 0.0243 | C: 1.27(1.03, 1.56) | 0.0984 | !T,T: 1.3(1, 1.69) | 0.0322 | C: 1.24(1.02, 1.51) |
| RS6716304 | 02: 179454567 | FLJ39502 | 0.0810 | G,G: 1.61(0.98, 2.67) | 0.0330 | G: 1.26(1.02, 1.55) | 0.0369 | !A,A: 1.33(1.03, 1.73) | 0.0127 | G: 1.29(1.06, 1.58) |
| RS1028179 | 02: 180044166 | ZNF533 | 0.0953 | T,T: 1.35(1.02, 1.78) | 0.0402 | T: 1.29(1.02, 1.63) | 0.0237 | !C,T: 1.38(1.04, 1.81) | 0.6939 | T: 1.05(0.84, 1.3) |
| RS7588905 | 02: 184246849 | | 0.1543 | A,A: 1.26(0.96, 1.65) | 0.0480 | A: 1.23(1, 1.51) | 0.0039 | !G,G: 2.09(1.3, 3.37) | 0.2216 | A: 1.13(0.93, 1.38) |
| RS1451487 | 02: 199703113 | | 0.0568 | A,A: 1.42(1.07, 1.89) | 0.0276 | A: 1.33(1.04, 1.7) | 0.0105 | A,A: 1.47(1.1, 1.95) | 0.0040 | A: 1.46(1.13, 1.88) |
| RS2243822 | 02: 201273695 | | 0.0300 | G,G: 2.2(1.19, 4.07) | 0.0490 | G: 1.25(1, 1.56) | 0.0279 | G,G: 2.18(1.21, 3.93) | 0.0989 | G: 1.2(0.97, 1.49) |
| RS7577449 | 02: 208475230 | LOC389072 | 0.0262 | !C,T: 1.47(1.11, 1.94) | 0.1656 | T: 1.17(0.94, 1.45) | 0.0586 | T,T: 1.34(1.03, 1.74) | 0.0173 | T: 1.28(1.05, 1.56) |
| RS4597456 | 02: 212033473 | ERBB4 | 0.0367 | !G,G: 1.49(1.1, 2.03) | 0.0369 | A: 1.23(1.02, 1.49) | 0.0121 | !A,A: 1.57(1.15, 2.16) | 0.0996 | G: 1.17(0.97, 1.41) |
| RS280626 | 02: 215272346 | | 0.0261 | !T,T: 1.48(1.11, 1.98) | 0.0176 | C: 1.37(1.06, 1.76) | 0.0165 | C,T: 1.39(1.05, 1.84) | 0.6806 | C: 1.05(0.83, 1.32) |
| RS1250085 | 02: 216246901 | LOC646324 | 0.0247 | G,G: 1.38(1.05, 1.8) | 0.0078 | G: 1.33(1.08, 1.64) | 0.0184 | !A,A: 1.84(1.15, 2.92) | 0.3637 | G: 1.1(0.9, 1.34) |
| RS12624027 | 02: 217105664 | | 0.0207 | !T,T: 1.53(1.11, 2.1) | 0.1092 | C: 1.17(0.97, 1.42) | 0.0081 | C,C: 1.58(1.18, 2.13) | 0.0186 | C: 1.25(1.04, 1.51) |
| RS6435962 | 02: 217659070 | | 0.0152 | G,G: 1.48(1.13, 1.94) | 0.0168 | G: 1.28(1.05, 1.58) | 0.0233 | !A,G: 1.43(1.1, 1.87) | 0.0415 | G: 1.24(1.01, 1.53) |
| RS6728963 | 02: 218071277 | | 0.0171 | C,C: 16.37(0.94, 284.49) | 0.3063 | C: 1.2(0.86, 1.68) | 0.0510 | !A,A: 1.59(1.08, 2.32) | 0.0235 | C: 1.53(1.07, 2.19) |
| RS3791911 | 02: 218502228 | TNS1 | 0.0716 | T,T: 1.35(1, 1.81) | 0.0250 | T: 1.34(1.04, 1.74) | 0.1278 | T,T: 1.29(0.98, 1.7) | 0.0414 | T: 1.28(1.01, 1.62) |
| RS1955117 | 02: 221779509 | | 0.0839 | !A,G: 1.49(1.04, 2.13) | 0.0450 | A: 1.4(1.01, 1.95) | 0.0489 | G,G: 5.92(1.04, 33.78) | 0.6557 | G: 1.08(0.81, 1.44) |
| RS6436944 | 02: 231084564 | SP100 | 0.0100 | !T,T: 3.28(1.34, 8.05) | 0.0099 | C: 1.42(1.09, 1.86) | 0.0184 | C,T: 1.52(1.13, 2.05) | 0.0414 | T: 1.31(1.01, 1.7) |
| RS699660 | 02: 231093030 | | 0.0107 | !C,C: 3.47(1.32, 9.13) | 0.0116 | T: 1.41(1.08, 1.85) | 0.0049 | C,T: 1.56(1.16, 2.09) | 0.0548 | C: 1.3(1, 1.68) |
| RS836237 | 02: 231114924 | | 0.0107 | !T,T: 3.47(1.32, 9.13) | 0.0116 | C: 1.41(1.08, 1.85) | 0.0138 | C,T: 1.52(1.13, 2.03) | 0.0571 | T: 1.29(1, 1.67) |
| RS17354508 | 02: 231138221 | | 0.0088 | A,G: 1.49(1.13, 1.96) | 0.4145 | A: 1.1(0.89, 1.36) | 0.0451 | A,G: 1.35(1.03, 1.76) | 0.9171 | A: 1.01(0.83, 1.24) |
| RS6753112 | 02: 231895399 | ARMC9 | 0.0439 | C,T: 1.41(1.07, 1.84) | 0.5858 | C: 1.06(0.87, 1.28) | 0.0216 | !C,C: 1.58(1.07, 2.32) | 0.4596 | T: 1.08(0.89, 1.31) |
| RS7570061 | 02: 233685562 | LOC653796 | 0.0487 | G,G: 2.58(1.16, 5.75) | 0.1166 | G: 1.22(0.95, 1.56) | 0.0529 | G,G: 2.42(1.07, 5.48) | 0.0459 | G: 1.29(1.01, 1.64) |
| RS10210816 | 02: 234634378 | SPP2 | 0.0163 | !C,C: 1.99(1.15, 3.43) | 0.5402 | T: 1.07(0.86, 1.33) | 0.0390 | !T,T: 1.4(1.08, 1.82) | 0.0140 | C: 1.31(1.06, 1.61) |
| RS4340455 | 02: 235688109 | | 0.0060 | C,T: 1.56(1.18, 2.05) | 0.0997 | C: 1.19(0.97, 1.45) | 0.0360 | !C,C: 1.65(1.11, 2.45) | 0.1353 | T: 1.16(0.96, 1.41) |
| RS1367882 | 02: 235859450 | | 0.1085 | !A,A: 1.3(0.98, 1.71) | 0.0397 | G: 1.23(1.01, 1.5) | 0.0340 | !A,A: 1.41(1.08, 1.83) | 0.0502 | G: 1.21(1, 1.47) |
| RS4663795 | 02: 238397755 | FLJ40411 | 0.0954 | !A,A: 1.4(1.03, 1.9) | 0.0433 | G: 1.33(1.01, 1.76) | 0.0419 | A,G: 1.46(1.08, 1.98) | 0.0322 | G: 1.34(1.03, 1.74) |
| RS9287640 | 02: 239377004 | | 0.0283 | G,T: 1.4(1.05, 1.85) | 0.6059 | G: 1.07(0.85, 1.34) | 0.0256 | G,T: 1.38(1.05, 1.82) | 0.6051 | G: 1.06(0.85, 1.33) |
| RS1465820 | 02: 240270700 | | 0.0632 | !A,A: 1.38(1.04, 1.83) | 0.0233 | G: 1.25(1.03, 1.52) | 0.1063 | G,G: 1.41(0.96, 2.08) | 0.0349 | G: 1.23(1.02, 1.49) |
| RS3843388 | 03: 003255819 | | 0.0441 | !G,G: 3.25(1.11, 9.52) | 0.6537 | T: 1.08(0.8, 1.44) | 0.0006 | !G,T: 1.88(1.35, 2.62) | 0.0028 | T: 1.56(1.17, 2.09) |
| RS1682913 | 03: 003821526 | LRRN1 | 0.1389 | !T,T: 1.37(0.98, 1.93) | 0.0490 | G: 1.38(1.01, 1.88) | 0.0278 | G,T: 1.55(1.12, 2.16) | 0.0197 | G: 1.43(1.07, 1.93) |
| RS345233 | 03: 006745752 | | 0.0100 | C,T: 1.54(1.16, 2.05) | 0.0216 | T: 1.32(1.04, 1.66) | 0.0173 | T,T: 2.09(1.22, 3.57) | 0.0150 | T: 1.31(1.05, 1.63) |
| RS2633748 | 03: 006760430 | | 0.0320 | !T,T: 5.37(0.93, 31.15) | 0.0437 | G: 1.36(1.02, 1.83) | 0.1069 | G,G: 1.39(1.01, 1.89) | 0.0379 | G: 1.36(1.02, 1.8) |
| RS6785425 | 03: 007100901 | GRM7 | 0.0942 | A,A: 1.54(1.02, 2.33) | 0.0310 | A: 1.53(1.04, 2.26) | 0.0108 | !A,G: 1.8(1.17, 2.77) | 0.0110 | A: 1.7(1.13, 2.56) |
| RS2928078 | 03: 011034847 | SLC6A1 | 0.0532 | T,T: 1.51(1.08, 2.13) | 0.0456 | T: 1.22(1.01, 1.48) | 0.0703 | !C,C: 1.32(1, 1.75) | 0.0232 | T: 1.24(1.03, 1.5) |
| RS11128706 | 03: 014562321 | | 0.0071 | !C,T: 1.61(1.19, 2.18) | 0.0095 | C: 1.4(1.09, 1.81) | 0.0193 | T,T: 2.75(1.28, 5.88) | 0.0503 | T: 1.26(1, 1.59) |
| RS9847186 | 03: 025056861 | | 0.0008 | !A,G: 1.66(1.27, 2.17) | 0.1039 | A: 1.18(0.97, 1.43) | 0.0017 | G,G: 1.88(1.32, 2.67) | 0.0156 | G: 1.26(1.05, 1.52) |
| RS7642232 | 03: 033633002 | CLASP2 | 0.0015 | !C,C: 3.18(1.44, 7.02) | 0.0011 | A: 1.49(1.17, 1.9) | 0.0482 | !A,C: 1.42(1.07, 1.89) | 0.1234 | A: 1.21(0.96, 1.52) |
| RS7629699 | 03: 033922976 | | 0.0273 | G,G: 1.86(1.17, 2.96) | 0.0774 | G: 1.21(0.98, 1.48) | 0.0135 | G,G: 1.89(1.23, 2.9) | 0.0304 | G: 1.25(1.02, 1.52) |
| RS1304138 | 03: 039979370 | MYRIP | 0.0284 | A,A: 1.68(1.11, 2.54) | 0.2894 | A: 1.12(0.92, 1.36) | 0.0033 | A,A: 1.81(1.19, 2.77) | 0.5503 | A: 1.06(0.88, 1.29) |
| RS6789549 | 03: 040996267 | | 0.0381 | !A,A: 1.43(1.09, 1.88) | 0.0239 | G: 1.26(1.03, 1.53) | 0.0342 | !A,A: 1.42(1.09, 1.85) | 0.0452 | G: 1.22(1.01, 1.48) |
| RS4130762 | 03: 041012673 | | 0.0444 | !A,A: 1.42(1.07, 1.88) | 0.0158 | G: 1.27(1.05, 1.54) | 2.40E−05 | !A,A: 1.86(1.42, 2.44) | 0.0007 | G: 1.4(1.15, 1.69) |
| RS6599132 | 03: 041014911 | | 0.0729 | A,A: 1.38(1.04, 1.83) | 0.0302 | A: 1.24(1.02, 1.51) | 0.0164 | !G,G: 1.6(1.16, 2.21) | 0.0211 | A: 1.24(1.03, 1.5) |
| RS9861853 | 03: 043929897 | | 0.0895 | A,A: 1.37(1.02, 1.85) | 0.0263 | A: 1.35(1.04, 1.74) | 0.0419 | !A,G: 1.36(1.02, 1.8) | 0.6793 | A: 1.06(0.84, 1.33) |
| RS1495366 | 03: 055777789 | CAST1 | 0.0799 | A,A: 1.58(1.03, 2.41) | 0.0453 | A: 1.23(1.01, 1.51) | 0.0820 | A,A: 1.56(1.01, 2.42) | 0.0391 | A: 1.23(1.01, 1.5) |
| RS13063937 | 03: 060717098 | FHIT | 0.0918 | A,A: 1.33(1.01, 1.74) | 0.0368 | A: 1.27(1.02, 1.59) | 0.0347 | !C,C: 2.19(1.16, 4.14) | 0.0589 | A: 1.23(0.99, 1.52) |
| RS6804478 | 03: 060860180 | FHIT | 0.0455 | !A,A: 1.5(1.09, 2.06) | 0.0369 | G: 1.23(1.02, 1.49) | 0.0271 | !A,A: 1.47(1.09, 1.98) | 0.1211 | G: 1.16(0.97, 1.39) |
| RS2365054 | 03: 061044854 | FHIT | 0.0046 | !A,A: 4.98(1.26, 19.67) | 0.0047 | C: 1.5(1.13, 1.98) | 0.0061 | !A,A: 2.78(1.25, 6.15) | 0.8979 | C: 1.02(0.8, 1.32) |
| RS6806415 | 03: 061078649 | FHIT | 0.0052 | !T,T: 2.84(1.32, 6.07) | 0.0048 | C: 1.41(1.11, 1.79) | 0.0499 | C,T: 1.33(1.01, 1.77) | 0.8639 | T: 1.02(0.82, 1.28) |
| RS1821942 | 03: 061091049 | FHIT | 0.0093 | !G,G: 2.59(1.35, 4.97) | 0.0232 | A: 1.31(1.04, 1.64) | 0.0309 | A,G: 1.43(1.09, 1.87) | 0.1701 | G: 1.16(0.94, 1.44) |
| RS9311951 | 03: 065663422 | MAGI1 | 0.0130 | !G,T: 1.49(1.13, 1.96) | 0.4928 | T: 1.08(0.88, 1.33) | 0.0960 | G,G: 1.5(0.98, 2.3) | 0.0476 | G: 1.22(1.01, 1.48) |
| RS264096 | 03: 065720475 | MAGI1 | 0.0235 | !A,A: 1.48(1.11, 1.97) | 0.0177 | G: 1.35(1.05, 1.72) | 0.0631 | !A,A: 1.38(1.05, 1.8) | 0.0213 | G: 1.31(1.04, 1.64) |
| RS7615745 | 03: 074236974 | | 0.0007 | C,C: 1.75(1.3, 2.35) | 0.0047 | C: 1.32(1.09, 1.6) | 0.0038 | !C,T: 1.51(1.16, 1.96) | 0.4500 | T: 1.08(0.9, 1.3) |
| RS1569067 | 03: 074897220 | | 0.0497 | !C,T: 1.46(1.08, 1.98) | 0.0480 | T: 1.31(1.01, 1.69) | 0.0306 | !C,C: 2.84(1.15, 7.01) | 0.0358 | T: 1.31(1.02, 1.69) |
| RS12490683 | 03: 075461775 | LOC652408 | 0.0467 | !A,A: 1.57(1.07, 2.32) | 0.0216 | G: 1.26(1.03, 1.53) | 0.0021 | !A,G: 1.55(1.19, 2.01) | 0.0456 | G: 1.21(1, 1.46) |
| RS1025946 | 03: 078747342 | LOC642132, | 0.0381 | C,C: 1.67(1.03, 2.71) | 0.0426 | C: 1.63(1.01, 2.62) | 0.0299 | C,C: 1.68(1.06, 2.65) | 0.0343 | C: 1.64(1.05, 2.56) |
| | | ROBO1 | | | | | | | | |
| RS1397977 | 03: 096938051 | | 0.0367 | A,A: 1.46(1.09, 1.96) | 0.0645 | A: 1.2(0.99, 1.45) | 0.1108 | !G,G: 1.37(1.01, 1.87) | 0.0483 | A: 1.21(1, 1.45) |
| RS1461155 | 03: 099731422 | | 0.0436 | !G,G: 11.75(0.65, 213.2) | 0.0918 | A: 1.42(0.96, 2.1) | 0.0066 | !A,G: 1.94(1.22, 3.1) | 0.0319 | A: 1.61(1.05, 2.48) |
| RS11925785 | 03: 105431807 | | 0.0081 | C,T: 1.51(1.15, 1.98) | 0.3562 | T: 1.1(0.91, 1.33) | 0.0005 | T,T: 1.8(1.31, 2.47) | 0.0271 | T: 1.23(1.03, 1.48) |
| RS4895051 | 03: 105472583 | | 0.0446 | !C,C: 1.53(1.09, 2.15) | 0.0880 | T: 1.18(0.98, 1.43) | 0.0002 | !C,T: 1.7(1.3, 2.2) | 0.0717 | T: 1.19(0.99, 1.43) |
| RS7645613 | 03: 116546362 | | 0.0152 | C,C: 4.3(1.34, 13.83) | 0.4991 | C: 1.1(0.85, 1.44) | 0.0747 | !T,T: 1.38(1, 1.89) | 0.0247 | C: 1.39(1.04, 1.85) |
| RS6763416 | 03: 116549440 | | 0.0244 | C,C: 4.02(1.24, 13.01) | 0.5875 | C: 1.08(0.83, 1.41) | 0.0842 | !T,T: 1.36(0.99, 1.86) | 0.0301 | C: 1.37(1.03, 1.82) |
| RS13320544 | 03: 125457097 | KALRN | 0.0127 | !G,G: 16.18(0.92, 284.21) | 0.6666 | A: 1.08(0.77, 1.52) | 0.0053 | !A,G: 1.84(1.25, 2.7) | 0.0054 | A: 1.65(1.16, 2.33) |
| RS6438839 | 03: 125536191 | KALRN | 0.0123 | !G,G: 6.85(1.22, 38.53) | 0.7925 | A: 1.05(0.74, 1.48) | 0.0614 | A,A: 1.49(1.04, 2.12) | 0.0243 | A: 1.47(1.06, 2.05) |
| RS4293718 | 03: 129411810 | EEFSEC | 0.0099 | !G,G: 1.52(1.16, 2) | 0.0061 | A: 1.36(1.09, 1.7) | 0.0904 | !G,G: 1.34(1.03, 1.74) | 0.0388 | A: 1.26(1.01, 1.55) |
| RS3828417 | 03: 129610944 | EEFSEC | 0.1288 | C,C: 1.45(1.01, 2.08) | 0.0474 | C: 1.41(1, 1.98) | 0.0095 | C,C: 1.76(1.16, 2.68) | 0.0064 | C: 1.76(1.17, 2.64) |
| RS6777158 | 03: 140999494 | | 0.0503 | !T,T: 1.57(1.08, 2.27) | 0.0321 | G: 1.24(1.02, 1.51) | 0.0023 | !T,T: 1.82(1.24, 2.67) | 0.0009 | G: 1.38(1.14, 1.67) |
| RS899348 | 03: 142311491 | SPSB4 | 0.0015 | !C,C: 2.63(1.43, 4.85) | 0.4308 | T: 1.1(0.88, 1.37) | 0.0534 | !T,T: 1.34(1.03, 1.74) | 0.0150 | C: 1.29(1.05, 1.59) |
| RS9289637 | 03: 142855081 | | 0.0275 | !A,A: 1.45(1.1, 1.92) | 0.0433 | C: 1.22(1.01, 1.48) | 0.0276 | A,C: 1.43(1.1, 1.86) | 0.3873 | C: 1.09(0.9, 1.31) |
| RS7431637 | 03: 144532459 | SLC9A9 | 0.0062 | !C,C: 1.58(1.19, 2.09) | 0.0042 | T: 1.33(1.1, 1.61) | 0.0028 | !T,T: 1.73(1.2, 2.48) | 0.2476 | C: 1.12(0.93, 1.36) |
| RS2174783 | 03: 147912451 | | 0.0805 | !C,C: 1.53(0.98, 2.4) | 0.0346 | A: 1.25(1.02, 1.53) | 0.1119 | A,A: 1.29(0.99, 1.68) | 0.0338 | A: 1.23(1.02, 1.5) |
| RS4681336 | 03: 147956833 | | 0.0420 | !G,G: 1.59(1.09, 2.31) | 0.0260 | A: 1.25(1.03, 1.51) | 0.1227 | !G,G: 1.36(0.98, 1.9) | 0.0460 | A: 1.21(1.01, 1.46) |
| RS6785504 | 03: 154155469 | | 0.0406 | G,G: 1.49(1.08, 2.05) | 0.0991 | G: 1.17(0.97, 1.42) | 0.0100 | !G,T: 1.49(1.15, 1.94) | 0.8509 | G: 1.02(0.85, 1.23) |
| RS1568790 | 03: 155009791 | | 0.0077 | !C,T: 1.77(1.18, 2.65) | 0.0145 | C: 1.62(1.1, 2.39) | 0.0127 | T,T: 13.56(0.76, 241.41) | 0.0295 | T: 1.45(1.04, 2.01) |
| RS7631400 | 03: 161011416 | SCHIP1 | 0.0022 | !A,A: 1.62(1.23, 2.13) | 0.0015 | G: 1.44(1.15, 1.79) | 0.1040 | !A,A: 1.34(1.02, 1.76) | 0.0457 | G: 1.27(1.01, 1.6) |
| RS4129765 | 03: 174037987 | | 0.0411 | !C,T: 1.47(1.09, 1.99) | 0.0923 | T: 1.24(0.97, 1.6) | 0.0795 | T,T: 1.36(1.03, 1.79) | 0.0272 | T: 1.3(1.03, 1.65) |
| RS1512744 | 03: 174364271 | | 0.0292 | A,G: 1.51(1.11, 2.06) | 0.0649 | A: 1.28(0.99, 1.66) | 0.0431 | A,G: 1.42(1.07, 1.89) | 0.1996 | A: 1.18(0.93, 1.49) |
| RS2861203 | 03: 174433247 | | 0.0294 | !A,A: 1.44(1.1, 1.88) | 0.0140 | G: 1.3(1.06, 1.61) | 0.0018 | A,G: 1.54(1.18, 2.01) | 0.8774 | G: 1.02(0.83, 1.24) |
| RS2046718 | 03: 174955021 | NLGN1 | 0.0306 | A,A: 1.94(1.09, 3.46) | 0.0111 | A: 1.34(1.07, 1.69) | 0.0114 | A,A: 2.51(1.31, 4.81) | 0.2811 | A: 1.14(0.91, 1.42) |
| RS2222447 | 03: 176063520 | NAALADL2 | 0.0905 | !A,A: 1.51(1.01, 2.25) | 0.0444 | C: 1.23(1.01, 1.5) | 0.0798 | !A,A: 1.51(1.04, 2.2) | 0.0460 | C: 1.22(1.01, 1.47) |
| RS2080787 | 03: 176541238 | NAALADL2 | 0.0130 | T,T: 1.59(1.16, 2.18) | 0.0469 | T: 1.21(1, 1.47) | 0.0409 | !C,C: 1.41(1.06, 1.89) | 0.1326 | T: 1.15(0.96, 1.39) |
| RS16825529 | 03: 176550912 | NAALADL2 | 0.0933 | A,A: 1.37(1.02, 1.86) | 0.0368 | A: 1.23(1.02, 1.49) | 0.0358 | !C,C: 1.48(1.08, 2.03) | 0.1457 | A: 1.15(0.95, 1.38) |
| RS4894485 | 03: 176560380 | NAALADL2 | 0.0060 | !C,C: 1.49(1.13, 1.98) | 0.0016 | T: 1.37(1.13, 1.66) | 0.0002 | !C,C: 1.73(1.32, 2.26) | 0.0026 | T: 1.34(1.11, 1.63) |
| RS7609811 | 03: 177341698 | | 0.1081 | T,T: 1.31(1, 1.72) | 0.0379 | T: 1.24(1.01, 1.52) | 0.0304 | !C,T: 1.42(1.09, 1.85) | 0.3314 | T: 1.1(0.91, 1.33) |
| RS6800635 | 03: 177560866 | | 0.1412 | A,A: 1.44(0.97, 2.15) | 0.0469 | A: 1.46(1.01, 2.13) | 0.0166 | A,A: 1.88(1.16, 3.05) | 0.0114 | A: 1.84(1.15, 2.93) |
| RS2130014 | 03: 178098965 | | 0.0343 | C,T: 1.36(1.03, 1.78) | 0.7237 | T: 1.04(0.85, 1.27) | 0.0282 | !C,C: 1.71(1.14, 2.55) | 0.0350 | T: 1.23(1.02, 1.5) |
| RS4254636 | 03: 180018514 | KCNMB2 | 0.0301 | A,G: 1.44(1.09, 1.89) | 0.3018 | G: 1.12(0.91, 1.39) | 0.0861 | !A,A: 1.27(0.98, 1.65) | 0.0344 | G: 1.26(1.02, 1.56) |
| RS6794192 | 03: 180510507 | | 0.0451 | !T,T: 1.71(1.11, 2.63) | 0.1523 | C: 1.16(0.95, 1.42) | 0.0346 | C,C: 1.41(1.08, 1.83) | 0.0567 | C: 1.21(1, 1.46) |
| RS9826864 | 03: 181241320 | | 0.0839 | A,A: 1.32(1.01, 1.73) | 0.0319 | A: 1.26(1.02, 1.56) | 0.0278 | !A,G: 1.43(1.1, 1.87) | 0.3551 | A: 1.1(0.9, 1.35) |
| RS4855011 | 03: 182234345 | | 0.0644 | T,T: 1.52(0.98, 2.34) | 0.0241 | T: 1.27(1.03, 1.55) | 0.0545 | !C,C: 1.38(1.06, 1.8) | 0.0395 | T: 1.23(1.01, 1.49) |
| RS2313212 | 03: 185183622 | ABCC5 | 0.0106 | !T,T: 1.57(1.17, 2.1) | 0.0124 | C: 1.28(1.06, 1.55) | 0.0319 | C,T: 1.38(1.06, 1.79) | 0.4802 | T: 1.07(0.89, 1.29) |
| RS2593813 | 03: 187815265 | AHSG | 0.0297 | !T,T: 1.69(1.11, 2.55) | 0.2416 | C: 1.13(0.93, 1.38) | 0.1307 | !T,T: 1.38(0.94, 2.01) | 0.0462 | C: 1.22(1, 1.47) |
| RS3816112 | 03: 187985359 | EIF4A2 | 0.0325 | C,C: 10.52(0.58, 190.81) | 0.7718 | T: 1.06(0.73, 1.55) | 0.0777 | T,T: 1.5(1.02, 2.21) | 0.0331 | T: 1.49(1.03, 2.16) |
| RS4475031 | 03: 188743651 | | 0.0188 | !C,C: 2.07(1.18, 3.66) | 0.0110 | T: 1.34(1.07, 1.67) | 0.0491 | !C,T: 1.34(1.02, 1.75) | 0.7411 | T: 1.04(0.84, 1.29) |
| RS4687002 | 03: 190104562 | | 0.0427 | !C,T: 1.42(1.08, 1.87) | 0.2552 | C: 1.13(0.92, 1.4) | 0.0644 | C,C: 1.36(1.05, 1.77) | 0.0242 | C: 1.27(1.04, 1.56) |
| RS12107377 | 03: 193633511 | FGF12 | 0.0196 | !A,A: 1.46(1.06, 2.01) | 0.0049 | G: 1.32(1.09, 1.6) | 0.1121 | !A,A: 1.31(0.97, 1.76) | 0.0386 | G: 1.22(1.01, 1.47) |
| RS4687332 | 03: 193637315 | FGF12 | 0.1254 | C,C: 1.31(0.97, 1.76) | 0.0461 | C: 1.22(1.01, 1.48) | 0.0322 | C,C: 1.49(1.1, 2.01) | 0.0243 | C: 1.24(1.03, 1.49) |
| RS2980206 | 04: 004386725 | | 0.0121 | !T,T: 1.77(1.12, 2.81) | 0.6363 | C: 1.06(0.86, 1.3) | 0.0521 | !T,T: 1.72(1.08, 2.75) | 0.0312 | C: 1.26(1.02, 1.55) |
| RS6826372 | 04: 005845805 | EVC | 0.0467 | G,G: 2.76(1.12, 6.83) | 0.5671 | G: 1.08(0.84, 1.39) | 0.0155 | !A,G: 1.52(1.13, 2.04) | 0.1026 | A: 1.23(0.96, 1.58) |
| RS17178569 | 04: 008115661 | ABLIM2 | 0.0148 | A,G: 1.51(1.13, 2.03) | 0.1166 | A: 1.23(0.96, 1.57) | 0.0464 | A,G: 1.4(1.05, 1.87) | 0.2180 | A: 1.17(0.92, 1.49) |
| RS2293229 | 04: 008458306 | ACOX3 | 0.1127 | T,T: 1.33(1.02, 1.74) | 0.0447 | T: 1.24(1.01, 1.54) | 0.1236 | T,T: 1.26(0.97, 1.64) | 0.0418 | T: 1.23(1.01, 1.5) |
| RS1464258 | 04: 009443205 | SLC2A9 | 0.0482 | !G,G: 1.38(1.04, 1.82) | 0.0165 | T: 1.28(1.05, 1.55) | 0.0193 | G,T: 1.45(1.12, 1.89) | 0.4071 | T: 1.09(0.9, 1.32) |
| RS6449068 | 04: 009473034 | SLC2A9 | 0.0096 | C,C: 1.77(1.13, 2.79) | 0.0032 | C: 1.36(1.11, 1.67) | 0.0422 | C,T: 1.39(1.07, 1.81) | 0.5146 | C: 1.07(0.88, 1.3) |
| RS1519098 | 04: 009490256 | SLC2A9 | 0.0463 | !G,G: 1.35(1.02, 1.78) | 0.0131 | T: 1.28(1.05, 1.56) | 0.0474 | G,T: 1.39(1.07, 1.8) | 0.5607 | T: 1.06(0.88, 1.28) |
| RS1606084 | 04: 013506439 | | 0.0203 | !C,T: 1.49(1.12, 1.99) | 0.0236 | C: 1.31(1.04, 1.66) | 0.1241 | C,C: 1.31(1.01, 1.72) | 0.0486 | C: 1.25(1, 1.56) |
| RS7676721 | 04: 013525449 | | 6.83E−05 | G,G: 2.83(1.56, 5.15) | 3.01E−05 | G: 1.6(1.28, 2) | 0.0808 | !T,T: 1.29(0.99, 1.68) | 0.0301 | G: 1.27(1.03, 1.57) |
| RS505703 | 04: 013530080 | | 0.0037 | G,G: 3.24(1.33, 7.86) | 0.0020 | G: 1.51(1.16, 1.96) | 0.0041 | !A,A: 1.62(1.22, 2.15) | 0.0018 | G: 1.49(1.16, 1.92) |
| RS6840951 | 04: 014053172 | | 0.0236 | C,T: 1.51(1.12, 2.04) | 0.0293 | C: 1.33(1.03, 1.71) | 0.0147 | !C,C: 2.74(1.33, 5.63) | 0.0545 | T: 1.27(1, 1.62) |
| RS10516278 | 04: 014129536 | | 0.0664 | G,T: 1.46(1.06, 2.03) | 0.0416 | G: 1.36(1.02, 1.81) | 0.0229 | !G,G: 3.36(1.29, 8.79) | 0.2926 | T: 1.17(0.89, 1.54) |
| RS7696128 | 04: 018697484 | | 0.0486 | G,G: 1.48(1.08, 2.03) | 0.0808 | G: 1.19(0.98, 1.44) | 0.0057 | !T,T: 1.69(1.22, 2.35) | 0.0299 | G: 1.23(1.02, 1.48) |
| RS4496580 | 04: 019278475 | | 0.0354 | !T,T: 2.84(1.21, 6.66) | 0.3383 | C: 1.13(0.88, 1.46) | 0.0660 | C,C: 1.34(1.01, 1.78) | 0.0299 | C: 1.33(1.03, 1.71) |
| RS1030349 | 04: 037504194 | PGM2 | 0.0979 | A,A: 1.3(0.98, 1.73) | 0.0393 | A: 1.3(1.02, 1.66) | 0.0770 | A,A: 1.34(1.01, 1.78) | 0.0273 | A: 1.33(1.04, 1.7) |
| RS2608307 | 04: 037514632 | PGM2 | 0.0902 | !G,G: 1.75(0.93, 3.28) | 0.0323 | A: 1.29(1.02, 1.63) | 0.1106 | A,A: 1.29(0.98, 1.7) | 0.0405 | A: 1.29(1.01, 1.63) |
| RS1377347 | 04: 041067359 | DKFZP686A01247 | 0.0027 | !G,G: 1.6(1.22, 2.09) | 0.0045 | T: 1.35(1.1, 1.66) | 0.0200 | !G,G: 1.44(1.11, 1.87) | 0.0086 | T: 1.32(1.07, 1.61) |
| RS13116757 | 04: 041093136 | DKFZP686A01247 | 0.0129 | !T,T: 1.5(1.14, 1.97) | 0.0086 | G: 1.34(1.08, 1.66) | 0.0905 | !T,T: 1.34(1.03, 1.74) | 0.0386 | G: 1.26(1.01, 1.55) |
| RS1028000 | 04: 054155288 | | 0.0569 | !C,C: 1.39(1.06, 1.82) | 0.0333 | T: 1.27(1.02, 1.58) | 0.0019 | T,T: 2.66(1.5, 4.71) | 0.0089 | T: 1.33(1.08, 1.65) |
| RS4131086 | 04: 057080212 | ARL9 | 0.0314 | A,G: 1.42(1.08, 1.87) | 0.4459 | G: 1.09(0.88, 1.35) | 1.03E−04 | !G,G: 2.83(1.69, 4.73) | 0.0004 | A: 1.45(1.18, 1.79) |
| RS10517437 | 04: 058703169 | | 0.1060 | G,G: 1.36(1, 1.83) | 0.0414 | G: 1.22(1.01, 1.48) | 0.0165 | G,G: 1.54(1.13, 2.11) | 0.0821 | G: 1.18(0.98, 1.42) |
| RS1505663 | 04: 061814685 | | 0.1218 | !A,A: 1.33(1, 1.77) | 0.0459 | G: 1.29(1.01, 1.65) | 0.0065 | A,G: 1.58(1.19, 2.09) | 0.0215 | G: 1.33(1.05, 1.68) |
| RS2034002 | 04: 077324198 | SCARB2 | 0.0122 | A,G: 1.81(1.17, 2.79) | 0.0200 | G: 1.61(1.07, 2.42) | 0.0406 | A,G: 1.54(0.99, 2.41) | 0.1649 | G: 1.35(0.89, 2.06) |
| RS10005531 | 04: 079039537 | MRPL1 | 0.0263 | G,G: 1.48(1.11, 1.96) | 0.0348 | G: 1.23(1.02, 1.49) | 0.0212 | !G,T: 1.44(1.11, 1.87) | 0.9251 | T: 1.01(0.84, 1.21) |
| RS10019920 | 04: 079085031 | MRPL1 | 0.0111 | G,G: 1.55(1.16, 2.06) | 0.0163 | G: 1.27(1.05, 1.54) | 0.0221 | !A,G: 1.44(1.11, 1.87) | 1.0000 | A: 1(0.83, 1.2) |
| RS7436080 | 04: 079096173 | | 0.0111 | C,C: 1.55(1.16, 2.06) | 0.0163 | C: 1.27(1.05, 1.54) | 0.0181 | !C,T: 1.46(1.12, 1.89) | 0.9625 | T: 1.01(0.84, 1.21) |
| RS979512 | 04: 079171790 | | 0.0413 | T,T: 1.47(1.09, 1.98) | 0.0414 | T: 1.22(1.01, 1.48) | 0.0023 | !C,T: 1.59(1.22, 2.06) | 0.8880 | T: 1.02(0.85, 1.22) |
| RS7376907 | 04: 083875498 | SCD5 | 0.0050 | !C,C: 1.61(1.14, 2.27) | 0.0012 | T: 1.38(1.14, 1.67) | 0.0308 | !C,C: 1.53(1.09, 2.14) | 0.0159 | T: 1.26(1.05, 1.52) |
| RS2622629 | 04: 089313088 | | 0.0392 | !T,T: 1.42(1.08, 1.86) | 0.0459 | C: 1.23(1.01, 1.51) | 0.0945 | !T,T: 1.28(0.98, 1.67) | 0.0373 | C: 1.23(1.02, 1.48) |
| RS12651141 | 04: 089506733 | | 0.0646 | A,A: 1.43(1.06, 1.92) | 0.0271 | A: 1.35(1.04, 1.76) | 0.0128 | A,A: 1.53(1.14, 2.04) | 0.0057 | A: 1.44(1.11, 1.87) |
| RS17248648 | 04: 091988638 | | 0.0082 | A,G: 1.75(1.22, 2.52) | 0.0112 | G: 1.53(1.1, 2.11) | 0.0267 | A,G: 1.44(1, 2.08) | 0.1898 | G: 1.26(0.89, 1.78) |
| RS6856278 | 04: 097787265 | LOC441031 | 0.0995 | !C,C: 1.47(1, 2.15) | 0.0465 | A: 1.22(1.01, 1.49) | 0.0791 | A,A: 1.3(0.98, 1.71) | 0.0257 | A: 1.24(1.03, 1.49) |
| RS1042364 | 04: 100264597 | ADH4 | 0.0426 | A,G: 1.37(1.04, 1.8) | 0.8339 | G: 1.02(0.83, 1.26) | 0.0339 | !G,G: 1.76(1.13, 2.76) | 0.0233 | A: 1.27(1.03, 1.55) |
| RS1126673 | 04: 100264639 | ADH4 | 0.0205 | A,G: 1.41(1.08, 1.85) | 0.9589 | G: 1.01(0.82, 1.23) | 0.0480 | !G,G: 1.59(1.05, 2.41) | 0.0176 | A: 1.28(1.05, 1.56) |
| RS1126672 | 04: 100266835 | ADH4 | 0.0423 | C,T: 1.38(1.05, 1.81) | 0.8337 | C: 1.03(0.84, 1.26) | 0.0345 | !C,C: 1.76(1.12, 2.75) | 0.0231 | T: 1.27(1.04, 1.56) |
| RS1126671 | 04: 100267437 | ADH4 | 0.0205 | A,G: 1.41(1.08, 1.85) | 0.9589 | G: 1.01(0.82, 1.23) | 0.0480 | !G,G: 1.59(1.05, 2.41) | 0.0176 | A: 1.28(1.05, 1.56) |
| RS1126670 | 04: 100271756 | ADH4 | 0.0305 | G,T: 1.39(1.06, 1.82) | 0.9589 | G: 1.01(0.82, 1.23) | 0.0356 | !G,G: 1.64(1.08, 2.48) | 0.0132 | T: 1.29(1.06, 1.57) |
| RS4128643 | 04: 101433596 | | 0.0933 | A,A: 1.33(0.97, 1.83) | 0.0331 | A: 1.23(1.02, 1.49) | 0.0907 | !G,G: 1.32(0.99, 1.77) | 0.0305 | A: 1.23(1.02, 1.48) |
| RS10516462 | 04: 101526697 | | 0.0621 | !G,G: 1.39(1.06, 1.82) | 0.0304 | A: 1.25(1.02, 1.52) | 0.0071 | !G,G: 1.53(1.17, 1.99) | 0.0059 | A: 1.31(1.08, 1.59) |
| RS1452689 | 04: 109970223 | COL25A1 | 0.0556 | A,A: 3.45(1.05, 11.36) | 0.0457 | A: 1.38(1.01, 1.9) | 0.0243 | !A,G: 1.4(1.02, 1.92) | 0.4369 | G: 1.12(0.85, 1.48) |
| RS12641791 | 04: 122118114 | | 0.0060 | !A,G: 2.16(1.26, 3.7) | 0.0372 | A: 1.71(1.05, 2.78) | 0.0148 | !A,G: 1.87(1.14, 3.06) | 0.0343 | A: 1.69(1.05, 2.71) |
| RS970349 | 04: 123091082 | | 0.1006 | G,G: 1.35(1.03, 1.77) | 0.0411 | G: 1.26(1.01, 1.58) | 0.0276 | !A,G: 1.45(1.1, 1.91) | 0.0324 | G: 1.27(1.02, 1.59) |
| RS1911014 | 04: 126993548 | | 0.0491 | !G,G: 3.25(1.11, 9.54) | 0.6847 | A: 1.06(0.81, 1.38) | 0.0724 | !G,G: 2.43(0.89, 6.6) | 0.0444 | A: 1.31(1.01, 1.69) |
| RS1032673 | 04: 129684939 | | 0.0435 | !C,C: 1.57(1.02, 2.43) | 0.6813 | A: 1.04(0.85, 1.28) | 0.0418 | !A,A: 1.39(1.07, 1.81) | 0.0181 | C: 1.28(1.05, 1.57) |
| RS1908039 | 04: 131347083 | LOC645995, | 0.1356 | !G,G: 1.37(0.96, 1.95) | 0.0480 | A: 1.39(1.01, 1.93) | 0.0400 | !G,G: 1.52(1.08, 2.12) | 0.0114 | A: 1.5(1.1, 2.05) |
| | | LOC648034 | | | | | | | | |
| RS1908038 | 04: 131352941 | LOC645995, | 0.1417 | !A,A: 1.36(0.96, 1.94) | 0.0495 | G: 1.39(1, 1.92) | 0.0351 | !A,A: 1.53(1.09, 2.15) | 0.0093 | G: 1.51(1.11, 2.07) |
| | | LOC648034 | | | | | | | | |
| RS11732021 | 04: 133738741 | | 0.0636 | !A,A: 1.38(1.05, 1.81) | 0.0246 | G: 1.26(1.03, 1.54) | 0.0692 | !A,A: 1.35(1.03, 1.77) | 0.0263 | G: 1.24(1.03, 1.5) |
| RS3796627 | 04: 140980727 | MAML3 | 0.0776 | !T,T: 1.57(1.03, 2.4) | 0.0492 | C: 1.22(1, 1.49) | 0.0229 | !T,T: 1.65(1.1, 2.47) | 0.0109 | C: 1.29(1.06, 1.56) |
| RS4696084 | 04: 151437383 | LRBA | 0.0461 | !A,A: 1.4(1.07, 1.85) | 0.0122 | C: 1.33(1.06, 1.67) | 0.0405 | A,C: 1.41(1.07, 1.86) | 0.0533 | C: 1.24(1, 1.54) |
| RS2054685 | 04: 173475035 | GALNT17 | 0.0301 | !T,T: 1.45(1.05, 2.01) | 0.0131 | C: 1.46(1.08, 1.97) | 0.0982 | !T,T: 1.39(1.02, 1.9) | 0.0362 | C: 1.36(1.02, 1.81) |
| RS6553010 | 04: 187693337 | MTNR1A | 0.0715 | !A,A: 1.36(1.02, 1.8) | 0.0184 | G: 1.33(1.05, 1.69) | 0.0180 | !A,A: 1.47(1.12, 1.93) | 0.0110 | G: 1.35(1.07, 1.69) |
| RS6857356 | 04: 188706532 | | 0.0719 | !A,A: 2.58(1.02, 6.5) | 0.0472 | G: 1.33(1, 1.75) | 0.0389 | !A,A: 3.17(1.21, 8.35) | 0.2312 | G: 1.19(0.9, 1.57) |
| RS6895153 | 05: 002586586 | | 0.0990 | T,T: 1.32(1, 1.74) | 0.0354 | T: 1.29(1.02, 1.63) | 0.0847 | T,T: 1.31(1, 1.71) | 0.0277 | T: 1.28(1.03, 1.6) |
| RS256358 | 05: 005723085 | | 0.0292 | !T,T: 2.18(1.18, 4) | 0.2777 | C: 1.13(0.91, 1.42) | 0.0466 | !T,T: 1.83(1.11, 3) | 0.0431 | C: 1.24(1.01, 1.53) |
| RS10040610 | 05: 006406815 | | 0.0137 | C,C: 5.44(0.95, 31.04) | 0.0090 | C: 1.52(1.11, 2.08) | 0.0352 | C,C: 3.97(0.98, 16.1) | 0.0314 | C: 1.36(1.03, 1.81) |
| RS4701724 | 05: 006418689 | | 0.0302 | C,C: 1.47(1.06, 2.03) | 0.0088 | C: 1.29(1.07, 1.56) | 0.0870 | !T,T: 1.4(1.03, 1.9) | 0.0486 | C: 1.2(1, 1.45) |
| RS7735244 | 05: 006637248 | LOC255167 | 0.0462 | T,T: 1.56(1.09, 2.22) | 0.0185 | T: 1.49(1.07, 2.08) | 0.0345 | !C,T: 1.53(1.07, 2.17) | 0.1042 | T: 1.31(0.95, 1.81) |
| RS446249 | 05: 008429813 | | 0.0555 | C,C: 1.6(1.01, 2.53) | 0.0234 | C: 1.26(1.03, 1.55) | 0.1001 | !T,T: 1.31(1.01, 1.71) | 0.0367 | C: 1.24(1.02, 1.5) |
| RS40708 | 05: 009317682 | SEMA5A | 0.0236 | !G,G: 1.66(1.06, 2.59) | 0.0145 | A: 1.71(1.11, 2.62) | 0.0131 | A,G: 1.77(1.13, 2.78) | 0.0297 | A: 1.62(1.05, 2.49) |
| RS465107 | 05: 009575303 | SEMA5A | 0.0253 | !A,A: 2.99(1.28, 6.98) | 0.2193 | G: 1.17(0.91, 1.51) | 0.1412 | G,G: 1.3(0.98, 1.72) | 0.0484 | G: 1.28(1, 1.63) |
| RS960567 | 05: 009751490 | LOC285692 | 0.0267 | !A,C: 1.47(1.11, 1.96) | 0.1434 | A: 1.18(0.95, 1.48) | 0.0017 | !A,C: 1.57(1.19, 2.06) | 0.4863 | A: 1.08(0.87, 1.33) |
| RS606490 | 05: 010371455 | | 0.0058 | C,C: 1.54(1.15, 2.06) | 0.0014 | C: 1.51(1.17, 1.94) | 0.0171 | !T,T: 3.89(1.37, 11.05) | 0.2787 | C: 1.16(0.9, 1.48) |
| RS1373992 | 05: 013511367 | | 0.0395 | A,A: 1.45(1.09, 1.92) | 0.0205 | A: 1.34(1.05, 1.72) | 0.0880 | A,A: 1.31(1, 1.72) | 0.0308 | A: 1.29(1.03, 1.63) |
| RS40979 | 05: 016774306 | MYO10 | 0.0566 | T,T: 2.61(1.05, 6.49) | 0.0385 | T: 1.33(1.02, 1.75) | 0.0032 | G,T: 1.62(1.19, 2.2) | 0.0560 | T: 1.3(1, 1.71) |
| RS4242060 | 05: 018932882 | | 0.1114 | A,A: 1.32(1, 1.75) | 0.0421 | A: 1.28(1.01, 1.62) | 0.0453 | A,A: 1.4(1.07, 1.84) | 0.0352 | A: 1.28(1.02, 1.62) |
| RS10062061 | 05: 027873351 | | 0.0707 | A,A: 1.38(1.05, 1.82) | 0.0321 | A: 1.24(1.02, 1.51) | 0.0139 | !A,C: 1.44(1.1, 1.87) | 0.9606 | A: 1.01(0.83, 1.22) |
| RS2287582 | 05: 031338045 | CDH6 | 0.0358 | !A,A: 1.47(1.08, 1.98) | 0.0196 | G: 1.26(1.04, 1.52) | 0.0251 | A,G: 1.4(1.08, 1.81) | 0.3478 | A: 1.09(0.91, 1.31) |
| RS2909570 | 05: 031354162 | CDH6 | 0.0328 | !T,T: 1.46(1.09, 1.97) | 0.0149 | G: 1.27(1.05, 1.54) | 0.0126 | G,T: 1.46(1.12, 1.89) | 0.5412 | T: 1.06(0.88, 1.28) |
| RS4867387 | 05: 031877036 | PDZD2 | 0.0659 | !C,C: 2.58(1.02, 6.52) | 0.0494 | T: 1.31(1, 1.71) | 0.0342 | !C,C: 3.17(1.21, 8.35) | 0.0775 | T: 1.26(0.97, 1.63) |
| RS1847610 | 05: 031975096 | PDZD2 | 0.0237 | !T,T: 1.5(1.12, 2.01) | 0.0410 | C: 1.22(1.01, 1.48) | 0.0209 | C,T: 1.44(1.11, 1.87) | 0.5096 | C: 1.07(0.89, 1.29) |
| RS7726919 | 05: 032002949 | PDZD2 | 0.0296 | G,T: 1.4(1.07, 1.83) | 0.1285 | G: 1.16(0.96, 1.41) | 0.0992 | !T,T: 1.29(0.98, 1.7) | 0.0318 | G: 1.23(1.02, 1.48) |
| RS7705676 | 05: 035273493 | | 0.0409 | !C,C: 1.49(1.08, 2.05) | 0.1312 | T: 1.16(0.96, 1.41) | 0.0214 | T,T: 1.45(1.09, 1.94) | 0.1317 | T: 1.15(0.96, 1.39) |
| RS7715492 | 05: 050516918 | | 0.0359 | !C,C: 1.67(1.06, 2.63) | 0.0212 | T: 1.69(1.08, 2.63) | 0.0350 | C,T: 1.5(0.96, 2.33) | 0.2929 | T: 1.26(0.83, 1.9) |
| RS10062839 | 05: 060139209 | ELOVL7 | 0.0250 | C,T: 1.37(1.03, 1.82) | 0.6765 | T: 1.06(0.84, 1.34) | 0.0256 | !T,T: 2.26(1.22, 4.17) | 0.1275 | C: 1.19(0.95, 1.48) |
| RS153373 | 05: 065461915 | | 0.0472 | !A,G: 1.49(1.08, 2.05) | 0.0865 | A: 1.28(0.97, 1.7) | 0.0064 | !A,G: 1.75(1.22, 2.5) | 0.0104 | A: 1.52(1.11, 2.09) |
| RS1466344 | 05: 071449384 | MAP1B | 0.0707 | A,A: 3.27(0.79, 13.46) | 0.0398 | A: 1.41(1.02, 1.96) | 0.0079 | !G,G: 1.63(1.18, 2.25) | 0.0028 | A: 1.58(1.18, 2.13) |
| RS3101187 | 05: 071452383 | MAP1B | 0.0056 | !G,G: 1.61(1.15, 2.24) | 0.0022 | A: 1.61(1.19, 2.19) | 0.0948 | !G,G: 1.39(1.03, 1.88) | 0.0379 | A: 1.34(1.02, 1.76) |
| RS16878312 | 05: 071741140 | | 0.0137 | C,C: 1.42(1.08, 1.85) | 0.0047 | C: 1.35(1.1, 1.66) | 0.1135 | !A,A: 1.46(0.94, 2.25) | 0.0362 | C: 1.24(1.01, 1.52) |
| RS191899 | 05: 073481573 | | 0.0217 | !A,A: 1.69(1.16, 2.47) | 0.0189 | G: 1.27(1.04, 1.54) | 0.0433 | G,G: 1.38(1.06, 1.8) | 0.0884 | G: 1.18(0.98, 1.43) |
| RS4425488 | 05: 075330102 | | 0.1057 | !T,T: 1.34(0.97, 1.87) | 0.0320 | C: 1.24(1.02, 1.5) | 0.0174 | C,C: 1.49(1.12, 1.98) | 0.0422 | C: 1.21(1.01, 1.46) |
| RS2287933 | 05: 076005975 | IQGAP2 | 0.0208 | !A,C: 1.66(1.09, 2.53) | 0.0975 | A: 1.38(0.94, 2.03) | 0.0482 | C,C: 11.45(0.63, 207.63) | 0.1143 | C: 1.32(0.93, 1.87) |
| RS2279095 | 05: 076745182 | PDE8B | 0.0084 | G,T: 1.53(1.07, 2.17) | 0.0976 | G: 1.32(0.96, 1.84) | 0.0067 | !T,T: 1.59(1.1, 2.3) | 0.0066 | G: 1.62(1.14, 2.29) |
| RS2044163 | 05: 077267160 | | 0.0286 | T,T: 2.01(1.07, 3.8) | 0.0307 | T: 1.98(1.06, 3.7) | 0.0124 | T,T: 1.89(1.14, 3.15) | 0.0142 | T: 1.85(1.12, 3.04) |
| RS423704 | 05: 082256836 | | 0.0115 | C,T: 1.75(1.19, 2.57) | 0.0184 | T: 1.53(1.08, 2.17) | 0.0068 | C,T: 1.6(1.14, 2.25) | 0.1423 | T: 1.26(0.93, 1.71) |
| RS871759 | 05: 082784704 | | 0.0773 | !C,C: 1.31(0.98, 1.75) | 0.0409 | T: 1.31(1.02, 1.68) | 0.0576 | !C,C: 1.31(0.99, 1.71) | 0.0240 | T: 1.31(1.04, 1.65) |
| RS1500226 | 05: 083652159 | EDIL3 | 0.0365 | !T,T: 1.37(1.02, 1.84) | 0.0128 | C: 1.28(1.06, 1.55) | 0.0272 | !C,T: 1.39(1.07, 1.8) | 0.6029 | C: 1.05(0.87, 1.27) |
| RS7717140 | 05: 083655286 | EDIL3 | 0.1180 | G,G: 1.43(0.94, 2.17) | 0.0416 | G: 1.23(1.01, 1.51) | 0.0425 | G,G: 1.57(1.05, 2.36) | 0.4936 | G: 1.07(0.88, 1.3) |
| RS10805837 | 05: 085947410 | | 0.0141 | !C,C: 1.84(1.21, 2.8) | 0.0291 | T: 1.25(1.03, 1.53) | 0.0222 | T,T: 1.43(1.1, 1.86) | 0.0417 | T: 1.23(1.01, 1.49) |
| RS1423595 | 05: 090831684 | | 0.0858 | G,G: 1.38(1, 1.9) | 0.0289 | G: 1.24(1.02, 1.5) | 0.0313 | A,G: 1.38(1.06, 1.79) | 0.1877 | G: 1.13(0.94, 1.36) |
| RS4916875 | 05: 091123834 | | 0.0820 | !A,A: 1.31(1, 1.72) | 0.0295 | G: 1.25(1.03, 1.53) | 0.0021 | !A,G: 1.55(1.19, 2.02) | 1.0000 | A: 1(0.83, 1.21) |
| RS11949638 | 05: 093933645 | MGC34713 | 0.0127 | A,A: 1.6(1.12, 2.28) | 0.3292 | A: 1.1(0.91, 1.34) | 0.0546 | !G,G: 1.39(1.05, 1.84) | 0.0203 | A: 1.25(1.04, 1.5) |
| RS257218 | 05: 097404940 | | 0.0195 | !G,G: 1.48(1.12, 1.96) | 0.0409 | A: 1.23(1.01, 1.5) | 0.0487 | !A,A: 1.54(1.06, 2.23) | 0.2865 | G: 1.11(0.92, 1.34) |
| RS10515303 | 05: 100303212 | | 0.0444 | !C,C: 1.66(1.08, 2.55) | 0.2757 | T: 1.12(0.92, 1.38) | 0.0033 | !T,T: 1.57(1.21, 2.05) | 0.0028 | C: 1.35(1.11, 1.64) |
| RS1422046 | 05: 102958169 | | 0.0416 | !C,C: 1.55(1.09, 2.21) | 0.0204 | T: 1.26(1.04, 1.53) | 0.0441 | T,T: 1.39(1.06, 1.84) | 0.1283 | T: 1.16(0.96, 1.4) |
| RS6859797 | 05: 104016997 | | 0.0372 | !A,C: 1.47(1.09, 1.98) | 0.0493 | A: 1.29(1, 1.65) | 0.0024 | !A,C: 1.65(1.23, 2.22) | 0.0462 | A: 1.29(1.01, 1.65) |
| RS1435194 | 05: 105481256 | | 0.0609 | !C,C: 1.38(1.04, 1.82) | 0.0203 | A: 1.33(1.05, 1.68) | 0.0051 | !C,C: 1.43(1.1, 1.86) | 0.0019 | A: 1.41(1.14, 1.76) |
| RS11959425 | 05: 109587365 | | 0.0621 | !G,G: 1.79(1.04, 3.07) | 0.0230 | A: 1.31(1.04, 1.65) | 0.0525 | A,A: 1.35(1.04, 1.76) | 0.0186 | A: 1.3(1.05, 1.61) |
| RS17133647 | 05: 111061008 | | 0.0589 | A,A: 4.79(0.83, 27.8) | 0.0478 | A: 1.44(1, 2.05) | 0.0138 | A,A: 3.74(1.14, 12.34) | 0.8231 | C: 1.04(0.77, 1.39) |
| RS10519637 | 05: 119905653 | LOC51334 | 0.0491 | A,G: 1.43(1.06, 1.94) | 0.1696 | G: 1.2(0.93, 1.55) | 0.0482 | G,G: 2.38(1.13, 4.98) | 0.0691 | G: 1.25(0.99, 1.59) |
| RS245184 | 05: 127190475 | | 0.0170 | G,G: 1.97(1.18, 3.3) | 0.3621 | G: 1.11(0.9, 1.37) | 0.0201 | !A,G: 1.45(1.11, 1.89) | 0.0383 | A: 1.25(1.01, 1.54) |
| RS2191081 | 05: 128053707 | | 0.0939 | !T,T: 1.36(1.02, 1.81) | 0.0485 | C: 1.22(1, 1.48) | 0.0144 | !T,T: 1.48(1.13, 1.93) | 0.0055 | C: 1.31(1.09, 1.59) |
| RS1019135 | 05: 128060202 | | 0.0939 | !A,A: 1.36(1.02, 1.81) | 0.0485 | G: 1.22(1, 1.48) | 0.0130 | !A,A: 1.48(1.13, 1.93) | 0.0047 | G: 1.32(1.09, 1.6) |
| RS958687 | 05: 132665816 | FSTL4 | 0.0153 | G,G: 1.57(1.15, 2.13) | 0.0102 | G: 1.28(1.06, 1.55) | 0.0563 | G,G: 1.42(1.07, 1.9) | 0.0344 | G: 1.22(1.02, 1.47) |
| RS31551 | 05: 135303229 | FBXL21 | 0.0495 | T,T: 1.42(1.07, 1.87) | 0.0394 | T: 1.23(1.01, 1.5) | 0.0417 | T,T: 1.39(1.07, 1.82) | 0.0147 | T: 1.27(1.05, 1.54) |
| RS30737 | 05: 135351333 | | 0.0765 | A,A: 1.39(1.04, 1.85) | 0.0417 | A: 1.3(1.01, 1.67) | 0.0697 | !C,C: 1.95(0.95, 4.01) | 0.0275 | A: 1.31(1.03, 1.67) |
| RS12519073 | 05: 136804661 | SPOCK1 | 0.0105 | !T,T: 1.52(1.15, 1.99) | 0.0051 | C: 1.38(1.1, 1.72) | 0.0215 | !C,T: 1.36(1.02, 1.79) | 1.0000 | T: 1(0.8, 1.25) |
| RS3734166 | 05: 137693222 | CDC25C | 0.0470 | A,G: 1.41(1.07, 1.86) | 0.1478 | A: 1.17(0.95, 1.44) | 0.0056 | !A,A: 2.74(1.41, 5.31) | 0.0204 | G: 1.29(1.04, 1.61) |
| RS1991800 | 05: 145733194 | | 0.0340 | C,T: 1.53(1.11, 2.12) | 0.0424 | C: 1.35(1.02, 1.8) | 0.0263 | C,T: 1.51(1.11, 2.06) | 0.1244 | C: 1.24(0.95, 1.61) |
| RS6868716 | 05: 145769452 | | 0.0096 | !C,C: 1.68(1.18, 2.4) | 0.0060 | T: 1.6(1.14, 2.23) | 0.0172 | C,T: 1.61(1.14, 2.28) | 0.1160 | T: 1.28(0.95, 1.71) |
| RS2288803 | 05: 146756043 | DPYSL3 | 0.0187 | A,A: 2.28(1.21, 4.29) | 0.0181 | A: 1.31(1.05, 1.65) | 0.0495 | !A,C: 1.4(1.07, 1.83) | 0.1300 | C: 1.18(0.96, 1.46) |
| RS352360 | 05: 148643981 | FLJ36748 | 0.0232 | !A,G: 1.43(1.09, 1.87) | 0.4099 | A: 1.08(0.9, 1.31) | 0.0257 | !A,G: 1.43(1.1, 1.86) | 0.8879 | G: 1.02(0.85, 1.22) |
| RS296171 | 05: 152584259 | | 0.0082 | C,T: 1.57(1.17, 2.1) | 0.0526 | C: 1.28(1, 1.64) | 0.0035 | !T,T: 1.49(1.14, 1.97) | 0.0013 | C: 1.48(1.17, 1.87) |
| RS6892282 | 05: 159293063 | ADRA1B | 0.0720 | T,T: 1.38(1.04, 1.83) | 0.0273 | T: 1.24(1.03, 1.51) | 0.0246 | !G,T: 1.39(1.07, 1.81) | 0.7027 | G: 1.04(0.86, 1.25) |
| RS7724146 | 05: 160827209 | GABRB2 | 0.0316 | C,T: 1.47(1.09, 1.97) | 0.0910 | C: 1.24(0.97, 1.58) | 0.0383 | C,T: 1.4(1.05, 1.87) | 0.4251 | C: 1.11(0.87, 1.41) |
| RS2431268 | 05: 161956558 | LOC651586, | 0.0955 | G,G: 1.34(1.02, 1.75) | 0.0386 | G: 1.25(1.01, 1.54) | 0.0121 | !A,A: 1.91(1.24, 2.96) | 0.0402 | G: 1.23(1.01, 1.5) |
| | | LOC644536 | | | | | | | | |
| RS4498268 | 05: 163768515 | | 0.0821 | !G,G: 1.39(1.04, 1.87) | 0.0303 | A: 1.33(1.03, 1.73) | 0.1023 | !G,G: 1.29(0.97, 1.71) | 0.0475 | A: 1.29(1, 1.66) |
| RS7718356 | 05: 163781686 | | 0.0310 | !G,G: 1.49(1.09, 2.04) | 0.0120 | T: 1.45(1.09, 1.92) | 0.0272 | !G,G: 1.46(1.08, 1.97) | 0.0090 | T: 1.44(1.1, 1.89) |
| RS165070 | 05: 163933613 | | 0.0468 | T,T: 2.38(1.14, 4.94) | 0.2464 | T: 1.17(0.91, 1.5) | 0.0397 | C,C: 1.42(1.08, 1.87) | 0.0237 | C: 1.32(1.04, 1.67) |
| RS2962867 | 05: 164254631 | | 0.1044 | !C,C: 1.34(1.02, 1.75) | 0.0438 | T: 1.25(1.01, 1.55) | 0.0481 | C,T: 1.41(1.07, 1.85) | 0.1903 | T: 1.17(0.93, 1.46) |
| RS2938767 | 05: 168212967 | SLIT3 | 0.0417 | !G,G: 1.54(1.09, 2.17) | 0.0276 | A: 1.24(1.03, 1.51) | 0.0430 | !G,G: 1.54(1.09, 2.17) | 0.1081 | A: 1.17(0.97, 1.4) |
| RS17646250 | 05: 169244766 | DOCK2 | 0.0431 | !C,T: 1.47(1.01, 2.13) | 0.2267 | C: 1.23(0.88, 1.73) | 0.0057 | !C,C: 1.64(1.14, 2.34) | 0.0024 | T: 1.68(1.2, 2.35) |
| RS4868141 | 05: 171456041 | STK10 | 0.0396 | !G,G: 2.23(1.05, 4.72) | 0.0211 | A: 1.34(1.05, 1.71) | 0.0834 | A,A: 1.35(1.02, 1.78) | 0.0279 | A: 1.31(1.03, 1.67) |
| RS522133 | 05: 172364187 | ATP6V0E | 0.0003 | C,T: 2.11(1.43, 3.1) | 0.0043 | C: 1.64(1.17, 2.29) | 0.0130 | C,C: 13.56(0.76, 241.41) | 0.0291 | C: 1.51(1.05, 2.16) |
| RS746215 | 05: 172943959 | | 0.0815 | !T,T: 1.89(0.96, 3.75) | 0.0419 | C: 1.28(1.02, 1.62) | 0.0288 | !C,T: 1.44(1.1, 1.9) | 0.1419 | C: 1.18(0.95, 1.48) |
| RS13164416 | 05: 173911212 | | 0.0675 | T,T: 1.42(1.01, 1.99) | 0.0217 | T: 1.26(1.04, 1.52) | 0.0327 | T,T: 1.61(1.12, 2.3) | 0.0390 | T: 1.22(1.01, 1.47) |
| RS6556186 | 05: 174478299 | | 0.0014 | A,G: 1.63(1.23, 2.15) | 0.3835 | G: 1.1(0.89, 1.36) | 0.0046 | G,G: 2.77(1.45, 5.26) | 0.0363 | G: 1.26(1.02, 1.57) |
| RS2471005 | 05: 174814813 | | 0.0166 | !C,T: 1.48(1.13, 1.94) | 0.3194 | T: 1.11(0.91, 1.35) | 0.0316 | !C,T: 1.37(1.05, 1.78) | 0.5684 | C: 1.06(0.88, 1.28) |
| RS2913816 | 05: 177559776 | | 0.0293 | !G,G: 1.81(1.14, 2.87) | 0.2085 | A: 1.15(0.93, 1.41) | 0.0426 | !A,A: 1.4(1.07, 1.82) | 0.0381 | G: 1.23(1.02, 1.5) |
| RS11738131 | 05: 177587800 | MGC15875 | 0.0750 | !A,C: 1.48(1.05, 2.1) | 0.0457 | A: 1.39(1.02, 1.9) | 0.0592 | A,A: 1.46(1.06, 2) | 0.0281 | A: 1.39(1.04, 1.86) |
| RS11242687 | 06: 001384975 | | 0.0030 | C,T: 1.52(1.16, 1.99) | 0.5867 | T: 1.06(0.87, 1.28) | 0.0586 | !T,T: 1.37(1.04, 1.8) | 0.0221 | C: 1.25(1.04, 1.5) |
| RS9328246 | 06: 004308481 | | 0.0453 | !C,C: 2.35(1.08, 5.13) | 0.7532 | T: 1.05(0.82, 1.34) | 0.0912 | T,T: 1.3(0.98, 1.72) | 0.0341 | T: 1.31(1.02, 1.67) |
| RS9350235 | 06: 010364661 | | 0.0303 | G,G: 1.47(1.1, 1.96) | 0.0132 | G: 1.38(1.07, 1.77) | 0.1292 | G,G: 1.32(1, 1.74) | 0.0497 | G: 1.27(1.01, 1.61) |
| RS1931017 | 06: 010368921 | | 0.0116 | G,G: 1.55(1.15, 2.09) | 0.0036 | G: 1.49(1.14, 1.94) | 0.1071 | G,G: 1.35(1.02, 1.8) | 0.0385 | G: 1.3(1.02, 1.67) |
| RS12213634 | 06: 010391324 | | 0.0108 | T,T: 1.56(1.15, 2.12) | 0.0032 | T: 1.52(1.15, 2.01) | 0.0355 | T,T: 1.41(1.05, 1.88) | 0.0112 | T: 1.4(1.09, 1.82) |
| RS9370735 | 06: 014352496 | | 0.0347 | !T,T: 3.26(1.23, 8.65) | 0.0899 | G: 1.28(0.97, 1.68) | 0.1212 | G,G: 1.37(1.01, 1.86) | 0.0489 | G: 1.33(1.01, 1.75) |
| RS719343 | 06: 014445172 | | 0.0668 | !C,C: 1.37(1.04, 1.8) | 0.0211 | A: 1.3(1.04, 1.62) | 0.0186 | A,A: 2.53(1.29, 4.97) | 0.0775 | A: 1.23(0.98, 1.53) |
| RS1923747 | 06: 017334064 | | 0.0294 | A,G: 1.44(1.1, 1.88) | 0.7682 | G: 1.03(0.85, 1.25) | 0.0067 | !A,A: 1.5(1.14, 1.96) | 0.0429 | G: 1.22(1.01, 1.47) |
| RS495768 | 06: 020143047 | | 0.0341 | !A,G: 1.45(1.09, 1.93) | 0.0421 | A: 1.27(1.01, 1.6) | 0.0531 | A,A: 1.39(1.06, 1.81) | 0.0291 | A: 1.28(1.03, 1.59) |
| RS2000191 | 06: 022612727 | | 0.0088 | !A,A: 1.79(1.16, 2.75) | 0.0076 | C: 1.75(1.16, 2.66) | 0.0447 | A,C: 1.58(1.01, 2.46) | 0.0505 | C: 1.54(1, 2.37) |
| RS537835 | 06: 024075102 | | 0.0226 | C,C: 1.73(1.09, 2.75) | 0.0179 | C: 1.72(1.1, 2.7) | 0.0596 | C,C: 1.66(1, 2.74) | 0.0390 | C: 1.66(1.02, 2.72) |
| RS12196939 | 06: 027895969 | | 0.0459 | !A,A: 1.42(1.07, 1.88) | 0.0153 | G: 1.35(1.06, 1.71) | 0.1006 | !A,A: 1.31(0.97, 1.76) | 0.0389 | G: 1.33(1.02, 1.72) |
| RS200991 | 06: 027923473 | | 0.0311 | G,G: 1.42(1.05, 1.93) | 0.0129 | G: 1.42(1.08, 1.86) | 0.1064 | G,G: 1.36(1.02, 1.81) | 0.0456 | G: 1.3(1.01, 1.68) |
| RS11752919 | 06: 028511582 | ZNF390 | 0.0230 | T,T: 1.46(1.11, 1.91) | 0.0139 | T: 1.3(1.06, 1.59) | 0.0423 | !C,T: 1.39(1.07, 1.81) | 0.3925 | T: 1.09(0.9, 1.31) |
| RS2191037 | 06: 028574967 | | 0.0207 | !T,T: 1.42(1.06, 1.9) | 0.0063 | C: 1.42(1.11, 1.83) | 0.0401 | !C,T: 1.36(1.01, 1.82) | 0.5258 | T: 1.09(0.85, 1.4) |
| RS6934993 | 06: 029445479 | | 0.0127 | !G,G: 2.9(1.3, 6.47) | 0.0267 | A: 1.31(1.04, 1.66) | 0.0375 | A,A: 1.37(1.05, 1.78) | 0.0105 | A: 1.32(1.07, 1.62) |
| RS2267633 | 06: 029678820 | GABBR1 | 0.0151 | G,G: 5.24(1.35, 20.32) | 0.0472 | G: 1.36(1.01, 1.84) | 0.0232 | !A,G: 1.39(1.03, 1.88) | 0.3803 | A: 1.13(0.87, 1.47) |
| RS2076483 | 06: 029679524 | GABBR1 | 0.0151 | C,C: 5.24(1.35, 20.32) | 0.0472 | C: 1.36(1.01, 1.84) | 0.0232 | !C,T: 1.39(1.03, 1.88) | 0.3803 | T: 1.13(0.87, 1.47) |
| RS3132685 | 06: 030053928 | HCG9, HLA- | 0.0071 | C,C: 1.67(1.17, 2.37) | 0.0059 | C: 1.59(1.15, 2.22) | 0.0801 | C,C: 1.38(1.03, 1.86) | 0.0268 | C: 1.35(1.04, 1.76) |
| | | A, HLA-G | | | | | | | | |
| RS8321 | 06: 030140501 | ZNRD1 | 0.0041 | G,G: 1.71(1.18, 2.48) | 0.0045 | G: 1.68(1.18, 2.39) | 0.0601 | G,G: 1.4(1.03, 1.89) | 0.0214 | G: 1.39(1.05, 1.83) |
| RS1245371 | 06: 030146331 | RNF39 | 0.0270 | C,C: 1.43(1.09, 1.88) | 0.0326 | C: 1.27(1.02, 1.57) | 0.0394 | !T,T: 1.75(1.11, 2.76) | 0.2196 | C: 1.13(0.93, 1.39) |
| RS9261290 | 06: 030146626 | RNF39 | 0.0041 | T,T: 1.71(1.18, 2.48) | 0.0045 | T: 1.68(1.18, 2.39) | 0.0601 | T,T: 1.4(1.03, 1.89) | 0.0214 | T: 1.39(1.05, 1.83) |
| RS3130380 | 06: 030387109 | HCG18 | 0.0231 | A,A: 1.6(1.1, 2.31) | 0.0135 | A: 1.57(1.11, 2.22) | 0.0801 | A,A: 1.37(1.01, 1.85) | 0.0301 | A: 1.36(1.03, 1.8) |
| RS3130350 | 06: 030435818 | | 0.0104 | T,T: 1.59(1.1, 2.29) | 0.0108 | T: 1.58(1.12, 2.24) | 0.0412 | T,T: 1.45(1.07, 1.96) | 0.0122 | T: 1.43(1.08, 1.88) |
| RS2535315 | 06: 031160106 | | 0.0416 | A,G: 1.41(1.08, 1.85) | 0.7324 | A: 1.04(0.86, 1.26) | 0.0258 | A,A: 1.7(1.11, 2.59) | 0.0154 | A: 1.28(1.05, 1.55) |
| RS2535311 | 06: 031160663 | | 0.0416 | C,T: 1.41(1.08, 1.85) | 0.7324 | T: 1.04(0.86, 1.26) | 0.0258 | T,T: 1.7(1.11, 2.59) | 0.0154 | T: 1.28(1.05, 1.55) |
| RS7750641 | 06: 031237289 | TCF19 | 0.1034 | T,T: 1.41(0.99, 2) | 0.0437 | T: 1.41(1.01, 1.96) | 0.0643 | T,T: 1.41(1.05, 1.88) | 0.0228 | T: 1.36(1.05, 1.75) |
| RS6457374 | 06: 031380240 | BAT1, HLA- | 0.0236 | !T,T: 2.03(1.1, 3.76) | 0.0108 | C: 1.35(1.08, 1.7) | 0.0152 | C,C: 1.42(1.09, 1.85) | 0.0046 | C: 1.33(1.09, 1.63) |
| | | B, HLA-C | | | | | | | | |
| RS2596501 | 06: 031429190 | BAT1, HLA- | 0.0127 | !G,G: 1.61(1.17, 2.23) | 0.0525 | A: 1.21(1, 1.46) | 0.0150 | A,A: 1.57(1.13, 2.19) | 0.0083 | A: 1.29(1.07, 1.55) |
| | | B, HLA-C | | | | | | | | |
| RS2523554 | 06: 031439808 | BAT1 | 0.0475 | A,A: 1.33(1.01, 1.75) | 0.0154 | A: 1.28(1.05, 1.56) | 0.0058 | A,A: 1.58(1.19, 2.09) | 0.0078 | A: 1.29(1.07, 1.55) |
| RS2844513 | 06: 031496193 | BAT1 | 0.0173 | !C,C: 1.55(1.13, 2.11) | 0.0116 | T: 1.28(1.06, 1.55) | 0.0354 | T,T: 1.61(1.11, 2.34) | 0.0308 | T: 1.23(1.02, 1.49) |
| RS2844511 | 06: 031497763 | BAT1 | 0.0069 | C,C: 1.54(1.16, 2.04) | 0.0022 | C: 1.36(1.12, 1.65) | 0.0184 | C,C: 1.5(1.1, 2.03) | 0.0056 | C: 1.3(1.08, 1.56) |
| RS2284178 | 06: 031540104 | HCP5 | 0.0188 | !T,T: 1.51(1.12, 2.03) | 0.0650 | C: 1.2(0.99, 1.45) | 0.0038 | !T,T: 1.57(1.17, 2.12) | 0.0014 | C: 1.35(1.12, 1.63) |
| RS3099844 | 06: 031556955 | BAT1 | 0.0630 | A,A: 1.49(1.06, 2.11) | 0.0284 | A: 1.44(1.05, 1.98) | 0.0280 | A,A: 1.5(1.11, 2.01) | 0.0091 | A: 1.42(1.09, 1.86) |
| RS2844498 | 06: 031584833 | BAT1, MICB | 0.0368 | A,A: 1.46(1.03, 2.06) | 0.0127 | A: 1.28(1.06, 1.55) | 0.0468 | A,A: 1.58(1.1, 2.27) | 0.0632 | A: 1.2(0.99, 1.44) |
| RS2857595 | 06: 031676448 | | 0.1069 | !T,T: 1.93(0.93, 4.03) | 0.0494 | C: 1.29(1, 1.65) | 0.0097 | !T,T: 2.3(1.17, 4.52) | 0.0042 | C: 1.4(1.12, 1.77) |
| RS2261033 | 06: 031711570 | BAT2 | 0.0147 | C,T: 1.45(1.1, 1.89) | 0.3325 | T: 1.1(0.91, 1.33) | 0.0093 | !C,C: 1.52(1.16, 2) | 0.0141 | T: 1.27(1.05, 1.53) |
| RS2734335 | 06: 032001923 | C2 | 0.0552 | !C,C: 1.49(1.06, 2.09) | 0.0249 | T: 1.25(1.03, 1.51) | 0.0410 | !C,C: 1.43(1.08, 1.89) | 0.0369 | T: 1.22(1.01, 1.47) |
| RS9276644 | 06: 032853021 | | 0.0352 | !C,C: 1.72(1.14, 2.59) | 0.0607 | T: 1.22(1, 1.48) | 0.0478 | T,T: 1.34(1.03, 1.74) | 0.0143 | T: 1.28(1.05, 1.56) |
| RS6924102 | 06: 032919361 | PSMB8, | 0.0345 | !G,G: 1.46(1.03, 2.07) | 0.0110 | A: 1.29(1.06, 1.56) | 0.0097 | !G,G: 1.58(1.13, 2.22) | 0.0033 | A: 1.32(1.1, 1.6) |
| | | TAP1 | | | | | | | | |
| RS2071538 | 06: 032926656 | TAP1 | 0.0033 | !C,C: 1.57(1.2, 2.07) | 0.0007 | T: 1.47(1.18, 1.84) | 0.0639 | !C,C: 1.33(1.02, 1.74) | 0.0233 | T: 1.3(1.04, 1.62) |
| RS9276814 | 06: 032933068 | | 0.0247 | !G,G: 2.02(1.2, 3.4) | 0.0475 | A: 1.24(1.01, 1.53) | 0.0250 | A,A: 1.38(1.06, 1.79) | 0.0079 | A: 1.32(1.08, 1.62) |
| RS241404 | 06: 032973975 | | 0.0042 | !G,G: 1.66(1.16, 2.38) | 0.0015 | A: 1.37(1.13, 1.66) | 0.0206 | !G,G: 1.63(1.15, 2.31) | 0.0453 | A: 1.21(1.01, 1.46) |
| RS154981 | 06: 032988971 | | 0.0025 | G,G: 1.61(1.17, 2.22) | 0.0006 | G: 1.4(1.16, 1.7) | 0.0295 | !A,A: 1.51(1.1, 2.07) | 0.0187 | G: 1.25(1.04, 1.5) |
| RS1061632 | 06: 036565462 | KCTD20 | 0.0335 | !T,T: 1.45(1.1, 1.92) | 0.0176 | C: 1.34(1.05, 1.71) | 0.0397 | !C,C: 2.24(1.07, 4.7) | 0.8114 | T: 1.03(0.82, 1.3) |
| RS626516 | 06: 036573358 | STK38 | 0.1384 | G,G: 1.28(0.98, 1.69) | 0.0489 | G: 1.26(1.01, 1.58) | 0.0478 | !A,A: 2.05(1.12, 3.75) | 0.0529 | G: 1.25(1, 1.56) |
| RS2300064 | 06: 036582119 | STK38 | 0.0335 | !C,C: 1.45(1.1, 1.92) | 0.0176 | T: 1.34(1.05, 1.71) | 0.0396 | !T,T: 2.24(1.07, 4.69) | 0.8582 | C: 1.03(0.81, 1.3) |
| RS7741267 | 06: 037216358 | | 0.0464 | !G,T: 1.33(1.01, 1.74) | 0.6495 | T: 1.05(0.86, 1.28) | 0.0178 | T,T: 1.85(1.2, 2.85) | 0.0658 | T: 1.2(0.99, 1.46) |
| RS16891295 | 06: 039015269 | DNAH8 | 0.0232 | G,G: 5.44(0.95, 31.04) | 0.9263 | A: 1.03(0.72, 1.47) | 0.0364 | !A,G: 1.57(1.05, 2.34) | 0.0919 | A: 1.38(0.95, 2) |
| RS6901007 | 06: 043000812 | PTCRA | 0.0449 | !G,T: 1.37(1.04, 1.79) | 0.4658 | T: 1.08(0.89, 1.3) | 0.0305 | T,T: 1.52(1.08, 2.12) | 0.2368 | T: 1.12(0.93, 1.35) |
| RS4714675 | 06: 043395871 | | 0.0715 | C,C: 1.46(1.05, 2.02) | 0.0269 | C: 1.41(1.04, 1.9) | 0.0014 | C,C: 1.64(1.17, 2.29) | 0.0008 | C: 1.69(1.24, 2.31) |
| RS873053 | 06: 045955093 | | 0.0274 | C,C: 1.91(1.11, 3.28) | 0.0135 | C: 1.31(1.06, 1.63) | 0.1312 | !T,T: 1.29(1, 1.68) | 0.0422 | C: 1.24(1.01, 1.52) |
| RS6901799 | 06: 052055216 | PKHD1 | 0.0308 | A,G: 1.4(1, 1.95) | 0.4500 | G: 1.13(0.84, 1.51) | 0.0565 | !G,G: 3.75(0.93, 15.19) | 0.0489 | A: 1.35(1, 1.82) |
| RS1316575 | 06: 054273259 | | 0.0683 | A,A: 1.33(1, 1.76) | 0.0261 | A: 1.25(1.03, 1.52) | 0.0218 | !A,G: 1.43(1.1, 1.86) | 0.1459 | A: 1.15(0.95, 1.4) |
| RS937147 | 06: 063118894 | | 0.0347 | !C,T: 1.42(1.09, 1.86) | 0.5143 | T: 1.07(0.88, 1.3) | 0.0773 | T,T: 1.3(0.99, 1.7) | 0.0267 | T: 1.24(1.03, 1.5) |
| RS9354216 | 06: 066053576 | | 0.0102 | C,T: 1.48(1.1, 1.98) | 0.2606 | C: 1.15(0.9, 1.47) | 0.0246 | C,T: 1.47(1.11, 1.94) | 0.0462 | C: 1.26(1.01, 1.58) |
| RS12213776 | 06: 079147541 | | 0.0921 | !T,T: 1.3(0.99, 1.7) | 0.0382 | G: 1.26(1.02, 1.56) | 0.0954 | !T,T: 1.33(1.03, 1.73) | 0.0488 | G: 1.24(1, 1.52) |
| RS13220420 | 06: 080923376 | BCKDHB | 0.0096 | T,T: 1.81(1.17, 2.8) | 0.0080 | T: 1.78(1.17, 2.72) | 0.0273 | !G,G: 10.81(0.6, 196.01) | 0.0468 | T: 1.43(1.02, 2.01) |
| RS10485285 | 06: 081535326 | | 0.0303 | T,T: 1.7(1.12, 2.58) | 0.2211 | T: 1.14(0.93, 1.39) | 0.0130 | C,C: 1.48(1.14, 1.93) | 0.0056 | C: 1.32(1.08, 1.6) |
| RS10755417 | 06: 082652678 | | 0.0908 | C,C: 1.39(1.01, 1.92) | 0.0327 | C: 1.23(1.02, 1.49) | 0.0263 | C,C: 1.52(1.12, 2.08) | 0.0541 | C: 1.2(1, 1.44) |
| RS13192303 | 06: 084052583 | ME1 | 0.0394 | A,G: 1.54(1.08, 2.2) | 0.1130 | G: 1.29(0.94, 1.76) | 0.0542 | A,G: 1.51(1.06, 2.14) | 0.0494 | G: 1.39(1, 1.91) |
| RS12527778 | 06: 086311241 | SNX14 | 0.0568 | !C,C: 2.75(1.1, 6.88) | 0.0446 | T: 1.36(1.01, 1.82) | 0.0310 | !C,T: 1.54(1.1, 2.14) | 0.0521 | T: 1.34(1, 1.8) |
| RS9390728 | 06: 101647596 | | 0.1181 | !C,C: 1.29(0.98, 1.69) | 0.0447 | A: 1.27(1.01, 1.59) | 0.0367 | !C,C: 1.41(1.08, 1.83) | 0.0152 | A: 1.32(1.06, 1.65) |
| RS1901708 | 06: 105025706 | | 0.0331 | !G,T: 1.46(1.08, 1.97) | 0.1554 | G: 1.2(0.93, 1.55) | 0.0735 | G,G: 1.33(0.99, 1.78) | 0.0293 | G: 1.34(1.03, 1.73) |
| RS352066 | 06: 114353200 | | 0.0229 | !C,C: 1.73(1.16, 2.57) | 0.0319 | T: 1.24(1.02, 1.51) | 0.0266 | C,T: 1.41(1.08, 1.83) | 0.0667 | C: 1.2(0.99, 1.46) |
| RS2499618 | 06: 114391946 | HDAC2 | 0.0407 | !C,C: 1.62(1.11, 2.35) | 0.0468 | T: 1.22(1.01, 1.49) | 0.0147 | C,T: 1.47(1.13, 1.91) | 0.1157 | C: 1.17(0.96, 1.42) |
| RS9489998 | 06: 120932004 | | 0.0083 | !G,G: 2.33(1.28, 4.27) | 0.4278 | T: 1.1(0.88, 1.37) | 0.0306 | !G,G: 2.12(1.18, 3.8) | 0.2328 | T: 1.14(0.92, 1.41) |
| RS11759649 | 06: 121974359 | | 0.0166 | !C,T: 1.39(1.06, 1.82) | 0.6492 | C: 1.05(0.86, 1.28) | 0.0114 | T,T: 1.44(1.11, 1.88) | 0.0024 | T: 1.35(1.11, 1.63) |
| RS9490609 | 06: 123298281 | | 0.0145 | !A,A: 1.76(1.17, 2.65) | 0.1710 | G: 1.15(0.94, 1.4) | 0.0202 | G,G: 1.47(1.12, 1.92) | 0.0173 | G: 1.26(1.04, 1.53) |
| RS1851780 | 06: 124668814 | TCBA1 | 0.0125 | A,G: 1.47(1.1, 1.97) | 0.4431 | G: 1.1(0.87, 1.39) | 0.0050 | !G,G: 2.85(1.39, 5.83) | 0.4156 | A: 1.1(0.88, 1.39) |
| RS9402011 | 06: 128347581 | PTPRK | 0.0603 | T,T: 1.38(1.06, 1.81) | 0.0345 | T: 1.26(1.02, 1.56) | 0.0100 | T,T: 1.43(1.1, 1.86) | 0.0031 | T: 1.36(1.11, 1.67) |
| RS9375543 | 06: 128352833 | PTPRK | 0.0138 | T,T: 1.53(1.15, 2.03) | 0.0156 | T: 1.27(1.05, 1.54) | 0.0108 | T,T: 1.52(1.15, 2) | 0.0056 | T: 1.31(1.08, 1.57) |
| RS3903663 | 06: 128354781 | PTPRK | 0.0506 | A,A: 1.4(1.07, 1.83) | 0.0337 | A: 1.26(1.02, 1.56) | 0.0177 | A,A: 1.39(1.07, 1.81) | 0.0058 | A: 1.34(1.09, 1.64) |
| RS6931735 | 06: 135666504 | AHI1 | 0.0066 | A,G: 1.47(1.12, 1.93) | 0.4618 | G: 1.08(0.89, 1.3) | 0.0639 | !A,A: 1.42(1.01, 1.99) | 0.0228 | G: 1.25(1.03, 1.5) |
| RS7738388 | 06: 141494838 | | 0.0051 | C,C: 2.17(1.33, 3.54) | 0.0917 | C: 1.2(0.97, 1.47) | 0.0225 | !T,T: 1.37(1.06, 1.78) | 0.0087 | C: 1.31(1.07, 1.61) |
| RS208704 | 06: 148939203 | | 0.0912 | !T,T: 1.56(0.98, 2.49) | 0.0402 | C: 1.25(1.01, 1.53) | 0.0361 | !T,T: 1.58(1.05, 2.38) | 0.0105 | C: 1.3(1.06, 1.58) |
| RS10214840 | 06: 149685402 | MAP3K7IP2 | 0.0432 | C,C: 1.79(1.1, 2.91) | 0.2565 | C: 1.13(0.92, 1.4) | 0.0350 | T,T: 1.41(1.08, 1.83) | 0.0310 | T: 1.27(1.02, 1.56) |
| RS2839947 | 06: 151230384 | MTHFD1L, | 0.0250 | !C,C: 1.55(1.1, 2.18) | 0.2229 | T: 1.13(0.93, 1.37) | 0.0647 | T,T: 1.36(1.03, 1.79) | 0.0256 | T: 1.24(1.03, 1.5) |
| | | LOC646099, | | | | | | | | |
| | | LOC652160 | | | | | | | | |
| RS4869953 | 06: 151251690 | MTHFD1L | 0.0094 | A,G: 1.47(1.12, 1.93) | 0.1446 | A: 1.16(0.96, 1.4) | 0.0139 | !G,G: 1.48(1.11, 1.96) | 0.0040 | A: 1.32(1.09, 1.58) |
| RS7745756 | 06: 155003154 | | 0.0227 | A,A: 1.52(1.12, 2.06) | 0.0099 | A: 1.42(1.09, 1.86) | 0.0397 | A,A: 1.47(1.09, 1.98) | 0.0176 | A: 1.39(1.06, 1.81) |
| RS9479923 | 06: 155012248 | | 0.1009 | A,A: 1.31(0.99, 1.75) | 0.0402 | A: 1.3(1.02, 1.66) | 0.0795 | A,A: 1.35(1.02, 1.78) | 0.0271 | A: 1.32(1.03, 1.68) |
| RS9365171 | 06: 160901726 | LPA | 0.0174 | A,C: 1.46(1.11, 1.91) | 0.9203 | C: 1.01(0.83, 1.23) | 0.0066 | !C,C: 1.96(1.27, 3.01) | 0.0157 | A: 1.28(1.05, 1.55) |
| RS3798953 | 06: 161609806 | AGPAT4 | 0.0197 | G,G: 3.8(1.34, 10.79) | 0.1089 | G: 1.25(0.96, 1.62) | 0.0035 | G,G: 4.62(1.64, 12.98) | 0.1986 | G: 1.2(0.92, 1.56) |
| RS10806758 | 06: 162761969 | PARK2 | 0.0336 | !C,T: 1.37(1.04, 1.79) | 0.6180 | C: 1.06(0.87, 1.28) | 0.0632 | !T,T: 1.32(1.01, 1.74) | 0.0189 | C: 1.25(1.04, 1.51) |
| RS6927758 | 06: 163639105 | PACRG | 0.0094 | !C,C: 1.67(1.2, 2.33) | 0.0072 | T: 1.31(1.08, 1.58) | 0.0176 | !T,T: 1.48(1.11, 1.96) | 0.0654 | C: 1.19(0.99, 1.44) |
| RS9459583 | 06: 166478154 | | 0.0381 | !C,T: 1.43(1.08, 1.91) | 0.2409 | T: 1.15(0.91, 1.45) | 0.0745 | !C,C: 1.96(0.98, 3.91) | 0.0411 | T: 1.28(1.01, 1.6) |
| RS9364336 | 06: 169114813 | | 0.0365 | !C,C: 1.37(1.03, 1.82) | 0.0107 | T: 1.29(1.06, 1.56) | 0.0404 | !C,C: 1.42(1.08, 1.87) | 0.0230 | T: 1.24(1.03, 1.5) |
| RS7452888 | 06: 169438189 | | 0.0295 | G,G: 1.53(1.12, 2.09) | 0.0327 | G: 1.24(1.02, 1.5) | 0.0278 | !A,A: 1.54(1.12, 2.13) | 0.0207 | G: 1.25(1.03, 1.5) |
| RS4721321 | 07: 002034996 | MAD1L1 | 0.0211 | !A,A: 1.45(1.11, 1.9) | 0.0304 | G: 1.26(1.02, 1.55) | 0.0726 | !A,A: 1.32(1.01, 1.72) | 0.0231 | G: 1.29(1.04, 1.6) |
| RS2906173 | 07: 002453336 | LOC641906, | 0.0344 | !C,T: 1.35(1.03, 1.77) | 0.7593 | T: 1.03(0.85, 1.26) | 0.0629 | !C,C: 1.32(1.01, 1.72) | 0.0219 | T: 1.25(1.04, 1.52) |
| | | AK024457, | | | | | | | | |
| | | FLJ00049 | | | | | | | | |
| RS9986825 | 07: 003117703 | | 0.0400 | !C,T: 1.42(1.08, 1.86) | 0.2209 | T: 1.14(0.93, 1.39) | 0.0659 | T,T: 1.33(1.02, 1.74) | 0.0200 | T: 1.25(1.04, 1.52) |
| RS4722435 | 07: 003119488 | | 0.0531 | C,C: 1.42(1.06, 1.9) | 0.0276 | C: 1.24(1.03, 1.51) | 0.0310 | !T,T: 1.51(1.1, 2.07) | 0.0187 | C: 1.25(1.04, 1.51) |
| RS4720539 | 07: 005234050 | WIPI2 | 0.0136 | !C,C: 1.6(1.15, 2.21) | 0.0056 | T: 1.31(1.08, 1.59) | 0.0069 | T,T: 1.53(1.17, 2.02) | 0.0190 | T: 1.26(1.04, 1.51) |
| RS7904 | 07: 005239647 | WIPI2 | 0.1399 | A,A: 1.39(0.94, 2.06) | 0.0485 | A: 1.23(1.01, 1.5) | 0.0136 | !G,G: 1.45(1.11, 1.89) | 0.0035 | A: 1.33(1.1, 1.61) |
| RS10486156 | 07: 007315158 | | 0.0237 | !C,C: 2(1.21, 3.31) | 0.0629 | T: 1.22(0.99, 1.5) | 0.0515 | T,T: 1.38(1.06, 1.79) | 0.0358 | T: 1.24(1.02, 1.52) |
| RS2057847 | 07: 009181854 | | 0.1041 | G,G: 1.34(1.02, 1.75) | 0.0472 | G: 1.23(1.01, 1.5) | 0.0213 | G,G: 1.4(1.07, 1.83) | 0.0061 | G: 1.31(1.08, 1.59) |
| RS1919315 | 07: 018218407 | LOC641939 | 0.0185 | C,T: 1.49(1.12, 1.97) | 0.1434 | T: 1.18(0.95, 1.47) | 0.0449 | !C,C: 1.39(1.07, 1.81) | 0.0162 | T: 1.3(1.05, 1.6) |
| RS7789412 | 07: 018224796 | LOC641939 | 0.0358 | C,T: 1.44(1.08, 1.91) | 0.2092 | C: 1.16(0.93, 1.45) | 0.0483 | !T,T: 1.39(1.07, 1.81) | 0.0209 | C: 1.29(1.04, 1.59) |
| RS1860875 | 07: 021078168 | | 0.0238 | C,T: 1.42(1.08, 1.86) | 0.7284 | C: 1.04(0.85, 1.26) | 0.0093 | T,T: 1.6(1.11, 2.3) | 0.0023 | T: 1.35(1.11, 1.63) |
| RS2107124 | 07: 025266514 | | 0.0234 | !A,A: 1.9(1.14, 3.16) | 0.0129 | G: 1.31(1.06, 1.62) | 0.0241 | !A,A: 2.06(1.2, 3.56) | 0.0351 | G: 1.26(1.02, 1.56) |
| RS2717907 | 07: 025269991 | | 0.0091 | C,C: 1.53(1.15, 2.03) | 0.0026 | C: 1.47(1.15, 1.88) | 0.0030 | !T,T: 3.65(1.52, 8.78) | 0.0066 | C: 1.4(1.1, 1.79) |
| RS2214548 | 07: 026484080 | LOC441204 | 0.0155 | !G,G: 1.49(1.13, 1.95) | 0.0077 | A: 1.33(1.08, 1.64) | 0.0113 | A,G: 1.49(1.14, 1.94) | 0.3261 | A: 1.11(0.91, 1.36) |
| RS917117 | 07: 028142830 | TCAG7.981 | 0.0077 | A,A: 2.2(1.19, 4.07) | 0.0026 | A: 1.42(1.13, 1.78) | 0.0116 | A,A: 3.08(1.4, 6.8) | 0.0941 | A: 1.23(0.97, 1.55) |
| RS849141 | 07: 028151616 | TCAG7.981 | 0.0012 | G,G: 1.64(1.25, 2.14) | 0.0035 | G: 1.38(1.11, 1.7) | 0.0456 | !A,G: 1.38(1.06, 1.8) | 0.0716 | G: 1.21(0.99, 1.48) |
| RS2730616 | 07: 043885791 | URG4 | 0.0417 | !T,T: 1.73(1.12, 2.69) | 0.0434 | C: 1.24(1.01, 1.52) | 0.0144 | !T,T: 1.74(1.12, 2.71) | 0.0057 | C: 1.32(1.09, 1.61) |
| RS2730617 | 07: 043894317 | URG4 | 0.0309 | !T,T: 1.77(1.14, 2.74) | 0.0294 | C: 1.26(1.03, 1.54) | 0.0158 | !T,T: 1.78(1.14, 2.76) | 0.0078 | C: 1.31(1.08, 1.6) |
| RS1894752 | 07: 043963600 | LOC650422 | 0.0310 | !T,T: 1.58(1.07, 2.34) | 0.0085 | C: 1.31(1.07, 1.59) | 0.0028 | !T,T: 2(1.33, 3) | 0.0164 | C: 1.27(1.05, 1.54) |
| RS7800244 | 07: 047746494 | | 0.1278 | !T,T: 1.31(0.99, 1.74) | 0.0475 | G: 1.29(1.01, 1.64) | 0.0035 | G,G: 4.17(1.62, 10.75) | 0.2191 | G: 1.17(0.92, 1.49) |
| RS12671939 | 07: 052666021 | | 0.0078 | G,G: 4.88(1.54, 15.5) | 0.1217 | G: 1.24(0.95, 1.61) | 0.0376 | G,G: 2.67(1.07, 6.69) | 0.9469 | G: 1.02(0.78, 1.32) |
| RS11978966 | 07: 053223557 | LOC653175 | 0.0063 | !T,T: 1.76(1.23, 2.53) | 0.0029 | C: 1.65(1.19, 2.3) | 0.0851 | !T,T: 1.36(0.97, 1.9) | 0.0401 | C: 1.39(1.02, 1.9) |
| RS7795810 | 07: 053358424 | | 0.0264 | C,C: 3.45(1.05, 11.36) | 0.0192 | C: 1.4(1.06, 1.86) | 0.0200 | !T,T: 1.57(1.14, 2.15) | 0.0060 | C: 1.49(1.12, 1.99) |
| RS10488321 | 07: 053428180 | | 0.0366 | G,G: 2.89(0.99, 8.48) | 0.0187 | G: 1.41(1.06, 1.87) | 0.0369 | !A,A: 1.5(1.09, 2.07) | 0.0099 | G: 1.48(1.1, 1.98) |
| RS1030306 | 07: 053606599 | | 0.0175 | C,T: 1.75(1.14, 2.66) | 0.0186 | C: 1.62(1.09, 2.4) | 0.0290 | C,T: 1.75(1.14, 2.67) | 0.0185 | C: 1.61(1.09, 2.37) |
| RS4718608 | 07: 066784667 | | 0.0144 | T,T: 1.55(1.14, 2.11) | 0.0053 | T: 1.49(1.13, 1.96) | 0.0169 | T,T: 1.43(1.06, 1.95) | 0.0079 | T: 1.46(1.11, 1.93) |
| RS4566929 | 07: 066832473 | | 0.0239 | A,A: 2.27(1.23, 4.18) | 0.1367 | A: 1.18(0.95, 1.47) | 0.0668 | !G,G: 1.32(1.01, 1.71) | 0.0186 | A: 1.28(1.04, 1.57) |
| RS6976905 | 07: 070399889 | WBSCR17 | 0.0434 | !A,C: 1.4(1.07, 1.84) | 0.8841 | A: 1.02(0.84, 1.23) | 0.0423 | !C,C: 1.39(1.04, 1.86) | 0.0127 | A: 1.27(1.05, 1.52) |
| RS37609 | 07: 073539675 | GTF2IRD1 | 0.0866 | !T,T: 1.35(1.03, 1.77) | 0.0328 | C: 1.24(1.02, 1.52) | 0.0361 | C,C: 1.69(1.12, 2.54) | 0.1539 | C: 1.15(0.95, 1.4) |
| RS798337 | 07: 077754218 | MAGI2 | 0.0756 | G,G: 1.53(0.98, 2.4) | 0.0243 | G: 1.27(1.03, 1.56) | 0.0052 | !G,T: 1.44(1.11, 1.87) | 0.8410 | G: 1.02(0.84, 1.24) |
| RS798343 | 07: 077762177 | MAGI2 | 0.1155 | !T,T: 1.31(1, 1.72) | 0.0370 | C: 1.24(1.01, 1.51) | 1.14E−05 | !C,T: 1.81(1.39, 2.36) | 0.9215 | T: 1.01(0.84, 1.23) |
| RS6966472 | 07: 084537323 | SEMA3D | 0.0932 | T,T: 1.38(1.01, 1.88) | 0.0439 | T: 1.34(1.01, 1.78) | 0.0353 | T,T: 1.49(1.09, 2.04) | 0.0104 | T: 1.45(1.1, 1.93) |
| RS6953717 | 07: 089661725 | | 0.0468 | T,T: 2(1.09, 3.66) | 0.0351 | T: 1.28(1.02, 1.6) | 0.0675 | !G,G: 1.3(0.99, 1.7) | 0.0273 | T: 1.3(1.03, 1.63) |
| RS194498 | 07: 089676093 | | 0.0624 | C,C: 1.93(1.05, 3.54) | 0.0407 | C: 1.26(1.01, 1.58) | 0.0675 | !T,T: 1.3(0.99, 1.7) | 0.0273 | C: 1.3(1.03, 1.63) |
| RS2188505 | 07: 095374205 | DYNC1I1 | 0.0054 | !C,C: 18.34(1.06, 318.73) | 0.0403 | T: 1.41(1.02, 1.95) | 0.0304 | C,T: 1.55(1.11, 2.15) | 0.0469 | C: 1.35(1.01, 1.81) |
| RS7799245 | 07: 105093792 | ATXN7L1 | 0.0582 | A,A: 1.56(1.07, 2.28) | 0.0363 | A: 1.24(1.02, 1.5) | 0.0067 | !G,G: 1.54(1.18, 2.02) | 0.0074 | A: 1.3(1.08, 1.58) |
| RS10953522 | 07: 106375662 | | 0.0238 | !A,G: 1.4(1.07, 1.84) | 0.6231 | G: 1.05(0.87, 1.27) | 0.0437 | A,A: 1.44(1.08, 1.92) | 0.0665 | A: 1.19(0.99, 1.43) |
| RS10499980 | 07: 109054772 | | 0.0076 | A,A: 1.64(1.19, 2.26) | 0.0291 | A: 1.24(1.03, 1.5) | 0.0120 | G,G: 1.55(1.15, 2.07) | 0.0084 | G: 1.28(1.07, 1.54) |
| RS6978286 | 07: 116404768 | ST7 | 0.0651 | !A,A: 1.36(1.04, 1.79) | 0.0186 | G: 1.31(1.05, 1.63) | 0.0705 | !A,A: 1.34(1.03, 1.74) | 0.0236 | G: 1.28(1.04, 1.58) |
| RS2896218 | 07: 116707214 | WNT2 | 0.0053 | !G,G: 1.72(1.21, 2.44) | 0.0024 | A: 1.35(1.11, 1.64) | 0.0098 | !G,G: 1.62(1.15, 2.3) | 0.1822 | A: 1.14(0.94, 1.37) |
| RS1443753 | 07: 121736554 | LOC154860 | 0.0445 | !T,T: 1.34(1.02, 1.75) | 0.0145 | C: 1.29(1.05, 1.57) | 0.0357 | C,C: 1.67(1.1, 2.53) | 0.0236 | C: 1.25(1.03, 1.52) |
| RS890406 | 07: 131904650 | PLXNA4B | 0.0256 | !A,G: 1.44(1.1, 1.88) | 1.0000 | G: 1(0.83, 1.21) | 0.0431 | A,A: 1.58(1.09, 2.29) | 0.0339 | A: 1.23(1.02, 1.49) |
| RS11768238 | 07: 134878053 | | 0.0042 | A,G: 1.54(1.17, 2.02) | 0.7206 | G: 1.04(0.85, 1.27) | 0.0590 | A,A: 1.35(1.04, 1.76) | 0.0207 | A: 1.27(1.04, 1.54) |
| RS6970530 | 07: 136616871 | PTN | 0.0322 | A,A: 1.71(1.05, 2.77) | 0.0250 | A: 1.71(1.07, 2.74) | 0.0572 | A,A: 1.57(0.99, 2.49) | 0.0438 | A: 1.6(1.03, 2.51) |
| RS1451526 | 07: 136731763 | DGKI | 0.0037 | !A,A: 1.53(1.16, 2.03) | 0.0008 | G: 1.5(1.19, 1.91) | 0.0628 | G,G: 2.16(0.98, 4.75) | 0.0375 | G: 1.28(1.01, 1.62) |
| RS10487553 | 07: 143358783 | | 0.0158 | G,G: 1.82(1.2, 2.75) | 0.0615 | G: 1.21(0.99, 1.48) | 0.0722 | G,G: 1.5(1.05, 2.16) | 0.0439 | G: 1.21(1.01, 1.47) |
| RS2107759 | 07: 143361152 | | 0.0153 | C,C: 1.82(1.2, 2.75) | 0.0688 | C: 1.21(0.99, 1.47) | 0.0730 | C,C: 1.5(1.04, 2.16) | 0.0439 | C: 1.22(1.01, 1.47) |
| RS826823 | 07: 146685306 | CNTNAP2 | 0.0376 | !C,T: 1.45(0.96, 2.2) | 0.2759 | T: 1.24(0.84, 1.83) | 0.0267 | T,T: 1.54(1.04, 2.29) | 0.0187 | T: 1.57(1.08, 2.28) |
| RS4314574 | 07: 146985864 | CNTNAP2 | 0.0087 | !G,G: 8.31(1.51, 45.83) | 0.1966 | A: 1.19(0.91, 1.56) | 0.0709 | A,A: 1.42(1.04, 1.93) | 0.0227 | A: 1.39(1.05, 1.85) |
| RS1225382 | 07: 155334646 | | 0.1093 | G,G: 1.3(0.99, 1.71) | 0.0383 | G: 1.23(1.01, 1.5) | 0.0282 | !A,G: 1.4(1.08, 1.81) | 0.7752 | A: 1.03(0.86, 1.24) |
| RS6559176 | 08: 006526697 | | 0.1032 | A,A: 1.35(1, 1.81) | 0.0367 | A: 1.23(1.01, 1.48) | 0.0375 | A,A: 1.46(1.09, 1.94) | 0.0534 | A: 1.2(1, 1.45) |
| RS11249877 | 08: 008572184 | | 0.0167 | !T,T: 1.61(1.15, 2.26) | 0.0081 | C: 1.53(1.12, 2.08) | 0.0199 | !T,T: 1.54(1.13, 2.09) | 0.0057 | C: 1.48(1.12, 1.96) |
| RS2898249 | 08: 010782996 | | 0.1216 | A,A: 1.55(0.96, 2.51) | 0.0479 | A: 1.24(1, 1.53) | 0.0493 | A,A: 1.9(1.13, 3.21) | 0.1558 | A: 1.16(0.95, 1.43) |
| RS10503426 | 08: 011626792 | GATA4 | 0.1142 | !C,C: 1.41(0.99, 2.01) | 0.0432 | A: 1.22(1.01, 1.49) | 0.0018 | A,C: 1.55(1.19, 2.01) | 0.4777 | A: 1.07(0.89, 1.29) |
| RS804280 | 08: 011650107 | GATA4 | 0.0066 | G,G: 1.51(1.13, 2) | 0.0016 | G: 1.37(1.13, 1.66) | 0.0220 | G,T: 1.44(1.11, 1.87) | 0.7761 | T: 1.03(0.85, 1.24) |
| RS4128298 | 08: 011860741 | | 0.0092 | !G,G: 2.22(1.29, 3.79) | 0.1541 | A: 1.17(0.95, 1.46) | 0.0548 | !G,G: 1.72(1.05, 2.8) | 0.0305 | A: 1.25(1.02, 1.53) |
| RS1481692 | 08: 013383739 | DLC1 | 0.0274 | !T,T: 1.57(1.13, 2.19) | 0.0132 | C: 1.48(1.09, 2.02) | 0.0430 | C,T: 1.5(1.09, 2.06) | 0.0400 | C: 1.34(1.02, 1.77) |
| RS6982196 | 08: 018848911 | PSD3 | 0.0136 | A,G: 1.48(1.12, 1.94) | 0.0421 | A: 1.24(1.01, 1.52) | 0.0143 | !A,A: 1.69(1.13, 2.54) | 0.3449 | G: 1.1(0.91, 1.34) |
| RS1025229 | 08: 018856300 | PSD3 | 0.0118 | !G,G: 1.49(1.13, 1.95) | 0.0261 | T: 1.26(1.03, 1.55) | 0.0287 | !T,T: 1.61(1.08, 2.39) | 0.3720 | G: 1.1(0.9, 1.33) |
| RS333047 | 08: 019094091 | | 0.0691 | !C,C: 1.33(1.01, 1.74) | 0.0254 | T: 1.27(1.03, 1.55) | 0.0392 | T,T: 1.69(1.08, 2.67) | 0.0200 | T: 1.27(1.04, 1.54) |
| RS2672245 | 08: 019119152 | | 0.0233 | !C,C: 1.57(1.08, 2.26) | 0.0094 | A: 1.59(1.13, 2.25) | 0.1164 | !C,C: 1.45(1.02, 2.06) | 0.0434 | A: 1.41(1.01, 1.97) |
| RS2631878 | 08: 021173339 | | 0.0200 | G,G: 5.44(0.95, 31.04) | 0.0261 | G: 1.39(1.04, 1.84) | 0.0136 | !A,A: 1.59(1.16, 2.19) | 0.0076 | G: 1.5(1.12, 2.01) |
| RS2631879 | 08: 021174982 | | 0.0236 | !T,T: 1.4(1.03, 1.91) | 0.0213 | C: 1.4(1.06, 1.87) | 0.0100 | !T,T: 1.62(1.18, 2.22) | 0.0044 | C: 1.52(1.14, 2.03) |
| RS1035722 | 08: 025689448 | | 0.0134 | A,A: 3.09(1.17, 8.14) | 0.0066 | A: 1.47(1.11, 1.94) | 0.0427 | A,G: 1.48(1.09, 2.01) | 0.0333 | A: 1.34(1.03, 1.74) |
| RS2681615 | 08: 031473005 | | 0.0315 | !A,A: 1.45(1.06, 1.99) | 0.0088 | G: 1.3(1.07, 1.57) | 0.0412 | !A,A: 1.37(1.02, 1.83) | 0.0098 | G: 1.28(1.06, 1.54) |
| RS16921196 | 08: 056207801 | XKR4 | 0.0474 | !A,C: 1.43(1.07, 1.92) | 0.0353 | A: 1.3(1.02, 1.66) | 0.0095 | C,C: 3.08(1.4, 6.8) | 0.2147 | C: 1.17(0.92, 1.49) |
| RS7460495 | 08: 059596784 | | 0.0108 | A,A: 1.7(1.2, 2.4) | 0.0161 | A: 1.27(1.05, 1.54) | 0.0363 | !A,G: 1.4(1.08, 1.82) | 0.1738 | G: 1.14(0.94, 1.38) |
| RS1834733 | 08: 059600018 | | 0.0052 | G,G: 1.76(1.25, 2.49) | 0.0094 | G: 1.29(1.07, 1.57) | 0.0425 | !A,G: 1.39(1.07, 1.8) | 0.1899 | A: 1.14(0.94, 1.38) |
| RS1582824 | 08: 059609008 | | 0.0057 | G,G: 1.75(1.24, 2.47) | 0.0110 | G: 1.28(1.06, 1.56) | 0.0318 | !A,G: 1.4(1.08, 1.82) | 0.1208 | A: 1.16(0.96, 1.41) |
| RS10504321 | 08: 061975455 | | 0.0070 | G,G: 8.08(1.48, 44.18) | 0.0483 | G: 1.34(1, 1.79) | 0.0015 | !T,T: 1.78(1.28, 2.47) | 0.0009 | G: 1.68(1.24, 2.29) |
| RS728032 | 08: 069279032 | DEPDC2 | 0.0233 | T,T: 4.44(1.12, 17.54) | 0.0371 | T: 1.34(1.02, 1.76) | 0.0536 | C,T: 1.46(1.07, 1.99) | 0.0276 | T: 1.37(1.04, 1.81) |
| RS1530241 | 08: 070573442 | SULF1 | 0.0640 | C,C: 1.41(1.05, 1.89) | 0.0255 | C: 1.24(1.03, 1.51) | 0.1021 | !A,A: 1.34(0.96, 1.88) | 0.0376 | C: 1.22(1.01, 1.47) |
| RS1946937 | 08: 070581000 | SULF1 | 0.0214 | !G,G: 1.48(1.12, 1.95) | 0.0113 | A: 1.35(1.07, 1.71) | 0.0031 | !A,A: 2.97(1.5, 5.89) | 0.1197 | G: 1.2(0.96, 1.51) |
| RS1524641 | 08: 086009342 | | 0.0005 | !C,T: 2.3(1.42, 3.75) | 0.0047 | C: 1.91(1.21, 3.02) | 0.0118 | !C,T: 1.92(1.19, 3.12) | 0.0249 | C: 1.69(1.08, 2.65) |
| RS6991672 | 08: 087721231 | CNGB3 | 0.0016 | C,T: 2.06(1.32, 3.22) | 0.0020 | C: 1.92(1.26, 2.93) | 0.0404 | !T,T: 1.48(1, 2.2) | 0.0352 | C: 1.51(1.04, 2.21) |
| RS1996637 | 08: 089227035 | MMP16 | 0.0589 | C,C: 1.41(1.05, 1.89) | 0.0249 | C: 1.25(1.03, 1.51) | 0.0165 | C,C: 1.53(1.14, 2.05) | 0.0085 | C: 1.28(1.07, 1.54) |
| RS2681297 | 08: 089238474 | MMP16 | 0.0587 | C,C: 1.41(1.05, 1.89) | 0.0249 | C: 1.25(1.03, 1.51) | 0.0252 | C,C: 1.49(1.11, 2.01) | 0.0128 | C: 1.27(1.05, 1.52) |
| RS2575738 | 08: 097599578 | SDC2 | 0.0233 | !C,T: 1.39(1.06, 1.82) | 1.0000 | T: 1(0.82, 1.23) | 0.0014 | T,T: 2.56(1.48, 4.42) | 0.1309 | T: 1.17(0.96, 1.44) |
| RS1503555 | 08: 107376981 | | 0.0456 | !C,C: 3.25(1.11, 9.52) | 0.0719 | T: 1.29(0.99, 1.7) | 0.0485 | !C,T: 1.45(1.06, 1.97) | 0.1154 | T: 1.25(0.95, 1.63) |
| RS10089853 | 08: 107395023 | | 0.0381 | !C,C: 3.86(1.17, 12.74) | 0.0871 | A: 1.3(0.97, 1.74) | 0.0264 | !A,C: 1.47(1.07, 2.02) | 0.1315 | A: 1.25(0.94, 1.66) |
| RS12682157 | 08: 107400829 | | 0.0345 | !G,G: 2.24(1.15, 4.35) | 0.0340 | A: 1.29(1.02, 1.62) | 0.0790 | A,A: 1.36(1.04, 1.77) | 0.0469 | A: 1.26(1, 1.57) |
| RS10505093 | 08: 107686536 | | 0.0078 | A,A: 1.54(1.17, 2.03) | 0.0053 | A: 1.33(1.09, 1.63) | 0.0845 | A,A: 1.34(1.03, 1.74) | 0.0327 | A: 1.24(1.02, 1.51) |
| RS1858342 | 08: 115003522 | | 0.1359 | !T,T: 1.52(0.97, 2.37) | 0.0437 | C: 1.55(1.01, 2.37) | 0.0381 | !T,T: 1.69(1.09, 2.64) | 0.0190 | C: 1.66(1.09, 2.55) |
| RS10108622 | 08: 115631498 | | 0.0202 | !G,G: 2.42(1.22, 4.79) | 0.0244 | T: 1.31(1.04, 1.65) | 0.0218 | !G,G: 2.25(1.24, 4.08) | 0.1064 | T: 1.2(0.97, 1.5) |
| RS4871533 | 08: 125831179 | | 0.0025 | C,C: 1.5(1.14, 1.98) | 0.0005 | C: 1.5(1.19, 1.88) | 0.0432 | !C,T: 1.41(1.08, 1.85) | 0.1957 | C: 1.15(0.93, 1.42) |
| RS6470459 | 08: 127771130 | | 0.0058 | A,A: 1.49(1.13, 1.95) | 0.0020 | A: 1.43(1.14, 1.78) | 0.0886 | A,A: 1.32(1.01, 1.73) | 0.0262 | A: 1.29(1.03, 1.62) |
| RS2648829 | 08: 129217093 | | 0.0662 | !T,T: 1.39(1.04, 1.85) | 0.0156 | C: 1.35(1.06, 1.73) | 0.0294 | C,C: 2.1(1.12, 3.93) | 0.6109 | C: 1.06(0.85, 1.33) |
| RS13257555 | 08: 130011810 | | 0.0363 | C,T: 1.55(1.03, 2.33) | 0.0780 | C: 1.44(0.97, 2.12) | 0.0215 | C,C: 13.56(0.76, 241.41) | 0.0654 | C: 1.42(0.99, 2.05) |
| RS9693430 | 08: 130034016 | | 0.0377 | !G,G: 9.61(0.52, 179.12) | 0.5791 | G: 1.12(0.78, 1.6) | 0.0062 | !T,T: 1.91(1.26, 2.88) | 0.0021 | G: 1.83(1.25, 2.69) |
| RS894263 | 08: 130072445 | | 0.0714 | T,T: 1.43(1.01, 2.02) | 0.0246 | T: 1.25(1.03, 1.51) | 0.0426 | T,T: 1.49(1.04, 2.13) | 0.0170 | T: 1.26(1.04, 1.52) |
| RS6981912 | 08: 134747299 | | 0.0583 | G,G: 1.74(1.08, 2.79) | 0.0406 | G: 1.25(1.01, 1.55) | 0.0254 | !A,A: 1.4(1.08, 1.82) | 0.0073 | G: 1.34(1.08, 1.65) |
| RS1123762 | 08: 135191884 | | 0.0652 | C,C: 3.26(0.79, 13.43) | 0.0457 | C: 1.34(1.01, 1.77) | 0.0256 | C,T: 1.47(1.08, 1.99) | 0.2371 | C: 1.18(0.91, 1.52) |
| RS9324520 | 08: 136123429 | | 0.0187 | T,T: 1.48(1.13, 1.93) | 0.0110 | T: 1.3(1.06, 1.6) | 0.0862 | T,T: 1.33(1.02, 1.73) | 0.0311 | T: 1.24(1.02, 1.52) |
| RS10104990 | 08: 136202757 | | 0.1112 | G,G: 1.36(1.01, 1.83) | 0.0464 | G: 1.21(1, 1.47) | 0.0193 | G,G: 1.46(1.1, 1.95) | 0.1090 | G: 1.16(0.97, 1.4) |
| RS4909885 | 08: 136203239 | | 0.1015 | G,G: 1.36(1.01, 1.83) | 0.0367 | G: 1.23(1.01, 1.48) | 0.0169 | G,G: 1.48(1.11, 1.97) | 0.0897 | G: 1.17(0.98, 1.41) |
| RS1009149 | 08: 136466442 | | 0.0244 | G,G: 1.82(1.11, 2.98) | 0.4453 | G: 1.09(0.88, 1.35) | 0.0160 | A,A: 1.46(1.12, 1.91) | 0.0194 | A: 1.31(1.05, 1.62) |
| RS9657469 | 08: 136467298 | | 0.0321 | G,G: 1.82(1.11, 2.98) | 0.3550 | G: 1.11(0.9, 1.38) | 0.0195 | T,T: 1.45(1.11, 1.89) | 0.0226 | T: 1.3(1.04, 1.61) |
| RS11780624 | 08: 138132395 | | 0.0284 | !T,T: 1.43(1.09, 1.88) | 0.0090 | C: 1.3(1.07, 1.59) | 0.0309 | !T,T: 1.39(1.06, 1.82) | 0.0092 | C: 1.29(1.06, 1.55) |
| RS11780634 | 08: 138190860 | | 0.0415 | G,G: 1.93(1.05, 3.55) | 0.0257 | G: 1.29(1.04, 1.61) | 0.1411 | !A,A: 1.26(0.97, 1.64) | 0.0463 | G: 1.25(1, 1.55) |
| RS11780467 | 08: 138268410 | | 0.0337 | !T,T: 1.45(1.09, 1.92) | 0.0134 | C: 1.37(1.07, 1.75) | 0.0705 | !T,T: 1.38(1.05, 1.83) | 0.0237 | C: 1.32(1.04, 1.68) |
| RS868421 | 08: 138668249 | | 0.0068 | !A,G: 1.55(1.18, 2.04) | 0.0987 | G: 1.2(0.97, 1.47) | 0.0207 | !A,A: 1.93(1.2, 3.11) | 0.0430 | G: 1.24(1.01, 1.52) |
| RS4243888 | 08: 139322987 | | 0.0314 | !T,T: 1.62(1.07, 2.46) | 0.4437 | C: 1.08(0.89, 1.32) | 0.0019 | !T,T: 2.15(1.36, 3.38) | 0.0026 | C: 1.36(1.11, 1.65) |
| RS2977451 | 08: 141603380 | LOC646107 | 0.0223 | !G,G: 1.48(1.09, 2.02) | 0.0064 | A: 1.47(1.12, 1.94) | 0.0751 | !G,G: 1.4(1.05, 1.87) | 0.0329 | A: 1.32(1.02, 1.7) |
| RS2977477 | 08: 141624141 | EIF2C2 | 0.1011 | !G,G: 1.36(1.01, 1.81) | 0.0337 | A: 1.32(1.03, 1.71) | 0.0840 | !G,G: 1.36(1.03, 1.79) | 0.0317 | A: 1.3(1.03, 1.64) |
| RS2280880 | 08: 143718931 | | 0.0974 | T,T: 1.55(0.99, 2.41) | 0.0449 | T: 1.23(1.01, 1.51) | 0.0484 | !G,G: 1.38(1.06, 1.8) | 0.0190 | T: 1.27(1.04, 1.54) |
| RS2382962 | 08: 144740535 | EEF1D | 0.0390 | A,A: 2.89(0.99, 8.48) | 1.0000 | A: 1.01(0.73, 1.39) | 0.0410 | !A,G: 1.53(1.03, 2.28) | 0.1653 | G: 1.3(0.9, 1.87) |
| RS896962 | 08: 144758235 | PYCRL | 0.0131 | G,G: 2.83(1.28, 6.24) | 0.4509 | G: 1.1(0.86, 1.41) | 0.0171 | !A,G: 1.54(1.14, 2.08) | 0.0326 | A: 1.33(1.03, 1.74) |
| RS1509197 | 09: 001009473 | | 0.0203 | T,T: 2.28(1.21, 4.29) | 0.3281 | T: 1.13(0.9, 1.41) | 0.0610 | C,C: 1.37(1.06, 1.79) | 0.0329 | C: 1.27(1.02, 1.57) |
| RS1535844 | 09: 002184805 | SMARCA2 | 0.0618 | !C,C: 1.32(1, 1.74) | 0.0290 | T: 1.31(1.03, 1.66) | 0.0443 | !C,C: 1.4(1.07, 1.84) | 0.0165 | T: 1.33(1.05, 1.67) |
| RS1535843 | 09: 002184871 | SMARCA2 | 0.0445 | !T,T: 1.33(1.01, 1.76) | 0.0215 | C: 1.33(1.04, 1.68) | 0.0443 | !T,T: 1.4(1.07, 1.84) | 0.0165 | C: 1.33(1.05, 1.67) |
| RS914494 | 09: 002459939 | FLJ35024 | 0.0094 | A,A: 1.51(1.15, 1.97) | 0.0026 | A: 1.39(1.12, 1.71) | 0.0105 | !A,G: 1.5(1.15, 1.97) | 0.0525 | A: 1.23(1, 1.51) |
| RS2027394 | 09: 004059657 | GLIS3 | 0.0356 | !G,G: 1.65(1.11, 2.44) | 0.1444 | A: 1.16(0.95, 1.41) | 0.0126 | A,G: 1.47(1.13, 1.9) | 0.6568 | G: 1.05(0.87, 1.27) |
| RS1931236 | 09: 011092971 | | 0.0221 | !T,T: 2.32(1.2, 4.51) | 0.0356 | G: 1.26(1.02, 1.57) | 0.0215 | !T,T: 2.34(1.25, 4.4) | 0.1674 | G: 1.17(0.94, 1.45) |
| RS7857061 | 09: 013506313 | | 0.0412 | !A,G: 1.42(1.08, 1.86) | 0.2185 | A: 1.14(0.93, 1.39) | 0.0200 | !G,G: 1.82(1.14, 2.92) | 0.0107 | A: 1.3(1.06, 1.59) |
| RS518054 | 09: 013679066 | | 0.0347 | !T,T: 2.59(1.2, 5.6) | 0.0741 | G: 1.24(0.98, 1.56) | 0.0307 | !T,T: 2.19(1.16, 4.14) | 0.0292 | G: 1.29(1.03, 1.62) |
| RS1318995 | 09: 024901674 | | 0.0030 | C,C: 1.64(1.22, 2.21) | 0.0198 | C: 1.26(1.04, 1.52) | 0.0077 | C,C: 1.59(1.16, 2.17) | 0.0026 | C: 1.33(1.11, 1.6) |
| RS2126176 | 09: 024912485 | | 0.0299 | A,A: 1.81(1.16, 2.82) | 0.0545 | A: 1.23(1, 1.5) | 0.0307 | A,A: 1.69(1.13, 2.53) | 0.0983 | A: 1.18(0.97, 1.44) |
| RS4978100 | 09: 026741013 | | 0.0279 | T,T: 1.97(1.16, 3.33) | 0.0395 | T: 1.25(1.01, 1.53) | 0.0211 | !C,C: 1.38(1.06, 1.79) | 0.0048 | T: 1.34(1.1, 1.64) |
| RS2070074 | 09: 034639442 | GALT | 0.0938 | !A,A: 1.47(1.03, 2.1) | 0.0321 | G: 1.43(1.03, 1.99) | 0.0765 | !A,A: 1.48(1.04, 2.09) | 0.0352 | G: 1.41(1.03, 1.95) |
| RS7036081 | 09: 037613913 | | 0.0396 | !C,C: 1.46(1.09, 1.96) | 0.0127 | T: 1.39(1.07, 1.8) | 0.0323 | T,T: 2.67(1.07, 6.69) | 0.0205 | T: 1.35(1.05, 1.74) |
| RS10973953 | 09: 038629853 | | 0.0337 | !A,G: 1.44(1.09, 1.89) | 0.1274 | A: 1.18(0.96, 1.47) | 0.0070 | G,G: 2.25(1.25, 4.04) | 0.0042 | G: 1.37(1.11, 1.7) |
| RS12342083 | 09: 077385731 | | 0.0241 | T,T: 1.55(1.12, 2.15) | 0.0125 | T: 1.47(1.09, 1.98) | 0.0465 | !C,C: 3.83(1.17, 12.51) | 0.1758 | T: 1.22(0.92, 1.62) |
| RS4745430 | 09: 077461845 | | 0.0175 | !T,T: 1.65(1.11, 2.44) | 0.0068 | C: 1.32(1.08, 1.6) | 0.0994 | C,C: 1.3(1, 1.7) | 0.0300 | C: 1.24(1.02, 1.5) |
| RS4745431 | 09: 077461915 | | 0.0706 | !T,T: 1.44(1.03, 2.02) | 0.0284 | C: 1.24(1.03, 1.5) | 0.0189 | C,C: 1.44(1.1, 1.89) | 0.0054 | C: 1.31(1.09, 1.58) |
| RS10869675 | 09: 077779855 | PCSK5 | 0.0393 | G,G: 1.81(1.13, 2.9) | 0.0449 | G: 1.24(1.01, 1.52) | 0.0193 | !A,A: 1.44(1.11, 1.87) | 0.0305 | G: 1.25(1.02, 1.53) |
| RS2007744 | 09: 078044132 | | 0.0278 | A,C: 1.53(1.12, 2.1) | 0.0665 | C: 1.29(0.99, 1.69) | 1.35E−05 | A,C: 2.01(1.5, 2.71) | 903E−05 | C: 1.65(1.28, 2.12) |
| RS7043482 | 09: 084325735 | | 0.0227 | A,C: 1.45(1.11, 1.9) | 0.1238 | C: 1.18(0.96, 1.44) | 0.0112 | A,C: 1.49(1.15, 1.95) | 0.1744 | C: 1.15(0.94, 1.4) |
| RS4242620 | 09: 085145258 | FRMD3 | 0.0878 | A,A: 1.34(1.03, 1.76) | 0.0333 | A: 1.27(1.02, 1.57) | 0.0693 | !G,G: 1.74(0.99, 3.05) | 0.0312 | A: 1.28(1.03, 1.58) |
| RS11140345 | 09: 085859150 | | 0.0241 | !G,G: 1.69(1.09, 2.61) | 0.5065 | A: 1.07(0.88, 1.31) | 0.0793 | A,A: 1.35(1.04, 1.76) | 0.0387 | A: 1.23(1.01, 1.49) |
| RS1599045 | 09: 088831397 | LOC440173 | 0.0038 | !T,T: 5.15(1.61, 16.46) | 0.0205 | C: 1.37(1.05, 1.77) | 0.1344 | C,C: 1.29(0.97, 1.7) | 0.0442 | C: 1.28(1.01, 1.63) |
| RS465855 | 09: 088988236 | | 0.0426 | !G,G: 1.52(1.09, 2.11) | 0.0582 | A: 1.2(1, 1.46) | 0.1336 | !G,G: 1.32(0.97, 1.8) | 0.0486 | A: 1.21(1, 1.45) |
| RS277722 | 09: 088990946 | | 0.0429 | !G,G: 1.52(1.09, 2.11) | 0.0520 | A: 1.21(1, 1.46) | 0.1045 | !G,G: 1.35(1, 1.84) | 0.0388 | A: 1.22(1.01, 1.46) |
| RS11265877 | 09: 091509347 | FLJ42342 | 0.0284 | !A,A: 1.53(1.05, 2.23) | 0.0239 | G: 1.52(1.06, 2.17) | 0.0138 | A,G: 1.52(1.06, 2.19) | 0.2085 | G: 1.24(0.89, 1.72) |
| RS7045961 | 09: 091753819 | | 0.0425 | G,G: 2.92(1.19, 7.17) | 0.1679 | G: 1.21(0.92, 1.59) | 0.0668 | T,T: 1.45(1.05, 2) | 0.0256 | T: 1.4(1.04, 1.88) |
| RS10993008 | 09: 092125202 | | 0.0407 | A,G: 1.47(1.08, 1.98) | 0.0283 | G: 1.34(1.03, 1.75) | 0.0101 | !G,G: 3.54(1.07, 11.66) | 0.5114 | G: 1.1(0.85, 1.42) |
| RS2296751 | 09: 092921175 | | 0.0555 | !G,T: 1.46(1.07, 2) | 0.0371 | T: 1.35(1.02, 1.77) | 0.0055 | !G,G: 6.2(1.62, 23.73) | 0.0848 | T: 1.27(0.97, 1.67) |
| RS12339155 | 09: 093434461 | | 0.0136 | C,C: 2.14(1.13, 4.04) | 0.9087 | C: 1.02(0.81, 1.28) | 0.0949 | !T,T: 1.31(1, 1.71) | 0.0290 | C: 1.28(1.03, 1.6) |
| RS7863890 | 09: 094465567 | IPPK | 0.0590 | G,G: 1.68(0.98, 2.87) | 0.0221 | G: 1.28(1.04, 1.59) | 0.0468 | A,G: 1.38(1.06, 1.8) | 0.0710 | G: 1.21(0.99, 1.48) |
| RS1758799 | 09: 100379511 | GABBR2 | 0.0134 | !C,T: 1.5(1.14, 1.97) | 0.1200 | C: 1.19(0.96, 1.46) | 0.0396 | !C,T: 1.39(1.07, 1.82) | 0.4754 | C: 1.08(0.88, 1.31) |
| RS914665 | 09: 100402769 | GABBR2 | 0.0419 | !C,C: 1.65(1.09, 2.51) | 0.0293 | T: 1.25(1.03, 1.53) | 0.0124 | T,T: 1.47(1.13, 1.91) | 0.0311 | T: 1.24(1.02, 1.5) |
| RS2846457 | 09: 112567386 | MUSK | 0.0586 | A,A: 2.18(1.04, 4.57) | 0.0491 | A: 1.27(1.01, 1.61) | 0.0439 | A,G: 1.42(1.08, 1.87) | 0.1405 | A: 1.19(0.95, 1.48) |
| RS6478269 | 09: 118814195 | ASTN2 | 0.0326 | G,G: 1.43(1.08, 1.89) | 0.0086 | G: 1.38(1.09, 1.76) | 0.0739 | G,G: 1.32(0.99, 1.76) | 0.0333 | G: 1.33(1.03, 1.71) |
| RS694428 | 09: 119653079 | LOC645358, | 0.0323 | T,T: 8.59(0.46, 159.96) | 0.5616 | C: 1.15(0.73, 1.81) | 0.0328 | C,C: 1.68(1.13, 2.49) | 0.0123 | C: 1.6(1.11, 2.32) |
| | | LOC647666 | | | | | | | | |
| RS7849366 | 09: 120152185 | | 0.0336 | G,G: 1.91(1.1, 3.31) | 0.0156 | G: 1.32(1.06, 1.64) | 0.0135 | !A,G: 1.39(1.05, 1.84) | 0.7733 | A: 1.04(0.83, 1.3) |
| RS10491527 | 09: 121216614 | | 0.0131 | T,T: 3.79(1.33, 10.77) | 0.4922 | T: 1.1(0.84, 1.44) | 0.0821 | !C,C: 1.37(1.02, 1.82) | 0.0237 | T: 1.34(1.04, 1.73) |
| RS4837693 | 09: 121812351 | | 0.0708 | C,C: 1.38(1.05, 1.82) | 0.0430 | C: 1.23(1.01, 1.5) | 0.1539 | C,C: 1.25(0.96, 1.62) | 0.0500 | C: 1.22(1, 1.47) |
| RS1537282 | 09: 122164325 | | 0.0438 | !A,G: 1.34(1.02, 1.76) | 0.6162 | A: 1.05(0.86, 1.28) | 0.0514 | !G,G: 1.36(1.03, 1.79) | 0.0170 | A: 1.26(1.04, 1.52) |
| RS2535741 | 09: 124596539 | | 0.0411 | !A,G: 1.4(1.02, 1.93) | 0.3967 | G: 1.13(0.86, 1.49) | 0.0667 | G,G: 1.35(1, 1.81) | 0.0253 | G: 1.36(1.04, 1.78) |
| RS2799466 | 09: 125587911 | DENND1A | 0.0119 | C,C: 6.1(1.08, 34.3) | 0.8667 | T: 1.03(0.74, 1.44) | 0.0370 | !C,T: 1.51(0.99, 2.29) | 0.4510 | T: 1.16(0.8, 1.68) |
| RS7873555 | 09: 131586137 | | 0.0345 | !T,T: 1.43(1.09, 1.87) | 0.0172 | C: 1.29(1.05, 1.59) | 0.0334 | !T,T: 1.41(1.08, 1.83) | 0.0367 | C: 1.24(1.02, 1.52) |
| RS12551315 | 09: 131742462 | FNBP1 | 0.0754 | A,A: 1.6(1.06, 2.41) | 0.0246 | A: 1.56(1.06, 2.29) | 0.0335 | G,G: 13.53(0.76, 240.88) | 0.6345 | G: 1.11(0.76, 1.6) |
| RS2991274 | 09: 132377530 | | 0.0898 | G,G: 1.42(1.03, 1.96) | 0.0454 | G: 1.36(1.01, 1.81) | 0.0979 | G,G: 1.4(1.03, 1.89) | 0.0425 | G: 1.34(1.01, 1.76) |
| RS7026553 | 09: 132462568 | FUBP3 | 0.0295 | A,A: 1.72(1.1, 2.68) | 0.0180 | A: 1.68(1.1, 2.58) | 0.0093 | !A,G: 1.8(1.18, 2.74) | 0.0097 | A: 1.7(1.14, 2.53) |
| RS11792889 | 09: 132769269 | FIBCD1 | 0.0101 | !A,G: 1.41(1.07, 1.86) | 0.9558 | A: 1.01(0.81, 1.26) | 0.0112 | G,G: 1.96(1.18, 3.28) | 0.0057 | G: 1.35(1.09, 1.66) |
| RS7074221 | 10: 000403440 | DIP2C | 0.0079 | T,T: 1.67(1.2, 2.34) | 0.0584 | T: 1.2(1, 1.46) | 0.0271 | C,C: 1.47(1.1, 1.97) | 0.0600 | C: 1.2(1, 1.44) |
| RS11250372 | 10: 001297456 | ADARB2 | 0.0123 | !G,G: 3.47(1.32, 9.13) | 0.0142 | A: 1.4(1.07, 1.83) | 0.0638 | A,A: 1.39(1.05, 1.85) | 0.0302 | A: 1.31(1.03, 1.68) |
| RS10794799 | 10: 001935387 | | 0.0442 | C,C: 1.35(1.03, 1.77) | 0.0111 | C: 1.3(1.06, 1.59) | 0.0531 | C,C: 1.36(1.04, 1.77) | 0.0169 | C: 1.27(1.04, 1.53) |
| RS1475420 | 10: 006738102 | | 0.0776 | G,G: 1.33(1.01, 1.76) | 0.0270 | G: 1.31(1.03, 1.65) | 0.0312 | !A,A: 1.92(1.13, 3.26) | 0.0229 | G: 1.27(1.03, 1.57) |
| RS12764177 | 10: 006832573 | | 0.0080 | A,C: 1.43(1.07, 1.91) | 1.0000 | A: 1(0.8, 1.26) | 0.0243 | A,C: 1.44(1.09, 1.92) | 0.2864 | C: 1.14(0.9, 1.43) |
| RS955921 | 10: 009465912 | | 0.0665 | A,G: 1.48(1.06, 2.08) | 0.0493 | G: 1.35(1, 1.83) | 0.0719 | A,G: 1.46(1.05, 2.02) | 0.0459 | G: 1.36(1.01, 1.81) |
| RS1414395 | 10: 013374142 | PHYH | 0.0559 | T,T: 1.41(1.06, 1.86) | 0.0330 | T: 1.24(1.02, 1.51) | 0.0466 | T,T: 1.39(1.06, 1.81) | 0.0809 | T: 1.19(0.98, 1.43) |
| RS17631759 | 10: 014002167 | FRMD4A | 0.0982 | A,A: 1.4(1.03, 1.91) | 0.0470 | A: 1.33(1.01, 1.76) | 0.1075 | A,A: 1.37(0.98, 1.9) | 0.0394 | A: 1.37(1.02, 1.86) |
| RS2457857 | 10: 014100685 | FRMD4A | 0.0839 | !A,A: 1.37(1.03, 1.84) | 0.0228 | C: 1.34(1.04, 1.72) | 0.0443 | !A,A: 1.38(1.05, 1.81) | 0.0104 | C: 1.35(1.07, 1.7) |
| RS6602180 | 10: 017232368 | DNMT2 | 0.0713 | !T,T: 1.34(1, 1.8) | 0.0222 | C: 1.25(1.03, 1.51) | 0.0440 | C,C: 1.46(1.07, 1.99) | 0.1448 | C: 1.15(0.95, 1.38) |
| RS11015065 | 10: 018928375 | NSUN6 | 0.0106 | T,T: 1.7(1.15, 2.52) | 0.0040 | T: 1.72(1.19, 2.5) | 0.0737 | T,T: 1.47(1, 2.16) | 0.0279 | T: 1.5(1.04, 2.17) |
| RS4636555 | 10: 018943611 | NSUN6 | 0.0479 | C,C: 1.53(1.04, 2.26) | 0.0229 | C: 1.26(1.04, 1.53) | 0.0212 | !A,A: 1.43(1.09, 1.88) | 0.0854 | C: 1.18(0.98, 1.43) |
| RS7895709 | 10: 018945941 | NSUN6 | 0.0477 | T,T: 1.48(1.01, 2.19) | 0.0174 | T: 1.27(1.04, 1.54) | 0.0247 | C,T: 1.4(1.08, 1.82) | 0.1033 | T: 1.17(0.97, 1.41) |
| RS7901343 | 10: 032029614 | | 0.1393 | !G,G: 1.29(0.98, 1.69) | 0.0436 | A: 1.25(1.01, 1.55) | 0.0066 | A,G: 1.43(1.1, 1.86) | 0.9189 | G: 1.01(0.83, 1.23) |
| RS733394 | 10: 049618247 | C10ORF64 | 0.0601 | C,C: 1.43(1.06, 1.91) | 0.0410 | C: 1.23(1.01, 1.48) | 0.0057 | C,C: 1.57(1.17, 2.1) | 0.0016 | C: 1.35(1.12, 1.62) |
| RS4838650 | 10: 049664276 | C10ORF64 | 0.0432 | G,G: 2.66(1.14, 6.21) | 0.0606 | G: 1.28(1, 1.66) | 0.0357 | !A,G: 1.51(1.1, 2.06) | 0.0589 | A: 1.31(0.99, 1.73) |
| RS12264297 | 10: 049695610 | | 0.0357 | C,C: 3.45(1.05, 11.36) | 0.0265 | C: 1.39(1.04, 1.87) | 0.0286 | !T,T: 1.4(1.03, 1.89) | 0.0106 | C: 1.44(1.09, 1.88) |
| RS3849150 | 10: 049779229 | WDFY4 | 0.0212 | !T,T: 1.54(1.13, 2.11) | 0.0073 | C: 1.47(1.11, 1.95) | 0.0486 | C,C: 2.28(1.05, 4.99) | 0.0189 | C: 1.37(1.05, 1.79) |
| RS2466756 | 10: 050046112 | C10ORF128 | 0.0853 | !T,T: 1.35(1.03, 1.77) | 0.0365 | G: 1.24(1.02, 1.52) | 0.0515 | !T,T: 1.38(1.06, 1.79) | 0.0211 | G: 1.26(1.04, 1.54) |
| RS2454566 | 10: 053403551 | PRKG1 | 0.0472 | G,G: 1.39(1.06, 1.83) | 0.0156 | G: 1.28(1.05, 1.56) | 0.0406 | !G,T: 1.36(1.05, 1.77) | 0.8832 | T: 1.02(0.84, 1.23) |
| RS12414156 | 10: 056612592 | | 0.0485 | A,A: 1.44(1.07, 1.94) | 0.0143 | A: 1.39(1.07, 1.8) | 0.0047 | !A,G: 1.57(1.19, 2.08) | 0.0676 | A: 1.25(0.99, 1.57) |
| RS3851256 | 10: 060363423 | | 0.0075 | !C,T: 1.56(1.18, 2.06) | 0.0834 | T: 1.21(0.97, 1.51) | 0.0209 | !C,C: 1.87(1.18, 2.98) | 0.1475 | T: 1.16(0.95, 1.43) |
| RS2664328 | 10: 064440254 | | 0.0031 | !C,C: 3.89(1.49, 10.11) | 0.0044 | T: 1.47(1.13, 1.9) | 0.0130 | !C,C: 2.99(1.13, 7.91) | 0.7501 | C: 1.04(0.81, 1.34) |
| RS4746906 | 10: 071185736 | | 0.0911 | T,T: 1.38(1.02, 1.85) | 0.0289 | T: 1.34(1.04, 1.74) | 0.0083 | !C,C: 3.15(1.44, 6.91) | 0.0897 | T: 1.23(0.97, 1.56) |
| RS11593153 | 10: 071269048 | COL13A1 | 0.0506 | !A,A: 1.34(1, 1.8) | 0.0224 | C: 1.35(1.04, 1.75) | 0.0053 | !A,A: 1.59(1.19, 2.12) | 0.0009 | C: 1.53(1.19, 1.97) |
| RS4747096 | 10: 072188015 | ADAMTS14 | 0.0038 | G,G: 3.24(1.33, 7.86) | 0.8945 | G: 1.02(0.79, 1.32) | 0.1094 | !A,A: 1.32(1, 1.74) | 0.0403 | G: 1.29(1.01, 1.65) |
| RS7098277 | 10: 076100549 | ADK | 0.0570 | !A,A: 2.01(1.07, 3.8) | 0.0451 | G: 1.26(1.01, 1.58) | 0.0405 | !A,G: 1.42(1.08, 1.86) | 0.1312 | G: 1.18(0.96, 1.46) |
| RS7097101 | 10: 078837535 | KCNMA1 | 0.0536 | !A,A: 1.47(1.07, 2.01) | 0.0272 | G: 1.38(1.04, 1.83) | 0.0237 | G,G: 6.64(1.18, 37.32) | 0.1004 | G: 1.29(0.96, 1.73) |
| RS2349157 | 10: 085169216 | | 0.0156 | !A,A: 4.5(1.39, 14.59) | 0.2868 | G: 1.17(0.88, 1.54) | 0.0473 | !A,A: 3.25(0.98, 10.83) | 1.0000 | A: 1.01(0.76, 1.35) |
| RS7394286 | 10: 088739172 | KIAA1975, | 0.0847 | !G,G: 1.5(1.02, 2.2) | 0.0410 | A: 1.23(1.01, 1.49) | 0.0086 | !A,G: 1.51(1.16, 1.97) | 0.2170 | A: 1.13(0.93, 1.37) |
| | | LOC643150 | | | | | | | | |
| RS816278 | 10: 096107550 | NOC3L | 0.0238 | A,G: 1.45(1.11, 1.91) | 0.3826 | A: 1.1(0.9, 1.34) | 0.0425 | !A,A: 1.65(1.1, 2.47) | 0.1721 | G: 1.15(0.95, 1.39) |
| RS1035209 | 10: 101335356 | | 0.0278 | !C,T: 1.43(1.07, 1.92) | 0.2275 | T: 1.17(0.91, 1.5) | 0.0043 | !C,T: 1.59(1.2, 2.1) | 0.0217 | T: 1.32(1.05, 1.66) |
| RS2273696 | 10: 102249696 | SEC31L2 | 0.0832 | T,T: 1.32(1.01, 1.73) | 0.0296 | T: 1.26(1.03, 1.54) | 0.0173 | T,T: 1.41(1.09, 1.84) | 0.0051 | T: 1.33(1.09, 1.62) |
| RS10509792 | 10: 107314115 | | 0.0146 | G,G: 3.46(1.33, 9) | 0.0392 | G: 1.31(1.02, 1.69) | 0.0014 | !G,T: 1.73(1.28, 2.34) | 0.0108 | T: 1.39(1.08, 1.79) |
| RS2475176 | 10: 114610872 | | 0.0701 | !C,C: 1.37(1.04, 1.8) | 0.0221 | A: 1.31(1.04, 1.64) | 0.0070 | !C,C: 1.43(1.1, 1.85) | 0.0025 | A: 1.39(1.12, 1.72) |
| RS1327694 | 10: 114627535 | | 0.0384 | A,A: 1.79(1.12, 2.85) | 0.0366 | A: 1.25(1.02, 1.54) | 0.0095 | !C,C: 1.46(1.12, 1.9) | 0.0034 | A: 1.35(1.11, 1.65) |
| RS11595716 | 10: 114633926 | | 0.0177 | C,C: 1.83(1.16, 2.9) | 0.0106 | C: 1.31(1.07, 1.61) | 0.0086 | !T,T: 1.47(1.13, 1.91) | 0.0029 | C: 1.35(1.11, 1.65) |
| RS9328842 | 10: 124755847 | ZNFN1A5 | 0.0985 | C,C: 1.33(1.02, 1.74) | 0.0400 | C: 1.25(1.01, 1.53) | 0.0081 | !C,T: 1.42(1.09, 1.86) | 0.7621 | T: 1.03(0.85, 1.26) |
| RS10901639 | 10: 128263139 | | 0.0144 | !A,C: 1.61(1.16, 2.24) | 0.0122 | A: 1.46(1.09, 1.95) | 0.0436 | !C,C: 6.26(1.11, 35.22) | 0.2149 | A: 1.22(0.9, 1.65) |
| RS6482689 | 10: 130291187 | | 0.0028 | A,A: 18.34(1.06, 316.05) | 1.0000 | A: 1(0.73, 1.37) | 0.0977 | !C,C: 1.4(1, 1.98) | 0.0361 | A: 1.41(1.03, 1.94) |
| RS9419165 | 10: 133708757 | | 0.0584 | !G,G: 1.35(0.99, 1.85) | 0.0367 | A: 1.36(1.03, 1.81) | 0.0396 | A,A: 3.14(0.93, 10.59) | 0.6284 | G: 1.07(0.82, 1.41) |
| RS4074794 | 11: 000399815 | SIGIRR | 0.0901 | T,T: 1.39(1.03, 1.9) | 0.0304 | T: 1.36(1.04, 1.79) | 0.0548 | T,T: 1.37(1.04, 1.81) | 0.0173 | T: 1.34(1.05, 1.71) |
| RS3059 | 11: 000830319 | CD151, | 0.1057 | !A,A: 1.28(0.97, 1.67) | 0.0350 | G: 1.24(1.02, 1.52) | 0.0088 | G,G: 1.98(1.26, 3.11) | 0.0728 | G: 1.21(0.99, 1.48) |
| | | POLR2L | | | | | | | | |
| RS10794350 | 11: 000936070 | AP2A2 | 0.0020 | G,G: 1.71(1.26, 2.32) | 0.0101 | G: 1.29(1.07, 1.56) | 0.0097 | !A,A: 1.51(1.09, 2.1) | 0.0026 | G: 1.33(1.11, 1.6) |
| RS6421967 | 11: 000973966 | AP2A2 | 0.0155 | !A,G: 1.46(1.11, 1.91) | 0.0802 | A: 1.19(0.98, 1.45) | 0.0024 | A,A: 1.54(1.17, 2.01) | 0.0008 | A: 1.39(1.15, 1.68) |
| RS4077293 | 11: 000986522 | AP2A2 | 0.0240 | C,C: 1.75(1.12, 2.74) | 0.0141 | C: 1.29(1.05, 1.58) | 0.0537 | !T,T: 1.36(1.05, 1.77) | 0.0200 | C: 1.26(1.04, 1.54) |
| RS4076950 | 11: 001005021 | LOC642724, | 0.0715 | !T,T: 1.34(1.02, 1.75) | 0.0261 | C: 1.26(1.03, 1.55) | 0.0505 | !T,T: 1.32(1.02, 1.72) | 0.0162 | C: 1.28(1.05, 1.55) |
| | | MUC6 | | | | | | | | |
| RS11246386 | 11: 001034938 | | 0.0089 | C,C: 1.52(1.15, 1.99) | 0.0127 | C: 1.33(1.07, 1.66) | 0.0615 | C,C: 1.37(1.05, 1.78) | 0.0462 | C: 1.24(1.01, 1.54) |
| RS1386153 | 11: 010986908 | | 0.0195 | !C,T: 1.47(1.12, 1.93) | 0.1607 | T: 1.16(0.95, 1.42) | 0.0569 | T,T: 1.38(1.06, 1.79) | 0.0388 | T: 1.24(1.01, 1.51) |
| RS4757289 | 11: 015112898 | | 0.0097 | C,T: 1.51(1.15, 1.98) | 0.8831 | T: 1.02(0.84, 1.23) | 0.0825 | !C,C: 1.32(1, 1.74) | 0.0280 | T: 1.24(1.03, 1.49) |
| RS6486334 | 11: 016972133 | PLEKHA7 | 0.0398 | !T,T: 1.4(1.07, 1.83) | 0.0106 | C: 1.3(1.06, 1.59) | 0.0748 | !T,T: 1.35(1.04, 1.76) | 0.0350 | C: 1.24(1.02, 1.51) |
| RS214097 | 11: 017248075 | | 0.0039 | C,C: 1.79(1.23, 2.62) | 0.0018 | C: 1.37(1.13, 1.66) | 0.0476 | C,C: 1.47(1.04, 2.08) | 0.3430 | C: 1.09(0.91, 1.32) |
| RS2072231 | 11: 017475485 | USH1C | 0.0237 | !G,G: 5.37(0.93, 31.15) | 0.6255 | G: 1.09(0.79, 1.5) | 0.0228 | !G,G: 6.26(1.11, 35.22) | 0.8792 | A: 1.03(0.76, 1.38) |
| RS7946133 | 11: 017748398 | KCNC1 | 0.0494 | T,T: 1.51(1.08, 2.12) | 0.0167 | T: 1.47(1.07, 2.01) | 0.0599 | T,T: 1.37(1.01, 1.84) | 0.0238 | T: 1.37(1.05, 1.8) |
| RS1015896 | 11: 017839962 | SERGEF | 0.0376 | C,C: 1.37(1.03, 1.83) | 0.0125 | C: 1.28(1.06, 1.55) | 0.0340 | C,C: 1.44(1.08, 1.92) | 0.0126 | C: 1.27(1.06, 1.53) |
| RS10833367 | 11: 020598084 | SLC6A5 | 0.0319 | !C,T: 1.41(1.08, 1.85) | 0.9607 | C: 1.01(0.83, 1.22) | 0.0191 | C,C: 1.53(1.08, 2.18) | 0.3634 | C: 1.09(0.91, 1.32) |
| RS1531005 | 11: 021283735 | NELL1 | 0.0048 | A,A: 1.72(1.23, 2.39) | 0.0222 | A: 1.25(1.03, 1.51) | 0.0297 | A,G: 1.39(1.07, 1.81) | 0.1851 | A: 1.14(0.94, 1.37) |
| RS4335544 | 11: 021286758 | NELL1 | 0.0476 | C,C: 1.53(1.09, 2.15) | 0.0503 | C: 1.21(1, 1.47) | 0.0178 | A,C: 1.43(1.1, 1.86) | 0.1270 | C: 1.16(0.96, 1.4) |
| RS1871953 | 11: 023149253 | | 0.0069 | C,C: 14.41(0.82, 253.09) | 0.6090 | T: 1.08(0.81, 1.44) | 0.0134 | !C,T: 1.48(1.06, 2.07) | 0.4631 | T: 1.13(0.84, 1.5) |
| RS11027043 | 11: 023169469 | | 0.0401 | C,C: 10.52(0.58, 190.81) | 0.7426 | T: 1.06(0.77, 1.47) | 0.0194 | C,C: 2.91(0.98, 8.62) | 0.6949 | T: 1.07(0.79, 1.45) |
| RS1982099 | 11: 028993215 | | 0.0875 | !C,C: 1.34(1.02, 1.75) | 0.0290 | A: 1.27(1.03, 1.56) | 0.1189 | !C,C: 1.3(1, 1.69) | 0.0446 | A: 1.24(1.01, 1.53) |
| RS262418 | 11: 035981694 | LDLRAD3 | 0.0316 | T,T: 14.41(0.82, 253.09) | 0.3351 | T: 1.19(0.84, 1.67) | 0.0096 | T,T: 13.56(0.76, 241.41) | 0.0165 | T: 1.58(1.09, 2.28) |
| RS2173553 | 11: 036048464 | LDLRAD3 | 0.0046 | !T,T: 2.3(1.36, 3.89) | 0.0148 | C: 1.3(1.06, 1.6) | 0.0318 | !C,T: 1.42(1.09, 1.85) | 0.2572 | C: 1.13(0.92, 1.38) |
| RS331455 | 11: 036497125 | | 0.0382 | !T,T: 1.87(1.13, 3.09) | 0.2165 | C: 1.15(0.93, 1.41) | 0.0082 | !T,T: 2.17(1.31, 3.62) | 0.0192 | C: 1.28(1.04, 1.58) |
| RS4755854 | 11: 044605807 | | 0.0194 | !C,T: 1.47(1.12, 1.92) | 0.2766 | C: 1.11(0.92, 1.35) | 0.0015 | !C,T: 1.62(1.24, 2.1) | 0.2331 | C: 1.12(0.93, 1.35) |
| RS7124245 | 11: 044900176 | TSPAN18 | 0.0059 | C,C: 3.79(1.33, 10.77) | 0.9455 | C: 1.02(0.78, 1.33) | 0.0396 | C,C: 3.39(1.17, 9.85) | 0.4849 | C: 1.11(0.84, 1.45) |
| RS10769258 | 11: 047347615 | SPI1 | 0.0942 | C,C: 1.31(1, 1.72) | 0.0314 | C: 1.25(1.02, 1.53) | 0.0534 | C,C: 1.34(1.03, 1.74) | 0.0162 | C: 1.28(1.05, 1.55) |
| RS755553 | 11: 047388879 | SLC39A13 | 0.0585 | C,C: 1.36(1.04, 1.79) | 0.0184 | C: 1.27(1.04, 1.56) | 0.0595 | C,C: 1.34(1.03, 1.74) | 0.0186 | C: 1.27(1.04, 1.55) |
| RS1015195 | 11: 056300593 | GENE_FOR_TAR92809 | 0.0146 | T,T: 1.96(1.15, 3.36) | 0.0064 | T: 1.35(1.09, 1.67) | 0.0013 | T,T: 2.99(1.55, 5.8) | 0.0046 | T: 1.36(1.1, 1.69) |
| RS518385 | 11: 056301321 | GENE_FOR_TAR92809 | 0.0413 | !T,T: 1.5(1.08, 2.08) | 0.1454 | C: 1.16(0.96, 1.4) | 0.0864 | C,C: 1.37(1.02, 1.85) | 0.0308 | C: 1.23(1.02, 1.47) |
| RS569798 | 11: 056308204 | | 0.0484 | !G,G: 1.58(1.07, 2.33) | 0.2734 | A: 1.12(0.92, 1.36) | 0.0146 | !G,G: 1.54(1.09, 2.17) | 0.0037 | A: 1.32(1.09, 1.59) |
| RS1788998 | 11: 056344551 | GENE_FOR_TAR92812 | 0.0291 | !C,C: 1.64(1.11, 2.42) | 0.2315 | A: 1.13(0.93, 1.37) | 0.0197 | !C,C: 1.5(1.06, 2.12) | 0.0049 | A: 1.31(1.09, 1.58) |
| RS1943624 | 11: 056349021 | | 0.0133 | !A,A: 1.62(1.17, 2.24) | 0.0174 | G: 1.26(1.04, 1.53) | 0.0042 | !A,A: 1.59(1.17, 2.14) | 0.0012 | G: 1.36(1.13, 1.64) |
| RS764855 | 11: 056366988 | | 0.0193 | !C,C: 1.51(1.13, 2.01) | 0.0109 | A: 1.29(1.06, 1.56) | 0.0382 | !C,C: 1.37(1.04, 1.81) | 0.0104 | A: 1.28(1.06, 1.54) |
| RS1947298 | 11: 057728736 | OR1S2, | 0.0153 | !A,A: 1.83(1.18, 2.84) | 0.2341 | G: 1.13(0.93, 1.39) | 0.1292 | G,G: 1.3(1, 1.69) | 0.0474 | G: 1.22(1, 1.48) |
| | | GENE_FOR_TAR92825 | | | | | | | | |
| RS7109608 | 11: 061960452 | AHNAK | 0.0309 | !T,T: 1.46(1.1, 1.94) | 0.0091 | C: 1.39(1.08, 1.77) | 0.0026 | !C,C: 3.03(1.38, 6.67) | 0.8061 | T: 1.04(0.81, 1.32) |
| RS7122303 | 11: 068782158 | | 0.0361 | C,T: 1.42(1.04, 1.92) | 0.2481 | C: 1.18(0.9, 1.54) | 0.0262 | !T,T: 1.46(1.1, 1.93) | 0.0192 | C: 1.34(1.05, 1.71) |
| RS1939469 | 11: 075913868 | C11ORF30 | 0.0131 | T,T: 3.79(1.33, 10.77) | 0.0283 | T: 1.34(1.03, 1.75) | 0.0296 | C,T: 1.46(1.08, 1.96) | 0.1225 | T: 1.22(0.95, 1.57) |
| RS2372897 | 11: 077420474 | | 0.0036 | A,A: 1.57(1.18, 2.08) | 0.0011 | A: 1.38(1.14, 1.68) | 0.0436 | !G,G: 1.47(1.02, 2.13) | 0.0160 | A: 1.27(1.05, 1.53) |
| RS230669 | 11: 077553034 | | 0.0005 | C,C: 1.74(1.31, 2.31) | 0.0005 | C: 1.42(1.17, 1.72) | 0.0071 | C,C: 1.43(1.09, 1.87) | 0.0016 | C: 1.36(1.12, 1.65) |
| RS1015197 | 11: 092490826 | | 0.0056 | T,T: 18.34(1.06, 316.05) | 0.7549 | T: 1.06(0.78, 1.44) | 0.0429 | !C,C: 1.41(1.04, 1.91) | 0.0162 | T: 1.41(1.07, 1.86) |
| RS649944 | 11: 094006944 | | 0.0656 | G,G: 1.44(1.05, 1.98) | 0.0329 | G: 1.23(1.02, 1.49) | 0.0149 | !A,G: 1.47(1.13, 1.91) | 0.9251 | A: 1.01(0.84, 1.22) |
| RS528098 | 11: 095594860 | MAML2 | 0.0228 | C,T: 1.48(1.11, 1.98) | 0.0227 | C: 1.33(1.05, 1.7) | 0.0066 | C,C: 6.13(1.59, 23.63) | 0.1235 | C: 1.22(0.95, 1.57) |
| RS562833 | 11: 095599955 | MAML2 | 0.0187 | C,T: 1.5(1.13, 2) | 0.0191 | T: 1.34(1.05, 1.71) | 0.0441 | T,T: 4.4(1.1, 17.59) | 0.1496 | T: 1.22(0.94, 1.57) |
| RS682837 | 11: 102558888 | DYNC2H1 | 0.0876 | !T,T: 1.49(1.02, 2.18) | 0.0364 | C: 1.47(1.03, 2.12) | 0.0492 | !T,T: 1.44(1, 2.08) | 0.0502 | C: 1.44(1.01, 2.04) |
| RS1791459 | 11: 105925316 | | 0.0629 | A,A: 1.42(1.06, 1.92) | 0.0234 | A: 1.36(1.05, 1.77) | 0.0229 | A,A: 1.48(1.11, 1.98) | 0.0064 | A: 1.42(1.11, 1.83) |
| RS585287 | 11: 113549649 | ZBTB16 | 0.0493 | C,C: 1.75(1.07, 2.85) | 0.0320 | C: 1.25(1.02, 1.54) | 0.0286 | C,T: 1.43(1.1, 1.86) | 0.1997 | C: 1.15(0.94, 1.4) |
| RS560195 | 11: 113651461 | | 0.0261 | !A,G: 1.56(1.04, 2.35) | 0.0972 | G: 1.39(0.94, 2.04) | 0.1255 | G,G: 1.45(0.98, 2.15) | 0.0424 | G: 1.46(1.01, 2.11) |
| RS12361635 | 11: 130186012 | | 0.0352 | A,G: 1.5(1.09, 2.06) | 0.0967 | A: 1.26(0.96, 1.66) | 0.0302 | !G,G: 1.47(1.1, 1.96) | 0.0114 | A: 1.39(1.08, 1.79) |
| RS7943936 | 11: 130214277 | | 0.0149 | !T,T: 1.49(1.08, 2.06) | 0.0057 | C: 1.31(1.09, 1.59) | 0.0162 | !T,T: 1.51(1.12, 2.03) | 0.0042 | C: 1.31(1.09, 1.57) |
| RS7943757 | 11: 130221367 | LOC399978 | 0.0171 | T,T: 1.45(1.07, 1.96) | 0.0056 | T: 1.31(1.08, 1.59) | 0.0176 | !C,C: 1.49(1.08, 2.05) | 0.0047 | T: 1.31(1.09, 1.57) |
| RS7119425 | 11: 130238395 | LOC399978 | 0.0066 | T,T: 1.53(1.15, 2.04) | 0.0017 | T: 1.36(1.12, 1.65) | 0.0469 | !C,C: 1.5(1.08, 2.08) | 0.0296 | T: 1.23(1.02, 1.48) |
| RS6590535 | 11: 130274243 | SNX19 | 0.0385 | !T,T: 1.43(1.08, 1.89) | 0.0130 | C: 1.36(1.07, 1.72) | 0.1238 | !T,T: 1.33(1.01, 1.76) | 0.0465 | C: 1.29(1.01, 1.64) |
| RS574322 | 11: 131326210 | HNT | 0.0640 | !C,C: 2.65(0.97, 7.21) | 0.0386 | T: 1.35(1.02, 1.78) | 0.0092 | !C,T: 1.6(1.18, 2.17) | 0.0114 | T: 1.41(1.09, 1.84) |
| RS3099792 | 11: 131612962 | HNT | 0.0099 | !C,T: 1.5(1.14, 1.97) | 0.9208 | T: 1.01(0.83, 1.23) | 0.0780 | T,T: 1.36(1.04, 1.78) | 0.0422 | T: 1.22(1.01, 1.47) |
| RS4482038 | 11: 132422822 | OPCML | 0.0165 | !A,G: 1.71(1.17, 2.49) | 0.0166 | A: 1.51(1.08, 2.12) | 0.0288 | A,A: 1.6(1.12, 2.27) | 0.0092 | A: 1.54(1.12, 2.13) |
| RS7126587 | 11: 132433645 | OPCML | 0.0540 | G,G: 1.49(1.08, 2.06) | 0.0247 | G: 1.41(1.05, 1.89) | 0.0020 | G,G: 1.7(1.25, 2.3) | 0.0004 | G: 1.64(1.25, 2.15) |
| RS3920986 | 11: 132653665 | OPCML | 0.0867 | G,G: 1.34(1.02, 1.76) | 0.0314 | G: 1.27(1.02, 1.58) | 0.0154 | !T,T: 2.19(1.18, 4.05) | 0.5686 | G: 1.07(0.86, 1.34) |
| RS2041382 | 12: 004824812 | | 0.0175 | A,G: 1.44(1.1, 1.88) | 0.0935 | G: 1.18(0.97, 1.43) | 0.0929 | !A,A: 1.35(1.02, 1.77) | 0.0401 | G: 1.22(1.01, 1.47) |
| RS10492062 | 12: 004907076 | | 0.0442 | !G,G: 3.24(0.96, 10.94) | 0.9357 | G: 1.03(0.75, 1.41) | 0.0175 | A,G: 1.66(1.15, 2.39) | 0.0111 | G: 1.54(1.1, 2.16) |
| RS2969097 | 12: 008782794 | FAM80B | 0.0015 | !T,T: 1.55(1.16, 2.06) | 0.0004 | C: 1.42(1.17, 1.72) | 0.0487 | !C,T: 1.36(1.05, 1.77) | 0.8831 | T: 1.02(0.84, 1.23) |
| RS1990479 | 12: 008802742 | FAM80B | 0.0065 | !A,A: 1.71(1.18, 2.48) | 0.0021 | G: 1.36(1.12, 1.65) | 0.0386 | !A,G: 1.4(1.08, 1.82) | 0.5371 | G: 1.06(0.88, 1.28) |
| RS7979266 | 12: 010124344 | CLEC1A | 0.1110 | A,A: 1.28(0.97, 1.69) | 0.0453 | A: 1.27(1.01, 1.6) | 0.0183 | A,G: 1.4(1.06, 1.84) | 0.6576 | G: 1.05(0.85, 1.31) |
| RS2541128 | 12: 011708010 | ETV6 | 0.0474 | !G,G: 1.48(1.08, 2.03) | 0.0174 | A: 1.42(1.07, 1.89) | 0.0018 | !A,A: 7.02(1.85, 26.57) | 0.1105 | G: 1.25(0.95, 1.65) |
| RS1861482 | 12: 011724925 | ETV6 | 0.0173 | !T,T: 1.57(1.15, 2.14) | 0.0071 | G: 1.48(1.11, 1.95) | 0.0011 | !G,G: 7.45(1.98, 28.09) | 0.0608 | T: 1.3(0.99, 1.72) |
| RS7132921 | 12: 012632336 | | 0.0278 | !C,T: 1.42(1.08, 1.86) | 0.1856 | T: 1.14(0.94, 1.38) | 0.0488 | !C,T: 1.37(1.06, 1.78) | 0.2323 | T: 1.12(0.93, 1.35) |
| RS10841260 | 12: 019597931 | | 0.0064 | T,T: 1.57(1.18, 2.09) | 0.0031 | T: 1.34(1.11, 1.63) | 0.0338 | !C,T: 1.35(1.04, 1.75) | 0.7335 | C: 1.04(0.86, 1.25) |
| RS952658 | 12: 020756568 | SLCO1C1 | 0.0068 | !T,T: 1.54(1.16, 2.02) | 0.0013 | C: 1.45(1.16, 1.83) | 0.1511 | !T,T: 1.28(0.98, 1.68) | 0.0497 | C: 1.25(1.01, 1.56) |
| RS10842096 | 12: 023067472 | | 0.0739 | !T,T: 1.37(1.04, 1.8) | 0.0427 | G: 1.26(1.01, 1.57) | 0.0317 | !T,T: 1.41(1.09, 1.84) | 0.0121 | G: 1.32(1.06, 1.63) |
| RS472100 | 12: 024370761 | SOX5 | 0.0615 | !T,T: 2.41(0.95, 6.14) | 0.0413 | C: 1.29(1.01, 1.64) | 0.1025 | C,C: 1.33(1, 1.78) | 0.0407 | C: 1.31(1.01, 1.7) |
| RS2955479 | 12: 024583650 | SOX5 | 2.10E−04 | !G,G: 1.77(1.31, 2.38) | 3.97E−05 | A: 1.72(1.33, 2.24) | 0.0426 | A,A: 2.07(1, 4.26) | 1.0000 | G: 1(0.79, 1.28) |
| RS1487657 | 12: 024613521 | FLJ32894 | 0.0136 | !T,T: 1.44(1.09, 1.89) | 0.0034 | G: 1.35(1.1, 1.64) | 0.0866 | !T,T: 1.34(1.03, 1.75) | 0.0400 | G: 1.22(1.01, 1.48) |
| RS10505945 | 12: 024803300 | | 0.0618 | !G,G: 1.67(1.03, 2.71) | 0.0224 | A: 1.28(1.04, 1.59) | 0.0190 | !G,G: 2.03(1.21, 3.39) | 0.1536 | A: 1.17(0.95, 1.44) |
| RS7304591 | 12: 044769621 | | 0.0568 | T,T: 1.48(1.03, 2.12) | 0.0245 | T: 1.48(1.05, 2.08) | 0.0274 | !G,G: 3.75(0.93, 15.19) | 0.6623 | G: 1.08(0.77, 1.52) |
| RS10506265 | 12: 044802761 | | 0.0132 | T,T: 1.72(1.11, 2.66) | 0.0087 | T: 1.76(1.16, 2.68) | 0.0744 | T,T: 1.58(1.04, 2.4) | 0.0274 | T: 1.57(1.06, 2.35) |
| RS10875798 | 12: 047019151 | ZNF641 | 0.0075 | !C,T: 1.52(1.15, 1.99) | 0.3320 | T: 1.11(0.9, 1.38) | 0.0414 | !C,C: 1.58(1.03, 2.42) | 0.0134 | T: 1.29(1.06, 1.57) |
| RS2272306 | 12: 051840550 | CSAD | 0.0852 | !A,A: 1.4(1.03, 1.89) | 0.0334 | G: 1.34(1.02, 1.76) | 0.0242 | G,G: 4.06(1.24, 13.26) | 0.0613 | G: 1.3(0.99, 1.71) |
| RS4422358 | 12: 054155985 | | 0.0325 | A,A: 1.44(1.09, 1.9) | 0.0129 | A: 1.28(1.06, 1.56) | 0.0567 | !G,G: 1.42(1, 2.02) | 0.0196 | A: 1.25(1.04, 1.51) |
| RS894010 | 12: 060670094 | FAM19A2 | 0.0082 | !A,A: 1.63(1.19, 2.23) | 0.0029 | G: 1.54(1.16, 2.04) | 0.0120 | G,G: 4.12(1.45, 11.72) | 0.1138 | G: 1.24(0.96, 1.61) |
| RS1245653 | 12: 060886900 | | 0.0228 | !A,G: 1.62(1.13, 2.31) | 0.0620 | A: 1.37(0.99, 1.88) | 0.0212 | G,G: 15.68(0.89, 275.38) | 0.5134 | G: 1.1(0.83, 1.46) |
| RS1565730 | 12: 065143477 | GRIP1 | 0.0413 | A,G: 1.42(1.08, 1.87) | 0.2801 | G: 1.12(0.91, 1.39) | 0.0218 | !G,G: 2.06(1.2, 3.56) | 0.2377 | A: 1.14(0.92, 1.4) |
| RS11615839 | 12: 069133885 | | 0.0331 | !A,A: 1.41(1.05, 1.88) | 0.0113 | G: 1.4(1.08, 1.81) | 0.0103 | !G,G: 2.31(1.14, 4.68) | 1.0000 | G: 1(0.79, 1.26) |
| RS11115162 | 12: 080950597 | | 0.0357 | T,T: 1.43(1.09, 1.89) | 0.0132 | T: 1.34(1.06, 1.69) | 0.0146 | !C,T: 1.49(1.14, 1.96) | 0.1070 | T: 1.2(0.97, 1.5) |
| RS10777084 | 12: 086904225 | C12ORF50 | 0.0016 | !C,C: 22.77(1.33, 389.74) | 0.4792 | T: 1.12(0.82, 1.52) | 0.0403 | T,T: 1.45(1.06, 1.98) | 0.0159 | T: 1.43(1.07, 1.92) |
| RS11107018 | 12: 092338033 | UBE2N | 0.0417 | !G,G: 2.58(1.02, 6.52) | 0.0293 | A: 1.33(1.03, 1.7) | 0.0263 | !G,G: 2.77(1.25, 6.14) | 0.0986 | A: 1.22(0.97, 1.54) |
| RS1319893 | 12: 097960713 | ANKS1B | 0.0168 | !G,T: 1.45(1.11, 1.9) | 0.0622 | G: 1.21(0.99, 1.47) | 0.0180 | T,T: 1.75(1.16, 2.63) | 0.1818 | T: 1.15(0.94, 1.39) |
| RS10860593 | 12: 099351399 | | 0.0999 | !T,T: 1.41(1.02, 1.95) | 0.0316 | C: 1.38(1.03, 1.85) | 0.0006 | C,T: 1.86(1.33, 2.61) | 0.0028 | C: 1.6(1.18, 2.16) |
| RS11110385 | 12: 099376836 | | 0.0046 | T,T: 1.53(1.16, 2.01) | 0.0010 | T: 1.4(1.14, 1.7) | 0.0983 | T,T: 1.27(0.97, 1.66) | 0.0331 | T: 1.23(1.02, 1.49) |
| RS7969185 | 12: 100080857 | SLC5A8 | 0.0463 | A,G: 1.46(1.07, 1.98) | 0.0320 | G: 1.34(1.03, 1.75) | 0.0211 | !G,G: 2.7(1.27, 5.76) | 0.2278 | A: 1.16(0.91, 1.47) |
| RS2133298 | 12: 101784764 | PAH | 0.0316 | !G,G: 1.52(1.06, 2.17) | 0.0145 | A: 1.28(1.05, 1.54) | 0.0130 | A,A: 1.47(1.11, 1.95) | 0.0888 | A: 1.18(0.98, 1.42) |
| RS1718301 | 12: 101795323 | PAH | 0.0724 | !A,A: 1.43(0.99, 2.05) | 0.0312 | G: 1.24(1.02, 1.5) | 0.0074 | !A,G: 1.47(1.13, 1.92) | 0.1074 | G: 1.17(0.97, 1.41) |
| RS1863538 | 12: 106267905 | BTBD11 | 0.0181 | T,T: 1.49(1.12, 1.99) | 0.0625 | T: 1.2(0.99, 1.46) | 0.0980 | T,T: 1.29(0.98, 1.71) | 0.0295 | T: 1.23(1.02, 1.48) |
| RS1861586 | 12: 106438271 | BTBD11 | 0.0062 | !A,A: 1.91(1.22, 2.99) | 0.3533 | G: 1.11(0.9, 1.35) | 0.0967 | G,G: 1.33(1.02, 1.74) | 0.0404 | G: 1.22(1.01, 1.48) |
| RS4964598 | 12: 106460457 | BTBD11 | 0.0023 | !G,G: 3.33(1.58, 7.04) | 0.1746 | A: 1.18(0.93, 1.48) | 0.0437 | !G,G: 1.9(1.09, 3.3) | 0.0293 | A: 1.27(1.03, 1.58) |
| RS6539407 | 12: 106944648 | | 0.0263 | !C,T: 1.38(1.04, 1.83) | 0.6000 | T: 1.07(0.85, 1.34) | 0.0251 | C,C: 2.18(1.21, 3.93) | 0.1804 | C: 1.17(0.94, 1.45) |
| RS886476 | 12: 111803854 | RPH3A | 0.0916 | !C,C: 1.29(0.98, 1.7) | 0.0316 | T: 1.24(1.02, 1.51) | 0.0473 | T,T: 1.48(1.06, 2.05) | 0.0151 | T: 1.26(1.05, 1.52) |
| RS7978582 | 12: 112035775 | RASAL1 | 0.0258 | !A,A: 1.4(1.03, 1.9) | 0.0126 | G: 1.42(1.08, 1.87) | 0.0894 | G,G: 1.86(0.96, 3.59) | 0.0411 | G: 1.29(1.02, 1.63) |
| RS1878420 | 12: 116537177 | KSR2 | 0.0348 | !T,T: 1.49(1.09, 2.06) | 0.1209 | C: 1.16(0.96, 1.41) | 0.0125 | C,C: 1.53(1.12, 2.08) | 0.0048 | C: 1.31(1.09, 1.57) |
| RS4767658 | 12: 117003824 | FLJ20674 | 0.0120 | !T,T: 1.53(1.16, 2.03) | 0.0079 | C: 1.3(1.07, 1.58) | 0.0389 | C,C: 1.53(1.07, 2.19) | 0.0137 | C: 1.27(1.05, 1.54) |
| RS10849646 | 12: 118262051 | CCDC60 | 0.0020 | C,C: 1.76(1.28, 2.42) | 0.0077 | C: 1.3(1.07, 1.57) | 0.0013 | T,T: 1.63(1.22, 2.18) | 0.0664 | T: 1.19(0.99, 1.43) |
| RS4760974 | 12: 126603427 | | 0.0425 | !T,T: 1.42(1.08, 1.88) | 0.0323 | C: 1.24(1.02, 1.51) | 0.0846 | C,C: 1.45(0.98, 2.15) | 0.0298 | C: 1.25(1.03, 1.51) |
| RS4411335 | 12: 128273807 | TMEM132D | 0.0166 | !T,T: 1.49(1.13, 1.95) | 0.0098 | C: 1.31(1.07, 1.59) | 0.0031 | !T,T: 1.59(1.21, 2.09) | 0.0060 | C: 1.3(1.08, 1.58) |
| RS1482294 | 12: 128276744 | TMEM132D | 0.0108 | !G,G: 1.51(1.13, 2.01) | 0.0037 | A: 1.33(1.1, 1.62) | 0.0138 | !G,G: 1.49(1.13, 1.98) | 0.0052 | A: 1.31(1.08, 1.57) |
| RS6486483 | 12: 128591932 | TMEM132D | 0.0020 | !G,G: 1.73(1.27, 2.36) | 0.0026 | A: 1.34(1.11, 1.62) | 0.0352 | A,A: 1.51(1.1, 2.07) | 0.0307 | A: 1.23(1.02, 1.48) |
| RS11831111 | 12: 131205371 | | 0.0008 | !A,A: 1.71(1.27, 2.29) | 0.0189 | G: 1.26(1.04, 1.53) | 0.0278 | G,G: 1.48(1.06, 2.06) | 0.3443 | G: 1.1(0.91, 1.32) |
| RS6490774 | 13: 022529340 | | 0.0295 | C,T: 1.83(1.09, 3.08) | 0.1217 | C: 1.48(0.92, 2.38) | 0.0420 | !T,T: 1.78(1.04, 3.05) | 0.0261 | C: 1.84(1.09, 3.12) |
| RS9512417 | 13: 026306722 | | 0.0660 | G,G: 1.38(1.05, 1.81) | 0.0314 | G: 1.27(1.02, 1.58) | 0.0915 | !A,A: 1.62(0.98, 2.67) | 0.0361 | G: 1.26(1.02, 1.55) |
| RS12583235 | 13: 029655737 | | 0.0213 | A,G: 1.46(1.11, 1.93) | 0.3461 | G: 1.11(0.9, 1.36) | 0.0517 | G,G: 1.57(1.07, 2.31) | 0.0284 | G: 1.24(1.03, 1.5) |
| RS9508795 | 13: 030022651 | | 0.0499 | C,C: 1.34(1.01, 1.78) | 0.0139 | C: 1.28(1.05, 1.55) | 0.0843 | C,C: 1.3(0.99, 1.71) | 0.0274 | C: 1.24(1.03, 1.5) |
| RS9529615 | 13: 034205066 | | 0.0270 | C,C: 1.69(1.14, 2.5) | 0.1209 | C: 1.17(0.96, 1.42) | 0.0227 | A,C: 1.44(1.11, 1.87) | 0.1699 | C: 1.15(0.95, 1.39) |
| RS275966 | 13: 035184806 | | 0.0752 | T,T: 1.38(1.03, 1.83) | 0.0309 | T: 1.24(1.02, 1.5) | 0.1048 | T,T: 1.3(0.99, 1.7) | 0.0337 | T: 1.23(1.02, 1.48) |
| RS9533366 | 13: 042527595 | DNAJC15 | 0.0844 | !A,A: 1.28(0.98, 1.68) | 0.0279 | G: 1.26(1.03, 1.55) | 0.0580 | !A,A: 1.37(1.05, 1.77) | 0.0471 | G: 1.23(1, 1.51) |
| RS9533385 | 13: 042567737 | DNAJC15 | 0.0980 | !T,T: 1.28(0.98, 1.68) | 0.0318 | C: 1.25(1.02, 1.54) | 0.0484 | !T,T: 1.38(1.07, 1.8) | 0.0362 | C: 1.25(1.02, 1.54) |
| RS9533819 | 13: 043796037 | | 0.0052 | T,T: 1.54(1.18, 2.02) | 0.0015 | T: 1.4(1.14, 1.73) | 0.0291 | !C,C: 1.76(1.13, 2.76) | 0.2674 | T: 1.12(0.92, 1.37) |
| RS9567459 | 13: 044145334 | | 0.0977 | A,A: 1.33(1.01, 1.74) | 0.0314 | A: 1.27(1.02, 1.58) | 0.0536 | !C,C: 1.63(1.02, 2.59) | 0.0215 | A: 1.27(1.04, 1.55) |
| RS474863 | 13: 045958364 | | 0.0305 | !G,G: 2.47(1.14, 5.37) | 0.0213 | A: 1.35(1.05, 1.73) | 0.0843 | A,A: 1.3(0.99, 1.7) | 0.0277 | A: 1.3(1.03, 1.63) |
| RS842383 | 13: 046157226 | LRCH1 | 0.0396 | !C,C: 1.39(1.06, 1.82) | 0.0108 | T: 1.33(1.07, 1.66) | 0.0195 | T,T: 2.25(1.23, 4.1) | 0.2728 | T: 1.13(0.91, 1.4) |
| RS2273816 | 13: 049192057 | KPNA3 | 0.0025 | A,G: 1.53(1.12, 2.1) | 0.4142 | G: 1.13(0.86, 1.47) | 0.0573 | A,A: 1.4(1.03, 1.89) | 0.0196 | A: 1.38(1.06, 1.82) |
| RS9316484 | 13: 049543165 | DLEU2 | 0.0888 | !C,C: 2.01(0.94, 4.3) | 0.0496 | T: 1.28(1.01, 1.63) | 0.0272 | !C,C: 2.3(1.17, 4.52) | 0.0274 | T: 1.29(1.03, 1.61) |
| RS4886077 | 13: 051896698 | VPS36 | 0.0126 | !G,T: 1.47(1.12, 1.93) | 0.0724 | G: 1.2(0.98, 1.46) | 0.0788 | G,G: 1.36(1.04, 1.78) | 0.0426 | G: 1.22(1.01, 1.47) |
| RS6561671 | 13: 052080315 | LOC653891 | 0.0315 | G,G: 1.56(1.11, 2.18) | 0.0720 | G: 1.2(0.99, 1.45) | 0.0565 | !A,A: 1.34(1, 1.79) | 0.0187 | G: 1.25(1.04, 1.5) |
| RS9536470 | 13: 053058723 | | 0.0436 | A,A: 1.42(1.08, 1.86) | 0.0199 | A: 1.31(1.05, 1.63) | 0.0473 | C,C: 2.42(1.07, 5.48) | 0.8566 | C: 1.03(0.81, 1.3) |
| RS4322543 | 13: 056500910 | | 0.0255 | G,G: 1.38(1.05, 1.81) | 0.0108 | G: 1.34(1.07, 1.67) | 0.0033 | !A,A: 2.27(1.36, 3.81) | 0.0051 | G: 1.34(1.09, 1.64) |
| RS577241 | 13: 056596687 | | 0.0408 | G,G: 1.38(1.05, 1.81) | 0.0151 | G: 1.32(1.06, 1.65) | 0.0024 | !A,A: 2.33(1.38, 3.94) | 0.0036 | G: 1.36(1.11, 1.67) |
| RS9570776 | 13: 062481608 | | 0.1219 | !T,T: 1.33(0.98, 1.82) | 0.0469 | C: 1.22(1.01, 1.47) | 0.0146 | !C,T: 1.47(1.13, 1.9) | 0.7781 | C: 1.03(0.86, 1.24) |
| RS9598990 | 13: 065001632 | | 0.0377 | !C,C: 9.61(0.52, 179.12) | 0.5791 | C: 1.12(0.78, 1.6) | 0.0744 | !A,A: 1.53(1.02, 2.28) | 0.0337 | C: 1.52(1.04, 2.23) |
| RS1325678 | 13: 066726347 | | 0.0281 | T,T: 1.58(1.13, 2.22) | 0.0512 | T: 1.21(1, 1.47) | 0.0205 | T,T: 1.55(1.13, 2.12) | 0.0127 | T: 1.27(1.05, 1.52) |
| RS7983142 | 13: 066740589 | | 0.0211 | T,T: 1.83(1.16, 2.9) | 0.0168 | T: 1.29(1.05, 1.58) | 0.0198 | T,T: 1.76(1.18, 2.64) | 0.0255 | T: 1.25(1.03, 1.52) |
| RS1146878 | 13: 075772974 | | 0.0511 | G,G: 1.45(1.02, 2.06) | 0.0161 | G: 1.27(1.05, 1.54) | 0.0498 | G,G: 1.47(1.08, 2.01) | 0.0343 | G: 1.22(1.02, 1.47) |
| RS9546644 | 13: 083751649 | | 0.0039 | T,T: 1.74(1.25, 2.41) | 0.0056 | T: 1.31(1.09, 1.59) | 0.0076 | !C,C: 1.64(1.2, 2.25) | 0.0271 | T: 1.23(1.03, 1.48) |
| RS3861162 | 13: 083801107 | | 0.0387 | T,T: 1.5(1.09, 2.05) | 0.0227 | T: 1.25(1.03, 1.51) | 0.0037 | !C,C: 1.7(1.24, 2.34) | 0.0144 | T: 1.26(1.05, 1.52) |
| RS9546953 | 13: 084412352 | | 0.0215 | !A,A: 1.76(1.15, 2.69) | 0.2024 | G: 1.14(0.94, 1.39) | 0.0111 | !A,A: 1.99(1.26, 3.15) | 0.0439 | G: 1.23(1.01, 1.5) |
| RS4076203 | 13: 086794849 | | 0.0412 | A,C: 1.4(1.02, 1.93) | 0.3658 | A: 1.14(0.86, 1.49) | 0.0360 | !A,A: 2.59(1.21, 5.54) | 0.1277 | C: 1.22(0.95, 1.57) |
| RS2148229 | 13: 092281547 | GPC5 | 0.0081 | G,G: 1.6(1.15, 2.22) | 0.1076 | G: 1.17(0.97, 1.42) | 0.0410 | G,G: 1.45(1.06, 1.97) | 0.1884 | G: 1.13(0.94, 1.36) |
| RS9590101 | 13: 094341946 | | 0.0324 | !A,G: 1.41(1.05, 1.88) | 0.3884 | A: 1.12(0.88, 1.42) | 0.0887 | A,A: 1.33(1.02, 1.74) | 0.0302 | A: 1.28(1.03, 1.61) |
| RS1806507 | 13: 096836578 | MBNL2 | 0.1061 | !G,G: 1.29(0.97, 1.71) | 0.0391 | A: 1.23(1.01, 1.49) | 0.1107 | !G,G: 1.27(0.97, 1.66) | 0.0376 | A: 1.23(1.02, 1.48) |
| RS1328657 | 13: 096981887 | | 0.0224 | !G,G: 1.43(1.09, 1.87) | 0.0053 | A: 1.34(1.09, 1.65) | 0.0282 | !G,G: 1.38(1.06, 1.79) | 0.0071 | A: 1.32(1.08, 1.62) |
| RS9513260 | 13: 097079638 | | 0.1405 | !T,T: 1.28(0.98, 1.67) | 0.0446 | C: 1.23(1.01, 1.51) | 0.0832 | !T,T: 1.32(1.02, 1.71) | 0.0248 | C: 1.26(1.03, 1.54) |
| RS4772260 | 13: 099443724 | | 0.0086 | C,C: 1.73(1.21, 2.48) | 0.0083 | C: 1.3(1.07, 1.57) | 0.0219 | !T,T: 1.44(1.09, 1.91) | 0.0962 | C: 1.18(0.98, 1.42) |
| RS8002928 | 13: 099743604 | PCCA | 0.0865 | G,G: 1.4(1.03, 1.9) | 0.0416 | G: 1.22(1.01, 1.48) | 0.0882 | G,G: 1.34(0.99, 1.8) | 0.0307 | G: 1.23(1.02, 1.48) |
| RS12874523 | 13: 105487642 | | 0.0498 | A,A: 1.42(1.07, 1.9) | 0.0156 | A: 1.36(1.06, 1.75) | 0.0470 | !A,C: 1.4(1.05, 1.86) | 0.2536 | A: 1.15(0.91, 1.45) |
| RS9521112 | 13: 108414403 | RP11- | 0.0062 | !A,G: 1.74(1.22, 2.48) | 0.0248 | G: 1.45(1.06, 1.99) | 0.0099 | A,A: 17.81(1.03, 309.53) | 0.5522 | A: 1.1(0.82, 1.48) |
| | | 54H7.1 | | | | | | | | |
| RS9588148 | 13: 109819082 | COL4A2 | 0.0377 | T,T: 1.44(1.08, 1.92) | 0.0268 | T: 1.32(1.04, 1.68) | 0.0269 | T,T: 1.44(1.1, 1.88) | 0.0089 | T: 1.36(1.08, 1.71) |
| RS9521748 | 13: 109860681 | COL4A2 | 0.0037 | A,A: 2.06(1.32, 3.22) | 0.0070 | A: 1.32(1.08, 1.61) | 0.1040 | A,A: 1.45(0.97, 2.18) | 0.0424 | A: 1.23(1.01, 1.49) |
| RS11841484 | 13: 110771589 | C13ORF16 | 0.0756 | !T,T: 1.47(1.03, 2.09) | 0.0255 | C: 1.46(1.05, 2.03) | 0.0211 | !T,T: 1.61(1.13, 2.3) | 0.0064 | C: 1.58(1.14, 2.19) |
| RS7140768 | 14: 019966697 | LOC123103, | 0.0017 | T,T: 2.02(1.35, 3.02) | 0.0049 | T: 1.32(1.09, 1.61) | 0.0187 | T,T: 1.66(1.16, 2.38) | 0.0625 | T: 1.2(0.99, 1.44) |
| | | LOC650481 | | | | | | | | |
| RS17277375 | 14: 019979312 | LOC441672 | 0.0857 | !T,T: 1.36(1.04, 1.79) | 0.0489 | C: 1.22(1, 1.49) | 0.0044 | C,T: 1.52(1.17, 1.97) | 0.6944 | C: 1.04(0.86, 1.26) |
| RS433673 | 14: 022920742 | MYH6, | 0.0068 | !T,T: 18.34(1.06, 318.73) | 0.6312 | C: 1.09(0.8, 1.49) | 0.0277 | !T,T: 3.75(0.93, 15.19) | 0.5134 | T: 1.1(0.83, 1.46) |
| | | CMTM5 | | | | | | | | |
| RS3825581 | 14: 023067853 | ZFHX2, | 0.1392 | !A,A: 1.29(0.99, 1.69) | 0.0473 | G: 1.24(1, 1.53) | 0.0475 | G,G: 1.87(1.12, 3.13) | 0.2267 | G: 1.14(0.92, 1.4) |
| | | ZNF409 | | | | | | | | |
| RS3794485 | 14: 026129186 | NOVA1 | 0.1034 | T,T: 1.36(1.02, 1.8) | 0.0464 | T: 1.29(1.01, 1.65) | 0.0336 | T,T: 1.39(1.05, 1.83) | 0.0119 | T: 1.37(1.07, 1.74) |
| RS1955773 | 14: 029800649 | | 0.0380 | G,G: 1.43(1.09, 1.88) | 0.0284 | G: 1.25(1.03, 1.53) | 0.0444 | G,G: 1.39(1.07, 1.82) | 0.0210 | G: 1.26(1.04, 1.53) |
| RS1431062 | 14: 033752846 | LOC651341, | 0.0885 | A,A: 1.7(1, 2.88) | 0.0472 | A: 1.25(1.01, 1.55) | 0.0067 | A,A: 2.46(1.38, 4.38) | 0.0478 | A: 1.25(1.01, 1.55) |
| | | LOC644347 | | | | | | | | |
| RS1431063 | 14: 033753171 | LOC644347, | 0.0833 | G,G: 1.69(1, 2.87) | 0.0369 | G: 1.26(1.02, 1.56) | 0.0070 | G,G: 2.45(1.37, 4.37) | 0.0423 | G: 1.25(1.01, 1.55) |
| | | LOC651341 | | | | | | | | |
| RS2010608 | 14: 034289096 | | 0.0375 | A,A: 1.42(1.01, 1.98) | 0.0263 | A: 1.44(1.05, 1.97) | 0.0464 | A,A: 1.45(1.08, 1.95) | 0.0180 | A: 1.38(1.06, 1.81) |
| RS8015303 | 14: 034316223 | BAZ1A | 0.0598 | A,A: 1.53(0.99, 2.37) | 0.0443 | A: 1.55(1.02, 2.36) | 0.0371 | A,A: 1.58(1.08, 2.31) | 0.0100 | A: 1.59(1.11, 2.27) |
| RS712332 | 14: 036336400 | SLC25A21 | 0.0121 | T,T: 4.3(1.34, 13.83) | 0.6289 | T: 1.07(0.82, 1.4) | 0.1067 | C,C: 1.37(1.01, 1.86) | 0.0432 | C: 1.33(1.01, 1.76) |
| RS1266264 | 14: 045254345 | | 0.0994 | !T,T: 1.29(0.98, 1.69) | 0.0348 | C: 1.24(1.02, 1.52) | 0.0242 | C,C: 1.67(1.09, 2.55) | 0.4855 | C: 1.08(0.88, 1.31) |
| RS981951 | 14: 045397270 | | 0.0197 | T,T: 2.01(1.16, 3.47) | 0.0144 | T: 1.31(1.06, 1.62) | 0.0834 | !C,C: 1.34(1.03, 1.75) | 0.0393 | T: 1.23(1.01, 1.5) |
| RS2933187 | 14: 046447470 | MAMDC1 | 0.0531 | C,C: 1.36(1.04, 1.79) | 0.0163 | C: 1.31(1.05, 1.63) | 0.1047 | C,C: 1.26(0.97, 1.63) | 0.0386 | C: 1.25(1.01, 1.54) |
| RS4901053 | 14: 050287324 | NIN | 0.1025 | !G,G: 1.37(1, 1.87) | 0.0417 | T: 1.22(1.01, 1.48) | 0.0011 | !G,G: 1.68(1.22, 2.29) | 0.0003 | T: 1.41(1.17, 1.69) |
| RS7140492 | 14: 057590253 | C14ORF37 | 0.0315 | G,G: 8.59(0.46, 159.96) | 0.5076 | T: 1.17(0.76, 1.8) | 0.0005 | T,T: 2.23(1.44, 3.46) | 1.24E−04 | T: 2.23(1.47, 3.39) |
| RS393170 | 14: 059688357 | DHRS7 | 0.0088 | T,T: 1.59(1.14, 2.22) | 0.0025 | T: 1.61(1.18, 2.19) | 0.0351 | T,T: 1.42(1.03, 1.95) | 0.0155 | T: 1.44(1.08, 1.92) |
| RS10483727 | 14: 060142628 | | 0.0759 | !C,C: 1.36(1.02, 1.8) | 0.0299 | T: 1.24(1.02, 1.51) | 0.0375 | C,T: 1.41(1.08, 1.83) | 0.5630 | T: 1.06(0.88, 1.28) |
| RS1270013 | 14: 061572437 | SYT16 | 0.0835 | !C,C: 1.35(1.03, 1.77) | 0.0339 | T: 1.26(1.02, 1.56) | 0.0981 | !C,C: 1.28(0.98, 1.66) | 0.0386 | T: 1.26(1.01, 1.57) |
| RS4078408 | 14: 065342587 | | 0.0268 | A,G: 1.45(1.1, 1.91) | 0.0615 | G: 1.23(0.99, 1.53) | 0.0215 | !G,G: 1.82(1.14, 2.92) | 0.3472 | A: 1.11(0.9, 1.36) |
| RS8013361 | 14: 065483143 | | 0.0028 | !C,C: 1.78(1.24, 2.57) | 0.0009 | T: 1.4(1.15, 1.7) | 0.0452 | !C,C: 1.6(1.07, 2.38) | 0.0248 | T: 1.25(1.03, 1.51) |
| RS2238195 | 14: 071873833 | RGS6 | 0.0288 | !T,T: 1.35(1.02, 1.77) | 0.0123 | C: 1.34(1.07, 1.67) | 0.1025 | C,C: 1.65(0.97, 2.82) | 0.0429 | C: 1.25(1.01, 1.55) |
| RS847330 | 14: 071899350 | RGS6 | 0.0460 | C,C: 1.79(1.06, 3.01) | 0.0244 | C: 1.28(1.03, 1.57) | 0.0727 | C,C: 1.57(1, 2.47) | 0.0259 | C: 1.26(1.03, 1.55) |
| RS1014087 | 14: 073035327 | MGC48595 | 0.0322 | G,G: 3.73(1.14, 12.18) | 0.7797 | G: 1.04(0.79, 1.37) | 0.0011 | G,G: 5.95(1.89, 18.7) | 0.1444 | G: 1.23(0.94, 1.62) |
| RS3784011 | 14: 075001425 | JDP2 | 0.0337 | !C,C: 1.42(1.08, 1.86) | 0.0126 | T: 1.3(1.06, 1.6) | 0.0981 | !C,C: 1.27(0.97, 1.66) | 0.0365 | T: 1.23(1.02, 1.49) |
| RS11623206 | 14: 075819928 | | 0.0267 | !T,T: 2.45(1.17, 5.14) | 0.6317 | C: 1.07(0.84, 1.35) | 0.0526 | C,C: 1.3(1, 1.7) | 0.0274 | C: 1.29(1.03, 1.61) |
| RS850748 | 14: 076187303 | | 0.0294 | !G,G: 1.42(1.09, 1.86) | 0.0098 | A: 1.32(1.07, 1.62) | 0.0284 | A,G: 1.33(1.02, 1.74) | 0.8355 | G: 1.02(0.84, 1.26) |
| RS1950265 | 14: 077664518 | | 0.1003 | G,G: 1.41(0.99, 2.01) | 0.0390 | G: 1.23(1.01, 1.49) | 0.0102 | !G,T: 1.49(1.15, 1.94) | 0.9250 | T: 1.01(0.84, 1.21) |
| RS8022732 | 14: 086360515 | | 0.0386 | !T,T: 1.42(1.08, 1.86) | 0.0168 | C: 1.28(1.05, 1.56) | 0.0252 | C,C: 1.71(1.16, 2.53) | 0.0388 | C: 1.23(1.01, 1.49) |
| RS2254354 | 14: 086375280 | | 0.1130 | !C,C: 1.32(0.99, 1.76) | 0.0444 | T: 1.22(1.01, 1.48) | 0.0300 | T,T: 1.57(1.12, 2.21) | 0.0192 | T: 1.26(1.04, 1.51) |
| RS10498611 | 14: 086598814 | | 0.0435 | !C,T: 1.45(1.08, 1.94) | 0.1032 | T: 1.23(0.97, 1.58) | 0.0248 | !C,T: 1.42(1.07, 1.89) | 0.5186 | T: 1.08(0.86, 1.36) |
| RS1288709 | 14: 087291337 | | 0.0232 | !T,T: 2.54(1.25, 5.13) | 0.2357 | C: 1.15(0.91, 1.45) | 0.0121 | C,C: 1.52(1.15, 2.01) | 0.0035 | C: 1.43(1.13, 1.82) |
| RS737076 | 14: 095032380 | | 0.0690 | !G,G: 1.34(1.01, 1.77) | 0.0199 | A: 1.26(1.04, 1.53) | 0.0394 | !G,G: 1.42(1.08, 1.88) | 0.0718 | A: 1.19(0.99, 1.43) |
| RS10498648 | 14: 095714790 | | 0.0249 | A,A: 1.45(1.11, 1.91) | 0.0150 | A: 1.29(1.05, 1.58) | 0.0990 | !C,C: 1.57(0.99, 2.49) | 0.0469 | A: 1.23(1, 1.5) |
| RS1109302 | 14: 095728883 | | 0.1075 | !T,T: 1.36(1.01, 1.84) | 0.0418 | C: 1.22(1.01, 1.47) | 0.0107 | !T,T: 1.59(1.17, 2.14) | 0.0213 | C: 1.24(1.04, 1.5) |
| RS2400352 | 14: 098022578 | | 0.0458 | A,G: 1.54(1.07, 2.23) | 0.0918 | A: 1.34(0.96, 1.87) | 0.0143 | A,G: 1.61(1.13, 2.31) | 0.0348 | A: 1.43(1.03, 2) |
| RS7493924 | 14: 100110151 | | 0.0225 | C,C: 3.65(0.9, 14.8) | 0.4870 | T: 1.12(0.83, 1.51) | 0.0722 | T,T: 1.34(0.99, 1.83) | 0.0334 | T: 1.36(1.03, 1.8) |
| RS2269318 | 14: 102478372 | CDC42BPB | 0.0815 | !A,A: 1.45(1.02, 2.06) | 0.0311 | G: 1.44(1.04, 2) | 0.0054 | !A,A: 1.56(1.14, 2.14) | 0.0040 | G: 1.54(1.15, 2.07) |
| RS875014 | 14: 102748906 | | 0.0222 | A,A: 10.52(0.58, 190.81) | 0.0272 | A: 1.52(1.05, 2.19) | 0.0005 | !A,G: 2.01(1.28, 3.13) | 0.1157 | G: 1.37(0.93, 2.03) |
| RS2841231 | 14: 104411688 | KIAA0284 | 0.0237 | T,T: 1.73(1.16, 2.57) | 0.0446 | T: 1.23(1.01, 1.49) | 0.0722 | !C,C: 1.36(1.04, 1.77) | 0.0347 | T: 1.23(1.02, 1.49) |
| RS12906657 | 15: 021878752 | | 0.0373 | C,T: 1.41(1.08, 1.85) | 0.3533 | T: 1.1(0.9, 1.33) | 0.0528 | !C,C: 1.38(1.04, 1.82) | 0.0177 | T: 1.25(1.04, 1.51) |
| RS3101683 | 15: 025554889 | | 0.0648 | !G,G: 1.49(1.03, 2.15) | 0.0255 | A: 1.25(1.03, 1.52) | 0.0468 | !A,G: 1.38(1.06, 1.79) | 0.1555 | A: 1.15(0.95, 1.39) |
| RS1456212 | 15: 029211346 | | 0.0409 | A,A: 3.34(1.16, 9.62) | 0.0740 | A: 1.3(0.98, 1.72) | 0.0533 | !G,G: 1.37(1.03, 1.83) | 0.0228 | A: 1.35(1.05, 1.75) |
| RS4780094 | 15: 031287357 | LOC649021 | 0.1095 | !A,A: 1.36(1.02, 1.81) | 0.0398 | G: 1.29(1.01, 1.65) | 0.0022 | !A,A: 1.51(1.15, 1.98) | 0.0006 | G: 1.49(1.19, 1.87) |
| RS8037685 | 15: 035565656 | | 0.0350 | !A,A: 1.54(1.1, 2.14) | 0.0127 | G: 1.47(1.09, 1.98) | 0.0391 | !G,G: 4.15(1.04, 16.6) | 0.9404 | A: 1.01(0.76, 1.36) |
| RS10518743 | 15: 040017144 | EHD4 | 0.0419 | !T,T: 1.42(1.02, 1.98) | 0.0176 | C: 1.45(1.07, 1.97) | 0.1114 | !T,T: 1.36(0.99, 1.86) | 0.0414 | C: 1.35(1.01, 1.8) |
| RS11854484 | 15: 043332770 | SLC28A2 | 0.0701 | !C,C: 1.36(1.03, 1.8) | 0.0280 | T: 1.25(1.03, 1.52) | 0.0921 | T,T: 1.48(1, 2.2) | 0.0377 | T: 1.23(1.02, 1.5) |
| RS2444043 | 15: 053321634 | RAB27A | 0.0278 | !T,T: 1.44(1.07, 1.93) | 0.0075 | C: 1.3(1.07, 1.57) | 0.0446 | C,C: 1.48(1.09, 2.01) | 0.0387 | C: 1.22(1.01, 1.46) |
| RS7175728 | 15: 053681850 | | 0.0038 | !C,C: 1.78(1.24, 2.55) | 0.0020 | T: 1.36(1.12, 1.64) | 0.0162 | T,T: 1.42(1.09, 1.86) | 0.0042 | T: 1.32(1.09, 1.6) |
| RS1509405 | 15: 054109146 | LOC440280 | 0.1001 | G,G: 1.38(1.02, 1.88) | 0.0466 | G: 1.22(1.01, 1.47) | 0.0444 | !A,A: 1.39(1.04, 1.86) | 0.2031 | G: 1.13(0.94, 1.36) |
| RS3191402 | 15: 057216452 | MYO1E | 0.0080 | !C,T: 1.5(1.15, 1.97) | 1.0000 | C: 1(0.82, 1.22) | 0.1076 | C,C: 1.32(1, 1.74) | 0.0449 | C: 1.21(1.01, 1.46) |
| RS2960807 | 15: 057957580 | | 0.0164 | !G,G: 1.47(1.13, 1.93) | 0.0142 | T: 1.3(1.05, 1.6) | 0.0367 | !T,T: 1.88(1.05, 3.36) | 0.8315 | G: 1.02(0.83, 1.26) |
| RS10519070 | 15: 058856045 | RORA | 0.0664 | !T,T: 1.42(1.04, 1.93) | 0.0197 | C: 1.4(1.05, 1.85) | 0.0435 | C,T: 1.49(1.09, 2.05) | 0.0276 | C: 1.37(1.04, 1.81) |
| RS4984247 | 15: 061545700 | | 0.0164 | C,T: 1.55(1.12, 2.13) | 0.0906 | C: 1.28(0.97, 1.68) | 0.0963 | !T,T: 1.35(1, 1.83) | 0.0366 | C: 1.34(1.02, 1.77) |
| RS8041258 | 15: 067423676 | PAQR5 | 0.0160 | C,T: 1.92(1.03, 3.6) | 0.3205 | T: 1.35(0.77, 2.37) | 0.0352 | C,T: 1.81(1.1, 2.98) | 0.0244 | T: 1.72(1.07, 2.76) |
| RS4886630 | 15: 071882793 | | 0.0443 | C,C: 6.1(1.08, 34.3) | 0.2245 | C: 1.21(0.9, 1.63) | 0.0434 | C,C: 9.34(0.5, 174.08) | 0.5668 | A: 1.11(0.8, 1.53) |
| RS950169 | 15: 082497465 | ADAMTSL3 | 0.0011 | !A,A: 2.23(1.33, 3.74) | 0.0006 | G: 1.44(1.17, 1.78) | 0.0170 | G,G: 1.44(1.1, 1.87) | 0.0046 | G: 1.38(1.11, 1.72) |
| RS2135551 | 15: 082498855 | ADAMTSL3 | 0.0010 | !G,G: 2.15(1.3, 3.54) | 0.0004 | A: 1.46(1.18, 1.79) | 0.0122 | A,A: 1.43(1.1, 1.87) | 0.0033 | A: 1.39(1.12, 1.73) |
| RS1911155 | 15: 082578639 | FAM42B | 0.0044 | A,A: 1.45(1.11, 1.9) | 0.0015 | A: 1.4(1.14, 1.73) | 0.0132 | A,A: 1.44(1.11, 1.88) | 0.0033 | A: 1.39(1.12, 1.73) |
| RS12910334 | 15: 082749322 | KIAA1920 | 0.0049 | A,A: 1.5(1.14, 1.96) | 0.0016 | A: 1.4(1.14, 1.72) | 0.0192 | A,A: 1.4(1.07, 1.83) | 0.0049 | A: 1.37(1.1, 1.7) |
| RS3743162 | 15: 083231973 | SLC28A1 | 0.1254 | T,T: 1.32(1, 1.75) | 0.0477 | T: 1.28(1.01, 1.61) | 0.0855 | !C,C: 1.9(0.99, 3.63) | 0.0475 | T: 1.27(1.01, 1.59) |
| RS2173091 | 15: 084598403 | | 0.0693 | C,T: 1.42(1.05, 1.93) | 0.0444 | T: 1.32(1.01, 1.71) | 0.1192 | !C,C: 1.34(1.01, 1.79) | 0.0469 | T: 1.29(1, 1.66) |
| RS11855600 | 15: 086764263 | | 0.0189 | C,C: 2.8(1.21, 6.5) | 0.7077 | C: 1.05(0.82, 1.34) | 0.0536 | C,C: 2.16(1.06, 4.44) | 0.0403 | C: 1.27(1.01, 1.6) |
| RS2589949 | 15: 088756019 | IQGAP1 | 0.1088 | !A,A: 1.48(0.98, 2.25) | 0.0453 | G: 1.23(1.01, 1.51) | 0.0178 | A,G: 1.39(1.07, 1.82) | 0.8347 | A: 1.02(0.83, 1.26) |
| RS2388814 | 15: 092874459 | | 0.0452 | !A,G: 1.38(1.04, 1.84) | 0.3760 | A: 1.11(0.88, 1.4) | 0.0422 | G,G: 1.85(1.06, 3.24) | 0.6595 | G: 1.05(0.85, 1.3) |
| RS8044035 | 16: 003191523 | | 0.0116 | A,A: 1.52(1.15, 1.99) | 0.0061 | A: 1.37(1.09, 1.72) | 0.0006 | !A,G: 1.7(1.3, 2.24) | 0.0169 | A: 1.3(1.05, 1.61) |
| RS548927 | 16: 003197869 | | 0.0440 | !A,G: 1.78(1.07, 2.94) | 0.0306 | A: 1.69(1.04, 2.73) | 0.0453 | A,A: 1.46(0.99, 2.16) | 0.0377 | A: 1.49(1.02, 2.17) |
| RS7201758 | 16: 008512159 | | 0.0210 | !A,C: 1.45(1.09, 1.93) | 0.3769 | A: 1.11(0.88, 1.4) | 0.0784 | A,A: 1.36(1.04, 1.78) | 0.0493 | A: 1.26(1, 1.58) |
| RS7187654 | 16: 010558868 | EMP2 | 0.0825 | A,A: 1.38(1.03, 1.83) | 0.0396 | A: 1.23(1.01, 1.49) | 0.0435 | !A,G: 1.39(1.07, 1.81) | 0.3379 | A: 1.1(0.91, 1.33) |
| RS1548956 | 16: 010664693 | FLJ32871 | 0.0208 | !T,T: 1.74(1.12, 2.7) | 0.0106 | C: 1.3(1.06, 1.59) | 0.0028 | C,T: 1.56(1.2, 2.03) | 0.0584 | T: 1.21(1, 1.46) |
| RS3213646 | 16: 020717568 | EXOD1, | 0.1325 | !C,C: 1.28(0.97, 1.7) | 0.0431 | T: 1.22(1.01, 1.49) | 0.0611 | T,T: 1.44(1.01, 2.06) | 0.0217 | T: 1.25(1.03, 1.51) |
| | | ACSM3, | | | | | | | | |
| | | LOC653783 | | | | | | | | |
| RS11074709 | 16: 025640038 | HS3ST4 | 0.0277 | !A,C: 1.48(1.11, 1.98) | 0.0527 | C: 1.27(1, 1.61) | 0.0069 | !A,C: 1.44(1.09, 1.9) | 0.5561 | C: 1.08(0.86, 1.36) |
| RS4238915 | 16: 025643984 | HS3ST4 | 0.0265 | !G,T: 1.48(1.11, 1.96) | 0.0518 | T: 1.26(1, 1.59) | 0.0075 | !G,T: 1.42(1.08, 1.88) | 0.5610 | T: 1.07(0.86, 1.35) |
| RS4787948 | 16: 027248560 | IL4R | 0.0140 | !A,A: 1.49(1.14, 1.95) | 0.0143 | G: 1.31(1.06, 1.61) | 0.0395 | G,G: 1.69(1.09, 2.62) | 0.0192 | G: 1.27(1.04, 1.55) |
| RS772859 | 16: 027701027 | KIAA0556 | 0.1301 | !T,T: 1.31(0.99, 1.73) | 0.0477 | C: 1.22(1.01, 1.48) | 0.0105 | C,T: 1.49(1.15, 1.94) | 0.6672 | C: 1.04(0.87, 1.26) |
| RS4788009 | 16: 027849046 | | 0.0144 | !A,A: 1.96(1.23, 3.1) | 0.0384 | G: 1.24(1.01, 1.52) | 0.0531 | G,G: 1.36(1.05, 1.77) | 0.0149 | G: 1.28(1.05, 1.57) |
| RS151230 | 16: 028490716 | CCDC101 | 0.0417 | C,T: 1.51(1.09, 2.1) | 0.0231 | T: 1.4(1.05, 1.87) | 0.0103 | T,T: 4.68(1.46, 15.05) | 0.0438 | T: 1.34(1.01, 1.78) |
| RS1362567 | 16: 045782194 | ITFG1 | 0.0562 | !C,T: 1.46(1.07, 2) | 0.0346 | C: 1.35(1.03, 1.79) | 0.0197 | C,C: 1.54(1.14, 2.09) | 0.0076 | C: 1.45(1.1, 1.9) |
| RS10521169 | 16: 047463980 | | 0.0214 | C,C: 2.12(1.17, 3.87) | 0.0140 | C: 1.33(1.06, 1.67) | 0.0124 | C,T: 1.51(1.15, 1.98) | 0.0482 | C: 1.26(1.01, 1.57) |
| RS12930072 | 16: 047510482 | | 0.0012 | !C,T: 1.68(1.27, 2.22) | 0.0324 | T: 1.27(1.02, 1.57) | 0.0291 | !C,C: 1.83(1.11, 3) | 0.0157 | T: 1.29(1.05, 1.59) |
| RS8053786 | 16: 047566891 | | 0.0911 | A,A: 1.35(1.03, 1.78) | 0.0324 | A: 1.27(1.02, 1.59) | 0.0124 | !G,G: 2.24(1.25, 4.01) | 0.0160 | A: 1.31(1.05, 1.62) |
| RS2200537 | 16: 053769135 | | 0.1613 | T,T: 1.28(0.97, 1.69) | 0.0482 | T: 1.26(1, 1.58) | 0.0431 | !C,C: 2.31(1.14, 4.68) | 0.0829 | T: 1.22(0.98, 1.51) |
| RS728571 | 16: 053771594 | | 0.0134 | G,G: 1.54(1.15, 2.07) | 0.0054 | G: 1.43(1.11, 1.83) | 0.0105 | G,G: 1.41(1.07, 1.86) | 0.0058 | G: 1.41(1.11, 1.79) |
| RS1436413 | 16: 061775362 | | 0.0661 | G,G: 1.37(1.05, 1.8) | 0.0287 | G: 1.26(1.02, 1.54) | 0.0721 | !T,T: 1.48(1.01, 2.17) | 0.0296 | G: 1.24(1.02, 1.49) |
| RS427576 | 16: 072386771 | | 0.1091 | A,A: 1.36(1.01, 1.82) | 0.0413 | A: 1.22(1.01, 1.48) | 0.0400 | A,A: 1.41(1.04, 1.9) | 0.0129 | A: 1.26(1.05, 1.52) |
| RS725710 | 16: 073431014 | | 0.0100 | C,C: 1.61(1.18, 2.21) | 0.0047 | C: 1.5(1.13, 1.98) | 0.0253 | C,C: 1.43(1.06, 1.92) | 0.0091 | C: 1.42(1.09, 1.86) |
| RS1390907 | 16: 075501858 | | 0.0331 | A,A: 1.44(1.09, 1.89) | 0.0247 | A: 1.26(1.03, 1.54) | 0.0017 | !A,G: 1.61(1.24, 2.09) | 0.2033 | A: 1.14(0.94, 1.38) |
| RS8059438 | 16: 081773975 | CDH13 | 0.0080 | !T,T: 1.63(1.16, 2.31) | 0.0037 | C: 1.61(1.17, 2.23) | 0.0656 | !T,T: 1.38(0.99, 1.92) | 0.0300 | C: 1.41(1.04, 1.92) |
| RS3829514 | 16: 083652499 | KIAA0513 | 0.0103 | T,T: 1.89(1.15, 3.09) | 0.0121 | T: 1.84(1.13, 2.97) | 0.0281 | !C,T: 1.61(1.03, 2.51) | 0.1339 | T: 1.4(0.92, 2.14) |
| RS11117407 | 16: 084411812 | | 0.0488 | !T,T: 1.35(1.02, 1.8) | 0.0205 | C: 1.34(1.05, 1.72) | 0.0355 | C,C: 3.01(1.22, 7.43) | 0.1148 | C: 1.22(0.95, 1.57) |
| RS2965946 | 16: 088044113 | ANKRD11 | 0.0437 | !C,T: 1.35(1.03, 1.77) | 0.7851 | C: 1.04(0.84, 1.28) | 0.0431 | T,T: 1.82(1.13, 2.92) | 0.1352 | T: 1.17(0.96, 1.43) |
| RS8060934 | 16: 088447526 | SPIRE2 | 0.1179 | T,T: 1.34(0.96, 1.85) | 0.0411 | T: 1.22(1.01, 1.48) | 0.1277 | T,T: 1.32(0.97, 1.8) | 0.0433 | T: 1.21(1.01, 1.46) |
| RS8072032 | 17: 004895930 | | 0.0568 | !G,T: 1.57(1.05, 2.34) | 0.0378 | G: 1.49(1.03, 2.16) | 0.0027 | G,G: 1.65(1.13, 2.41) | 0.0026 | G: 1.72(1.2, 2.46) |
| RS4790743 | 17: 004940854 | ZFP3 | 0.0526 | A,A: 1.6(1.01, 2.54) | 0.0416 | A: 1.6(1.02, 2.51) | 0.0108 | A,A: 1.69(1.1, 2.59) | 0.0078 | A: 1.75(1.16, 2.64) |
| RS1619016 | 17: 007491278 | | 0.0223 | !A,A: 1.49(1.08, 2.05) | 0.0097 | G: 1.47(1.1, 1.97) | 0.1412 | !A,A: 1.35(0.98, 1.85) | 0.0476 | G: 1.34(1, 1.79) |
| RS714407 | 17: 008801218 | | 0.0223 | !A,A: 3.5(1.2, 10.15) | 0.7768 | G: 1.05(0.79, 1.38) | 0.0401 | !G,G: 1.42(1.07, 1.88) | 0.0141 | A: 1.37(1.07, 1.75) |
| RS11653099 | 17: 009811124 | GAS7 | 0.0589 | T,T: 1.43(1.06, 1.94) | 0.0289 | T: 1.24(1.03, 1.5) | 0.0278 | !C,T: 1.39(1.07, 1.8) | 0.2801 | C: 1.11(0.92, 1.33) |
| RS719470 | 17: 014147087 | HS3ST3B1 | 0.0814 | C,C: 1.39(0.98, 1.98) | 0.0271 | C: 1.25(1.03, 1.51) | 0.1185 | !T,T: 1.32(0.99, 1.74) | 0.0466 | C: 1.21(1, 1.46) |
| RS2191176 | 17: 014745432 | LOC440402 | 0.0316 | !T,T: 1.4(1.07, 1.83) | 0.0093 | C: 1.33(1.08, 1.65) | 0.0371 | C,C: 1.71(1.04, 2.84) | 0.0123 | C: 1.31(1.06, 1.61) |
| RS230977 | 17: 015199094 | | 0.0137 | A,G: 1.4(1.03, 1.91) | 0.4963 | G: 1.11(0.85, 1.44) | 0.0475 | !A,A: 1.41(1.06, 1.89) | 0.0158 | G: 1.38(1.07, 1.79) |
| RS4613115 | 17: 027953003 | MYO1D | 0.0327 | A,G: 1.38(1.06, 1.81) | 0.4092 | G: 1.09(0.9, 1.31) | 0.0367 | !G,G: 1.43(1.08, 1.9) | 0.0142 | A: 1.26(1.05, 1.52) |
| RS4796217 | 17: 031893304 | LOC441792 | 0.0344 | !C,T: 1.43(1.09, 1.88) | 0.1248 | C: 1.18(0.96, 1.45) | 0.0867 | C,C: 1.33(1.03, 1.74) | 0.0310 | C: 1.27(1.02, 1.56) |
| RS713101 | 17: 040548347 | PLCD3 | 0.0102 | A,A: 1.67(1.18, 2.35) | 0.0637 | A: 1.2(0.99, 1.46) | 0.0487 | A,A: 1.54(1.09, 2.17) | 0.0632 | A: 1.2(0.99, 1.44) |
| RS9896945 | 17: 051713575 | ANKFN1 | 0.0481 | !G,G: 1.59(1.09, 2.31) | 0.1132 | A: 1.17(0.96, 1.42) | 0.0090 | A,A: 1.52(1.16, 1.99) | 0.0070 | A: 1.3(1.08, 1.57) |
| RS2270150 | 17: 056915467 | TBX4 | 0.0237 | A,A: 3.57(1.25, 10.22) | 0.5570 | A: 1.08(0.84, 1.4) | 0.0525 | G,G: 1.44(1.07, 1.95) | 0.0281 | G: 1.36(1.04, 1.78) |
| RS8080666 | 17: 059840612 | | 0.0127 | !A,G: 1.46(1.11, 1.91) | 0.3827 | G: 1.09(0.9, 1.32) | 0.1032 | !A,A: 1.33(0.98, 1.8) | 0.0347 | G: 1.22(1.02, 1.47) |
| RS9303513 | 17: 062462327 | | 0.0491 | !A,A: 1.67(1.1, 2.53) | 0.1517 | G: 1.16(0.95, 1.42) | 0.0423 | !A,A: 1.68(1.11, 2.53) | 0.0685 | G: 1.2(0.99, 1.46) |
| RS1540373 | 17: 066573542 | | 0.0066 | A,G: 1.42(1.08, 1.87) | 0.9569 | G: 1.01(0.82, 1.25) | 0.0104 | !A,A: 2.06(1.2, 3.56) | 0.5165 | G: 1.08(0.87, 1.33) |
| RS8064529 | 17: 071031008 | TSEN54 | 0.0083 | T,T: 2(1.27, 3.17) | 0.0119 | T: 1.31(1.06, 1.61) | 0.0449 | !C,T: 1.33(1.03, 1.74) | 0.9591 | C: 1.01(0.82, 1.23) |
| RS7502321 | 17: 076608112 | | 0.0597 | !C,C: 1.38(1.04, 1.84) | 0.0232 | T: 1.25(1.03, 1.52) | 0.0398 | T,T: 1.63(1.1, 2.41) | 0.0281 | T: 1.24(1.03, 1.5) |
| RS1662813 | 18: 003309915 | | 0.0239 | A,G: 1.53(1.13, 2.09) | 0.0404 | G: 1.32(1.02, 1.71) | 0.0344 | A,G: 1.46(1.09, 1.96) | 0.0268 | G: 1.35(1.04, 1.74) |
| RS2847108 | 18: 004582823 | | 0.0164 | G,G: 1.55(1.14, 2.1) | 0.0325 | G: 1.23(1.02, 1.49) | 0.0855 | G,G: 1.36(1.03, 1.8) | 0.0411 | G: 1.21(1.01, 1.46) |
| RS12455648 | 18: 008465207 | | 0.0031 | !T,T: 2.13(1.28, 3.55) | 0.4186 | C: 1.09(0.89, 1.35) | 0.0547 | !T,T: 1.65(1.03, 2.64) | 0.0231 | C: 1.27(1.03, 1.55) |
| RS9962727 | 18: 010822851 | C18ORF58 | 0.0177 | !C,C: 1.81(1.13, 2.91) | 0.0070 | T: 1.33(1.08, 1.64) | 0.1098 | T,T: 1.32(1.02, 1.71) | 0.0442 | T: 1.23(1.01, 1.51) |
| RS4796974 | 18: 013249774 | C18ORF1, | 0.0127 | C,C: 1.62(1.11, 2.34) | 0.3207 | C: 1.1(0.91, 1.34) | 0.0209 | C,C: 1.54(1.08, 2.19) | 0.3378 | C: 1.1(0.91, 1.33) |
| | | LOC651125 | | | | | | | | |
| RS7232054 | 18: 013261319 | C18ORF1 | 0.0016 | !A,G: 1.61(1.22, 2.11) | 0.7308 | A: 1.04(0.85, 1.26) | 0.0135 | !A,G: 1.45(1.11, 1.88) | 0.7752 | A: 1.03(0.86, 1.24) |
| RS2042743 | 18: 013417800 | C18ORF1 | 0.0423 | A,A: 1.47(1.09, 2) | 0.0525 | A: 1.21(1, 1.46) | 0.0425 | !A,C: 1.38(1.06, 1.79) | 0.5415 | C: 1.06(0.88, 1.28) |
| RS1941611 | 18: 020830023 | | 0.0137 | G,G: 1.49(1.14, 1.96) | 0.0099 | G: 1.32(1.07, 1.63) | 0.0367 | G,G: 1.36(1.05, 1.77) | 0.0114 | G: 1.31(1.07, 1.61) |
| RS5022348 | 18: 020893235 | | 0.0501 | T,T: 1.38(1.03, 1.84) | 0.0145 | T: 1.28(1.05, 1.54) | 0.0041 | !C,C: 1.65(1.17, 2.32) | 0.0012 | T: 1.37(1.13, 1.65) |
| RS12966353 | 18: 024297001 | | 0.0192 | A,A: 1.73(1.13, 2.66) | 0.0075 | A: 1.32(1.08, 1.61) | 0.0932 | A,A: 1.46(1.01, 2.11) | 0.0433 | A: 1.22(1.01, 1.47) |
| RS4799700 | 18: 025807829 | | 0.0387 | !C,C: 1.5(1.1, 2.05) | 0.0589 | T: 1.2(0.99, 1.45) | 0.0708 | T,T: 1.36(1.01, 1.84) | 0.0243 | T: 1.24(1.03, 1.49) |
| RS7244533 | 18: 025834901 | | 0.0362 | C,T: 1.4(1.06, 1.85) | 0.4779 | C: 1.08(0.87, 1.34) | 0.0509 | C,C: 1.73(1.01, 2.97) | 0.0198 | C: 1.29(1.04, 1.6) |
| RS1260719 | 18: 026824023 | DSC3 | 0.0138 | !T,T: 1.52(1.14, 2.03) | 0.0070 | G: 1.31(1.08, 1.58) | 0.0095 | G,T: 1.45(1.12, 1.89) | 0.7717 | T: 1.03(0.85, 1.24) |
| RS638252 | 18: 027628181 | | 0.0609 | G,G: 1.46(1.03, 2.06) | 0.0215 | G: 1.25(1.03, 1.52) | 0.0277 | !A,G: 1.43(1.1, 1.86) | 0.6690 | A: 1.04(0.86, 1.26) |
| RS2391 | 18: 031222379 | | 0.0043 | G,G: 12.46(0.7, 221.86) | 0.4083 | A: 1.18(0.82, 1.68) | 0.0508 | !A,A: 1.53(1.06, 2.2) | 0.0157 | G: 1.52(1.08, 2.14) |
| RS519309 | 18: 032638832 | C18ORF10 | 0.0193 | C,C: 6.1(1.08, 34.3) | 1.0000 | T: 1.01(0.75, 1.35) | 0.0489 | C,C: 4.4(1.1, 17.59) | 0.5321 | C: 1.11(0.82, 1.51) |
| RS323285 | 18: 032989897 | KIAA1328 | 0.0257 | T,T: 6.11(1.09, 34.38) | 0.9402 | T: 1.02(0.76, 1.37) | 0.0399 | T,T: 4.4(1.1, 17.59) | 0.6955 | T: 1.07(0.79, 1.45) |
| RS11082000 | 18: 033171021 | BRUNOL4 | 0.0243 | !G,G: 2.42(1.22, 4.79) | 0.0373 | A: 1.28(1.01, 1.62) | 0.0172 | A,A: 1.46(1.12, 1.91) | 0.0141 | A: 1.32(1.06, 1.64) |
| RS7242236 | 18: 034485243 | | 0.1121 | T,T: 1.35(1, 1.83) | 0.0466 | T: 1.22(1.01, 1.47) | 0.0404 | T,T: 1.45(1.09, 1.93) | 0.0424 | T: 1.21(1.01, 1.46) |
| RS1943413 | 18: 034491676 | | 0.0401 | A,A: 1.58(1.08, 2.33) | 0.0194 | A: 1.26(1.04, 1.54) | 0.0483 | A,A: 1.5(1.07, 2.1) | 0.1844 | A: 1.14(0.94, 1.37) |
| RS987380 | 18: 037702007 | | 0.0227 | A,A: 1.63(1.14, 2.33) | 0.0077 | A: 1.57(1.13, 2.18) | 0.0251 | !G,G: 6.94(1.25, 38.58) | 0.2320 | A: 1.22(0.89, 1.67) |
| RS872035 | 18: 041552646 | | 0.0588 | !G,G: 1.37(1.03, 1.81) | 0.0203 | A: 1.33(1.05, 1.7) | 0.0191 | !G,G: 1.49(1.13, 1.96) | 0.0085 | A: 1.38(1.09, 1.76) |
| RS4624284 | 18: 042584602 | ST8SIA5 | 0.0217 | T,T: 2.12(1.21, 3.69) | 0.1528 | T: 1.17(0.95, 1.46) | 0.0338 | T,T: 2.05(1.13, 3.71) | 0.0357 | T: 1.26(1.02, 1.55) |
| RS9953229 | 18: 044135485 | | 0.0529 | C,C: 1.43(1.06, 1.93) | 0.0225 | C: 1.25(1.03, 1.51) | 0.0167 | !T,T: 1.49(1.1, 2.03) | 0.0041 | C: 1.31(1.09, 1.58) |
| RS745611 | 18: 044163038 | | 0.0010 | C,C: 1.66(1.27, 2.18) | 0.0006 | C: 1.44(1.17, 1.77) | 0.0625 | !A,A: 1.64(1.05, 2.56) | 0.0325 | C: 1.25(1.02, 1.52) |
| RS1222938 | 18: 048195164 | DCC | 0.0415 | A,G: 1.33(1.01, 1.75) | 1.0000 | A: 1.01(0.81, 1.25) | 0.0230 | A,G: 1.45(1.1, 1.9) | 0.2298 | G: 1.14(0.92, 1.42) |
| RS4309482 | 18: 050901467 | | 0.1012 | A,A: 1.32(1, 1.74) | 0.0334 | A: 1.24(1.02, 1.5) | 0.1318 | A,A: 1.26(0.97, 1.66) | 0.0398 | A: 1.22(1.01, 1.47) |
| RS337718 | 18: 067925258 | | 0.0231 | !C,T: 1.42(1.08, 1.86) | 0.6710 | C: 1.05(0.85, 1.29) | 0.0274 | !C,T: 1.37(1.05, 1.78) | 0.8745 | C: 1.02(0.83, 1.26) |
| RS10871696 | 18: 068024582 | | 0.0124 | !A,C: 1.5(1.15, 1.97) | 0.4245 | C: 1.08(0.89, 1.32) | 0.0289 | !A,C: 1.41(1.08, 1.84) | 0.1171 | C: 1.17(0.97, 1.42) |
| RS4536550 | 18: 069099471 | | 0.0232 | !A,G: 1.46(1.11, 1.91) | 0.1037 | A: 1.19(0.97, 1.46) | 0.1540 | A,A: 1.25(0.97, 1.63) | 0.0457 | A: 1.23(1, 1.5) |
| RS999647 | 18: 070700538 | ZNF407 | 0.0167 | !T,T: 1.51(1.13, 2) | 0.0271 | C: 1.25(1.03, 1.51) | 0.1135 | !T,T: 1.3(1, 1.71) | 0.0353 | C: 1.23(1.02, 1.48) |
| RS9960454 | 18: 070798925 | | 0.0094 | !A,A: 1.55(1.17, 2.06) | 0.0156 | G: 1.27(1.05, 1.54) | 0.1092 | !A,A: 1.31(1, 1.72) | 0.0345 | G: 1.23(1.02, 1.48) |
| RS906516 | 18: 070916956 | | 0.0272 | !A,G: 1.41(1.07, 1.84) | 0.7294 | G: 1.04(0.86, 1.26) | 0.0107 | !A,A: 1.5(1.13, 1.99) | 0.0033 | G: 1.32(1.1, 1.59) |
| RS2732239 | 18: 071762651 | | 0.0394 | A,A: 2.52(1.18, 5.4) | 0.0981 | A: 1.23(0.97, 1.57) | 0.0217 | A,C: 1.45(1.09, 1.93) | 0.2309 | A: 1.16(0.92, 1.47) |
| RS2926622 | 18: 071764657 | | 0.0403 | C,C: 2.52(1.18, 5.39) | 0.0861 | C: 1.24(0.97, 1.58) | 0.0217 | C,T: 1.45(1.09, 1.93) | 0.2309 | C: 1.16(0.92, 1.47) |
| RS2612377 | 18: 071771223 | | 0.0398 | G,G: 2.52(1.18, 5.4) | 0.0973 | G: 1.23(0.97, 1.57) | 0.0217 | A,G: 1.45(1.09, 1.93) | 0.2309 | G: 1.16(0.92, 1.47) |
| RS689902 | 18: 072248957 | ZNF516 | 0.0572 | !C,C: 1.42(1.07, 1.89) | 0.0443 | T: 1.22(1.01, 1.48) | 0.1113 | !C,C: 1.33(1.01, 1.74) | 0.0446 | T: 1.21(1.01, 1.47) |
| RS1789107 | 18: 072993175 | | 0.0281 | !G,T: 1.43(1.09, 1.88) | 0.0790 | G: 1.2(0.98, 1.47) | 0.0244 | !T,T: 1.71(1.13, 2.58) | 0.1799 | G: 1.14(0.94, 1.39) |
| RS4541161 | 18: 073966360 | | 0.0170 | T,T: 1.91(1.21, 3.01) | 0.0389 | T: 1.24(1.01, 1.52) | 0.0128 | C,C: 1.47(1.13, 1.91) | 0.0256 | C: 1.25(1.03, 1.52) |
| RS11878115 | 18: 074498350 | | 0.0406 | !A,G: 1.41(1.07, 1.87) | 0.2884 | A: 1.13(0.91, 1.41) | 0.0036 | !G,G: 2.72(1.47, 5.06) | 0.0439 | A: 1.25(1.01, 1.55) |
| RS7227107 | 18: 075328464 | NFATC1 | 0.1217 | !A,A: 1.26(0.96, 1.65) | 0.0465 | G: 1.23(1, 1.52) | 0.0286 | G,G: 1.84(1.16, 2.93) | 0.0551 | G: 1.22(1, 1.48) |
| RS160189 | 18: 075333697 | NFATC1 | 0.0239 | G,G: 1.83(1.16, 2.9) | 0.0216 | G: 1.28(1.04, 1.57) | 0.0118 | G,G: 1.93(1.24, 3) | 0.0396 | G: 1.23(1.01, 1.5) |
| RS8102902 | 19: 003720049 | RAXL1, | 0.0159 | !G,G: 3.14(1.35, 7.3) | 0.0774 | T: 1.24(0.98, 1.58) | 0.0619 | T,T: 1.37(1.04, 1.81) | 0.0196 | T: 1.32(1.05, 1.68) |
| | | MRPL54 | | | | | | | | |
| RS10518246 | 19: 015020981 | | 0.0211 | !A,G: 1.51(1.12, 2.04) | 0.0855 | A: 1.25(0.97, 1.6) | 0.0999 | A,A: 1.35(1.02, 1.78) | 0.0295 | A: 1.3(1.03, 1.66) |
| RS3181163 | 19: 015027162 | CASP14 | 0.0182 | !C,T: 1.5(1.12, 2.02) | 0.1472 | T: 1.2(0.94, 1.54) | 0.1277 | T,T: 1.33(1.01, 1.75) | 0.0426 | T: 1.27(1.01, 1.61) |
| RS8108738 | 19: 018116359 | MAST3 | 0.0066 | A,G: 1.5(1.15, 1.97) | 0.1734 | A: 1.15(0.95, 1.39) | 0.0834 | !A,A: 1.41(1.04, 1.91) | 0.0433 | G: 1.21(1.01, 1.46) |
| RS8103146 | 19: 020582566 | | 0.0501 | C,C: 1.46(1.07, 1.97) | 0.0262 | C: 1.37(1.04, 1.8) | 0.1089 | C,C: 1.39(1.02, 1.91) | 0.0496 | C: 1.34(1.01, 1.79) |
| RS10756 | 19: 035196656 | C19ORF2 | 0.0560 | C,C: 1.39(1.06, 1.82) | 0.0253 | C: 1.26(1.03, 1.55) | 0.0463 | !C,T: 1.38(1.06, 1.8) | 0.4396 | C: 1.08(0.89, 1.33) |
| RS10415219 | 19: 043842075 | ACTN4 | 0.0312 | A,G: 1.42(1.08, 1.86) | 0.3814 | G: 1.09(0.9, 1.32) | 0.0687 | !A,A: 1.38(1.03, 1.85) | 0.0241 | G: 1.24(1.03, 1.49) |
| RS2279144 | 19: 043893621 | ACTN4 | 0.0270 | C,T: 1.43(1.09, 1.88) | 0.4088 | C: 1.09(0.9, 1.32) | 0.0410 | !T,T: 1.43(1.06, 1.92) | 0.0146 | C: 1.26(1.05, 1.52) |
| RS2604879 | 19: 045887119 | NUMBL | 0.0390 | !G,G: 1.43(1.08, 1.89) | 0.0198 | A: 1.32(1.05, 1.68) | 0.0484 | A,G: 1.38(1.03, 1.84) | 0.4663 | A: 1.1(0.87, 1.39) |
| RS659555 | 19: 054521637 | SLC6A16, | 0.0515 | G,G: 1.4(1.07, 1.83) | 0.0396 | G: 1.25(1.01, 1.53) | 0.0052 | !A,G: 1.52(1.17, 1.98) | 0.0223 | G: 1.27(1.04, 1.55) |
| | | AK097351 | | | | | | | | |
| RS10403271 | 19: 055778845 | | 0.0941 | A,A: 1.35(1.03, 1.77) | 0.0445 | A: 1.25(1.01, 1.56) | 0.0252 | !G,G: 2.06(1.18, 3.6) | 0.0283 | A: 1.28(1.03, 1.59) |
| RS9917107 | 19: 056306273 | | 0.0152 | !G,G: 3.11(1.26, 7.69) | 0.6444 | T: 1.06(0.82, 1.38) | 0.0028 | !G,G: 6.61(1.74, 25.18) | 0.0295 | T: 1.37(1.04, 1.8) |
| RS2062655 | 19: 056495675 | | 0.0147 | A,C: 1.49(1.14, 1.95) | 0.6965 | A: 1.04(0.86, 1.26) | 0.0226 | !A,A: 1.58(1.14, 2.19) | 0.0300 | C: 1.23(1.02, 1.48) |
| RS6509525 | 19: 056509963 | LOC402665 | 0.0394 | !T,T: 1.45(1.05, 2) | 0.2639 | C: 1.12(0.92, 1.35) | 0.0509 | !C,C: 1.43(1.07, 1.91) | 0.0384 | T: 1.22(1.01, 1.46) |
| RS3848559 | 19: 056526006 | LOC402665, | 0.0197 | !C,C: 1.53(1.13, 2.08) | 0.0586 | T: 1.21(1, 1.46) | 0.0285 | !C,C: 1.48(1.07, 2.05) | 0.2392 | T: 1.12(0.93, 1.34) |
| | | LOC147645 | | | | | | | | |
| RS2288522 | 19: 060400287 | PTPRH | 0.0423 | C,C: 4.79(0.83, 27.8) | 0.6180 | A: 1.08(0.82, 1.42) | 0.0476 | A,A: 1.39(1.03, 1.89) | 0.0158 | A: 1.4(1.07, 1.84) |
| RS8106321 | 19: 061466373 | | 0.0107 | T,T: 1.58(1.16, 2.14) | 0.0369 | T: 1.23(1.02, 1.49) | 0.0456 | !G,T: 1.38(1.06, 1.79) | 0.6725 | T: 1.04(0.87, 1.25) |
| RS6048226 | 20: 000233323 | | 0.0751 | A,A: 1.36(1.02, 1.79) | 0.0229 | A: 1.25(1.03, 1.52) | 0.0362 | !G,G: 1.58(1.1, 2.27) | 0.1131 | A: 1.17(0.97, 1.41) |
| RS6108123 | 20: 000821277 | ANGPT4 | 0.0541 | A,A: 1.38(1.04, 1.83) | 0.0177 | A: 1.27(1.04, 1.54) | 0.0793 | A,A: 1.35(1.04, 1.76) | 0.0437 | A: 1.22(1.01, 1.49) |
| RS7268029 | 20: 004466594 | | 0.0214 | A,G: 1.45(1.03, 2.05) | 0.4916 | G: 1.12(0.83, 1.51) | 0.0051 | A,G: 1.68(1.17, 2.39) | 0.0748 | G: 1.34(0.98, 1.84) |
| RS6053111 | 20: 005029937 | C20ORF30 | 0.1392 | C,C: 1.29(0.98, 1.7) | 0.0496 | C: 1.22(1, 1.49) | 0.0735 | C,C: 1.34(1.03, 1.75) | 0.0252 | C: 1.25(1.03, 1.51) |
| RS6053268 | 20: 005226993 | | 0.0239 | T,T: 2.12(1.21, 3.69) | 0.0635 | T: 1.23(0.99, 1.54) | 0.0061 | C,C: 1.54(1.18, 2.01) | 0.0034 | C: 1.39(1.12, 1.73) |
| RS2182712 | 20: 006316089 | | 0.0388 | !T,T: 1.39(1.06, 1.82) | 0.0136 | G: 1.3(1.06, 1.6) | 0.0476 | !T,T: 1.38(1.06, 1.79) | 0.0207 | G: 1.27(1.04, 1.56) |
| RS10485749 | 20: 011374730 | | 0.0170 | A,G: 1.37(1.04, 1.8) | 0.8735 | G: 1.02(0.83, 1.25) | 0.0961 | !G,G: 1.31(1.01, 1.7) | 0.0312 | A: 1.25(1.02, 1.53) |
| RS730183 | 20: 011390419 | | 0.0170 | C,T: 1.37(1.04, 1.8) | 0.8735 | T: 1.02(0.83, 1.25) | 0.0872 | !T,T: 1.32(1.02, 1.71) | 0.0274 | C: 1.26(1.03, 1.54) |
| RS4814019 | 20: 011456980 | | 0.0248 | !A,A: 1.46(1.11, 1.91) | 0.0145 | C: 1.29(1.05, 1.57) | 0.0003 | !A,A: 1.55(1.19, 2.01) | 5.62E−05 | C: 1.52(1.24, 1.85) |
| RS293569 | 20: 030568637 | | 0.0091 | !C,T: 1.7(1.14, 2.53) | 0.0214 | T: 1.56(1.07, 2.29) | 0.0169 | !C,T: 1.71(1.13, 2.58) | 0.0266 | T: 1.55(1.05, 2.27) |
| RS6093318 | 20: 038337505 | | 0.1103 | !A,A: 1.37(1.02, 1.85) | 0.0424 | G: 1.33(1.01, 1.73) | 0.0123 | !G,G: 2.84(1.15, 7.01) | 0.8515 | G: 1.03(0.8, 1.31) |
| RS1014748 | 20: 038821628 | | 0.0295 | A,A: 4.84(1.24, 18.93) | 0.3812 | A: 1.15(0.86, 1.53) | 0.0447 | A,A: 2.84(1.14, 7.06) | 0.0774 | A: 1.27(0.98, 1.64) |
| RS2284271 | 20: 043038835 | STK4 | 0.0352 | !T,T: 1.56(1.07, 2.27) | 0.0135 | C: 1.57(1.1, 2.23) | 0.0561 | !T,T: 1.43(1.02, 2) | 0.0255 | C: 1.44(1.05, 1.97) |
| RS2743312 | 20: 043369569 | MATN4, | 0.0046 | G,G: 1.6(1.19, 2.14) | 0.0285 | G: 1.24(1.03, 1.5) | 0.0475 | !A,A: 1.47(1.08, 2.01) | 0.0663 | G: 1.19(0.99, 1.44) |
| | | RBPSUHL | | | | | | | | |
| RS231584 | 20: 048773002 | | 0.0309 | !C,C: 1.67(1.07, 2.6) | 0.0186 | T: 1.68(1.09, 2.58) | 0.0265 | C,T: 1.55(1.08, 2.22) | 0.0250 | T: 1.48(1.05, 2.08) |
| RS427034 | 20: 054901032 | | 0.0011 | G,G: 1.66(1.26, 2.18) | 0.0022 | G: 1.43(1.14, 1.8) | 0.0385 | !A,A: 1.78(1.09, 2.93) | 0.0209 | G: 1.28(1.04, 1.57) |
| RS6064592 | 20: 055788953 | | 0.0071 | !G,G: 2.68(1.14, 6.34) | 0.6993 | G: 1.06(0.82, 1.36) | 0.0636 | !A,A: 1.4(1.05, 1.86) | 0.0344 | G: 1.31(1.02, 1.69) |
| RS183777 | 20: 057244560 | C20ORF174 | 0.1191 | !T,T: 1.36(0.99, 1.87) | 0.0414 | C: 1.22(1.01, 1.48) | 0.0245 | !C,T: 1.42(1.09, 1.85) | 0.6384 | C: 1.05(0.87, 1.26) |
| RS6089479 | 20: 059710358 | CDH4 | 0.0440 | A,A: 1.4(1.07, 1.84) | 0.0411 | A: 1.26(1.01, 1.58) | 0.0261 | !A,G: 1.43(1.09, 1.87) | 0.3533 | A: 1.11(0.9, 1.38) |
| RS6062821 | 20: 061246930 | | 0.0192 | A,A: 1.48(1.1, 2) | 0.1201 | A: 1.16(0.96, 1.41) | 0.0395 | !A,G: 1.4(1.08, 1.82) | 0.9251 | A: 1.01(0.84, 1.22) |
| RS6090265 | 20: 061249931 | | 0.0194 | T,T: 1.63(1.14, 2.34) | 0.1412 | T: 1.16(0.96, 1.41) | 0.0168 | !C,T: 1.42(1.09, 1.84) | 0.7029 | T: 1.04(0.86, 1.25) |
| RS2315656 | 20: 061888781 | BTBD4 | 0.0978 | G,G: 1.3(0.99, 1.72) | 0.0366 | G: 1.23(1.02, 1.5) | 0.0237 | !A,G: 1.44(1.11, 1.86) | 0.1863 | G: 1.14(0.94, 1.38) |
| RS8130901 | 21: 013879844 | | 0.0235 | A,G: 1.43(1.09, 1.88) | 0.8444 | A: 1.02(0.84, 1.24) | 0.0236 | !G,G: 1.6(1.14, 2.24) | 0.0290 | A: 1.23(1.02, 1.48) |
| RS2205533 | 21: 015674652 | LOC388814 | 0.0137 | !A,G: 1.49(1.13, 1.95) | 0.4968 | G: 1.07(0.89, 1.3) | 0.0393 | G,G: 1.36(1.02, 1.81) | 0.0123 | G: 1.27(1.05, 1.53) |
| RS2823252 | 21: 015694445 | | 0.0114 | !A,G: 1.5(1.14, 1.96) | 0.3295 | A: 1.1(0.91, 1.33) | 0.0780 | A,A: 1.32(1, 1.74) | 0.0291 | A: 1.23(1.02, 1.49) |
| RS859164 | 21: 017530786 | | 0.0686 | T,T: 1.35(1.03, 1.77) | 0.0243 | T: 1.3(1.04, 1.64) | 0.0473 | !C,T: 1.33(1.01, 1.76) | 0.7762 | T: 1.04(0.83, 1.29) |
| RS2825682 | 21: 019912457 | | 0.0442 | A,A: 1.37(1.02, 1.83) | 0.0180 | A: 1.37(1.06, 1.77) | 0.0051 | A,C: 1.56(1.14, 2.14) | 0.0998 | C: 1.27(0.96, 1.68) |
| RS2826406 | 21: 020870513 | | 0.1234 | C,C: 1.34(0.95, 1.89) | 0.0400 | C: 1.22(1.01, 1.48) | 0.1152 | !T,T: 1.27(0.97, 1.66) | 0.0473 | C: 1.22(1.01, 1.47) |
| RS416707 | 21: 021048681 | | 0.0908 | !G,G: 1.36(1.03, 1.79) | 0.0379 | T: 1.27(1.01, 1.6) | 0.0119 | !T,T: 2.12(1.12, 4.01) | 0.9557 | T: 1.01(0.81, 1.25) |
| RS2831449 | 21: 028359620 | LOC284825 | 0.1252 | !T,T: 1.44(0.98, 2.12) | 0.0446 | G: 1.45(1.01, 2.09) | 0.0893 | !T,T: 1.55(1.04, 2.32) | 0.0295 | G: 1.53(1.05, 2.22) |
| RS2832238 | 21: 029471232 | C21ORF7 | 0.0054 | !G,T: 1.6(1.2, 2.13) | 0.0141 | T: 1.33(1.06, 1.68) | 0.0145 | G,G: 2.51(1.31, 4.81) | 0.1384 | G: 1.19(0.95, 1.49) |
| RS2834732 | 21: 035308597 | RUNX1 | 0.1152 | T,T: 1.31(1, 1.71) | 0.0438 | T: 1.24(1.01, 1.51) | 0.0095 | !C,T: 1.5(1.15, 1.94) | 0.1179 | T: 1.17(0.96, 1.42) |
| RS2835404 | 21: 036905003 | | 0.1216 | !T,T: 1.39(1, 1.92) | 0.0408 | G: 1.37(1.01, 1.85) | 0.0148 | G,G: 5.26(1.35, 20.6) | 0.5878 | G: 1.08(0.83, 1.41) |
| RS2409948 | 21: 038315359 | | 0.0227 | !T,T: 3.55(1.06, 11.82) | 0.0148 | C: 1.45(1.08, 1.93) | 0.0388 | !T,T: 2.68(1.08, 6.66) | 0.0344 | C: 1.32(1.03, 1.7) |
| RS596958 | 21: 039945361 | | 0.0176 | C,T: 1.69(1.09, 2.61) | 0.0213 | C: 1.64(1.07, 2.49) | 0.0060 | !T,T: 1.72(1.14, 2.58) | 0.0045 | C: 1.77(1.19, 2.62) |
| RS2239385 | 21: 039949617 | B3GALT5 | 0.0094 | !G,G: 1.52(1.16, 2) | 0.0058 | A: 1.35(1.09, 1.67) | 0.0093 | !G,G: 1.5(1.15, 1.95) | 0.0028 | A: 1.39(1.12, 1.73) |
| RS2254583 | 21: 040373134 | DSCAM | 0.0703 | G,G: 2.88(0.85, 9.73) | 0.0343 | G: 1.41(1.03, 1.92) | 0.0679 | !A,A: 1.43(1.04, 1.98) | 0.0202 | G: 1.43(1.06, 1.92) |
| RS3746889 | 21: 041573816 | | 0.0477 | !T,T: 1.72(1.01, 2.94) | 0.0219 | C: 1.82(1.08, 3.06) | 0.0239 | C,T: 2.04(1.11, 3.75) | 0.0589 | C: 1.78(0.99, 3.18) |
| RS233274 | 21: 044813916 | C21ORF29 | 0.0028 | G,T: 1.52(1.16, 1.99) | 0.2845 | G: 1.11(0.92, 1.35) | 0.0294 | G,T: 1.42(1.09, 1.84) | 0.4503 | T: 1.08(0.9, 1.3) |
| RS2238732 | 22: 017295347 | PRODH | 0.0498 | T,T: 1.6(1.01, 2.54) | 0.0324 | T: 1.63(1.04, 2.56) | 0.0458 | T,T: 1.7(1.03, 2.8) | 0.0389 | T: 1.7(1.05, 2.77) |
| RS5748218 | 22: 017831186 | UFD1L | 0.1002 | C,C: 1.37(1.02, 1.83) | 0.0332 | C: 1.32(1.02, 1.7) | 0.0339 | A,A: 2.67(1.07, 6.69) | 1.0000 | C: 1(0.79, 1.28) |
| RS5752534 | 22: 026122500 | | 0.0659 | A,A: 1.37(1.04, 1.8) | 0.0245 | A: 1.29(1.04, 1.61) | 0.0138 | A,A: 1.47(1.13, 1.91) | 0.0043 | A: 1.36(1.1, 1.69) |
| RS12537 | 22: 028753460 | LOC400924, | 0.0479 | !T,T: 1.41(1.06, 1.87) | 0.0832 | C: 1.19(0.98, 1.45) | 0.0101 | C,T: 1.43(1.1, 1.86) | 0.6959 | T: 1.04(0.86, 1.26) |
| | | MTMR3 | | | | | | | | |
| RS139998 | 22: 035192447 | TXN2 | 0.0809 | !T,T: 1.32(0.99, 1.76) | 0.0286 | C: 1.32(1.03, 1.69) | 0.0361 | C,T: 1.45(1.09, 1.94) | 0.0341 | C: 1.3(1.02, 1.65) |
| RS5756219 | 22: 035221804 | FOXRED2 | 0.0008 | A,A: 1.78(1.27, 2.51) | 0.0002 | A: 1.44(1.19, 1.75) | 0.0094 | !C,C: 1.56(1.17, 2.07) | 0.0162 | A: 1.26(1.05, 1.52) |
| RS2076083 | 22: 035242670 | EIF3S7 | 0.0277 | !T,T: 1.5(1.11, 2.02) | 0.0142 | C: 1.39(1.07, 1.81) | 0.0378 | C,T: 1.45(1.08, 1.94) | 0.1805 | C: 1.18(0.93, 1.5) |
| RS2009667 | 22: 035501557 | RABL4 | 0.0045 | A,A: 1.64(1.18, 2.29) | 0.0016 | A: 1.36(1.13, 1.65) | 0.0437 | A,A: 1.43(1.04, 1.98) | 0.3209 | A: 1.1(0.91, 1.32) |
| RS135720 | 22: 036993765 | KIAA1660, | 0.0424 | !A,A: 1.49(1.03, 2.16) | 0.0130 | G: 1.28(1.06, 1.56) | 0.0289 | G,G: 1.4(1.07, 1.83) | 0.0080 | G: 1.3(1.07, 1.57) |
| | | C22ORF5 | | | | | | | | |
| RS132451 | 22: 043535767 | TIG48667, | 0.0510 | !G,G: 1.59(1.06, 2.39) | 0.0183 | A: 1.27(1.04, 1.56) | 0.0492 | !G,G: 1.75(1.11, 2.76) | 0.1216 | A: 1.17(0.96, 1.42) |
| | | ARHGAP8, | | | | | | | | |
| | | LOC553158 | | | | | | | | |
| RS7410678 | 22: 043605937 | LOC553158, | 0.0625 | T,T: 1.35(1.02, 1.79) | 0.0199 | T: 1.33(1.05, 1.7) | 0.0409 | !C,C: 2.44(1.17, 5.07) | 0.2726 | T: 1.14(0.91, 1.43) |
| | | ARHGAP8, | | | | | | | | |
| | | TIG48667 | | | | | | | | |
| RS5765043 | 22: 043610547 | TIG48667, | 0.0035 | T,T: 1.62(1.2, 2.19) | 0.0009 | T: 1.57(1.2, 2.05) | 0.0087 | !C,C: 4.71(1.48, 15.05) | 0.3897 | T: 1.12(0.87, 1.46) |
| | | ARHGAP8, | | | | | | | | |
| | | LOC553158 | | | | | | | | |
| RS2285119 | 22: 043615277 | ARHGAP8, | 0.0122 | !A,G: 1.69(1.16, 2.45) | 0.0090 | A: 1.58(1.12, 2.23) | 0.0293 | !G,G: 14.8(0.84, 259.93) | 0.2531 | A: 1.21(0.88, 1.67) |
| | | LOC553158, | | | | | | | | |
| | | TIG48667 | | | | | | | | |
| RS132146 | 22: 046452725 | LOC284930 | 0.0796 | !C,C: 1.31(1, 1.71) | 0.0268 | T: 1.27(1.03, 1.56) | 0.0124 | T,T: 1.7(1.12, 2.57) | 0.4208 | T: 1.09(0.89, 1.32) |
| RS916249 | 22: 047995693 | LOC150400 | 0.0231 | A,A: 1.49(1.07, 2.09) | 0.3055 | A: 1.11(0.92, 1.34) | 0.0151 | !A,C: 1.46(1.12, 1.89) | 0.8504 | A: 1.02(0.85, 1.23) |
| RS8140508 | 22: 047996263 | LOC150400 | 0.0121 | C,C: 1.55(1.1, 2.17) | 0.2616 | C: 1.12(0.92, 1.35) | 0.0179 | !C,T: 1.44(1.11, 1.87) | 0.8135 | C: 1.03(0.85, 1.23) |
| RS761878 | 22: 048635681 | ZBED4 | 0.0162 | A,G: 1.6(1.15, 2.24) | 0.0199 | A: 1.42(1.06, 1.92) | 0.0307 | A,A: 3.64(1.26, 10.47) | 0.2829 | A: 1.16(0.89, 1.51) |
| RS138229 | 22: 048899534 | MOV10L1 | 0.0169 | A,A: 1.52(1.14, 2.04) | 0.0127 | A: 1.28(1.06, 1.55) | 0.0095 | A,A: 1.56(1.15, 2.1) | 0.0042 | A: 1.31(1.09, 1.58) |
| RS84504 | 22: 048901374 | MOV10L1 | 0.0450 | T,T: 1.48(1.07, 2.06) | 0.0197 | T: 1.26(1.04, 1.52) | 0.0177 | T,T: 1.61(1.15, 2.27) | 0.0119 | T: 1.27(1.05, 1.53) |
| RS5915682 | X: 003966940 | | 0.0359 | G,G: 2.75(1.04, 7.28) | 0.4698 | G: 1.12(0.84, 1.48) | 0.1174 | A,A: 1.7(1.03, 2.81) | 0.0326 | A: 1.4(1.03, 1.89) |
| RS1552186 | X: 005708640 | | 0.0061 | A,A: 2.24(1.36, 3.71) | 0.0016 | A: 1.44(1.15, 1.81) | 0.0310 | !A,C: 1.89(1.17, 3.06) | 0.9532 | A: 1.01(0.8, 1.27) |
| RS996407 | X: 005709394 | | 0.0091 | T,T: 2.17(1.31, 3.57) | 0.0023 | T: 1.42(1.13, 1.78) | 0.0310 | !C,T: 1.89(1.17, 3.06) | 0.9532 | T: 1.01(0.8, 1.27) |
| RS1016627 | X: 009791571 | APXL | 0.0792 | !A,A: 1.51(0.99, 2.29) | 0.0243 | G: 1.32(1.04, 1.67) | 0.5576 | !A,A: 1.27(0.78, 2.05) | 0.0411 | G: 1.3(1.02, 1.65) |
| RS4830433 | X: 009906656 | XM066971 | 0.0203 | !G,G: 2.01(1.19, 3.41) | 0.2068 | A: 1.16(0.93, 1.45) | 0.0026 | A,A: 2.47(1.44, 4.23) | 1.0000 | A: 1(0.8, 1.26) |
| RS3027323 | X: 014471436 | GLRA2 | 0.1080 | !A,G: 1.55(1.02, 2.35) | 0.0199 | A: 1.33(1.05, 1.7) | 0.0275 | !G,G: 2.97(1.2, 7.37) | 0.1186 | A: 1.22(0.96, 1.56) |
| RS7391251 | X: 017130740 | | 0.0795 | G,G: 1.57(1.03, 2.38) | 0.0388 | G: 1.31(1.02, 1.69) | 0.0597 | !A,G: 1.79(1.1, 2.93) | 0.0140 | G: 1.38(1.07, 1.78) |
| RS5955936 | X: 018046471 | | 0.0377 | G,G: 1.95(1.14, 3.35) | 0.4851 | G: 1.09(0.87, 1.37) | 0.1882 | !A,A: 1.57(0.94, 2.62) | 0.0159 | G: 1.33(1.06, 1.68) |
| RS2285563 | X: 024936914 | ARX | 0.1146 | A,A: 2.23(1, 4.98) | 0.0158 | A: 1.38(1.07, 1.78) | 0.2219 | A,C: 1.48(0.9, 2.43) | 0.0235 | A: 1.36(1.05, 1.76) |
| RS7063687 | X: 024940207 | ARX | 0.4957 | C,C: 1.76(0.66, 4.68) | 0.0035 | C: 1.52(1.15, 2.02) | 0.0328 | !C,C: 3.78(0.94, 15.15) | 0.1908 | C: 1.22(0.92, 1.62) |
| RS2765386 | X: 032975809 | DMD | 0.0082 | !C,C: 1.9(1.25, 2.89) | 0.0196 | T: 1.36(1.05, 1.75) | 0.1205 | !C,C: 1.64(1.01, 2.65) | 0.0389 | T: 1.32(1.02, 1.71) |
| RS5972756 | X: 033049643 | DMD | 0.0201 | !C,T: 1.8(1.09, 2.98) | 0.3951 | C: 1.16(0.83, 1.62) | 0.0446 | T,T: 12.09(0.64, 226.77) | 0.6399 | T: 1.1(0.76, 1.59) |
| RS5971730 | X: 033398410 | | 0.0466 | C,C: 1.65(1.06, 2.56) | 0.2056 | C: 1.16(0.93, 1.46) | 0.0003 | !T,T: 3.22(1.69, 6.13) | 0.1281 | C: 1.2(0.95, 1.51) |
| RS2168227 | X: 039308165 | | 0.0170 | !A,A: 5.22(1.36, 20.06) | 0.0940 | G: 1.25(0.97, 1.61) | 0.4433 | !A,A: 1.57(0.66, 3.72) | 0.0074 | G: 1.43(1.11, 1.85) |
| RS4615740 | X: 040266364 | | 0.0208 | !T,T: 3.17(1.3, 7.69) | 0.4243 | C: 1.1(0.87, 1.41) | 0.0031 | C,C: 2.28(1.4, 3.69) | 0.0415 | C: 1.29(1.01, 1.64) |
| RS714472 | X: 042108199 | | 0.0093 | !G,T: 1.9(1.24, 2.93) | 0.0003 | T: 1.56(1.22, 1.98) | 0.0317 | G,G: 2.95(1.25, 6.96) | 0.1431 | G: 1.21(0.94, 1.55) |
| RS4335267 | X: 044940333 | CXORF36 | 0.0176 | C,C: 2.56(1.17, 5.63) | 0.0053 | C: 1.41(1.11, 1.78) | 0.0224 | C,T: 1.94(1.2, 3.14) | 0.5395 | C: 1.08(0.85, 1.37) |
| RS2005947 | X: 048176609 | | 0.3799 | !G,G: 1.34(0.87, 2.07) | 0.0201 | A: 1.32(1.05, 1.66) | 0.0204 | A,G: 1.97(1.21, 3.19) | 0.8152 | G: 1.03(0.82, 1.3) |
| RS880441 | X: 053360748 | | 0.0040 | !C,C: 19.61(1.15, 333.8) | 0.1603 | A: 1.22(0.93, 1.61) | 0.0748 | A,A: 1.75(1.04, 2.94) | 0.0077 | A: 1.49(1.11, 2) |
| RS5914700 | X: 056577077 | | 0.0459 | !T,T: 2.61(1.06, 6.45) | 0.3394 | T: 1.13(0.88, 1.45) | 0.0342 | C,T: 1.87(1.14, 3.05) | 0.1826 | T: 1.21(0.92, 1.59) |
| RS4826478 | X: 056594703 | | 0.0404 | !T,T: 2.61(1.06, 6.45) | 0.3093 | T: 1.15(0.89, 1.47) | 0.0275 | G,T: 1.93(1.18, 3.15) | 0.1815 | T: 1.21(0.92, 1.6) |
| RS5914710 | X: 056616214 | | 0.0320 | !A,A: 2.61(1.06, 6.45) | 0.2813 | A: 1.15(0.9, 1.48) | 0.0342 | A,C: 1.87(1.14, 3.05) | 0.2050 | A: 1.2(0.91, 1.58) |
| RS1925233 | X: 068529091 | | 0.0067 | T,T: 2.68(1.43, 5.03) | 0.0374 | T: 1.28(1.02, 1.62) | 0.5504 | T,T: 1.42(0.71, 2.87) | 0.0408 | T: 1.29(1.02, 1.63) |
| RS5937220 | X: 068557242 | | 0.0346 | A,A: 2.62(1.23, 5.57) | 0.1051 | A: 1.23(0.96, 1.57) | 0.6592 | A,A: 1.43(0.67, 3.05) | 0.0341 | A: 1.31(1.02, 1.67) |
| RS2411961 | X: 078395137 | | 0.0546 | G,G: 1.68(1.06, 2.65) | 0.0159 | G: 1.45(1.08, 1.95) | 0.1980 | G,G: 1.51(0.88, 2.59) | 0.0383 | G: 1.45(1.03, 2.04) |
| RS5940328 | X: 092860053 | | 0.0199 | !A,A: 1.84(1.12, 3.04) | 0.1543 | G: 1.3(0.92, 1.84) | 0.0022 | !A,A: 2.62(1.46, 4.68) | 0.1233 | G: 1.33(0.93, 1.9) |
| RS5990454 | X: 095049095 | | 0.0157 | !G,G: 11.48(0.65, 202.51) | 0.1587 | A: 1.25(0.92, 1.7) | 0.0136 | !A,G: 2.12(1.22, 3.67) | 0.0276 | A: 1.44(1.04, 1.99) |
| RS5921266 | X: 096196914 | DIAPH2 | 0.0691 | A,A: 1.57(1.02, 2.41) | 0.0419 | A: 1.27(1.01, 1.59) | 0.0172 | !A,G: 1.97(1.21, 3.2) | 1.0000 | A: 1(0.79, 1.26) |
| RS720403 | X: 099654701 | | 0.0939 | G,G: 1.48(0.98, 2.24) | 0.0168 | G: 1.34(1.05, 1.71) | 0.0488 | !A,G: 1.79(1.1, 2.9) | 0.3197 | G: 1.14(0.89, 1.45) |
| RS12156914 | X: 100220184 | TMEM35 | 0.0063 | !A,A: 2.9(1.41, 5.96) | 0.0093 | G: 1.36(1.08, 1.72) | 0.0026 | G,G: 2.25(1.37, 3.7) | 0.3146 | G: 1.13(0.9, 1.42) |
| RS7058109 | X: 113114824 | | 0.0134 | G,G: 2.99(1.22, 7.33) | 0.3976 | G: 1.12(0.87, 1.44) | 0.0321 | A,A: 1.79(1.1, 2.9) | 0.0034 | A: 1.5(1.14, 1.96) |
| RS7888180 | X: 113116314 | | 0.0254 | !G,T: 1.92(1.09, 3.38) | 0.6320 | T: 1.11(0.77, 1.62) | 0.0660 | T,T: 2.08(1.09, 3.98) | 0.0136 | T: 1.65(1.11, 2.45) |
| RS4621978 | X: 113134583 | | 0.0028 | T,T: 4.73(1.61, 13.9) | 0.3339 | T: 1.15(0.87, 1.5) | 0.0463 | G,G: 1.72(1.05, 2.83) | 0.0022 | G: 1.58(1.18, 2.11) |
| RS543229 | X: 113727242 | LOC653062, | 0.0420 | !T,T: 4.86(0.86, 27.56) | 0.0187 | C: 1.45(1.06, 1.98) | 0.0223 | T,T: 9.24(0.47, 180.7) | 0.1332 | C: 1.29(0.93, 1.79) |
| | | HTR2C, | | | | | | | | |
| | | LOC653976 | | | | | | | | |
| RS5988087 | X: 113841112 | LOC653062, | 0.0247 | !C,C: 5.4(0.96, 30.27) | 0.0042 | T: 1.59(1.16, 2.18) | 0.0208 | C,C: 9.24(0.47, 180.7) | 0.1080 | T: 1.32(0.95, 1.85) |
| | | HTR2C, | | | | | | | | |
| | | LOC653976 | | | | | | | | |
| RS11167436 | X: 113850316 | LOC653062, | 0.0247 | !C,C: 5.4(0.96, 30.27) | 0.0032 | A: 1.61(1.17, 2.22) | 0.0208 | C,C: 9.24(0.47, 180.7) | 0.1080 | A: 1.32(0.95, 1.85) |
| | | HTR2C, | | | | | | | | |
| | | LOC653976 | | | | | | | | |
| RS5987947 | X: 114764437 | PLS3 | 0.1069 | C,C: 1.72(0.93, 3.18) | 0.0435 | C: 1.27(1.01, 1.61) | 0.0069 | C,C: 3.18(1.51, 6.7) | 0.8067 | C: 1.03(0.81, 1.31) |
| RS7885278 | X: 120144651 | | 0.1938 | C,C: 1.65(0.89, 3.03) | 0.0190 | C: 1.33(1.05, 1.68) | 0.5595 | C,C: 1.44(0.7, 2.95) | 0.0278 | C: 1.31(1.03, 1.67) |
| RS3005849 | X: 122795009 | | 0.0354 | !C,C: 2.03(1.16, 3.56) | 0.4179 | T: 1.1(0.88, 1.38) | 0.3113 | !C,T: 1.4(0.87, 2.25) | 0.0039 | T: 1.41(1.12, 1.78) |
| RS5958427 | X: 123192472 | | 0.0229 | C,C: 9.64(0.49, 188.09) | 0.7754 | C: 1.06(0.73, 1.54) | 0.0091 | !A,A: 2.43(1.27, 4.66) | 0.0311 | C: 1.54(1.05, 2.27) |
| RS3810726 | X: 130050974 | RP13- | 0.0522 | !A,G: 1.88(1.04, 3.39) | 0.0297 | A: 1.59(1.05, 2.4) | 0.0498 | G,G: 9.24(0.47, 180.7) | 0.2638 | A: 1.22(0.87, 1.72) |
| | | 102H20.1 | | | | | | | | |
| RS5920535 | X: 143615817 | | 0.0374 | !A,G: 1.68(1.1, 2.57) | 0.9028 | G: 1.02(0.8, 1.29) | 0.1133 | G,G: 2.59(0.9, 7.49) | 0.0027 | G: 1.48(1.15, 1.91) |
| RS1029098 | X: 143994013 | | 0.0458 | A,A: 1.95(1.14, 3.35) | 0.0918 | A: 1.22(0.97, 1.54) | 0.0189 | A,G: 1.91(1.18, 3.08) | 0.3473 | A: 1.12(0.89, 1.41) |

¹Significant Concordant Association was defined as having a genotypic and/or allelic exact p-value ≦ 0.05 in 1) the Munich collection, 2) Aberdeen collection and 3) the combined set of all Munich + Aberdeen subjects.
²PolyName are RS identifiers.
³See Table 4 for a description of genes listed in this table.
⁴Key denoting strength of association
p-value ≦ 0.0005 in bold underline
0.0005 < p-value ≦ 0.005 in bold
0.005 < p-value ≦ 0.05 in italic
⁵For example, if a SNP had genotypes AA, AG and GG, 3 chi-square tests were performed contrasting cases and controls: 1) AA vs AG + GG, 2) AG vs AA + GG and 3) GG vs AA + AG. An odds ratio was then calculated for the test with the largest chi-square statistic. If the odds ratio was >1, this genotype was reported as the risk genotype. If the odds ratio was <1, then 1) the risk genotype was reported as “!” (“!” means “not”) this genotype and 2) a new odds ratio was calculated as the inverse of the original odds ratio. This new odds ratio was reported.

TABLE 3
Schizophrenia SNPs Significantly Associated and Concordant in Both Collections - Results from
the Combined Munich + Aberdeen Collection¹.

| | | | Combined | | Combined | | | | | Aberdeen |
|---|---|---|---|---|---|---|---|---|---|---|
| | | | Genotypic | Combined Risk | Allelic | Combined Risk | Munich 300K | Munich 300K | Aberdeen 550K | 550K |
| | NCBI 36 | Gene | Global | Genotype & Odds | Global | Allele & Odds | Genotypic | Allelic | Genotypic | Allelic |
| Poly Name² | Position | Symbols³ | Exact P⁴ | Ratio (95% CI)⁵ | Exact P⁴ | Ratio (95% CI) | Exact P⁴ | Exact P⁴ | Exact P⁴ | Exact P⁴ |
| RS12026226 | 01: 004787770 | | 0.0144 | !A,C: 1.42(1.1, 1.84) | 0.0373 | A: 1.3(1.02, 1.65) | 0.0093 | 0.3112 | 0.0529 | 0.0414 |
| RS2011197 | 01: 005942458 | NPHP4 | 0.0183 | !A,A: 1.28(1.04, 1.56) | 0.0054 | G: 1.28(1.08, 1.52) | 0.0595 | 0.0425 | 0.0255 | 0.0552 |
| RS2047811 | 01: 005954440 | NPHP4 | 0.0183 | !C,C: 1.28(1.04, 1.56) | 0.0054 | A: 1.28(1.08, 1.52) | 0.0595 | 0.0425 | 0.0255 | 0.0552 |
| RS1294028 | 01: 009287221 | SPSB1 | 0.0039 | !C,C: 1.35(1.11, 1.64) | 0.001 | T: 1.26(1.1, 1.44) | 0.0738 | 0.0468 | 0.0223 | 0.0082 |
| RS1133398 | 01: 011737218 | | 0.0053 | !A,A: 1.36(1.13, 1.64) | 0.0024 | G: 1.25(1.08, 1.44) | 0.0038 | 0.0026 | 0.0135 | 0.2029 |
| RS3765379 | 01: 015063006 | KIAA1026 | 0.0395 | !G,T: 1.29(1.06, 1.57) | 0.1372 | T: 1.13(0.96, 1.33) | 0.0439 | 1.0000 | 0.0838 | 0.0329 |
| RS12740975 | 01: 019310510 | ZUBR1 | 0.0043 | !A,A: 1.33(1.11, 1.61) | 0.0012 | G: 1.27(1.1, 1.47) | 0.0317 | 0.0139 | 0.0612 | 0.0295 |
| RS1009806 | 01: 019373138 | ZUBR1 | 0.0056 | !C,C: 1.33(1.1, 1.6) | 0.0016 | T: 1.26(1.09, 1.46) | 0.0317 | 0.0139 | 0.0825 | 0.0381 |
| RS2273046 | 01: 019438883 | KIAA0090 | 0.0044 | !A,A: 1.33(1.1, 1.61) | 0.0013 | G: 1.29(1.11, 1.51) | 0.0476 | 0.0179 | 0.0483 | 0.0253 |
| RS7523192 | 01: 019835634 | | 0.0022 | !C,T: 1.38(1.12, 1.7) | 0.3765 | T: 1.09(0.91, 1.29) | 0.0413 | 0.1789 | 0.0364 | 1.0000 |
| RS2473277 | 01: 022234432 | | 0.0027 | !C,C: 1.4(1.12, 1.76) | 0.0006 | T: 1.26(1.1, 1.44) | 0.0172 | 0.0057 | 0.0591 | 0.0382 |
| RS3737807 | 01: 028134489 | SMPDL3B | 0.0007 | G,T: 1.44(1.19, 1.73) | 0.7089 | G: 1.03(0.9, 1.17) | 0.0416 | 0.8070 | 0.0117 | 0.4220 |
| RS4949294 | 01: 031008523 | | 0.0004 | !G,T: 1.43(1.19, 1.73) | 0.1559 | G: 1.1(0.97, 1.26) | 0.0326 | 0.8079 | 0.0079 | 0.0907 |
| RS10914202 | 01: 031024793 | | 0.0006 | !C,T: 1.42(1.17, 1.71) | 0.1466 | C: 1.1(0.97, 1.26) | 0.0262 | 0.9226 | 0.0109 | 0.0602 |
| RS747020 | 01: 032470521 | LOC339483, | 0.0021 | G,G: 1.62(1.22, 2.15) | 0.0008 | G: 1.59(1.21, 2.09) | 0.0559 | 0.0323 | 0.0285 | 0.0130 |
| | | EIF3S2 | | | | | | | | |
| RS2135592 | 01: 036930532 | | 0.0228 | !C,C: 1.37(1.09, 1.72) | 0.01 | T: 1.32(1.07, 1.62) | 0.0275 | 0.1164 | 0.0820 | 0.0340 |
| RS12734651 | 01: 042103172 | HIVEP3 | 0.0094 | A,A: 1.31(1.09, 1.58) | 0.0022 | A: 1.25(1.09, 1.45) | 0.0789 | 0.0252 | 0.1029 | 0.0318 |
| RS557435 | 01: 055293452 | PCSK9, | 0.0057 | !A,A: 1.37(1.13, 1.66) | 0.0019 | G: 1.3(1.1, 1.54) | 0.0600 | 0.0265 | 0.0829 | 0.0288 |
| | | TIG44321 | | | | | | | | |
| RS12144887 | 01: 068116230 | | 0.0047 | C,C: 1.3(1.08, 1.57) | 0.0011 | C: 1.26(1.1, 1.45) | 0.0549 | 0.0223 | 0.0739 | 0.0264 |
| RS4287129 | 01: 074788735 | | 0.028 | G,G: 2.37(1.11, 5.1) | 0.0288 | G: 1.24(1.02, 1.49) | 0.0188 | 0.3176 | 0.0962 | 0.0419 |
| RS6703099 | 01: 075306907 | | 0.006 | C,C: 1.39(1.14, 1.71) | 0.0061 | C: 1.2(1.05, 1.37) | 0.0375 | 0.1598 | 0.0398 | 0.0118 |
| RS2734700 | 01: 086722908 | CLCA1 | 0.0048 | !G,G: 1.38(1.13, 1.67) | 0.0018 | A: 1.3(1.1, 1.53) | 0.0472 | 0.0170 | 0.1033 | 0.0427 |
| RS6693438 | 01: 091935762 | TGFBR3 | 0.0016 | A,A: 1.37(1.13, 1.67) | 0.0003 | A: 1.36(1.15, 1.61) | 0.0096 | 0.0042 | 0.0240 | 0.0272 |
| RS2046736 | 01: 091947596 | TGFBR3 | 0.0017 | T,T: 1.37(1.13, 1.66) | 0.0003 | T: 1.34(1.15, 1.57) | 0.0195 | 0.0076 | 0.0429 | 0.0152 |
| RS17513917 | 01: 091977785 | TGFBR3 | 0.0003 | !C,C: 1.69(1.27, 2.25) | 9.22E−05 | T: 1.32(1.15, 1.52) | 0.0374 | 0.0110 | 0.0054 | 0.0029 |
| RS2029356 | 01: 091981899 | TGFBR3 | 0.0005 | !T,T: 1.88(1.28, 2.76) | 0.0002 | C: 1.34(1.15, 1.56) | 0.1231 | 0.0472 | 0.0007 | 0.0009 |
| RS309058 | 01: 099174812 | PAP2D | 0.0022 | !A,A: 1.54(1.2, 1.97) | 0.0014 | G: 1.25(1.09, 1.43) | 0.0030 | 0.0104 | 0.1458 | 0.0492 |
| RS1376367 | 01: 105251544 | | 0.0735 | A,A: 1.26(1, 1.57) | 0.026 | A: 1.27(1.03, 1.55) | 0.0157 | 0.0384 | 0.0253 | 0.2778 |
| RS10158153 | 01: 106780559 | | 0.0066 | !C,C: 1.81(1.16, 2.82) | 0.7406 | T: 1.03(0.87, 1.21) | 0.0118 | 0.0275 | 0.0140 | 0.0861 |
| RS1444038 | 01: 107782839 | NTNG1 | 0.0127 | G,G: 1.31(1.07, 1.6) | 0.0032 | G: 1.31(1.1, 1.56) | 0.0959 | 0.0284 | 0.1086 | 0.0479 |
| RS1146335 | 01: 118804322 | | 0.0013 | !T,T: 1.42(1.15, 1.77) | 0.0003 | C: 1.28(1.12, 1.46) | 0.0185 | 0.0327 | 0.0007 | 0.0036 |
| RS1146324 | 01: 118814926 | | 0.0014 | !G,G: 1.42(1.15, 1.77) | 0.0003 | A: 1.28(1.12, 1.46) | 0.0351 | 0.0522 | 0.0005 | 0.0022 |
| RS1146320 | 01: 118817763 | | 0.0012 | !G,G: 1.43(1.15, 1.77) | 0.0003 | T: 1.28(1.12, 1.46) | 0.0240 | 0.0414 | 0.0005 | 0.0026 |
| RS1229119 | 01: 118825163 | | 8.44E−05 | !C,C: 1.63(1.26, 2.12) | 1.81E−05 | T: 1.35(1.18, 1.54) | 0.0156 | 0.0067 | 0.0010 | 0.0012 |
| RS1146313 | 01: 118827613 | | 0.0004 | !G,G: 1.46(1.16, 1.83) | 7.60E−05 | A: 1.31(1.15, 1.49) | 0.0133 | 0.0404 | 0.0002 | 0.0006 |
| RS12408596 | 01: 118836057 | | 0.0019 | !C,C: 2.06(1.26, 3.39) | 0.0011 | T: 1.31(1.12, 1.55) | 0.1172 | 0.0409 | 0.0032 | 0.0130 |
| RS17257972 | 01: 118908135 | | 0.002 | A,A: 1.42(1.14, 1.77) | 0.0005 | A: 1.26(1.11, 1.44) | 0.0022 | 0.1459 | 0.0002 | 0.0008 |
| RS2798445 | 01: 118931337 | | 0.0028 | T,T: 1.37(1.13, 1.66) | 0.0007 | T: 1.27(1.11, 1.45) | 0.0590 | 0.0438 | 0.0209 | 0.0066 |
| RS4329498 | 01: 118935132 | | 0.0005 | !T,T: 1.45(1.17, 1.8) | 1.05E−04 | C: 1.3(1.14, 1.48) | 0.0164 | 0.0655 | 4.68E−05 | 0.0004 |
| RS1009458 | 01: 118959432 | LOC644094 | 0.0027 | C,C: 1.36(1.13, 1.65) | 0.0006 | C: 1.27(1.11, 1.46) | 0.0595 | 0.0343 | 0.0236 | 0.0076 |
| RS10923658 | 01: 119074302 | | 0.002 | !A,A: 1.39(1.14, 1.7) | 0.0004 | G: 1.27(1.11, 1.46) | 0.0386 | 0.0322 | 0.0021 | 0.0057 |
| RS1766807 | 01: 119208283 | | 0.0025 | A,A: 1.4(1.14, 1.71) | 0.0006 | A: 1.37(1.15, 1.64) | 0.0603 | 0.0181 | 0.0514 | 0.0176 |
| RS4845771 | 01: 150697304 | | 0.0338 | !T,T: 1.49(1.1, 2.01) | 0.0526 | C: 1.15(1, 1.32) | 0.0938 | 0.0300 | 0.0113 | 0.5829 |
| RS4656888 | 01: 158395295 | ATP1A4 | 0.0013 | !A,G: 1.42(1.17, 1.71) | 0.9731 | A: 1(0.88, 1.15) | 0.0428 | 0.2067 | 0.0096 | 0.2220 |
| RS10494387 | 01: 161524157 | | 0.0007 | A,G: 1.4(1.16, 1.69) | 0.2118 | G: 1.09(0.95, 1.24) | 0.0040 | 0.0259 | 0.0457 | 0.7774 |
| RS10800048 | 01: 163014032 | PBX1 | 0.0281 | A,A: 1.29(1.07, 1.56) | 0.0084 | A: 1.23(1.06, 1.44) | 0.0589 | 0.0319 | 0.0436 | 0.1247 |
| RS545833 | 01: 166956564 | DPT | 0.0014 | C,T: 1.42(1.17, 1.73) | 0.0099 | T: 1.22(1.05, 1.42) | 0.0046 | 0.0022 | 0.0464 | 0.5580 |
| RS859398 | 01: 173642425 | TNR | 0.0254 | G,G: 1.45(1.11, 1.91) | 0.039 | G: 1.16(1.01, 1.33) | 0.0229 | 0.4183 | 0.1073 | 0.0372 |
| RS10913008 | 01: 173798257 | | 0.0009 | A,G: 1.58(1.24, 2.01) | 0.0013 | G: 1.44(1.15, 1.79) | 0.0312 | 0.0291 | 0.0261 | 0.0181 |
| RS1908645 | 01: 185539963 | | 1.26E−06 | A,A: 1.72(1.39, 2.14) | 0.0021 | A: 1.23(1.08, 1.41) | 2.56E−05 | 0.0019 | 0.0229 | 0.2402 |
| RS1170919 | 01: 188882260 | LOC440704 | 0.0119 | !C,C: 1.33(1.1, 1.6) | 0.0086 | T: 1.21(1.05, 1.4) | 0.0477 | 0.2920 | 0.0300 | 0.0086 |
| RS6428282 | 01: 193533695 | | 0.0013 | A,C: 1.39(1.16, 1.68) | 0.0761 | A: 1.13(0.99, 1.29) | 0.0172 | 0.4010 | 0.0345 | 0.0891 |
| RS11584286 | 01: 198686624 | | 0.0116 | !A,G: 1.33(1.04, 1.69) | 0.2894 | A: 1.13(0.9, 1.4) | 0.0364 | 0.6412 | 0.0339 | 0.0367 |
| RS2887480 | 01: 203147961 | NFASC | 0.0131 | !C,C: 1.35(1.07, 1.7) | 0.0036 | T: 1.22(1.07, 1.39) | 0.1081 | 0.0410 | 0.1248 | 0.0479 |
| RS1567190 | 01: 205702412 | CR2 | 0.0009 | C,T: 1.43(1.19, 1.73) | 0.8135 | T: 1.02(0.89, 1.16) | 0.0241 | 0.2073 | 0.0141 | 0.3978 |
| RS2785622 | 01: 206281127 | PLXNA2 | 0.004 | T,T: 1.38(1.13, 1.68) | 0.001 | T: 1.34(1.13, 1.59) | 0.1110 | 0.0500 | 0.0225 | 0.0087 |
| RS841865 | 01: 206292532 | PLXNA2 | 0.0051 | A,A: 1.36(1.12, 1.66) | 0.001 | A: 1.33(1.12, 1.58) | 0.0372 | 0.0136 | 0.0881 | 0.0374 |
| RS2921129 | 01: 210563370 | PPP2R5A | 0.0253 | !T,T: 1.31(1.07, 1.59) | 0.0088 | C: 1.25(1.06, 1.48) | 0.0052 | 0.0062 | 0.0366 | 0.2741 |
| RS3121291 | 01: 210572483 | PPP2R5A | 0.0253 | !G,G: 1.31(1.07, 1.59) | 0.0088 | T: 1.25(1.06, 1.48) | 0.0052 | 0.0062 | 0.0366 | 0.2741 |
| RS701907 | 01: 210588027 | PPP2R5A | 0.0287 | !T,T: 1.3(1.07, 1.58) | 0.01 | C: 1.25(1.06, 1.47) | 0.0056 | 0.0075 | 0.0366 | 0.2741 |
| RS792442 | 01: 210592911 | PPP2R5A | 0.0155 | !A,A: 1.31(1.08, 1.58) | 0.0044 | G: 1.26(1.08, 1.48) | 0.0024 | 0.0059 | 0.0438 | 0.1656 |
| RS1655996 | 01: 211558022 | | 0.0016 | T,T: 1.52(1.19, 1.96) | 0.0008 | T: 1.26(1.1, 1.44) | 0.1236 | 0.0496 | 0.0096 | 0.0067 |
| RS4615815 | 01: 212670859 | PTPN14 | 0.0022 | !G,G: 1.37(1.13, 1.67) | 0.0007 | A: 1.27(1.11, 1.46) | 0.0711 | 0.0304 | 0.0142 | 0.0105 |
| RS4390149 | 01: 214057466 | USH2A | 0.037 | A,A: 1.38(1.08, 1.76) | 0.0651 | A: 1.14(0.99, 1.3) | 0.0462 | 0.5547 | 0.1268 | 0.0415 |
| RS10495071 | 01: 215958401 | SPATA17 | 0.0023 | A,A: 1.43(1.17, 1.75) | 0.0012 | A: 1.34(1.12, 1.6) | 0.0864 | 0.0448 | 0.0052 | 0.0128 |
| RS7546839 | 01: 216133915 | | 0.0044 | !T,T: 1.44(1.16, 1.8) | 0.0018 | G: 1.37(1.12, 1.67) | 0.0573 | 0.0441 | 0.0604 | 0.0203 |
| RS7536586 | 01: 217417479 | LYPLAL1 | 0.0267 | G,G: 1.52(1.11, 2.08) | 0.1055 | G: 1.13(0.98, 1.3) | 0.0969 | 0.0293 | 0.0348 | 1.0000 |
| RS1890758 | 01: 220315446 | | 0.0026 | G,G: 1.48(1.17, 1.89) | 0.0825 | G: 1.13(0.99, 1.29) | 0.0325 | 0.0192 | 0.0087 | 0.9620 |
| RS1591912 | 01: 220322763 | | 0.0046 | G,G: 1.46(1.15, 1.85) | 0.0612 | G: 1.14(0.99, 1.3) | 0.0405 | 0.0283 | 0.0379 | 0.7032 |
| RS17163737 | 01: 221369774 | TLR5 | 0.0044 | !T,T: 1.47(1.15, 1.89) | 0.0012 | G: 1.47(1.16, 1.86) | 0.0203 | 0.0273 | 0.0639 | 0.0218 |
| RS10916197 | 01: 222296930 | | 0.0052 | !C,C: 1.33(1.08, 1.63) | 0.0013 | T: 1.24(1.09, 1.42) | 0.0461 | 0.0174 | 0.0956 | 0.0372 |
| RS7517868 | 01: 222318289 | | 0.0012 | !A,A: 1.49(1.17, 1.91) | 0.0003 | G: 1.28(1.12, 1.46) | 0.0138 | 0.0038 | 0.0715 | 0.0333 |
| RS9435806 | 01: 227522499 | C1ORF96 | 0.0064 | T,T: 1.32(1.08, 1.62) | 0.0016 | T: 1.24(1.09, 1.42) | 0.0827 | 0.0281 | 0.0720 | 0.0233 |
| RS1890350 | 01: 228960988 | CAPN9 | 0.0017 | !A,A: 1.49(1.2, 1.87) | 0.0017 | G: 1.24(1.08, 1.41) | 0.0333 | 0.1202 | 0.0184 | 0.0048 |
| RS4847011 | 01: 228966634 | CAPN9 | 0.0018 | !C,C: 1.68(1.25, 2.24) | 0.0051 | T: 1.22(1.06, 1.4) | 0.0450 | 0.0822 | 0.0438 | 0.0307 |
| RS3828128 | 01: 228995933 | CAPN9 | 0.0007 | !G,G: 2.11(1.42, 3.13) | 0.0144 | A: 1.21(1.04, 1.4) | 0.0388 | 0.3792 | 0.0084 | 0.0107 |
| RS1202524 | 01: 229017795 | | 0.0002 | !A,A: 1.92(1.39, 2.66) | 0.0013 | G: 1.26(1.1, 1.46) | 0.0133 | 0.1292 | 0.0075 | 0.0032 |
| RS722732 | 01: 229027992 | | 0.0006 | !A,A: 1.93(1.36, 2.73) | 0.0022 | G: 1.26(1.09, 1.45) | 0.0461 | 0.2176 | 0.0064 | 0.0026 |
| RS946131 | 01: 230505719 | | 0.0003 | C,T: 1.45(1.18, 1.78) | 0.1596 | T: 1.13(0.95, 1.35) | 0.0141 | 0.0815 | 0.0140 | 0.8097 |
| RS16835237 | 01: 235653007 | RYR2 | 0.0084 | !C,C: 1.43(1.12, 1.83) | 0.0035 | T: 1.42(1.12, 1.78) | 0.0162 | 0.0331 | 0.0539 | 0.0470 |
| RS790900 | 01: 236023932 | RYR2 | 0.0035 | !T,T: 1.35(1.12, 1.63) | 0.0008 | G: 1.28(1.11, 1.47) | 0.0218 | 0.0053 | 0.1154 | 0.0466 |
| RS12732104 | 01: 236455086 | | 0.004 | !T,T: 1.6(1.19, 2.14) | 0.0936 | C: 1.13(0.98, 1.3) | 0.0134 | 0.7951 | 0.0215 | 0.0121 |
| RS6682403 | 01: 236483906 | | 0.0019 | A,G: 1.37(1.13, 1.65) | 0.5615 | G: 1.04(0.91, 1.19) | 0.0006 | 0.0440 | 0.0057 | 0.0059 |
| RS4658504 | 01: 241107670 | | 5.78E−05 | C,C: 1.53(1.26, 1.85) | 0.0007 | C: 1.27(1.1, 1.45) | 0.0052 | 0.0176 | 0.0084 | 0.0137 |
| RS1173656 | 01: 243689684 | | 0.0007 | G,G: 1.89(1.24, 2.87) | 0.9361 | G: 1.01(0.86, 1.18) | 0.0198 | 0.9080 | 0.0320 | 0.9557 |
| RS7561317 | 02: 000634953 | | 0.0013 | !A,G: 1.47(1.19, 1.81) | 0.0088 | G: 1.27(1.06, 1.52) | 0.0275 | 0.0532 | 0.0483 | 0.0763 |
| RS357958 | 02: 003761591 | | 0.1253 | !A,A: 1.22(1, 1.49) | 0.0488 | G: 1.14(1, 1.31) | 0.0350 | 0.1570 | 0.0112 | 0.1851 |
| RS2249380 | 02: 003767258 | | 0.0039 | !A,A: 1.44(1.13, 1.84) | 0.0011 | C: 1.25(1.09, 1.43) | 0.0654 | 0.0211 | 0.0274 | 0.0227 |
| RS1564332 | 02: 004592098 | | 0.0153 | G,G: 1.35(1.09, 1.66) | 0.0068 | G: 1.2(1.05, 1.37) | 0.0382 | 0.0326 | 0.0057 | 0.0901 |
| RS2163027 | 02: 005890517 | | 0.0036 | !T,T: 1.32(1.09, 1.59) | 0.0008 | G: 1.28(1.11, 1.47) | 0.0606 | 0.0296 | 0.0320 | 0.0109 |
| RS2709460 | 02: 005896443 | | 0.0047 | !C,C: 1.35(1.12, 1.63) | 0.0014 | T: 1.27(1.1, 1.46) | 0.1000 | 0.0378 | 0.0442 | 0.0129 |
| RS2357889 | 02: 011117422 | | 0.001 | !T,T: 1.41(1.16, 1.71) | 0.0002 | C: 1.29(1.13, 1.48) | 0.0028 | 0.0019 | 0.1053 | 0.0420 |
| RS983037 | 02: 012997671 | | 6.97E−05 | !C,C: 1.72(1.3, 2.29) | 2.03E−05 | A: 1.35(1.18, 1.55) | 0.0059 | 0.0012 | 0.0032 | 0.0052 |
| RS12692488 | 02: 013052254 | | 0.0099 | !G,G: 1.49(1.13, 1.97) | 0.0043 | A: 1.22(1.07, 1.41) | 0.0081 | 0.0079 | 0.0018 | 0.1630 |
| RS1818595 | 02: 013085683 | | 0.0016 | !G,G: 1.5(1.17, 1.92) | 0.0007 | A: 1.26(1.11, 1.44) | 0.0322 | 0.0969 | 0.0042 | 0.0014 |
| RS1976809 | 02: 017329493 | LOC645521 | 0.0486 | G,G: 1.36(1.05, 1.78) | 0.2526 | G: 1.08(0.95, 1.24) | 0.0573 | 0.0254 | 0.0244 | 0.7034 |
| RS7583409 | 02: 025344560 | DNMT3A | 0.0217 | G,G: 1.52(1.1, 2.09) | 0.3094 | G: 1.08(0.93, 1.24) | 0.0311 | 0.0195 | 0.0322 | 0.3871 |
| RS12621279 | 02: 031195804 | GALNT14 | 0.0261 | T,T: 1.28(1.06, 1.55) | 0.0638 | T: 1.14(0.99, 1.31) | 0.1066 | 0.0267 | 0.0164 | 0.6971 |
| RS2568465 | 02: 032715454 | TTC27 | 0.0026 | C,C: 1.39(1.15, 1.68) | 0.0011 | C: 1.27(1.1, 1.46) | 0.0560 | 0.0188 | 0.0334 | 0.0207 |
| RS1000951 | 02: 033115912 | LTBP1 | 0.0019 | G,G: 1.46(1.18, 1.8) | 0.0013 | G: 1.36(1.13, 1.65) | 0.0263 | 0.0115 | 0.0663 | 0.0494 |
| RS10490450 | 02: 033186442 | LTBP1 | 0.0008 | T,T: 1.47(1.2, 1.81) | 0.0002 | T: 1.4(1.17, 1.67) | 0.0666 | 0.0206 | 0.0107 | 0.0051 |
| RS1446297 | 02: 038061737 | FAM82A | 0.0068 | A,G: 1.35(1.12, 1.63) | 0.0321 | G: 1.17(1.01, 1.36) | 0.0106 | 0.7131 | 0.0307 | 0.0093 |
| RS2373327 | 02: 038075590 | FAM82A | 0.0242 | !A,A: 1.29(1.07, 1.55) | 0.0357 | G: 1.17(1.01, 1.35) | 0.0483 | 0.6379 | 0.0482 | 0.0147 |
| RS6749927 | 02: 043338060 | THADA | 0.0007 | G,G: 1.6(1.22, 2.09) | 0.1267 | G: 1.11(0.97, 1.28) | 0.0118 | 0.8041 | 0.0076 | 0.0171 |
| RS9309199 | 02: 050867151 | NRXN1 | 0.0277 | G,G: 1.35(1.08, 1.69) | 0.0429 | G: 1.15(1.01, 1.31) | 0.0168 | 0.4377 | 0.1110 | 0.0433 |
| RS10164725 | 02: 053559728 | | 0.0007 | C,T: 1.44(1.14, 1.81) | 0.1085 | C: 1.18(0.97, 1.45) | 0.0533 | 0.0258 | 0.0007 | 0.9426 |
| RS13405071 | 02: 057946258 | LOC647021, | 0.004 | !C,T: 1.57(1.18, 2.1) | 0.0192 | T: 1.37(1.06, 1.79) | 0.0703 | 0.0444 | 0.0249 | 0.2253 |
| | | LOC652202 | | | | | | | | |
| RS11887238 | 02: 057996771 | | 0.0007 | !A,C: 1.77(1.28, 2.45) | 0.0043 | A: 1.54(1.14, 2.08) | 0.0117 | 0.0126 | 0.0207 | 0.1316 |
| RS6758100 | 02: 058968243 | FLJ30838, | 0.0071 | C,T: 1.28(1.06, 1.54) | 0.745 | C: 1.02(0.89, 1.18) | 0.0391 | 0.1037 | 0.0113 | 0.2509 |
| | | LOC647029, | | | | | | | | |
| | | LOC652214 | | | | | | | | |
| RS2422008 | 02: 063634109 | | 0.0672 | !T,T: 1.27(1.04, 1.55) | 0.0482 | G: 1.15(1, 1.31) | 0.0318 | 0.0142 | 0.0455 | 0.7048 |
| RS1477054 | 02: 065758368 | FLJ16124 | 0.0563 | !G,G: 1.4(1.05, 1.88) | 0.0145 | A: 1.42(1.07, 1.87) | 0.0472 | 0.0560 | 0.0414 | 0.1522 |
| RS2872075 | 02: 069922139 | GMCL1 | 0.0116 | !T,T: 1.35(1.1, 1.66) | 0.0045 | G: 1.22(1.06, 1.39) | 0.0077 | 0.0797 | 0.0314 | 0.0237 |
| RS10496175 | 02: 070007352 | MXD1 | 0.0268 | C,T: 1.3(1.07, 1.58) | 0.0472 | C: 1.17(1, 1.37) | 0.0471 | 0.4912 | 0.1041 | 0.0369 |
| RS11893500 | 02: 070007691 | MXD1 | 0.0715 | !T,T: 1.26(1.03, 1.53) | 0.0292 | C: 1.16(1.02, 1.33) | 0.0252 | 0.3031 | 0.0446 | 0.0416 |
| RS6741449 | 02: 070010093 | MXD1 | 0.0586 | !A,A: 1.27(1.04, 1.55) | 0.0246 | G: 1.17(1.02, 1.33) | 0.0234 | 0.2596 | 0.0476 | 0.0418 |
| RS7580273 | 02: 071130206 | | 0.0114 | A,G: 1.32(1.09, 1.6) | 0.258 | G: 1.09(0.94, 1.26) | 0.0197 | 0.0176 | 0.0375 | 0.4932 |
| RS11126315 | 02: 071144566 | | 0.0122 | C,T: 1.32(1.09, 1.6) | 0.2748 | C: 1.09(0.94, 1.26) | 0.0220 | 0.0204 | 0.0376 | 0.4937 |
| RS898238 | 02: 071867166 | | 0.0007 | A,A: 1.49(1.21, 1.83) | 0.0003 | A: 1.39(1.16, 1.66) | 0.0156 | 0.0146 | 0.0305 | 0.0085 |
| RS10496214 | 02: 079271451 | | 0.0004 | !G,T: 1.76(1.3, 2.37) | 0.0008 | T: 1.62(1.22, 2.16) | 0.0116 | 0.1303 | 0.0025 | 0.0017 |
| RS17016549 | 02: 079286292 | | 0.0006 | !A,G: 1.7(1.27, 2.28) | 0.0036 | A: 1.5(1.14, 1.98) | 0.0081 | 0.3316 | 0.0022 | 0.0016 |
| RS1549765 | 02: 079670651 | CTNNA2 | 0.0382 | !C,C: 2.1(1.11, 3.96) | 0.7543 | T: 1.03(0.86, 1.23) | 0.0511 | 0.0441 | 0.0366 | 0.1384 |
| RS2902106 | 02: 079960523 | CTNNA2 | 0.0007 | !G,G: 2.77(1.59, 4.84) | 0.0143 | A: 1.24(1.04, 1.47) | 0.0210 | 0.0257 | 0.0245 | 0.2526 |
| RS333228 | 02: 107997483 | SLC5A7 | 0.0012 | A,A: 1.39(1.15, 1.67) | 0.0003 | A: 1.33(1.14, 1.55) | 0.0535 | 0.0156 | 0.0208 | 0.0072 |
| RS1384795 | 02: 111488118 | ACOXL | 0.0032 | !G,G: 1.48(1.17, 1.87) | 0.0009 | A: 1.44(1.16, 1.79) | 0.0916 | 0.0361 | 0.0327 | 0.0096 |
| RS2062374 | 02: 114612733 | | 0.0003 | !A,A: 2.64(1.57, 4.44) | 0.2536 | G: 1.1(0.93, 1.3) | 0.0250 | 0.3285 | 0.0098 | 0.5577 |
| RS10864971 | 02: 118717122 | | 0.0057 | T,T: 1.36(1.12, 1.65) | 0.0027 | T: 1.23(1.08, 1.41) | 0.0806 | 0.0474 | 0.0131 | 0.0343 |
| RS1371562 | 02: 119449418 | MARCO | 0.0022 | !G,G: 1.46(1.15, 1.85) | 0.1348 | T: 1.11(0.97, 1.27) | 0.0426 | 0.3284 | 0.0495 | 0.2763 |
| RS2422392 | 02: 119514743 | | 0.0023 | !C,C: 1.4(1.16, 1.68) | 0.0011 | T: 1.28(1.11, 1.49) | 0.0300 | 0.0084 | 0.0421 | 0.0377 |
| RS4662817 | 02: 128908256 | | 0.0036 | !A,A: 1.41(1.15, 1.72) | 0.0022 | G: 1.31(1.1, 1.56) | 0.0134 | 0.0465 | 0.0609 | 0.0198 |
| RS714527 | 02: 128920942 | | 0.0025 | A,G: 1.5(1.19, 1.88) | 0.0043 | G: 1.34(1.1, 1.64) | 0.0261 | 0.1425 | 0.0308 | 0.0099 |
| RS12611515 | 02: 133258045 | NAP5 | 0.0388 | !C,C: 1.55(1.1, 2.17) | 0.0757 | T: 1.14(0.99, 1.32) | 0.0391 | 0.0125 | 0.0218 | 0.9579 |
| RS579076 | 02: 134983030 | TMEM163 | 0.0124 | C,C: 1.51(1.15, 1.99) | 0.0231 | C: 1.17(1.02, 1.35) | 0.1241 | 0.0464 | 0.0332 | 0.2391 |
| RS6734966 | 02: 135262217 | | 0.0016 | !T,T: 1.41(1.14, 1.74) | 0.0005 | C: 1.4(1.16, 1.69) | 0.0262 | 0.0101 | 0.0861 | 0.0321 |
| RS2176717 | 02: 136616329 | | 3.50E−05 | !T,T: 1.62(1.28, 2.05) | 8.76E−06 | C: 1.35(1.19, 1.55) | 0.0027 | 0.0013 | 0.0108 | 0.0031 |
| RS882300 | 02: 136692725 | | 6.81E−05 | !A,A: 1.59(1.26, 2.02) | 1.57E−05 | G: 1.34(1.18, 1.54) | 0.0042 | 0.0026 | 0.0106 | 0.0030 |
| RS10496767 | 02: 137581892 | KIAA1679 | 0.0154 | T,T: 1.79(1.19, 2.69) | 0.0386 | T: 1.18(1.01, 1.37) | 0.0201 | 0.4749 | 0.0715 | 0.0252 |
| RS1370327 | 02: 142185062 | LRP1B | 0.0005 | !G,G: 1.52(1.22, 1.88) | 0.0046 | A: 1.21(1.06, 1.38) | 0.0349 | 0.0371 | 0.0145 | 0.0602 |
| RS6744928 | 02: 142549797 | LRP1B | 0.0087 | !C,C: 1.34(1.1, 1.63) | 0.003 | T: 1.23(1.07, 1.41) | 0.0401 | 0.0257 | 0.1288 | 0.0493 |
| RS354690 | 02: 143534188 | | 0.0003 | T,T: 1.65(1.29, 2.11) | 0.001 | T: 1.25(1.1, 1.43) | 0.0115 | 0.0116 | 0.0207 | 0.0296 |
| RS3768655 | 02: 152422269 | CACNB4 | 0.0014 | !A,A: 1.46(1.13, 1.9) | 0.001 | C: 1.51(1.18, 1.93) | 0.0564 | 0.0175 | 0.0076 | 0.0234 |
| RS3791626 | 02: 153336153 | | 0.0456 | !C,C: 2.04(1.14, 3.65) | 0.2778 | T: 1.11(0.93, 1.32) | 0.0370 | 0.3765 | 0.0225 | 0.0114 |
| RS10179554 | 02: 158778702 | LOC130940 | 0.0016 | C,T: 1.9(1.29, 2.8) | 0.0016 | C: 1.82(1.25, 2.65) | 0.0157 | 0.0094 | 0.0394 | 0.0845 |
| RS888629 | 02: 159931293 | BAZ2B | 0.0009 | !A,A: 1.57(1.23, 2.02) | 0.0002 | G: 1.55(1.23, 1.95) | 0.0600 | 0.0282 | 0.0051 | 0.0020 |
| RS2042772 | 02: 160370304 | LY75 | 0.0086 | !T,T: 1.3(1.07, 1.57) | 0.0024 | C: 1.27(1.09, 1.48) | 0.1190 | 0.0455 | 0.0595 | 0.0216 |
| RS1990842 | 02: 169916281 | LRP2 | 0.0021 | A,A: 1.4(1.16, 1.69) | 0.0008 | A: 1.27(1.1, 1.47) | 0.0223 | 0.0067 | 0.0214 | 0.0528 |
| RS4145305 | 02: 174694824 | PTD004 | 0.0047 | !A,A: 1.42(1.15, 1.74) | 0.0017 | G: 1.34(1.12, 1.61) | 0.0935 | 0.0267 | 0.0152 | 0.0215 |
| RS2119137 | 02: 174725960 | PTD004 | 0.0005 | G,G: 1.64(1.25, 2.15) | 0.0002 | G: 1.3(1.13, 1.49) | 0.0115 | 0.0160 | 0.0194 | 0.0046 |
| RS2588868 | 02: 177709231 | | 0.005 | !C,C: 1.43(1.14, 1.79) | 0.0013 | T: 1.4(1.14, 1.72) | 0.0538 | 0.0155 | 0.1189 | 0.0478 |
| RS4894058 | 02: 179446750 | FLJ39502 | 0.0075 | C,C: 1.49(1.1, 2.02) | 0.0022 | C: 1.25(1.09, 1.44) | 0.0531 | 0.0243 | 0.0984 | 0.0322 |
| RS6716304 | 02: 179454567 | FLJ39502 | 0.0035 | G,G: 1.59(1.13, 2.23) | 0.0012 | G: 1.27(1.1, 1.47) | 0.0810 | 0.0330 | 0.0369 | 0.0127 |
| RS1028179 | 02: 180044166 | ZNF533 | 0.0133 | !C,T: 1.34(1.1, 1.63) | 0.0841 | T: 1.16(0.98, 1.36) | 0.0953 | 0.0402 | 0.0237 | 0.6939 |
| RS7588905 | 02: 184246849 | | 0.0067 | !G,G: 1.65(1.21, 2.27) | 0.0263 | A: 1.18(1.02, 1.36) | 0.1543 | 0.0480 | 0.0039 | 0.2216 |
| RS1451487 | 02: 199703113 | | 0.0013 | A,A: 1.44(1.17, 1.76) | 0.0003 | A: 1.38(1.16, 1.65) | 0.0568 | 0.0276 | 0.0105 | 0.0040 |
| RS2243822 | 02: 201273695 | | 0.0008 | G,G: 2.21(1.44, 3.38) | 0.0105 | G: 1.23(1.05, 1.43) | 0.0300 | 0.0490 | 0.0279 | 0.0989 |
| RS7577449 | 02: 208475230 | LOC389072 | 0.0063 | T,T: 1.35(1.12, 1.63) | 0.0051 | T: 1.23(1.07, 1.43) | 0.0262 | 0.1656 | 0.0586 | 0.0173 |
| RS4597456 | 02: 212033473 | ERBB4 | 0.0253 | A,G: 1.3(1.07, 1.56) | 0.7871 | A: 1.02(0.89, 1.16) | 0.0367 | 0.0369 | 0.0121 | 0.0996 |
| RS280626 | 02: 215272346 | | 0.0014 | C,T: 1.43(1.17, 1.76) | 0.0623 | C: 1.18(0.99, 1.4) | 0.0261 | 0.0176 | 0.0165 | 0.6806 |
| RS1250085 | 02: 216246901 | LOC646324 | 0.0036 | !A,A: 1.77(1.25, 2.48) | 0.0115 | G: 1.21(1.04, 1.39) | 0.0247 | 0.0078 | 0.0184 | 0.3637 |
| RS12624027 | 02: 217105664 | | 0.018 | !T,T: 1.31(1.05, 1.64) | 0.0046 | C: 1.21(1.06, 1.38) | 0.0207 | 0.1092 | 0.0081 | 0.0186 |
| RS6435962 | 02: 217659070 | | 0.0004 | G,G: 1.44(1.2, 1.74) | 0.002 | G: 1.26(1.09, 1.45) | 0.0152 | 0.0168 | 0.0233 | 0.0415 |
| RS6728963 | 02: 218071277 | | 0.0208 | C,C: 3.56(1.09, 11.7) | 0.0156 | C: 1.35(1.06, 1.73) | 0.0171 | 0.3063 | 0.0510 | 0.0235 |
| RS3791911 | 02: 218502228 | TNS1 | 0.0088 | T,T: 1.32(1.08, 1.62) | 0.0022 | T: 1.32(1.1, 1.57) | 0.0716 | 0.0250 | 0.1278 | 0.0414 |
| RS1955117 | 02: 221779509 | | 0.0342 | !A,G: 1.27(1, 1.61) | 0.3185 | A: 1.12(0.9, 1.39) | 0.0839 | 0.0450 | 0.0489 | 0.6557 |
| RS6436944 | 02: 231084564 | SP100 | 0.0163 | !T,T: 2.36(1.2, 4.61) | 0.7769 | C: 1.03(0.86, 1.24) | 0.0100 | 0.0099 | 0.0184 | 0.0414 |
| RS699660 | 02: 231093030 | | 0.0044 | !C,C: 3.1(1.42, 6.77) | 0.7397 | T: 1.03(0.86, 1.24) | 0.0107 | 0.0116 | 0.0049 | 0.0548 |
| RS836237 | 02: 231114924 | | 0.0095 | !T,T: 2.77(1.31, 5.87) | 0.7408 | C: 1.03(0.86, 1.24) | 0.0107 | 0.0116 | 0.0138 | 0.0571 |
| RS17354508 | 02: 231138221 | | 0.0004 | A,G: 1.42(1.17, 1.72) | 0.5226 | A: 1.05(0.91, 1.22) | 0.0088 | 0.4145 | 0.0451 | 0.9171 |
| RS6753112 | 02: 231895399 | ARMC9 | 0.0015 | C,T: 1.38(1.14, 1.67) | 0.9166 | T: 1.01(0.88, 1.16) | 0.0439 | 0.5858 | 0.0216 | 0.4596 |
| RS7570061 | 02: 233685562 | LOC653796 | 0.0025 | G,G: 2.56(1.43, 4.56) | 0.0103 | G: 1.26(1.06, 1.49) | 0.0487 | 0.1166 | 0.0529 | 0.0459 |
| RS10210816 | 02: 234634378 | SPP2 | 0.0241 | C,T: 1.31(1.08, 1.59) | 0.1775 | C: 1.11(0.95, 1.29) | 0.0163 | 0.5402 | 0.0390 | 0.0140 |
| RS4340455 | 02: 235688109 | | 0.0022 | C,T: 1.37(1.13, 1.65) | 0.9714 | C: 1(0.87, 1.16) | 0.0060 | 0.0997 | 0.0360 | 0.1353 |
| RS1367882 | 02: 235859450 | | 0.0071 | !A,A: 1.36(1.12, 1.64) | 0.0045 | G: 1.22(1.07, 1.4) | 0.1085 | 0.0397 | 0.0340 | 0.0502 |
| RS4663795 | 02: 238397755 | FLJ40411 | 0.0045 | !A,A: 1.42(1.15, 1.76) | 0.0028 | G: 1.34(1.11, 1.62) | 0.0954 | 0.0433 | 0.0419 | 0.0322 |
| RS9287640 | 02: 239377004 | | 0.0007 | G,T: 1.39(1.14, 1.7) | 0.4175 | G: 1.07(0.91, 1.25) | 0.0283 | 0.6059 | 0.0256 | 0.6051 |
| RS1465820 | 02: 240270700 | | 0.0069 | !A,A: 1.32(1.09, 1.6) | 0.0016 | G: 1.25(1.09, 1.43) | 0.0632 | 0.0233 | 0.1063 | 0.0349 |
| RS3843388 | 03: 003255819 | | 0.0445 | T,T: 1.32(1.05, 1.65) | 0.0123 | T: 1.3(1.06, 1.6) | 0.0441 | 0.6537 | 0.0006 | 0.0028 |
| RS1682913 | 03: 003821526 | LRRN1 | 0.0073 | !T,T: 1.45(1.15, 1.83) | 0.0019 | G: 1.41(1.14, 1.75) | 0.1389 | 0.0490 | 0.0278 | 0.0197 |
| RS345233 | 03: 006745752 | | 0.0043 | !C,C: 1.35(1.12, 1.64) | 0.0008 | T: 1.31(1.12, 1.54) | 0.0100 | 0.0216 | 0.0173 | 0.0150 |
| RS2633748 | 03: 006760430 | | 0.0077 | G,G: 1.36(1.09, 1.7) | 0.0032 | G: 1.36(1.11, 1.67) | 0.0320 | 0.0437 | 0.1069 | 0.0379 |
| RS6785425 | 03: 007100901 | GRM7 | 0.0025 | A,A: 1.65(1.23, 2.22) | 0.0009 | A: 1.61(1.21, 2.13) | 0.0942 | 0.0310 | 0.0108 | 0.0110 |
| RS2928078 | 03: 011034847 | SLC6A1 | 0.0057 | T,T: 1.44(1.13, 1.82) | 0.0023 | T: 1.23(1.08, 1.41) | 0.0532 | 0.0456 | 0.0703 | 0.0232 |
| RS11128706 | 03: 014562321 | | 0.0218 | T,T: 1.75(1.02, 3.01) | 0.7594 | C: 1.03(0.87, 1.22) | 0.0071 | 0.0095 | 0.0193 | 0.0503 |
| RS9847186 | 03: 025056861 | | 1.06E−04 | !A,G: 1.46(1.21, 1.77) | 0.5848 | G: 1.04(0.91, 1.19) | 0.0008 | 0.1039 | 0.0017 | 0.0156 |
| RS7642232 | 03: 033633002 | CLASP2 | 0.0028 | A,A: 1.4(1.15, 1.7) | 0.0007 | A: 1.34(1.13, 1.58) | 0.0015 | 0.0011 | 0.0482 | 0.1234 |
| RS7629699 | 03: 033922976 | | 0.0004 | G,G: 1.88(1.37, 2.58) | 0.0054 | G: 1.23(1.06, 1.41) | 0.0273 | 0.0774 | 0.0135 | 0.0304 |
| RS1304138 | 03: 039979370 | MYRIP | 1.12E−04 | A,A: 1.75(1.3, 2.36) | 0.2152 | A: 1.09(0.95, 1.26) | 0.0284 | 0.2894 | 0.0033 | 0.5503 |
| RS6789549 | 03: 040996267 | | 0.0014 | !A,A: 1.42(1.18, 1.72) | 0.0026 | G: 1.24(1.08, 1.42) | 0.0381 | 0.0239 | 0.0342 | 0.0452 |
| RS4130762 | 03: 041012673 | | 4.28E−06 | !A,A: 1.64(1.35, 1.99) | 2.86E−05 | G: 1.34(1.17, 1.53) | 0.0444 | 0.0158 | 2.40E−05 | 0.0007 |
| RS6599132 | 03: 041014911 | | 0.0036 | !G,G: 1.45(1.14, 1.84) | 0.0012 | A: 1.25(1.09, 1.43) | 0.0729 | 0.0302 | 0.0164 | 0.0211 |
| RS9861853 | 03: 043929897 | | 0.0247 | !A,G: 1.34(1.08, 1.65) | 0.0598 | A: 1.18(1, 1.41) | 0.0895 | 0.0263 | 0.0419 | 0.6793 |
| RS1495366 | 03: 055777789 | CAST1 | 0.006 | A,A: 1.58(1.16, 2.14) | 0.0036 | A: 1.24(1.07, 1.42) | 0.0799 | 0.0453 | 0.0820 | 0.0391 |
| RS13063937 | 03: 060717098 | FHIT | 0.0056 | !C,C: 1.84(1.17, 2.87) | 0.0044 | A: 1.25(1.07, 1.46) | 0.0918 | 0.0368 | 0.0347 | 0.0589 |
| RS6804478 | 03: 060860180 | FHIT | 0.0015 | !A,A: 1.49(1.2, 1.85) | 0.0085 | G: 1.2(1.05, 1.37) | 0.0455 | 0.0369 | 0.0271 | 0.1211 |
| RS2365054 | 03: 061044854 | FHIT | 0.0008 | !A,A: 3.42(1.7, 6.87) | 0.0368 | C: 1.22(1.01, 1.47) | 0.0046 | 0.0047 | 0.0061 | 0.8979 |
| RS6806415 | 03: 061078649 | FHIT | 0.0066 | !T,T: 2.04(1.3, 3.22) | 0.0687 | C: 1.16(0.99, 1.37) | 0.0052 | 0.0048 | 0.0499 | 0.8639 |
| RS1821942 | 03: 061091049 | FHIT | 0.0094 | !G,G: 1.82(1.18, 2.82) | 0.5497 | A: 1.05(0.9, 1.23) | 0.0093 | 0.0232 | 0.0309 | 0.1701 |
| RS9311951 | 03: 065663422 | MAGI1 | 0.0311 | G,G: 1.46(1.07, 1.99) | 0.3673 | G: 1.07(0.93, 1.23) | 0.0130 | 0.4928 | 0.0960 | 0.0476 |
| RS264096 | 03: 065720475 | MAGI1 | 0.0022 | !A,A: 1.42(1.17, 1.73) | 0.0011 | G: 1.32(1.12, 1.56) | 0.0235 | 0.0177 | 0.0631 | 0.0213 |
| RS7615745 | 03: 074236974 | | 7.91E−05 | !C,T: 1.5(1.25, 1.81) | 0.1647 | C: 1.1(0.96, 1.26) | 0.0007 | 0.0047 | 0.0038 | 0.4500 |
| RS1569067 | 03: 074897220 | | 0.0133 | T,T: 1.34(1.09, 1.64) | 0.0034 | T: 1.31(1.09, 1.57) | 0.0497 | 0.0480 | 0.0306 | 0.0358 |
| RS12490683 | 03: 075461775 | LOC652408 | 0.003 | G,G: 1.4(1.15, 1.7) | 0.0022 | G: 1.24(1.08, 1.42) | 0.0467 | 0.0216 | 0.0021 | 0.0456 |
| RS1025946 | 03: 078747342 | LOC642132, | 0.0026 | C,C: 1.68(1.2, 2.35) | 0.0033 | C: 1.64(1.18, 2.27) | 0.0381 | 0.0426 | 0.0299 | 0.0343 |
| | | ROBO1 | | | | | | | | |
| RS1397977 | 03: 096938051 | | 0.0187 | A,A: 1.32(1.08, 1.63) | 0.0056 | A: 1.21(1.06, 1.38) | 0.0367 | 0.0645 | 0.1108 | 0.0483 |
| RS1461155 | 03: 099731422 | | 0.02 | A,A: 1.51(1.12, 2.05) | 0.0057 | A: 1.5(1.12, 2) | 0.0436 | 0.0918 | 0.0066 | 0.0319 |
| RS11925785 | 03: 105431807 | | 0.0761 | T,T: 1.25(1, 1.56) | 0.0236 | T: 1.17(1.02, 1.33) | 0.0081 | 0.3562 | 0.0005 | 0.0271 |
| RS4895051 | 03: 105472583 | | 0.0168 | T,T: 1.34(1.1, 1.64) | 0.0131 | T: 1.19(1.04, 1.35) | 0.0446 | 0.0880 | 0.0002 | 0.0717 |
| RS7645613 | 03: 116546362 | | 0.0064 | C,C: 3.14(1.44, 6.85) | 0.0338 | C: 1.23(1.02, 1.5) | 0.0152 | 0.4991 | 0.0747 | 0.0247 |
| RS6763416 | 03: 116549440 | | 0.0102 | C,C: 3.02(1.38, 6.6) | 0.0482 | C: 1.22(1, 1.48) | 0.0244 | 0.5875 | 0.0842 | 0.0301 |
| RS13320544 | 03: 125457097 | KALRN | 0.0437 | A,A: 1.3(1.01, 1.69) | 0.0236 | A: 1.33(1.04, 1.69) | 0.0127 | 0.6666 | 0.0053 | 0.0054 |
| RS6438839 | 03: 125536191 | KALRN | 0.0174 | !G,G: 4.25(1.32, 13.72) | 0.0612 | A: 1.25(0.99, 1.59) | 0.0123 | 0.7925 | 0.0614 | 0.0243 |
| RS4293718 | 03: 129411810 | EEFSEC | 0.001 | !G,G: 1.43(1.18, 1.72) | 0.0007 | A: 1.31(1.12, 1.52) | 0.0099 | 0.0061 | 0.0904 | 0.0388 |
| RS3828417 | 03: 129610944 | EEFSEC | 0.0034 | C,C: 1.56(1.19, 2.05) | 0.0013 | C: 1.53(1.18, 1.99) | 0.1288 | 0.0474 | 0.0095 | 0.0064 |
| RS6777158 | 03: 140999494 | | 0.0002 | !T,T: 1.69(1.29, 2.2) | 1.02E−04 | G: 1.31(1.14, 1.5) | 0.0503 | 0.0321 | 0.0023 | 0.0009 |
| RS899348 | 03: 142311491 | SPSB4 | 0.046 | C,T: 1.27(1.05, 1.54) | 0.2479 | C: 1.09(0.94, 1.27) | 0.0015 | 0.4308 | 0.0534 | 0.0150 |
| RS9289637 | 03: 142855081 | | 0.0014 | A,C: 1.39(1.15, 1.67) | 0.0391 | C: 1.15(1.01, 1.32) | 0.0275 | 0.0433 | 0.0276 | 0.3873 |
| RS7431637 | 03: 144532459 | SLC9A9 | 0.0026 | C,T: 1.39(1.15, 1.68) | 0.2415 | T: 1.08(0.95, 1.24) | 0.0062 | 0.0042 | 0.0028 | 0.2476 |
| RS2174783 | 03: 147912451 | | 0.0096 | A,A: 1.28(1.06, 1.55) | 0.0024 | A: 1.24(1.08, 1.43) | 0.0805 | 0.0346 | 0.1119 | 0.0338 |
| RS4681336 | 03: 147956833 | | 0.0056 | !G,G: 1.47(1.14, 1.88) | 0.0023 | A: 1.23(1.08, 1.41) | 0.0420 | 0.0260 | 0.1227 | 0.0460 |
| RS6785504 | 03: 154155469 | | .0008 | !G,T: 1.39(1.15, 1.68) | 0.1996 | G: 1.09(0.96, 1.25) | 0.0406 | 0.0991 | 0.0100 | 0.8509 |
| RS1568790 | 03: 155009791 | | 0.0161 | T,T: 14.93(0.85, 261.84) | 1 | T: 1(0.78, 1.29) | 0.0077 | 0.0145 | 0.0127 | 0.0295 |
| RS7631400 | 03: 161011416 | SCHIP1 | 0.0003 | !A,A: 1.48(1.22, 1.79) | 1.47E−04 | G: 1.36(1.16, 1.6) | 0.0022 | 0.0015 | 0.1040 | 0.0457 |
| RS4129765 | 03: 174037987 | | 0.0064 | T,T: 1.37(1.13, 1.68) | 0.0056 | T: 1.28(1.08, 1.52) | 0.0411 | 0.0923 | 0.0795 | 0.0272 |
| RS1512744 | 03: 174364271 | | 0.0015 | A,G: 1.46(1.18, 1.8) | 0.0282 | A: 1.22(1.02, 1.45) | 0.0292 | 0.0649 | 0.0431 | 0.1996 |
| RS2861203 | 03: 174433247 | | 0.0008 | A,G: 1.44(1.19, 1.75) | 0.0694 | G: 1.15(0.99, 1.32) | 0.0294 | 0.0140 | 0.0018 | 0.8774 |
| RS2046718 | 03: 174955021 | NLGN1 | 0.001 | A,A: 2.21(1.43, 3.41) | 0.0096 | A: 1.23(1.05, 1.45) | 0.0306 | 0.0111 | 0.0114 | 0.2811 |
| RS2222447 | 03: 176063520 | NAALADL2 | 0.007 | !A,A: 1.52(1.15, 1.99) | 0.0042 | C: 1.22(1.07, 1.4) | 0.0905 | 0.0444 | 0.0798 | 0.0460 |
| RS2080787 | 03: 176541238 | NAALADL2 | 0.048 | !C,C: 1.24(1.01, 1.53) | 0.0125 | T: 1.18(1.04, 1.35) | 0.0130 | 0.0469 | 0.0409 | 0.1326 |
| RS16825529 | 03: 176550912 | NAALADL2 | 0.0202 | !C,C: 1.36(1.09, 1.7) | 0.0114 | A: 1.19(1.04, 1.36) | 0.0933 | 0.0368 | 0.0358 | 0.1457 |
| RS4894485 | 03: 176560380 | NAALADL2 | 6.71E−06 | !C,C: 1.62(1.33, 1.97) | 9.45E−06 | T: 1.36(1.19, 1.56) | 0.0060 | 0.0016 | 0.0002 | 0.0026 |
| RS7609811 | 03: 177341698 | | 0.0105 | T,T: 1.32(1.1, 1.6) | 0.0281 | T: 1.17(1.02, 1.34) | 0.1081 | 0.0379 | 0.0304 | 0.3314 |
| RS6800635 | 03: 177560866 | | 0.0081 | A,A: 1.59(1.17, 2.16) | 0.0017 | A: 1.59(1.19, 2.12) | 0.1412 | 0.0469 | 0.0166 | 0.0114 |
| RS2130014 | 03: 178098965 | | 0.0027 | !C,C: 1.62(1.22, 2.16) | 0.073 | T: 1.13(0.99, 1.3) | 0.0343 | 0.7237 | 0.0282 | 0.0350 |
| RS4254636 | 03: 180018514 | KCNMB2 | 0.0276 | !A,A: 1.29(1.07, 1.56) | 0.0235 | G: 1.19(1.03, 1.39) | 0.0301 | 0.3018 | 0.0861 | 0.0344 |
| RS6794192 | 03: 180510507 | | 0.0516 | C,C: 1.22(1.01, 1.48) | 0.0163 | C: 1.19(1.03, 1.36) | 0.0451 | 0.1523 | 0.0346 | 0.0567 |
| RS9826864 | 03: 181241320 | | 0.0145 | !A,G: 1.31(1.08, 1.59) | 0.0286 | A: 1.18(1.02, 1.36) | 0.0839 | 0.0319 | 0.0278 | 0.3551 |
| RS4855011 | 03: 182234345 | | 0.0072 | !C,C: 1.34(1.11, 1.62) | 0.0023 | T: 1.24(1.08, 1.43) | 0.0644 | 0.0241 | 0.0545 | 0.0395 |
| RS2313212 | 03: 185183622 | ABCC5 | 0.0054 | C,T: 1.35(1.12, 1.63) | 0.2491 | C: 1.08(0.95, 1.24) | 0.0106 | 0.0124 | 0.0319 | 0.4802 |
| RS2593813 | 03: 187815265 | AHSG | 0.0131 | !T,T: 1.52(1.15, 2.01) | 0.0223 | C: 1.18(1.03, 1.35) | 0.0297 | 0.2416 | 0.1307 | 0.0462 |
| RS3816112 | 03: 187985359 | EIF4A2 | 0.0379 | !C,T: 1.4(1.05, 1.86) | 0.0815 | T: 1.27(0.97, 1.65) | 0.0325 | 0.7718 | 0.0777 | 0.0331 |
| RS4475031 | 03: 188743651 | | 0.0882 | T,T: 1.24(1.02, 1.5) | 0.0403 | T: 1.18(1.01, 1.37) | 0.0188 | 0.0110 | 0.0491 | 0.7411 |
| RS4687002 | 03: 190104562 | | 0.007 | C,C: 1.33(1.11, 1.61) | 0.0144 | C: 1.2(1.04, 1.39) | 0.0427 | 0.2552 | 0.0644 | 0.0242 |
| RS12107377 | 03: 193633511 | FGF12 | 0.0022 | !A,A: 1.38(1.11, 1.71) | 0.0004 | G: 1.27(1.11, 1.45) | 0.0196 | 0.0049 | 0.1121 | 0.0386 |
| RS4687332 | 03: 193637315 | FGF12 | 0.0051 | C,C: 1.4(1.14, 1.73) | 0.0021 | C: 1.23(1.08, 1.41) | 0.1254 | 0.0461 | 0.0322 | 0.0243 |
| RS2980206 | 04: 004386725 | | 0.0026 | !T,T: 1.75(1.26, 2.43) | 0.0635 | C: 1.15(0.99, 1.33) | 0.0121 | 0.6363 | 0.0521 | 0.0312 |
| RS6826372 | 04: 005845805 | EVC | 0.0036 | !A,G: 1.32(1.08, 1.63) | 0.4747 | A: 1.07(0.9, 1.27) | 0.0467 | 0.5671 | 0.0155 | 0.1026 |
| RS17178569 | 04: 008115661 | ABLIM2 | 0.0007 | A,G: 1.46(1.19, 1.79) | 0.0432 | A: 1.2(1.01, 1.42) | 0.0148 | 0.1166 | 0.0464 | 0.2180 |
| RS2293229 | 04: 008458306 | ACOX3 | 0.0145 | T,T: 1.3(1.08, 1.57) | 0.0038 | T: 1.24(1.07, 1.44) | 0.1127 | 0.0447 | 0.1236 | 0.0418 |
| RS1464258 | 04: 009443205 | SLC2A9 | 0.0076 | !G,G: 1.35(1.11, 1.63) | 0.0213 | T: 1.18(1.03, 1.35) | 0.0482 | 0.0165 | 0.0193 | 0.4071 |
| RS6449068 | 04: 009473034 | SLC2A9 | 0.0163 | !T,T: 1.32(1.09, 1.59) | 0.0118 | C: 1.2(1.04, 1.38) | 0.0096 | 0.0032 | 0.0422 | 0.5146 |
| RS1519098 | 04: 009490256 | SLC2A9 | 0.0237 | !G,G: 1.3(1.08, 1.58) | 0.0289 | T: 1.17(1.02, 1.33) | 0.0463 | 0.0131 | 0.0474 | 0.5607 |
| RS1606084 | 04: 013506439 | | 0.0036 | C,C: 1.39(1.14, 1.68) | 0.0028 | C: 1.28(1.09, 1.5) | 0.0203 | 0.0236 | 0.1241 | 0.0486 |
| RS7676721 | 04: 013525449 | | 1.65E−05 | G,G: 2.14(1.42, 3.21) | 6.71E−06 | G: 1.42(1.22, 1.66) | 6.83E−05 | 3.01E−05 | 0.0808 | 0.0301 |
| RS505703 | 04: 013530080 | | 5.88E−05 | !A,A: 1.54(1.25, 1.89) | 1.12E−05 | G: 1.5(1.25, 1.8) | 0.0037 | 0.0020 | 0.0041 | 0.0018 |
| RS6840951 | 04: 014053172 | | 0.01 | !C,C: 1.86(1.1, 3.14) | 0.8937 | C: 1.01(0.85, 1.21) | 0.0236 | 0.0293 | 0.0147 | 0.0545 |
| RS10516278 | 04: 014129536 | | 0.0163 | G,T: 1.29(1.03, 1.62) | 0.5121 | G: 1.07(0.88, 1.3) | 0.0664 | 0.0416 | 0.0229 | 0.2926 |
| RS7696128 | 04: 018697484 | | 0.0241 | !T,T: 1.32(1.06, 1.64) | 0.0069 | G: 1.2(1.05, 1.37) | 0.0486 | 0.0808 | 0.0057 | 0.0299 |
| RS4496580 | 04: 019278475 | | 0.0089 | !T,T: 2.49(1.31, 4.74) | 0.027 | C: 1.22(1.03, 1.46) | 0.0354 | 0.3383 | 0.0660 | 0.0299 |
| RS1030349 | 04: 037504194 | PGM2 | 0.0084 | A,A: 1.32(1.08, 1.61) | 0.0026 | A: 1.31(1.1, 1.56) | 0.0979 | 0.0393 | 0.0770 | 0.0273 |
| RS2608307 | 04: 037514632 | PGM2 | 0.0109 | A,A: 1.28(1.06, 1.56) | 0.0035 | A: 1.28(1.09, 1.52) | 0.0902 | 0.0323 | 0.1106 | 0.0405 |
| RS1377347 | 04: 041067359 | DKFZP686A01247 | 7.36E−05 | !G,G: 1.52(1.26, 1.83) | 9.55E−05 | T: 1.33(1.15, 1.54) | 0.0027 | 0.0045 | 0.0200 | 0.0086 |
| RS13116757 | 04: 041093136 | DKFZP686A01247 | 0.0013 | !T,T: 1.42(1.18, 1.71) | 0.0008 | G: 1.3(1.12, 1.51) | 0.0129 | 0.0086 | 0.0905 | 0.0386 |
| RS1028000 | 04: 054155288 | | 0.0021 | T,T: 1.78(1.2, 2.65) | 0.0008 | T: 1.3(1.12, 1.52) | 0.0569 | 0.0333 | 0.0019 | 0.0089 |
| RS4131086 | 04: 057080212 | ARL9 | 0.0002 | !G,G: 2.06(1.44, 2.95) | 0.0453 | A: 1.16(1, 1.35) | 0.0314 | 0.4459 | 1.03E−04 | 0.0004 |
| RS10517437 | 04: 058703169 | | 0.0031 | G,G: 1.45(1.17, 1.8) | 0.0063 | G: 1.2(1.06, 1.38) | 0.1060 | 0.0414 | 0.0165 | 0.0821 |
| RS1505663 | 04: 061814685 | | 0.0021 | !A,A: 1.42(1.16, 1.73) | 0.0021 | G: 1.31(1.1, 1.55) | 0.1218 | 0.0459 | 0.0065 | 0.0215 |
| RS2034002 | 04: 077324198 | SCARB2 | 0.001 | A,G: 1.68(1.23, 2.3) | 0.008 | G: 1.49(1.11, 2) | 0.0122 | 0.0200 | 0.0406 | 0.1649 |
| RS10005531 | 04: 079039537 | MRPL1 | 0.0017 | !G,T: 1.39(1.15, 1.68) | 0.1541 | G: 1.1(0.97, 1.26) | 0.0263 | 0.0348 | 0.0212 | 0.9251 |
| RS10019920 | 04: 079085031 | MRPL1 | 0.0009 | !A,G: 1.4(1.16, 1.69) | 0.0902 | G: 1.12(0.98, 1.28) | 0.0111 | 0.0163 | 0.0221 | 1.0000 |
| RS7436080 | 04: 079096173 | | 0.0008 | !C,T: 1.41(1.17, 1.7) | 0.0968 | C: 1.12(0.98, 1.28) | 0.0111 | 0.0163 | 0.0181 | 0.9625 |
| RS979512 | 04: 079171790 | | 0.0004 | !C,T: 1.42(1.17, 1.71) | 0.1129 | T: 1.11(0.98, 1.27) | 0.0413 | 0.0414 | 0.0023 | 0.8880 |
| RS7376907 | 04: 083875498 | SCD5 | 0.0002 | !C,C: 1.57(1.23, 2) | 6.07E−05 | T: 1.32(1.15, 1.5) | 0.0050 | 0.0012 | 0.0308 | 0.0159 |
| RS2622629 | 04: 089313088 | | 0.0094 | !T,T: 1.34(1.1, 1.62) | 0.004 | C: 1.22(1.07, 1.41) | 0.0392 | 0.0459 | 0.0945 | 0.0373 |
| RS12651141 | 04: 089506733 | | 0.001 | A,A: 1.48(1.2, 1.82) | 0.0004 | A: 1.39(1.16, 1.68) | 0.0646 | 0.0271 | 0.0128 | 0.0057 |
| RS17248648 | 04: 091988638 | | 0.0009 | A,G: 1.6(1.23, 2.07) | 0.005 | G: 1.4(1.11, 1.78) | 0.0082 | 0.0112 | 0.0267 | 0.1898 |
| RS6856278 | 04: 097787265 | LOC441031 | 0.0075 | !C,C: 1.42(1.1, 1.83) | 0.0023 | A: 1.24(1.08, 1.41) | 0.0995 | 0.0465 | 0.0791 | 0.0257 |
| RS1042364 | 04: 100264597 | ADH4 | 0.008 | !G,G: 1.62(1.18, 2.23) | 0.1515 | A: 1.11(0.96, 1.29) | 0.0426 | 0.8339 | 0.0339 | 0.0233 |
| RS1126673 | 04: 100264639 | ADH4 | 0.0094 | !G,G: 1.57(1.16, 2.13) | 0.0973 | A: 1.13(0.98, 1.3) | 0.0205 | 0.9589 | 0.0480 | 0.0176 |
| RS1126672 | 04: 100266835 | ADH4 | 0.0083 | !C,C: 1.61(1.17, 2.22) | 0.1514 | T: 1.11(0.96, 1.29) | 0.0423 | 0.8337 | 0.0345 | 0.0231 |
| RS1126671 | 04: 100267437 | ADH4 | 0.0094 | !G,G: 1.57(1.16, 2.13) | 0.0973 | A: 1.13(0.98, 1.3) | 0.0205 | 0.9589 | 0.0480 | 0.0176 |
| RS1126670 | 04: 100271756 | ADH4 | 0.0096 | !G,G: 1.58(1.17, 2.14) | 0.0833 | T: 1.13(0.98, 1.31) | 0.0305 | 0.9589 | 0.0356 | 0.0132 |
| RS4128643 | 04: 101433596 | | 0.0077 | !G,G: 1.33(1.07, 1.64) | 0.0021 | A: 1.23(1.08, 1.41) | 0.0933 | 0.0331 | 0.0907 | 0.0305 |
| RS10516462 | 04: 101526697 | | 0.0005 | !G,G: 1.46(1.21, 1.77) | 0.0004 | A: 1.28(1.12, 1.47) | 0.0621 | 0.0304 | 0.0071 | 0.0059 |
| RS1452689 | 04: 109970223 | COL25A1 | 0.0078 | A,A: 3.15(1.38, 7.21) | 0.5255 | A: 1.07(0.87, 1.32) | 0.0556 | 0.0457 | 0.0243 | 0.4369 |
| RS12641791 | 04: 122118114 | | 1.38E−04 | !A,G: 2.01(1.4, 2.9) | 0.0022 | A: 1.71(1.21, 2.4) | 0.0060 | 0.0372 | 0.0148 | 0.0343 |
| RS970349 | 04: 123091082 | | 0.0035 | G,G: 1.38(1.14, 1.67) | 0.0031 | G: 1.27(1.08, 1.48) | 0.1006 | 0.0411 | 0.0276 | 0.0324 |
| RS1911014 | 04: 126993548 | | 0.0107 | !G,G: 2.88(1.37, 6.09) | 0.0752 | A: 1.18(0.99, 1.42) | 0.0491 | 0.6847 | 0.0724 | 0.0444 |
| RS1032673 | 04: 129684939 | | 0.0199 | A,C: 1.31(1.08, 1.58) | 0.1466 | C: 1.11(0.96, 1.28) | 0.0435 | 0.6813 | 0.0418 | 0.0181 |
| RS1908039 | 04: 131347083 | LOC645995, | 0.0053 | !G,G: 1.45(1.13, 1.85) | 0.0014 | A: 1.45(1.16, 1.82) | 0.1356 | 0.0480 | 0.0400 | 0.0114 |
| | | LOC648034 | | | | | | | | |
| RS1908038 | 04: 131352941 | LOC645995, | 0.0049 | !A,A: 1.45(1.14, 1.85) | 0.0012 | G: 1.45(1.16, 1.82) | 0.1417 | 0.0495 | 0.0351 | 0.0093 |
| | | LOC648034 | | | | | | | | |
| RS11732021 | 04: 133738741 | | 0.0054 | !A,A: 1.36(1.12, 1.64) | 0.0018 | G: 1.25(1.09, 1.43) | 0.0636 | 0.0246 | 0.0692 | 0.0263 |
| RS3796627 | 04: 140980727 | MAML3 | 0.0019 | !T,T: 1.62(1.21, 2.17) | 0.0014 | C: 1.25(1.09, 1.44) | 0.0776 | 0.0492 | 0.0229 | 0.0109 |
| RS4696084 | 04: 151437383 | LRBA | 0.0029 | !A,A: 1.39(1.15, 1.68) | 0.0017 | C: 1.28(1.1, 1.5) | 0.0461 | 0.0122 | 0.0405 | 0.0533 |
| RS2054685 | 04: 173475035 | GALNT17 | 0.0041 | !T,T: 1.42(1.13, 1.78) | 0.0012 | C: 1.41(1.14, 1.73) | 0.0301 | 0.0131 | 0.0982 | 0.0362 |
| RS6553010 | 04: 187693337 | MTNR1A | 0.0022 | !A,A: 1.42(1.16, 1.72) | 0.0005 | G: 1.34(1.14, 1.58) | 0.0715 | 0.0184 | 0.0180 | 0.0110 |
| RS6857356 | 04: 188706532 | | 0.0036 | !A,A: 2.93(1.49, 5.78) | 0.0245 | G: 1.26(1.03, 1.53) | 0.0719 | 0.0472 | 0.0389 | 0.2312 |
| RS6895153 | 05: 002586586 | | 0.0081 | T,T: 1.32(1.08, 1.6) | 0.002 | T: 1.29(1.1, 1.52) | 0.0990 | 0.0354 | 0.0847 | 0.0277 |
| RS256358 | 05: 005723085 | | 0.0017 | !T,T: 1.98(1.35, 2.91) | 0.0239 | C: 1.19(1.02, 1.39) | 0.0292 | 0.2777 | 0.0466 | 0.0431 |
| RS10040610 | 05: 006406815 | | 0.0005 | C,C: 5.02(1.59, 15.87) | 0.001 | C: 1.43(1.16, 1.76) | 0.0137 | 0.0090 | 0.0352 | 0.0314 |
| RS4701724 | 05: 006418689 | | 0.0054 | !T,T: 1.37(1.11, 1.7) | 0.0014 | C: 1.24(1.09, 1.42) | 0.0302 | 0.0088 | 0.0870 | 0.0486 |
| RS7735244 | 05: 006637248 | LOC255167 | 0.0029 | !C,T: 1.54(1.2, 1.99) | 0.0042 | T: 1.4(1.11, 1.76) | 0.0462 | 0.0185 | 0.0345 | 0.1042 |
| RS446249 | 05: 008429813 | | 0.0068 | !T,T: 1.3(1.08, 1.57) | 0.0021 | C: 1.25(1.09, 1.44) | 0.0555 | 0.0234 | 0.1001 | 0.0367 |
| RS40708 | 05: 009317682 | SEMA5A | 0.0018 | !G,G: 1.69(1.23, 2.33) | 0.001 | A: 1.67(1.23, 2.27) | 0.0236 | 0.0145 | 0.0131 | 0.0297 |
| RS465107 | 05: 009575303 | SEMA5A | 0.016 | !A,A: 2.1(1.21, 3.64) | 0.0227 | G: 1.23(1.03, 1.47) | 0.0253 | 0.2193 | 0.1412 | 0.0484 |
| RS960567 | 05: 009751490 | LOC285692 | 6.79E−05 | !A,C: 1.53(1.25, 1.86) | 0.1207 | A: 1.13(0.97, 1.32) | 0.0267 | 0.1434 | 0.0017 | 0.4863 |
| RS606490 | 05: 010371455 | | 0.001 | !T,T: 2.81(1.5, 5.29) | 0.0025 | C: 1.32(1.1, 1.57) | 0.0058 | 0.0014 | 0.0171 | 0.2787 |
| RS1373992 | 05: 013511367 | | 0.0046 | A,A: 1.38(1.13, 1.68) | 0.0012 | A: 1.32(1.12, 1.56) | 0.0395 | 0.0205 | 0.0880 | 0.0308 |
| RS40979 | 05: 016774306 | MYO10 | 0.0127 | !G,G: 1.38(1.11, 1.71) | 0.0048 | T: 1.32(1.09, 1.6) | 0.0566 | 0.0385 | 0.0032 | 0.0560 |
| RS4242060 | 05: 018932882 | | 0.008 | A,A: 1.36(1.12, 1.65) | 0.003 | A: 1.28(1.09, 1.51) | 0.1114 | 0.0421 | 0.0453 | 0.0352 |
| RS10062061 | 05: 027873351 | | 0.0109 | !A,C: 1.33(1.1, 1.61) | 0.1408 | A: 1.11(0.97, 1.28) | 0.0707 | 0.0321 | 0.0139 | 0.9606 |
| RS2287582 | 05: 031338045 | CDH6 | 0.0257 | A,G: 1.28(1.06, 1.54) | 0.3621 | G: 1.06(0.93, 1.22) | 0.0358 | 0.0196 | 0.0251 | 0.3478 |
| RS2909570 | 05: 031354162 | CDH6 | 0.0109 | G,T: 1.31(1.09, 1.58) | 0.2367 | G: 1.09(0.95, 1.24) | 0.0328 | 0.0149 | 0.0126 | 0.5412 |
| RS4867387 | 05: 031877036 | PDZD2 | 0.0023 | !C,C: 2.93(1.49, 5.79) | 0.0083 | T: 1.28(1.07, 1.55) | 0.0659 | 0.0494 | 0.0342 | 0.0775 |
| RS1847610 | 05: 031975096 | PDZD2 | 0.0009 | C,T: 1.38(1.15, 1.67) | 0.058 | C: 1.14(1, 1.3) | 0.0237 | 0.0410 | 0.0209 | 0.5096 |
| RS7726919 | 05: 032002949 | PDZD2 | 0.0106 | !T,T: 1.35(1.11, 1.65) | 0.0085 | G: 1.2(1.05, 1.37) | 0.0296 | 0.1285 | 0.0992 | 0.0318 |
| RS7705676 | 05: 035273493 | | 0.0981 | T,T: 1.22(1, 1.5) | 0.0303 | T: 1.16(1.01, 1.32) | 0.0409 | 0.1312 | 0.0214 | 0.1317 |
| RS7715492 | 05: 050516918 | | 0.0153 | C,T: 1.55(1.13, 2.13) | 0.0184 | T: 1.45(1.07, 1.96) | 0.0359 | 0.0212 | 0.0350 | 0.2929 |
| RS10062839 | 05: 060139209 | ELOVL7 | 0.0013 | !T,T: 2.15(1.35, 3.43) | 0.4361 | C: 1.07(0.91, 1.25) | 0.0250 | 0.6765 | 0.0256 | 0.1275 |
| RS153373 | 05: 065461915 | | 0.0006 | !A,G: 1.58(1.25, 2.01) | 0.0034 | A: 1.37(1.11, 1.69) | 0.0472 | 0.0865 | 0.0064 | 0.0104 |
| RS1466344 | 05: 071449384 | MAP1B | 0.0011 | !G,G: 1.5(1.18, 1.9) | 0.0003 | A: 1.5(1.2, 1.87) | 0.0707 | 0.0398 | 0.0079 | 0.0028 |
| RS3101187 | 05: 071452383 | MAP1B | 0.0013 | !G,G: 1.48(1.18, 1.85) | 0.0003 | A: 1.45(1.18, 1.78) | 0.0056 | 0.0022 | 0.0948 | 0.0379 |
| RS16878312 | 05: 071741140 | | 0.0021 | C,C: 1.32(1.1, 1.6) | 0.0005 | C: 1.29(1.12, 1.49) | 0.0137 | 0.0047 | 0.1135 | 0.0362 |
| RS191899 | 05: 073481573 | | 0.0168 | G,G: 1.29(1.07, 1.56) | 0.0043 | G: 1.22(1.07, 1.4) | 0.0217 | 0.0189 | 0.0433 | 0.0884 |
| RS4425488 | 05: 075330102 | | 0.0054 | C,C: 1.39(1.14, 1.7) | 0.0032 | C: 1.22(1.07, 1.4) | 0.1057 | 0.0320 | 0.0174 | 0.0422 |
| RS2287933 | 05: 076005975 | IQGAP2 | 0.0214 | C,C: 6.32(1.13, 35.45) | 1 | C: 1(0.78, 1.3) | 0.0208 | 0.0975 | 0.0482 | 0.1143 |
| RS2279095 | 05: 076745182 | PDE8B | 0.003 | !T,T: 1.52(1.18, 1.96) | 0.0017 | G: 1.46(1.15, 1.86) | 0.0084 | 0.0976 | 0.0067 | 0.0066 |
| RS2044163 | 05: 077267160 | | 0.0008 | T,T: 1.97(1.32, 2.93) | 0.001 | T: 1.92(1.3, 2.85) | 0.0286 | 0.0307 | 0.0124 | 0.0142 |
| RS423704 | 05: 082256836 | | 1.49E−04 | C,T: 1.66(1.29, 2.14) | 0.0078 | T: 1.37(1.09, 1.72) | 0.0115 | 0.0184 | 0.0068 | 0.1423 |
| RS871759 | 05: 082784704 | | 0.0048 | !C,C: 1.3(1.07, 1.59) | 0.0027 | T: 1.3(1.1, 1.55) | 0.0773 | 0.0409 | 0.0576 | 0.0240 |
| RS1500226 | 05: 083652159 | EDIL3 | 0.0119 | C,C: 1.45(1.13, 1.85) | 0.0324 | C: 1.16(1.01, 1.32) | 0.0365 | 0.0128 | 0.0272 | 0.6029 |
| RS7717140 | 05: 083655286 | EDIL3 | 0.0219 | G,G: 1.5(1.12, 2.01) | 0.0568 | G: 1.14(1, 1.31) | 0.1180 | 0.0416 | 0.0425 | 0.4936 |
| RS10805837 | 05: 085947410 | | 0.0113 | T,T: 1.29(1.07, 1.56) | 0.0029 | T: 1.24(1.08, 1.42) | 0.0141 | 0.0291 | 0.0222 | 0.0417 |
| RS1423595 | 05: 090831684 | | 0.0173 | !A,A: 1.35(1.1, 1.66) | 0.0136 | G: 1.18(1.04, 1.35) | 0.0858 | 0.0289 | 0.0313 | 0.1877 |
| RS4916875 | 05: 091123834 | | 0.0113 | G,G: 1.5(1.14, 1.99) | 0.1423 | G: 1.11(0.97, 1.27) | 0.0820 | 0.0295 | 0.0021 | 1.0000 |
| RS11949638 | 05: 093933645 | MGC34713 | 0.02 | A,A: 1.4(1.1, 1.79) | 0.0174 | A: 1.18(1.03, 1.34) | 0.0127 | 0.3292 | 0.0546 | 0.0203 |
| RS257218 | 05: 097404940 | | 0.0107 | A,G: 1.33(1.1, 1.6) | 0.5091 | A: 1.05(0.92, 1.2) | 0.0195 | 0.0409 | 0.0487 | 0.2865 |
| RS10515303 | 05: 100303212 | | 0.0132 | C,T: 1.33(1.1, 1.6) | 0.172 | C: 1.11(0.96, 1.27) | 0.0444 | 0.2757 | 0.0033 | 0.0028 |
| RS1422046 | 05: 102958169 | | 0.0183 | T,T: 1.31(1.08, 1.6) | 0.0056 | T: 1.21(1.06, 1.38) | 0.0416 | 0.0204 | 0.0441 | 0.1283 |
| RS6859797 | 05: 104016997 | | 1.30E−04 | !A,C: 1.56(1.26, 1.92) | 0.0051 | A: 1.29(1.08, 1.53) | 0.0372 | 0.0493 | 0.0024 | 0.0462 |
| RS1435194 | 05: 105481256 | | 0.0005 | !C,C: 1.4(1.16, 1.7) | 1.15E−04 | A: 1.37(1.17, 1.61) | 0.0609 | 0.0203 | 0.0051 | 0.0019 |
| RS11959425 | 05: 109587365 | | 0.0037 | A,A: 1.32(1.09, 1.59) | 0.0008 | A: 1.31(1.12, 1.53) | 0.0621 | 0.0230 | 0.0525 | 0.0186 |
| RS17133647 | 05: 111061008 | | 0.0055 | A,A: 4.35(1.55, 12.17) | 0.3295 | A: 1.12(0.9, 1.41) | 0.0589 | 0.0478 | 0.0138 | 0.8231 |
| RS10519637 | 05: 119905653 | LOC51334 | 0.0777 | !A,A: 1.25(1.02, 1.53) | 0.0234 | G: 1.22(1.03, 1.46) | 0.0491 | 0.1696 | 0.0482 | 0.0691 |
| RS245184 | 05: 127190475 | | 0.0027 | !A,G: 1.36(1.12, 1.64) | 0.4505 | A: 1.06(0.91, 1.23) | 0.0170 | 0.3621 | 0.0201 | 0.0383 |
| RS2191081 | 05: 128053707 | | 0.0012 | !T,T: 1.43(1.18, 1.74) | 0.0006 | C: 1.27(1.11, 1.45) | 0.0939 | 0.0485 | 0.0144 | 0.0055 |
| RS1019135 | 05: 128060202 | | 0.0011 | !A,A: 1.43(1.18, 1.74) | 0.0005 | G: 1.27(1.11, 1.46) | 0.0939 | 0.0485 | 0.0130 | 0.0047 |
| RS958687 | 05: 132665816 | FSTL4 | 0.001 | G,G: 1.48(1.2, 1.83) | 0.0009 | G: 1.25(1.1, 1.43) | 0.0153 | 0.0102 | 0.0563 | 0.0344 |
| RS31551 | 05: 135303229 | FBXL21 | 0.0023 | T,T: 1.4(1.16, 1.7) | 0.0013 | T: 1.25(1.09, 1.44) | 0.0495 | 0.0394 | 0.0417 | 0.0147 |
| RS30737 | 05: 135351333 | | 0.0095 | A,A: 1.34(1.1, 1.64) | 0.0027 | A: 1.31(1.1, 1.56) | 0.0765 | 0.0417 | 0.0697 | 0.0275 |
| RS12519073 | 05: 136804661 | SPOCK1 | 0.0588 | C,C: 1.58(1.06, 2.36) | 0.0419 | C: 1.18(1.01, 1.38) | 0.0105 | 0.0051 | 0.0215 | 1.0000 |
| RS3734166 | 05: 137693222 | CDC25C | 0.0352 | !A,A: 1.55(1.06, 2.27) | 0.6177 | G: 1.04(0.89, 1.21) | 0.0470 | 0.1478 | 0.0056 | 0.0204 |
| RS1991800 | 05: 145733194 | | 0.0009 | C,T: 1.52(1.21, 1.9) | 0.0123 | C: 1.29(1.06, 1.56) | 0.0340 | 0.0424 | 0.0263 | 0.1244 |
| RS6868716 | 05: 145769452 | | 0.0003 | C,T: 1.65(1.28, 2.12) | .0027 | T: 1.4(1.13, 1.75) | 0.0096 | 0.0060 | 0.0172 | 0.1160 |
| RS2288803 | 05: 146756043 | DPYSL3 | 0.0476 | A,A: 1.56(1.04, 2.35) | 0.6645 | A: 1.04(0.89, 1.21) | 0.0187 | 0.0181 | 0.0495 | 0.1300 |
| RS352360 | 05: 148643981 | FLJ36748 | 0.0009 | !A,G: 1.43(1.18, 1.72) | 0.6857 | A: 1.03(0.9, 1.18) | 0.0232 | 0.4099 | 0.0257 | 0.8879 |
| RS296171 | 05: 152584259 | | 0.0005 | !T,T: 1.48(1.21, 1.8) | .0002 | C: 1.38(1.16, 1.63) | 0.0082 | 0.0526 | 0.0035 | 0.0013 |
| RS6892282 | 05: 159293063 | ADRA1B | 0.0282 | !G,T: 1.28(1.06, 1.54) | 0.2183 | T: 1.09(0.95, 1.24) | 0.0720 | 0.0273 | 0.0246 | 0.7027 |
| RS7724146 | 05: 160827209 | GABRB2 | 0.0013 | C,T: 1.44(1.17, 1.77) | 0.0725 | C: 1.17(0.99, 1.39) | 0.0316 | 0.0910 | 0.0383 | 0.4251 |
| RS2431268 | 05: 161956558 | LOC651586, | 0.0056 | !A,A: 1.61(1.17, 2.22) | .0032 | G: 1.24(1.08, 1.43) | 0.0955 | 0.0386 | 0.0121 | 0.0402 |
| | | LOC644536 | | | | | | | | |
| RS4498268 | 05: 163768515 | | 0.0107 | !G,G: 1.34(1.09, 1.64) | .0033 | A: 1.31(1.1, 1.57) | 0.0821 | 0.0303 | 0.1023 | 0.0475 |
| RS7718356 | 05: 163781686 | | 0.0009 | !G,G: 1.48(1.19, 1.83) | .0002 | T: 1.44(1.19, 1.76) | 0.0310 | 0.0120 | 0.0272 | 0.0090 |
| RS165070 | 05: 163933613 | | 0.0346 | !C,T: 1.28(1.04, 1.57) | 0.3595 | C: 1.08(0.91, 1.29) | 0.0468 | 0.2464 | 0.0397 | 0.0237 |
| RS2962867 | 05: 164254631 | | 0.0096 | !C,C: 1.33(1.1, 1.61) | 0.0133 | T: 1.22(1.04, 1.42) | 0.1044 | 0.0438 | 0.0481 | 0.1903 |
| RS2938767 | 05: 168212967 | SLIT3 | 0.0022 | !G,G: 1.54(1.21, 1.96) | 0.0065 | A: 1.2(1.05, 1.38) | 0.0417 | 0.0276 | 0.0430 | 0.1081 |
| RS17646250 | 05: 169244766 | DOCK2 | 0.0276 | T,T: 4.99(1.28, 19.48) | 0.1847 | T: 1.17(0.93, 1.49) | 0.0431 | 0.2267 | 0.0057 | 0.0024 |
| RS4868141 | 05: 171456041 | STK10 | 0.0042 | A,A: 1.33(1.09, 1.62) | 0.0013 | A: 1.33(1.12, 1.57) | 0.0396 | 0.0211 | 0.0834 | 0.0279 |
| RS522133 | 05: 172364187 | ATP6V0E | 0.0008 | !T,T: 1.66(1.27, 2.16) | 0.0002 | C: 1.59(1.24, 2.03) | 0.0003 | 0.0043 | 0.0130 | 0.0291 |
| RS746215 | 05: 172943959 | | 0.022 | C,C: 1.31(1.08, 1.59) | 0.0122 | C: 1.23(1.05, 1.45) | 0.0815 | 0.0419 | 0.0288 | 0.1419 |
| RS13164416 | 05: 173911212 | | 0.0027 | T,T: 1.51(1.19, 1.93) | 0.0017 | T: 1.24(1.09, 1.42) | 0.0675 | 0.0217 | 0.0327 | 0.0390 |
| RS6556186 | 05: 174478299 | | 0.0605 | !A,A: 1.25(1.04, 1.51) | 0.0331 | G: 1.18(1.01, 1.37) | 0.0014 | 0.3835 | 0.0046 | 0.0363 |
| RS2471005 | 05: 174814813 | | 0.0008 | !C,T: 1.42(1.18, 1.72) | 0.7834 | T: 1.02(0.89, 1.17) | 0.0166 | 0.3194 | 0.0316 | 0.5684 |
| RS2913816 | 05: 177559776 | | 0.0402 | A,G: 1.27(1.05, 1.53) | 0.5658 | G: 1.04(0.91, 1.2) | 0.0293 | 0.2085 | 0.0426 | 0.0381 |
| RS11738131 | 05: 177587800 | MGC15875 | 0.0043 | A,A: 1.47(1.16, 1.85) | 0.0021 | A: 1.4(1.13, 1.73) | 0.0750 | 0.0457 | 0.0592 | 0.0281 |
| RS11242687 | 06: 001384975 | | 0.0162 | C,T: 1.31(1.08, 1.58) | 0.2044 | C: 1.09(0.95, 1.25) | 0.0030 | 0.5867 | 0.0586 | 0.0221 |
| RS9328246 | 06: 004308481 | | 0.02 | !C,C: 2.1(1.23, 3.6) | 0.0843 | T: 1.17(0.98, 1.39) | 0.0453 | 0.7532 | 0.0912 | 0.0341 |
| RS9350235 | 06: 010364661 | | 0.0045 | G,G: 1.39(1.14, 1.7) | 0.0013 | G: 1.32(1.11, 1.57) | 0.0303 | 0.0132 | 0.1292 | 0.0497 |
| RS1931017 | 06: 010368921 | | 0.0017 | G,G: 1.45(1.18, 1.78) | 0.0004 | G: 1.39(1.16, 1.66) | 0.0116 | 0.0036 | 0.1071 | 0.0385 |
| RS12213634 | 06: 010391324 | | 0.0004 | T,T: 1.48(1.2, 1.83) | 7.51E−05 | T: 1.46(1.21, 1.77) | 0.0108 | 0.0032 | 0.0355 | 0.0112 |
| RS9370735 | 06: 014352496 | | 0.0155 | !T,T: 2.28(1.13, 4.61) | 0.0095 | G: 1.3(1.07, 1.58) | 0.0347 | 0.0899 | 0.1212 | 0.0489 |
| RS719343 | 06: 014445172 | | 0.0059 | A,A: 1.82(1.18, 2.79) | 0.0035 | A: 1.27(1.08, 1.48) | 0.0668 | 0.0211 | 0.0186 | 0.0775 |
| RS1923747 | 06: 017334064 | | 0.0003 | A,G: 1.46(1.21, 1.76) | 0.0918 | G: 1.12(0.98, 1.29) | 0.0294 | 0.7682 | 0.0067 | 0.0429 |
| RS495768 | 06: 020143047 | | 0.0018 | A,A: 1.4(1.16, 1.7) | 0.0029 | A: 1.28(1.09, 1.49) | 0.0341 | 0.0421 | 0.0531 | 0.0291 |
| RS2000191 | 06: 022612727 | | 0.001 | !A,A: 1.69(1.24, 2.31) | 0.001 | C: 1.66(1.23, 2.24) | 0.0088 | 0.0076 | 0.0447 | 0.0505 |
| RS537835 | 06: 024075102 | | 0.0025 | C,C: 1.69(1.2, 2.38) | 0.0021 | C: 1.69(1.21, 2.36) | 0.0226 | 0.0179 | 0.0596 | 0.0390 |
| RS12196939 | 06: 027895969 | | 0.0047 | !A,A: 1.37(1.12, 1.68) | 0.0009 | G: 1.35(1.13, 1.61) | 0.0459 | 0.0153 | 0.1006 | 0.0389 |
| RS200991 | 06: 027923473 | | 0.0039 | G,G: 1.39(1.13, 1.72) | 0.0012 | G: 1.36(1.13, 1.64) | 0.0311 | 0.0129 | 0.1064 | 0.0456 |
| RS11752919 | 06: 028511582 | ZNF390 | 0.0012 | T,T: 1.4(1.16, 1.7) | 0.0145 | T: 1.19(1.04, 1.36) | 0.0230 | 0.0139 | 0.0423 | 0.3925 |
| RS2191037 | 06: 028574967 | | 0.0246 | C,C: 2.17(1.22, 3.85) | 0.139 | C: 1.14(0.96, 1.36) | 0.0207 | 0.0063 | 0.0401 | 0.5258 |
| RS6934993 | 06: 029445479 | | 0.001 | !G,G: 1.92(1.25, 2.96) | 0.0005 | A: 1.32(1.13, 1.55) | 0.0127 | 0.0267 | 0.0375 | 0.0105 |
| RS2267633 | 06: 029678820 | GABBR1 | 0.0019 | G,G: 3.8(1.59, 9.07) | 0.5447 | G: 1.06(0.87, 1.3) | 0.0151 | 0.0472 | 0.0232 | 0.3803 |
| RS2076483 | 06: 029679524 | GABBR1 | 0.0019 | C,C: 3.8(1.59, 9.07) | 0.5447 | C: 1.06(0.87, 1.3) | 0.0151 | 0.0472 | 0.0232 | 0.3803 |
| RS3132685 | 06: 030053928 | HCG9, HLA- | 0.0014 | C,C: 1.51(1.2, 1.89) | 0.0004 | C: 1.45(1.18, 1.79) | 0.0071 | 0.0059 | 0.0801 | 0.0268 |
| | | A, HLA-G | | | | | | | | |
| RS8321 | 06: 030140501 | ZNRD1 | 0.0007 | G,G: 1.53(1.21, 1.93) | 0.0002 | G: 1.51(1.21, 1.87) | 0.0041 | 0.0045 | 0.0601 | 0.0214 |
| RS1245371 | 06: 030146331 | RNF39 | 0.0482 | !T,T: 1.41(1.01, 1.97) | 0.0152 | C: 1.2(1.04, 1.39) | 0.0270 | 0.0326 | 0.0394 | 0.2196 |
| RS9261290 | 06: 030146626 | RNF39 | 0.0007 | T,T: 1.53(1.21, 1.93) | 0.0002 | T: 1.51(1.21, 1.87) | 0.0041 | 0.0045 | 0.0601 | 0.0214 |
| RS3130380 | 06: 030387109 | HCG18 | 0.0026 | A,A: 1.47(1.16, 1.85) | 0.0007 | A: 1.45(1.17, 1.8) | 0.0231 | 0.0135 | 0.0801 | 0.0301 |
| RS3130350 | 06: 030435818 | | 0.0007 | T,T: 1.51(1.2, 1.91) | 0.0002 | T: 1.5(1.21, 1.86) | 0.0104 | 0.0108 | 0.0412 | 0.0122 |
| RS2535315 | 06: 031160106 | | 0.0414 | !G,G: 1.28(1.06, 1.56) | 0.0349 | A: 1.16(1.01, 1.33) | 0.0416 | 0.7324 | 0.0258 | 0.0154 |
| RS2535311 | 06: 031160663 | | 0.0414 | !C,C: 1.28(1.06, 1.56) | 0.0349 | T: 1.16(1.01, 1.33) | 0.0416 | 0.7324 | 0.0258 | 0.0154 |
| RS7750641 | 06: 031237289 | TCF19 | 0.0056 | T,T: 1.42(1.13, 1.77) | 0.0015 | T: 1.39(1.14, 1.7) | 0.1034 | 0.0437 | 0.0643 | 0.0228 |
| RS6457374 | 06: 031380240 | BAT1, HLA- | 0.0003 | C,C: 1.39(1.15, 1.67) | 7.98E−05 | C: 1.35(1.16, 1.57) | 0.0236 | 0.0108 | 0.0152 | 0.0046 |
| | | B, HLA-C | | | | | | | | |
| RS2596501 | 06: 031429190 | BAT1, HLA- | 0.0019 | !G,G: 1.44(1.16, 1.79) | 0.0008 | A: 1.25(1.1, 1.43) | 0.0127 | 0.0525 | 0.0150 | 0.0083 |
| | | B, HLA-C | | | | | | | | |
| RS2523554 | 06: 031439808 | BAT1 | 0.0005 | A,A: 1.45(1.2, 1.77) | 0.0003 | A: 1.29(1.13, 1.48) | 0.0475 | 0.0154 | 0.0058 | 0.0078 |
| RS2844513 | 06: 031496193 | BAT1* | 0.0019 | !C,C: 1.35(1.1, 1.65) | 0.0006 | T: 1.26(1.11, 1.44) | 0.0173 | 0.0116 | 0.0354 | 0.0308 |
| RS2844511 | 06: 031497763 | BAT1 | 9.14E−05 | C,C: 1.53(1.24, 1.88) | 2.04E−05 | C: 1.33(1.17, 1.52) | 0.0069 | 0.0022 | 0.0184 | 0.0056 |
| RS2284178 | 06: 031540104 | HCP5 | 0.0003 | !T,T: 1.53(1.24, 1.89) | 0.0003 | C: 1.28(1.12, 1.46) | 0.0188 | 0.0650 | 0.0038 | 0.0014 |
| RS3099844 | 06: 031556955 | BAT1 | 0.0014 | A,A: 1.51(1.2, 1.88) | 0.0005 | A: 1.44(1.18, 1.77) | 0.0630 | 0.0284 | 0.0280 | 0.0091 |
| RS2844498 | 06: 031584833 | BAT1, MICB | 0.0023 | A,A: 1.52(1.18, 1.95) | 0.0017 | A: 1.24(1.08, 1.42) | 0.0368 | 0.0127 | 0.0468 | 0.0632 |
| RS2857595 | 06: 031676448 | | 0.001 | !T,T: 2.15(1.3, 3.56) | 0.0004 | C: 1.35(1.14, 1.6) | 0.1069 | 0.0494 | 0.0097 | 0.0042 |
| RS2261033 | 06: 031711570 | BAT2 | 1.41E−04 | !C,C: 1.51(1.23, 1.85) | 0.0101 | T: 1.19(1.04, 1.36) | 0.0147 | 0.3325 | 0.0093 | 0.0141 |
| RS2734335 | 06: 032001923 | C2 | 0.0019 | !C,C: 1.46(1.18, 1.81) | 0.0014 | T: 1.24(1.09, 1.42) | 0.0552 | 0.0249 | 0.0410 | 0.0369 |
| RS9276644 | 06: 032853021 | | 0.004 | !C,C: 1.59(1.18, 2.13) | 0.0022 | T: 1.24(1.08, 1.43) | 0.0352 | 0.0607 | 0.0478 | 0.0143 |
| RS6924102 | 06: 032919361 | PSMB8, | 0.0003 | !G,G: 1.53(1.2, 1.94) | 9.15E−05 | A: 1.31(1.14, 1.49) | 0.0345 | 0.0110 | 0.0097 | 0.0033 |
| | | TAP1 | | | | | | | | |
| RS2071538 | 06: 032926656 | TAP1 | 0.0002 | !C,C: 1.45(1.2, 1.75) | 5.34E−05 | T: 1.39(1.18, 1.62) | 0.0033 | 0.0007 | 0.0639 | 0.0233 |
| RS9276814 | 06: 032933068 | | 0.0013 | !G,G: 1.8(1.25, 2.6) | 0.001 | A: 1.28(1.11, 1.48) | 0.0247 | 0.0475 | 0.0250 | 0.0079 |
| RS241404 | 06: 032973975 | | 0.0002 | !G,G: 1.65(1.28, 2.12) | 0.0003 | A: 1.28(1.12, 1.47) | 0.0042 | 0.0015 | 0.0206 | 0.0453 |
| RS154981 | 06: 032988971 | | 0.0002 | !A,A: 1.51(1.21, 1.88) | 4.45E−05 | G: 1.32(1.15, 1.5) | 0.0025 | 0.0006 | 0.0295 | 0.0187 |
| RS1061632 | 06: 036565462 | KCTD20 | 0.0144 | C,T: 1.33(1.09, 1.63) | 0.136 | C: 1.14(0.96, 1.34) | 0.0335 | 0.0176 | 0.0397 | 0.8114 |
| RS626516 | 06: 036573358 | STK38 | 0.0106 | !A,A: 1.76(1.16, 2.68) | 0.0058 | G: 1.25(1.07, 1.47) | 0.1384 | 0.0489 | 0.0478 | 0.0529 |
| RS2300064 | 06: 036582119 | STK38 | 0.0141 | C,T: 1.33(1.09, 1.63) | 0.1364 | T: 1.14(0.96, 1.34) | 0.0335 | 0.0176 | 0.0396 | 0.8582 |
| RS7741267 | 06: 037216358 | | 0.0015 | T,T: 1.67(1.25, 2.25) | 0.0892 | T: 1.13(0.98, 1.3) | 0.0464 | 0.6495 | 0.0178 | 0.0658 |
| RS16891295 | 06: 039015269 | DNAH8 | 0.0012 | !A,G: 1.47(1.11, 1.96) | 0.2109 | A: 1.18(0.91, 1.53) | 0.0232 | 0.9263 | 0.0364 | 0.0919 |
| RS6901007 | 06: 043000812 | PTCRA | 0.0013 | T,T: 1.46(1.15, 1.84) | 0.1546 | T: 1.1(0.96, 1.26) | 0.0449 | 0.4658 | 0.0305 | 0.2368 |
| RS4714675 | 06: 043395871 | | 0.0003 | C,C: 1.54(1.22, 1.94) | 8.92E−05 | C: 1.54(1.24, 1.91) | 0.0715 | 0.0269 | 0.0014 | 0.0008 |
| RS873053 | 06: 045955093 | | 0.007 | !T,T: 1.29(1.07, 1.56) | 0.0017 | C: 1.27(1.1, 1.47) | 0.0274 | 0.0135 | 0.1312 | 0.0422 |
| RS6901799 | 06: 052055216 | PKHD1 | 0.0181 | !G,G: 3.46(1.33, 9) | 0.4223 | A: 1.09(0.89, 1.35) | 0.0308 | 0.4500 | 0.0565 | 0.0489 |
| RS1316575 | 06: 054273259 | | 0.0094 | A,A: 1.35(1.11, 1.64) | 0.0095 | A: 1.2(1.04, 1.37) | 0.0683 | 0.0261 | 0.0218 | 0.1459 |
| RS937147 | 06: 063118894 | | 0.0282 | T,T: 1.3(1.07, 1.57) | 0.0342 | T: 1.16(1.01, 1.33) | 0.0347 | 0.5143 | 0.0773 | 0.0267 |
| RS9354216 | 06: 066053576 | | 0.0007 | C,T: 1.47(1.2, 1.8) | 0.0287 | C: 1.2(1.02, 1.42) | 0.0102 | 0.2606 | 0.0246 | 0.0462 |
| RS12213776 | 06: 079147541 | | 0.0115 | !T,T: 1.32(1.09, 1.59) | 0.0038 | G: 1.25(1.08, 1.45) | 0.0921 | 0.0382 | 0.0954 | 0.0488 |
| RS13220420 | 06: 080923376 | BCKDHB | 0.0005 | T,T: 1.55(1.17, 2.05) | 0.0008 | T: 1.57(1.21, 2.05) | 0.0096 | 0.0080 | 0.0273 | 0.0468 |
| RS10485285 | 06: 081535326 | | 0.0316 | !C,T: 1.29(1.07, 1.55) | 0.2713 | C: 1.08(0.94, 1.24) | 0.0303 | 0.2211 | 0.0130 | 0.0056 |
| RS10755417 | 06: 082652678 | | 0.0038 | C,C: 1.46(1.17, 1.82) | 0.0041 | C: 1.22(1.06, 1.39) | 0.0908 | 0.0327 | 0.0263 | 0.0541 |
| RS13192303 | 06: 084052583 | ME1 | 0.0026 | A,G: 1.53(1.19, 1.96) | 0.0121 | G: 1.34(1.07, 1.68) | 0.0394 | 0.1130 | 0.0542 | 0.0494 |
| RS12527778 | 06: 086311241 | SNX14 | 0.0251 | T,T: 1.36(1.08, 1.71) | 0.0052 | T: 1.35(1.09, 1.66) | 0.0568 | 0.0446 | 0.0310 | 0.0521 |
| RS9390728 | 06: 101647596 | | 0.0048 | !C,C: 1.35(1.11, 1.63) | 0.0015 | A: 1.3(1.11, 1.52) | 0.1181 | 0.0447 | 0.0367 | 0.0152 |
| RS1901708 | 06: 105025706 | | 0.0218 | G,G: 1.33(1.08, 1.64) | 0.0126 | G: 1.26(1.05, 1.51) | 0.0331 | 0.1554 | 0.0735 | 0.0293 |
| RS352066 | 06: 114353200 | | 0.028 | C,T: 1.26(1.04, 1.52) | 0.8883 | T: 1.01(0.88, 1.16) | 0.0229 | 0.0319 | 0.0266 | 0.0667 |
| RS2499618 | 06: 114391946 | HDAC2 | 0.0137 | C,T: 1.29(1.07, 1.56) | 0.8069 | T: 1.02(0.89, 1.17) | 0.0407 | 0.0468 | 0.0147 | 0.1157 |
| RS9489998 | 06: 120932004 | | 0.0003 | !G,G: 2.24(1.47, 3.41) | 0.1473 | T: 1.12(0.96, 1.31) | 0.0083 | 0.4278 | 0.0306 | 0.2328 |
| RS11759649 | 06: 121974359 | | 0.0258 | T,T: 1.28(1.06, 1.55) | 0.0675 | T: 1.14(0.99, 1.31) | 0.0166 | 0.6492 | 0.0114 | 0.0024 |
| RS9490609 | 06: 123298281 | | 0.0174 | !A,A: 1.43(1.08, 1.9) | 0.0069 | G: 1.21(1.05, 1.39) | 0.0145 | 0.1710 | 0.0202 | 0.0173 |
| RS1851780 | 06: 124668814 | TCBA1 | 0.0002 | !G,G: 2.15(1.35, 3.43) | 1 | A: 1(0.85, 1.18) | 0.0125 | 0.4431 | 0.0050 | 0.4156 |
| RS9402011 | 06: 128347581 | PTPRK | 0.0009 | T,T: 1.41(1.17, 1.7) | 0.0003 | T: 1.31(1.13, 1.52) | 0.0603 | 0.0345 | 0.0100 | 0.0031 |
| RS9375543 | 06: 128352833 | PTPRK | 0.0002 | T,T: 1.52(1.25, 1.86) | 0.0002 | T: 1.29(1.13, 1.48) | 0.0138 | 0.0156 | 0.0108 | 0.0056 |
| RS3903663 | 06: 128354781 | PTPRK | 0.0015 | A,A: 1.4(1.16, 1.69) | 0.0005 | A: 1.3(1.12, 1.51) | 0.0506 | 0.0337 | 0.0177 | 0.0058 |
| RS6931735 | 06: 135666504 | AHI1 | 0.0046 | !A,A: 1.5(1.17, 1.93) | 0.0291 | G: 1.16(1.02, 1.33) | 0.0066 | 0.4618 | 0.0639 | 0.0228 |
| RS7738388 | 06: 141494838 | | 0.0006 | C,C: 1.93(1.36, 2.75) | 0.002 | C: 1.26(1.09, 1.45) | 0.0051 | 0.0917 | 0.0225 | 0.0087 |
| RS208704 | 06: 148939203 | | 0.0031 | !T,T: 1.58(1.16, 2.15) | 0.001 | C: 1.27(1.1, 1.47) | 0.0912 | 0.0402 | 0.0361 | 0.0105 |
| RS10214840 | 06: 149685402 | MAP3K7IP2 | 0.0163 | !C,T: 1.3(1.07, 1.57) | 0.5166 | T: 1.05(0.91, 1.22) | 0.0432 | 0.2565 | 0.0350 | 0.0310 |
| RS2839947 | 06: 151230384 | MTHFD1L, | 0.0156 | !C,C: 1.42(1.11, 1.82) | 0.0143 | T: 1.18(1.04, 1.35) | 0.0250 | 0.2229 | 0.0647 | 0.0256 |
| | | LOC646099, | | | | | | | | |
| | | LOC652160 | | | | | | | | |
| RS4869953 | 06: 151251690 | MTHFD1L | 0.0007 | !G,G: 1.49(1.21, 1.83) | 0.0018 | A: 1.24(1.08, 1.41) | 0.0094 | 0.1446 | 0.0139 | 0.0040 |
| RS7745756 | 06: 155003154 | | 0.001 | A,A: 1.49(1.21, 1.85) | 0.0005 | A: 1.4(1.16, 1.69) | 0.0227 | 0.0099 | 0.0397 | 0.0176 |
| RS9479923 | 06: 155012248 | | 0.0083 | A,A: 1.33(1.09, 1.62) | 0.0023 | A: 1.31(1.1, 1.55) | 0.1009 | 0.0402 | 0.0795 | 0.0271 |
| RS9365171 | 06: 160901726 | LPA | 0.0015 | !C,C: 1.64(1.23, 2.18) | 0.1143 | A: 1.12(0.98, 1.28) | 0.0174 | 0.9203 | 0.0066 | 0.0157 |
| RS3798953 | 06: 161609806 | AGPAT4 | 7.63E−05 | G,G: 4.39(2.07, 9.31) | 0.0326 | G: 1.23(1.02, 1.48) | 0.0197 | 0.1089 | 0.0035 | 0.1986 |
| RS10806758 | 06: 162761969 | PARK2 | 0.0246 | C,C: 1.43(1.1, 1.85) | 0.0416 | C: 1.15(1.01, 1.32) | 0.0336 | 0.6180 | 0.0632 | 0.0189 |
| RS6927758 | 06: 163639105 | PACRG | 0.0297 | C,T: 1.27(1.05, 1.53) | 0.5869 | T: 1.04(0.91, 1.19) | 0.0094 | 0.0072 | 0.0176 | 0.0654 |
| RS9459583 | 06: 166478154 | | 0.0409 | T,T: 1.28(1.06, 1.55) | 0.0229 | T: 1.21(1.03, 1.42) | 0.0381 | 0.2409 | 0.0745 | 0.0411 |
| RS9364336 | 06: 169114813 | | 0.0025 | !C,C: 1.4(1.15, 1.71) | 0.0007 | T: 1.26(1.1, 1.44) | 0.0365 | 0.0107 | 0.0404 | 0.0230 |
| RS7452888 | 06: 169438189 | | 0.0073 | G,G: 1.33(1.08, 1.65) | 0.0019 | G: 1.24(1.08, 1.41) | 0.0295 | 0.0327 | 0.0278 | 0.0207 |
| RS4721321 | 07: 002034996 | MAD1L1 | 0.0027 | !A,A: 1.39(1.15, 1.68) | 0.0012 | G: 1.28(1.1, 1.49) | 0.0211 | 0.0304 | 0.0726 | 0.0231 |
| RS2906173 | 07: 002453336 | LOC641906, | 0.0282 | T,T: 1.48(1.11, 1.98) | 0.0617 | T: 1.14(0.99, 1.31) | 0.0344 | 0.7593 | 0.0629 | 0.0219 |
| | | AK024457, | | | | | | | | |
| | | FLJ00049 | | | | | | | | |
| RS9986825 | 07: 003117703 | | 0.0099 | T,T: 1.34(1.11, 1.62) | 0.0103 | T: 1.2(1.05, 1.38) | 0.0400 | 0.2209 | 0.0659 | 0.0200 |
| RS4722435 | 07: 003119488 | | 0.0037 | !T,T: 1.39(1.1, 1.76) | 0.001 | C: 1.25(1.1, 1.43) | 0.0531 | 0.0276 | 0.0310 | 0.0187 |
| RS4720539 | 07: 005234050 | WIPI2 | 0.0016 | T,T: 1.41(1.15, 1.72) | 0.0004 | T: 1.27(1.11, 1.46) | 0.0136 | 0.0056 | 0.0069 | 0.0190 |
| RS7904 | 07: 005239647 | WIPI2 | 0.0027 | !G,G: 1.35(1.11, 1.63) | 0.0005 | A: 1.28(1.11, 1.47) | 0.1399 | 0.0485 | 0.0136 | 0.0035 |
| RS10486156 | 07: 007315158 | | 0.0171 | T,T: 1.25(1.04, 1.51) | 0.0051 | T: 1.23(1.07, 1.42) | 0.0237 | 0.0629 | 0.0515 | 0.0358 |
| RS2057847 | 07: 009181854 | | 0.0024 | G,G: 1.37(1.13, 1.66) | 0.0007 | G: 1.27(1.11, 1.46) | 0.1041 | 0.0472 | 0.0213 | 0.0061 |
| RS1919315 | 07: 018218407 | LOC641939 | 0.0027 | !C,C: 1.38(1.14, 1.66) | 0.0059 | T: 1.24(1.06, 1.44) | 0.0185 | 0.1434 | 0.0449 | 0.0162 |
| RS7789412 | 07: 018224796 | LOC641939 | 0.0043 | C,T: 1.38(1.13, 1.68) | 0.011 | C: 1.22(1.05, 1.42) | 0.0358 | 0.2092 | 0.0483 | 0.0209 |
| RS1860875 | 07: 021078168 | | 0.0397 | !C,C: 1.28(1.05, 1.55) | 0.0486 | T: 1.15(1, 1.31) | 0.0238 | 0.7284 | 0.0093 | 0.0023 |
| RS2107124 | 07: 025266514 | | 0.0007 | !A,A: 1.97(1.36, 2.87) | 0.0013 | G: 1.28(1.1, 1.49) | 0.0234 | 0.0129 | 0.0241 | 0.0351 |
| RS2717907 | 07: 025269991 | | 7.97E−05 | C,C: 1.42(1.16, 1.73) | 5.15E−05 | C: 1.43(1.21, 1.71) | 0.0091 | 0.0026 | 0.0030 | 0.0066 |
| RS2214548 | 07: 026484080 | LOC441204 | 0.0007 | A,G: 1.44(1.19, 1.75) | 0.0104 | A: 1.21(1.05, 1.4) | 0.0155 | 0.0077 | 0.0113 | 0.3261 |
| RS917117 | 07: 028142830 | TCAG7.981 | 0.0002 | A,A: 2.56(1.57, 4.17) | 0.0007 | A: 1.33(1.13, 1.57) | 0.0077 | 0.0026 | 0.0116 | 0.0941 |
| RS849141 | 07: 028151616 | TCAG7.981 | 8.36E−05 | G,G: 1.49(1.24, 1.8) | 0.0008 | G: 1.29(1.11, 1.49) | 0.0012 | 0.0035 | 0.0456 | 0.0716 |
| RS2730616 | 07: 043885791 | URG4 | 0.0008 | !T,T: 1.74(1.27, 2.38) | 0.0006 | C: 1.28(1.11, 1.48) | 0.0417 | 0.0434 | 0.0144 | 0.0057 |
| RS2730617 | 07: 043894317 | URG4 | 0.0005 | !T,T: 1.77(1.3, 2.42) | 0.0005 | C: 1.29(1.12, 1.48) | 0.0309 | 0.0294 | 0.0158 | 0.0078 |
| RS1894752 | 07: 043963600 | LOC650422 | 0.0002 | !T,T: 1.78(1.34, 2.35) | 0.0004 | C: 1.29(1.12, 1.48) | 0.0310 | 0.0085 | 0.0028 | 0.0164 |
| RS7800244 | 07: 047746494 | | 0.0048 | G,G: 2.41(1.37, 4.24) | 0.0208 | G: 1.23(1.03, 1.46) | 0.1278 | 0.0475 | 0.0035 | 0.2191 |
| RS12671939 | 07: 052666021 | | 0.0005 | G,G: 3.58(1.73, 7.4) | 0.2369 | G: 1.12(0.93, 1.35) | 0.0078 | 0.1217 | 0.0376 | 0.9469 |
| RS11978966 | 07: 053223557 | LOC653175 | 0.0018 | !T,T: 1.54(1.2, 1.96) | 0.0004 | C: 1.51(1.2, 1.89) | 0.0063 | 0.0029 | 0.0851 | 0.0401 |
| RS7795810 | 07: 053358424 | | 0.0013 | !T,T: 1.47(1.17, 1.83) | 0.0003 | C: 1.45(1.19, 1.77) | 0.0264 | 0.0192 | 0.0200 | 0.0060 |
| RS10488321 | 07: 053428180 | | 0.0015 | !A,A: 1.44(1.15, 1.8) | 0.0003 | G: 1.45(1.18, 1.77) | 0.0366 | 0.0187 | 0.0369 | 0.0099 |
| RS1030306 | 07: 053606599 | | 0.0007 | C,T: 1.75(1.3, 2.37) | 0.0007 | C: 1.62(1.23, 2.14) | 0.0175 | 0.0186 | 0.0290 | 0.0185 |
| RS4718608 | 07: 066784667 | | 0.0004 | T,T: 1.49(1.2, 1.85) | 1.17E−04 | T: 1.47(1.21, 1.79) | 0.0144 | 0.0053 | 0.0169 | 0.0079 |
| RS4566929 | 07: 066832473 | | 0.0068 | A,A: 1.74(1.2, 2.52) | 0.0063 | A: 1.23(1.06, 1.43) | 0.0239 | 0.1367 | 0.0668 | 0.0186 |
| RS6976905 | 07: 070399889 | WBSCR17 | 0.0421 | A,A: 1.34(1.06, 1.68) | 0.0585 | A: 1.14(1, 1.3) | 0.0434 | 0.8841 | 0.0423 | 0.0127 |
| RS37609 | 07: 073539675 | GTF2IRD1 | 0.0221 | C,C: 1.46(1.1, 1.95) | 0.0121 | C: 1.2(1.04, 1.37) | 0.0866 | 0.0328 | 0.0361 | 0.1539 |
| RS798337 | 07: 077754218 | MAGI2 | 0.0066 | G,G: 1.65(1.2, 2.26) | 0.0955 | G: 1.13(0.98, 1.3) | 0.0756 | 0.0243 | 0.0052 | 0.8410 |
| RS798343 | 07: 077762177 | MAGI2 | 0.0022 | C,C: 1.58(1.19, 2.1) | 0.1789 | C: 1.1(0.96, 1.26) | 0.1155 | 0.0370 | 1.14E−05 | 0.9215 |
| RS6966472 | 07: 084537323 | SEMA3D | 0.0037 | T,T: 1.43(1.15, 1.78) | 0.0012 | T: 1.39(1.14, 1.7) | 0.0932 | 0.0439 | 0.0353 | 0.0104 |
| RS6953717 | 07: 089661725 | | 0.0029 | T,T: 1.95(1.24, 3.08) | 0.0018 | T: 1.29(1.1, 1.51) | 0.0468 | 0.0351 | 0.0675 | 0.0273 |
| RS194498 | 07: 089676093 | | 0.0038 | C,C: 1.91(1.21, 3.02) | 0.0023 | C: 1.28(1.09, 1.51) | 0.0624 | 0.0407 | 0.0675 | 0.0273 |
| RS2188505 | 07: 095374205 | DYNC1I1 | 0.0455 | !C,C: 2.6(1.04, 6.48) | 0.9564 | C: 1.01(0.82, 1.25) | 0.0054 | 0.0403 | 0.0304 | 0.0469 |
| RS7799245 | 07: 105093792 | ATXN7L1 | 0.0024 | !G,G: 1.36(1.13, 1.65) | 0.0006 | A: 1.27(1.11, 1.46) | 0.0582 | 0.0363 | 0.0067 | 0.0074 |
| RS10953522 | 07: 106375662 | | 0.0085 | !A,G: 1.33(1.11, 1.61) | 0.2927 | A: 1.08(0.94, 1.23) | 0.0238 | 0.6231 | 0.0437 | 0.0665 |
| RS10499980 | 07: 109054772 | | 0.0336 | !A,G: 1.28(1.06, 1.55) | 0.7612 | G: 1.02(0.89, 1.17) | 0.0076 | 0.0291 | 0.0120 | 0.0084 |
| RS6978286 | 07: 116404768 | ST7 | 0.0046 | !A,A: 1.35(1.12, 1.63) | 0.0011 | G: 1.29(1.11, 1.51) | 0.0651 | 0.0186 | 0.0705 | 0.0236 |
| RS2896218 | 07: 116707214 | WNT2 | 0.0002 | !G,G: 1.67(1.3, 2.14) | 0.0021 | A: 1.23(1.08, 1.41) | 0.0053 | 0.0024 | 0.0098 | 0.1822 |
| RS1443753 | 07: 121736554 | LOC154860 | 0.0019 | C,C: 1.6(1.19, 2.15) | 0.0009 | C: 1.27(1.1, 1.46) | 0.0445 | 0.0145 | 0.0357 | 0.0236 |
| RS890406 | 07: 131904650 | PLXNA4B | 0.0071 | A,A: 1.49(1.14, 1.93) | 0.122 | A: 1.11(0.97, 1.28) | 0.0256 | 1.0000 | 0.0431 | 0.0339 |
| RS11768238 | 07: 134878053 | | 0.0479 | !G,G: 1.46(1.07, 1.98) | 0.185 | A: 1.1(0.96, 1.27) | 0.0042 | 0.7206 | 0.0590 | 0.0207 |
| RS6970530 | 07: 136616871 | PTN | 0.0027 | A,A: 1.64(1.17, 2.3) | 0.0026 | A: 1.66(1.2, 2.3) | 0.0322 | 0.0250 | 0.0572 | 0.0438 |
| RS1451526 | 07: 136731763 | DGKI | 0.0003 | !A,A: 1.39(1.14, 1.69) | 1.04E−04 | G: 1.39(1.18, 1.64) | 0.0037 | 0.0008 | 0.0628 | 0.0375 |
| RS10487553 | 07: 143358783 | | 0.0019 | G,G: 1.63(1.24, 2.14) | 0.0067 | G: 1.21(1.05, 1.39) | 0.0158 | 0.0615 | 0.0722 | 0.0439 |
| RS2107759 | 07: 143361152 | | 0.002 | C,C: 1.63(1.24, 2.14) | 0.0074 | C: 1.21(1.05, 1.38) | 0.0153 | 0.0688 | 0.0730 | 0.0439 |
| RS826823 | 07: 146685306 | CNTNAP2 | 0.0253 | !C,T: 1.47(1.1, 1.95) | 0.0138 | T: 1.41(1.07, 1.84) | 0.0376 | 0.2759 | 0.0267 | 0.0187 |
| RS4314574 | 07: 146985864 | CNTNAP2 | 0.0068 | !G,G: 3.09(1.35, 7.1) | 0.0123 | A: 1.28(1.05, 1.55) | 0.0087 | 0.1966 | 0.0709 | 0.0227 |
| RS1225382 | 07: 155334646 | | 0.0431 | !A,G: 1.26(1.05, 1.52) | 0.2131 | G: 1.09(0.95, 1.25) | 0.1093 | 0.0383 | 0.0282 | 0.7752 |
| RS6559176 | 08: 006526697 | | 0.0055 | A,A: 1.4(1.14, 1.73) | 0.0041 | A: 1.22(1.06, 1.39) | 0.1032 | 0.0367 | 0.0375 | 0.0534 |
| RS11249877 | 08: 008572184 | | 0.0005 | !T,T: 1.57(1.25, 1.97) | 1.30E−04 | C: 1.5(1.22, 1.85) | 0.0167 | 0.0081 | 0.0199 | 0.0057 |
| RS2898249 | 08: 010782996 | | 0.0087 | A,A: 1.72(1.2, 2.45) | 0.0164 | A: 1.2(1.04, 1.39) | 0.1216 | 0.0479 | 0.0493 | 0.1558 |
| RS10503426 | 08: 011626792 | GATA4 | 0.0024 | !C,C: 1.51(1.19, 1.93) | 0.0478 | A: 1.15(1, 1.31) | 0.1142 | 0.0432 | 0.0018 | 0.4777 |
| RS804280 | 08: 011650107 | GATA4 | 0.0277 | !T,T: 1.4(1.09, 1.79) | 0.0475 | G: 1.15(1, 1.31) | 0.0066 | 0.0016 | 0.0220 | 0.7761 |
| RS4128298 | 08: 011860741 | | 0.0012 | !G,G: 1.94(1.35, 2.79) | 0.0087 | A: 1.22(1.05, 1.41) | 0.0092 | 0.1541 | 0.0548 | 0.0305 |
| RS1481692 | 08: 013383739 | DLC1 | 0.0013 | C,T: 1.53(1.21, 1.93) | 0.0014 | C: 1.4(1.14, 1.72) | 0.0274 | 0.0132 | 0.0430 | 0.0400 |
| RS6982196 | 08: 018848911 | PSD3 | 0.0015 | A,G: 1.4(1.16, 1.69) | 0.4944 | A: 1.05(0.91, 1.21) | 0.0136 | 0.0421 | 0.0143 | 0.3449 |
| RS1025229 | 08: 018856300 | PSD3 | 0.0031 | G,T: 1.38(1.14, 1.67) | 0.3903 | T: 1.06(0.93, 1.22) | 0.0118 | 0.0261 | 0.0287 | 0.3720 |
| RS333047 | 08: 019094091 | | 0.003 | T,T: 1.59(1.14, 2.21) | 0.0011 | T: 1.27(1.1, 1.46) | 0.0691 | 0.0254 | 0.0392 | 0.0200 |
| RS2672245 | 08: 019119152 | | 0.003 | !C,C: 1.51(1.17, 1.94) | 0.0009 | A: 1.5(1.18, 1.9) | 0.0233 | 0.0094 | 0.1164 | 0.0434 |
| RS2631878 | 08: 021173339 | | 0.001 | !A,A: 1.48(1.18, 1.85) | 0.0004 | G: 1.45(1.18, 1.77) | 0.0200 | 0.0261 | 0.0136 | 0.0076 |
| RS2631879 | 08: 021174982 | | 0.0007 | !T,T: 1.51(1.21, 1.89) | 0.0002 | C: 1.47(1.2, 1.8) | 0.0236 | 0.0213 | 0.0100 | 0.0044 |
| RS1035722 | 08: 025689448 | | 0.0032 | !G,G: 1.43(1.15, 1.77) | 0.0006 | A: 1.4(1.15, 1.69) | 0.0134 | 0.0066 | 0.0427 | 0.0333 |
| RS2681615 | 08: 031473005 | | 0.0011 | !A,A: 1.41(1.14, 1.75) | 0.0002 | G: 1.29(1.13, 1.47) | 0.0315 | 0.0088 | 0.0412 | 0.0098 |
| RS16921196 | 08: 056207801 | XKR4 | 0.0153 | !A,C: 1.28(1.04, 1.57) | 0.5989 | A: 1.05(0.89, 1.25) | 0.0474 | 0.0353 | 0.0095 | 0.2147 |
| RS7460495 | 08: 059596784 | | 0.0065 | !A,G: 1.3(1.08, 1.57) | 0.4278 | A: 1.06(0.92, 1.21) | 0.0108 | 0.0161 | 0.0363 | 0.1738 |
| RS1834733 | 08: 059600018 | | 0.0043 | !A,G: 1.3(1.08, 1.57) | 0.3353 | G: 1.07(0.93, 1.22) | 0.0052 | 0.0094 | 0.0425 | 0.1899 |
| RS1582824 | 08: 059609008 | | 0.0047 | !A,G: 1.32(1.09, 1.59) | 0.4497 | G: 1.05(0.92, 1.21) | 0.0057 | 0.0110 | 0.0318 | 0.1208 |
| RS10504321 | 08: 061975455 | | 0.0002 | !T,T: 1.48(1.18, 1.86) | 0.0002 | G: 1.5(1.21, 1.85) | 0.0070 | 0.0483 | 0.0015 | 0.0009 |
| RS728032 | 08: 069279032 | DEPDC2 | 0.0038 | !C,C: 1.38(1.11, 1.71) | 0.0021 | T: 1.36(1.12, 1.66) | 0.0233 | 0.0371 | 0.0536 | 0.0276 |
| RS1530241 | 08: 070573442 | SULF1 | 0.0091 | C,C: 1.34(1.1, 1.65) | 0.0026 | C: 1.23(1.08, 1.4) | 0.0640 | 0.0255 | 0.1021 | 0.0376 |
| RS1946937 | 08: 070581000 | SULF1 | 0.0088 | A,G: 1.3(1.06, 1.58) | 0.5101 | A: 1.06(0.9, 1.24) | 0.0214 | 0.0113 | 0.0031 | 0.1197 |
| RS1524641 | 08: 086009342 | | 1.50E−05 | !C,T: 2.11(1.5, 2.98) | 0.0003 | C: 1.8(1.31, 2.48) | 0.0005 | 0.0047 | 0.0118 | 0.0249 |
| RS6991672 | 08: 087721231 | CNGB3 | 0.0005 | !T,T: 1.71(1.28, 2.3) | 0.0003 | C: 1.69(1.27, 2.24) | 0.0016 | 0.0020 | 0.0404 | 0.0352 |
| RS1996637 | 08: 089227035 | MMP16 | 0.0009 | C,C: 1.47(1.2, 1.81) | 0.0004 | C: 1.27(1.11, 1.45) | 0.0589 | 0.0249 | 0.0165 | 0.0085 |
| RS2681297 | 08: 089238474 | MMP16 | 0.0013 | C,C: 1.46(1.18, 1.79) | 0.0006 | C: 1.26(1.1, 1.44) | 0.0587 | 0.0249 | 0.0252 | 0.0128 |
| RS2575738 | 08: 097599578 | SDC2 | 9.80E−05 | T,T: 1.96(1.38, 2.78) | 0.2548 | T: 1.09(0.94, 1.26) | 0.0233 | 1.0000 | 0.0014 | 0.1309 |
| RS1503555 | 08: 107376981 | | 0.0508 | T,T: 1.3(1.05, 1.6) | 0.015 | T: 1.27(1.05, 1.53) | 0.0456 | 0.0719 | 0.0485 | 0.1154 |
| RS10089853 | 08: 107395023 | | 0.0584 | A,A: 1.3(1.04, 1.62) | 0.0201 | A: 1.28(1.04, 1.56) | 0.0381 | 0.0871 | 0.0264 | 0.1315 |
| RS12682157 | 08: 107400829 | | 0.0124 | A,A: 1.3(1.07, 1.57) | 0.0037 | A: 1.27(1.08, 1.49) | 0.0345 | 0.0340 | 0.0790 | 0.0469 |
| RS10505093 | 08: 107686536 | | 0.0009 | A,A: 1.43(1.18, 1.73) | 0.0005 | A: 1.28(1.12, 1.47) | 0.0078 | 0.0053 | 0.0845 | 0.0327 |
| RS1858342 | 08: 115003522 | | 0.0072 | !T,T: 1.61(1.17, 2.21) | 0.002 | C: 1.61(1.19, 2.18) | 0.1359 | 0.0437 | 0.0381 | 0.0190 |
| RS10108622 | 08: 115631498 | | 0.0005 | !G,G: 2.35(1.5, 3.7) | 0.0058 | T: 1.25(1.07, 1.47) | 0.0202 | 0.0244 | 0.0218 | 0.1064 |
| RS4871533 | 08: 125831179 | | 0.002 | C,C: 1.4(1.16, 1.7) | .0007 | C: 1.31(1.12, 1.52) | 0.0025 | 0.0005 | 0.0432 | 0.1957 |
| RS6470459 | 08: 127771130 | | 0.0008 | A,A: 1.4(1.15, 1.69) | .0002 | A: 1.35(1.16, 1.59) | 0.0058 | 0.0020 | 0.0886 | 0.0262 |
| RS2648829 | 08: 129217093 | | 0.0261 | C,C: 1.84(1.16, 2.92) | 0.0498 | C: 1.18(1, 1.39) | 0.0662 | 0.0156 | 0.0294 | 0.6109 |
| RS13257555 | 08: 130011810 | | 0.0157 | !T,T: 1.41(1.07, 1.86) | 0.0089 | C: 1.43(1.1, 1.86) | 0.0363 | 0.0780 | 0.0215 | 0.0654 |
| RS9693430 | 08: 130034016 | | 0.005 | G,T: 1.58(1.19, 2.11) | 0.0094 | G: 1.42(1.09, 1.85) | 0.0377 | 0.5791 | 0.0062 | 0.0021 |
| RS894263 | 08: 130072445 | | 0.0027 | T,T: 1.47(1.14, 1.88) | 0.0009 | T: 1.26(1.1, 1.44) | 0.0714 | 0.0246 | 0.0426 | 0.0170 |
| RS6981912 | 08: 134747299 | | 0.0024 | G,G: 1.67(1.18, 2.37) | 0.0006 | G: 1.3(1.12, 1.51) | 0.0583 | 0.0406 | 0.0254 | 0.0073 |
| RS1123762 | 08: 135191884 | | 0.0229 | C,T: 1.36(1.09, 1.69) | 0.0263 | C: 1.24(1.03, 1.5) | 0.0652 | 0.0457 | 0.0256 | 0.2371 |
| RS9324520 | 08: 136123429 | | 0.0019 | T,T: 1.4(1.16, 1.69) | 0.0008 | T: 1.27(1.11, 1.47) | 0.0187 | 0.0110 | 0.0862 | 0.0311 |
| RS10104990 | 08: 136202757 | | 0.0042 | G,G: 1.41(1.15, 1.73) | 0.0112 | G: 1.19(1.04, 1.36) | 0.1112 | 0.0464 | 0.0193 | 0.1090 |
| RS4909885 | 08: 136203239 | | 0.0038 | G,G: 1.42(1.15, 1.74) | 0.0075 | G: 1.2(1.05, 1.37) | 0.1015 | 0.0367 | 0.0169 | 0.0897 |
| RS1009149 | 08: 136466442 | | 0.0028 | !A,G: 1.38(1.13, 1.67) | 0.3121 | A: 1.08(0.93, 1.26) | 0.0244 | 0.4453 | 0.0160 | 0.0194 |
| RS9657469 | 08: 136467298 | | 0.0051 | !G,T: 1.35(1.11, 1.64) | 0.3931 | T: 1.07(0.92, 1.25) | 0.0321 | 0.3550 | 0.0195 | 0.0226 |
| RS11780624 | 08: 138132395 | | 0.001 | !T,T: 1.41(1.16, 1.71) | 0.0003 | C: 1.29(1.13, 1.48) | 0.0284 | 0.0090 | 0.0309 | 0.0092 |
| RS11780634 | 08: 138190860 | | 0.0066 | G,G: 1.69(1.12, 2.54) | 0.0025 | G: 1.27(1.09, 1.48) | 0.0415 | 0.0257 | 0.1411 | 0.0463 |
| RS11780467 | 08: 138268410 | | 0.0023 | !T,T: 1.42(1.16, 1.73) | 0.0007 | C: 1.35(1.14, 1.6) | 0.0337 | 0.0134 | 0.0705 | 0.0237 |
| RS868421 | 08: 138668249 | | 0.0296 | G,G: 1.28(1.06, 1.54) | 0.0093 | G: 1.22(1.05, 1.41) | 0.0068 | 0.0987 | 0.0207 | 0.0430 |
| RS4243888 | 08: 139322987 | | 0.0004 | !T,T: 1.84(1.36, 2.51) | 0.0073 | C: 1.21(1.05, 1.4) | 0.0314 | 0.4437 | 0.0019 | 0.0026 |
| RS2977451 | 08: 141603380 | LOC646107 | 0.0032 | !G,G: 1.43(1.16, 1.77) | 0.0007 | A: 1.38(1.15, 1.67) | 0.0223 | 0.0064 | 0.0751 | 0.0329 |
| RS2977477 | 08: 141624141 | EIF2C2 | 0.01 | !G,G: 1.35(1.11, 1.65) | 0.0022 | A: 1.31(1.1, 1.55) | 0.1011 | 0.0337 | 0.0840 | 0.0317 |
| RS2280880 | 08: 143718931 | | 0.0077 | !G,G: 1.31(1.08, 1.58) | 0.0021 | T: 1.25(1.09, 1.44) | 0.0974 | 0.0449 | 0.0484 | 0.0190 |
| RS2382962 | 08: 144740535 | EEF1D | 0.0018 | !A,G: 1.43(1.09, 1.88) | 0.4241 | G: 1.11(0.87, 1.41) | 0.0390 | 1.0000 | 0.0410 | 0.1653 |
| RS896962 | 08: 144758235 | PYCRL | 0.001 | !A,G: 1.37(1.11, 1.69) | 0.4106 | A: 1.08(0.9, 1.29) | 0.0131 | 0.4509 | 0.0171 | 0.0326 |
| RS1509197 | 09: 001009473 | | 0.0329 | !C,T: 1.27(1.05, 1.54) | 0.3835 | C: 1.07(0.92, 1.25) | 0.0203 | 0.3281 | 0.0610 | 0.0329 |
| RS1535844 | 09: 002184805 | SMARCA2 | 0.004 | !C,C: 1.36(1.12, 1.65) | 0.0012 | T: 1.32(1.12, 1.56) | 0.0618 | 0.0290 | 0.0443 | 0.0165 |
| RS1535843 | 09: 002184871 | SMARCA2 | 0.003 | !T,T: 1.37(1.13, 1.66) | 0.0009 | C: 1.33(1.12, 1.56) | 0.0445 | 0.0215 | 0.0443 | 0.0165 |
| RS914494 | 09: 002459939 | FLJ35024 | 0.0003 | A,A: 1.47(1.22, 1.77) | 0.0004 | A: 1.3(1.13, 1.51) | 0.0094 | 0.0026 | 0.0105 | 0.0525 |
| RS2027394 | 09: 004059657 | GLIS3 | 0.0012 | A,G: 1.34(1.11, 1.62) | 0.526 | A: 1.05(0.91, 1.2) | 0.0356 | 0.1444 | 0.0126 | 0.6568 |
| RS1931236 | 09: 011092971 | | 0.0006 | !T,T: 2.36(1.49, 3.74) | 0.0139 | G: 1.21(1.04, 1.41) | 0.0221 | 0.0356 | 0.0215 | 0.1674 |
| RS7857061 | 09: 013506313 | | 0.0202 | A,A: 1.3(1.08, 1.57) | 0.0083 | A: 1.21(1.05, 1.4) | 0.0412 | 0.2185 | 0.0200 | 0.0107 |
| RS518054 | 09: 013679066 | | 0.001 | !T,T: 2.39(1.46, 3.92) | 0.0049 | G: 1.26(1.07, 1.49) | 0.0347 | 0.0741 | 0.0307 | 0.0292 |
| RS1318995 | 09: 024901674 | | 4.63E−05 | C,C: 1.63(1.31, 2.02) | 1.20E−04 | C: 1.3(1.14, 1.48) | .0030 | 0.0198 | 0.0077 | 0.0026 |
| RS2126176 | 09: 024912485 | | 0.0011 | A,A: 1.74(1.29, 2.35) | 0.0116 | A: 1.2(1.04, 1.38) | 0.0299 | 0.0545 | 0.0307 | 0.0983 |
| RS4978100 | 09: 026741013 | | 0.0011 | T,T: 1.72(1.23, 2.4) | 0.0005 | T: 1.29(1.12, 1.5) | 0.0279 | 0.0395 | 0.0211 | 0.0048 |
| RS2070074 | 09: 034639442 | GALT | 0.008 | !A,A: 1.48(1.15, 1.89) | 0.0026 | G: 1.43(1.13, 1.79) | 0.0938 | 0.0321 | 0.0765 | 0.0352 |
| RS7036081 | 09: 037613913 | | 0.0022 | !C,C: 1.39(1.13, 1.7) | 0.0007 | T: 1.37(1.14, 1.64) | 0.0396 | 0.0127 | 0.0323 | 0.0205 |
| RS10973953 | 09: 038629853 | | 0.0471 | G,G: 1.59(1.08, 2.35) | 0.3533 | G: 1.08(0.93, 1.25) | 0.0337 | 0.1274 | 0.0070 | 0.0042 |
| RS12342083 | 09: 077385731 | | 0.0119 | T,T: 1.32(1.05, 1.65) | 0.006 | T: 1.33(1.09, 1.64) | 0.0241 | 0.0125 | 0.0465 | 0.1758 |
| RS4745430 | 09: 077461845 | | 0.0026 | C,C: 1.32(1.09, 1.59) | 0.0007 | C: 1.27(1.11, 1.46) | 0.0175 | 0.0068 | 0.0994 | 0.0300 |
| RS4745431 | 09: 077461915 | | 0.0024 | C,C: 1.35(1.1, 1.64) | 0.0005 | C: 1.27(1.11, 1.45) | 0.0706 | 0.0284 | 0.0189 | 0.0054 |
| RS10869675 | 09: 077779855 | PCSK5 | 0.0109 | !A,A: 1.3(1.08, 1.57) | 0.0028 | G: 1.25(1.08, 1.44) | 0.0393 | 0.0449 | 0.0193 | 0.0305 |
| RS2007744 | 09: 078044132 | | 1.13E−06 | A,C: 1.77(1.42, 2.19) | 3.84E−05 | C: 1.47(1.22, 1.76) | 0.0278 | 0.0665 | 1.35E−05 | 9.03E−05 |
| RS7043482 | 09: 084325735 | | 0.0002 | A,C: 1.48(1.22, 1.79) | 0.0372 | C: 1.16(1.01, 1.34) | 0.0227 | 0.1238 | 0.0112 | 0.1744 |
| RS4242620 | 09: 085145258 | FRMD3 | 0.0095 | A,A: 1.29(1.07, 1.56) | 0.0025 | A: 1.27(1.09, 1.48) | 0.0878 | 0.0333 | 0.0693 | 0.0312 |
| RS11140345 | 09: 085859150 | | 0.0621 | !G,G: 1.4(1.05, 1.88) | 0.0472 | A: 1.15(1, 1.32) | 0.0241 | 0.5065 | 0.0793 | 0.0387 |
| RS1599045 | 09: 088831397 | LOC440173 | 0.0023 | !T,T: 2.35(1.33, 4.15) | 0.002 | C: 1.32(1.11, 1.58) | 0.0038 | 0.0205 | 0.1344 | 0.0442 |
| RS465855 | 09: 088988236 | | 0.0083 | !G,G: 1.41(1.13, 1.77) | 0.0056 | A: 1.21(1.06, 1.38) | 0.0426 | 0.0582 | 0.1336 | 0.0486 |
| RS277722 | 09: 088990946 | | 0.0059 | !G,G: 1.43(1.14, 1.79) | 0.0041 | A: 1.22(1.06, 1.39) | 0.0429 | 0.0520 | 0.1045 | 0.0388 |
| RS11265877 | 09: 091509347 | FLJ42342 | 0.0056 | A,G: 1.5(1.16, 1.95) | 0.0139 | G: 1.36(1.06, 1.73) | 0.0284 | 0.0239 | 0.0138 | 0.2085 |
| RS7045961 | 09: 091753819 | | 0.0492 | G,G: 1.88(0.94, 3.76) | 0.6858 | T: 1.05(0.86, 1.28) | 0.0425 | 0.1679 | 0.0668 | 0.0256 |
| RS10993008 | 09: 092125202 | | 0.0022 | A,G: 1.42(1.15, 1.76) | 0.0398 | G: 1.21(1.01, 1.46) | 0.0407 | 0.0283 | 0.0101 | 0.5114 |
| RS2296751 | 09: 092921175 | | 0.0126 | T,T: 1.29(1.04, 1.6) | 0.0062 | T: 1.31(1.08, 1.59) | 0.0555 | 0.0371 | 0.0055 | 0.0848 |
| RS12339155 | 09: 093434461 | | 0.0277 | C,C: 1.73(1.15, 2.61) | 0.0925 | C: 1.15(0.98, 1.34) | 0.0136 | 0.9087 | 0.0949 | 0.0290 |
| RS7863890 | 09: 094465567 | IPPK | 0.0109 | !A,A: 1.33(1.1, 1.6) | 0.0044 | G: 1.24(1.07, 1.44) | 0.0590 | 0.0221 | 0.0468 | 0.0710 |
| RS1758799 | 09: 100379511 | GABBR2 | 0.0007 | !C,T: 1.44(1.19, 1.75) | 0.104 | C: 1.13(0.98, 1.31) | 0.0134 | 0.1200 | 0.0396 | 0.4754 |
| RS914665 | 09: 100402769 | GABBR2 | 0.0063 | T,T: 1.35(1.11, 1.63) | 0.0019 | T: 1.25(1.09, 1.43) | 0.0419 | 0.0293 | 0.0124 | 0.0311 |
| RS2846457 | 09: 112567386 | MUSK | 0.0346 | !G,G: 1.29(1.06, 1.56) | 0.0137 | A: 1.23(1.04, 1.44) | 0.0586 | 0.0491 | 0.0439 | 0.1405 |
| RS6478269 | 09: 118814195 | ASTN2 | .0034 | G,G: 1.37(1.12, 1.67) | 0.0009 | G: 1.35(1.13, 1.6) | 0.0326 | 0.0086 | 0.0739 | 0.0333 |
| RS694428 | 09: 119653079 | LOC645358, | 0.0054 | !C,T: 1.63(1.19, 2.23) | 0.0167 | C: 1.42(1.07, 1.89) | 0.0323 | 0.5616 | 0.0328 | 0.0123 |
| | | LOC647666 | | | | | | | | |
| RS7849366 | 09: 120152185 | | 0.0046 | G,G: 1.95(1.29, 2.96) | 0.1171 | G: 1.14(0.97, 1.33) | 0.0336 | 0.0156 | 0.0135 | 0.7733 |
| RS10491527 | 09: 121216614 | | 0.0168 | T,T: 2.33(1.25, 4.35) | 0.038 | T: 1.22(1.01, 1.47) | 0.0131 | 0.4922 | 0.0821 | 0.0237 |
| RS4837693 | 09: 121812351 | | 0.0151 | C,C: 1.31(1.08, 1.58) | 0.0044 | C: 1.22(1.07, 1.41) | 0.0708 | 0.0430 | 0.1539 | 0.0500 |
| RS1537282 | 09: 122164325 | | 0.0353 | A,A: 1.4(1.08, 1.82) | 0.0416 | A: 1.15(1.01, 1.32) | 0.0438 | 0.6162 | 0.0514 | 0.0170 |
| RS2535741 | 09: 124596539 | | 0.0453 | !A,G: 1.32(1.06, 1.65) | 0.0246 | G: 1.25(1.03, 1.51) | 0.0411 | 0.3967 | 0.0667 | 0.0253 |
| RS2799466 | 09: 125587911 | DENND1A | 0.0006 | C,C: 4.73(1.49, 15.03) | 0.5726 | T: 1.08(0.84, 1.38) | 0.0119 | 0.8667 | 0.0370 | 0.4510 |
| RS7873555 | 09: 131586137 | | 0.0013 | !T,T: 1.42(1.17, 1.71) | 0.0014 | C: 1.27(1.1, 1.46) | 0.0345 | 0.0172 | 0.0334 | 0.0367 |
| RS12551315 | 09: 131742462 | FNBP1 | 0.043 | !A,G: 1.35(1.01, 1.8) | 0.2464 | A: 1.18(0.9, 1.54) | 0.0754 | 0.0246 | 0.0335 | 0.6345 |
| RS2991274 | 09: 132377530 | | 0.0085 | G,G: 1.41(1.13, 1.76) | 0.0037 | G: 1.35(1.1, 1.65) | 0.0898 | 0.0454 | 0.0979 | 0.0425 |
| RS7026553 | 09: 132462568 | FUBP3 | 0.0005 | A,A: 1.76(1.3, 2.39) | 0.0003 | A: 1.7(1.27, 2.28) | 0.0295 | 0.0180 | 0.0093 | 0.0097 |
| RS11792889 | 09: 132769269 | FIBCD1 | 0.0021 | G,G: 1.93(1.32, 2.83) | 0.0553 | G: 1.16(1, 1.35) | 0.0101 | 0.9558 | 0.0112 | 0.0057 |
| RS7074221 | 10: 000403440 | DIP2C | 0.0118 | !C,T: 1.33(1.1, 1.6) | 0.9731 | C: 1(0.88, 1.15) | 0.0079 | 0.0584 | 0.0271 | 0.0600 |
| RS11250372 | 10: 001297456 | ADARB2 | 0.0034 | A,A: 1.37(1.11, 1.68) | 0.001 | A: 1.36(1.13, 1.62) | 0.0123 | 0.0142 | 0.0638 | 0.0302 |
| RS10794799 | 10: 001935387 | | 0.0022 | C,C: 1.36(1.12, 1.64) | 0.0004 | C: 1.28(1.12, 1.47) | 0.0442 | 0.0111 | 0.0531 | 0.0169 |
| RS1475420 | 10: 006738102 | | 0.0025 | !A,A: 1.83(1.21, 2.76) | 0.0012 | G: 1.3(1.11, 1.51) | 0.0776 | 0.0270 | 0.0312 | 0.0229 |
| RS12764177 | 10: 006832573 | | 0.0002 | A,C: 1.44(1.17, 1.76) | 0.4527 | C: 1.07(0.91, 1.26) | 0.0080 | 1.0000 | 0.0243 | 0.2864 |
| RS955921 | 10: 009465912 | | 0.0049 | A,G: 1.47(1.16, 1.86) | 0.0043 | G: 1.36(1.1, 1.67) | 0.0665 | 0.0493 | 0.0719 | 0.0459 |
| RS1414395 | 10: 013374142 | PHYH | 0.003 | T,T: 1.4(1.15, 1.69) | 0.0062 | T: 1.21(1.06, 1.39) | 0.0559 | 0.0330 | 0.0466 | 0.0809 |
| RS17631759 | 10: 014002167 | FRMD4A | 0.0184 | A,A: 1.38(1.1, 1.72) | 0.005 | A: 1.34(1.09, 1.65) | 0.0982 | 0.0470 | 0.1075 | 0.0394 |
| RS2457857 | 10: 014100685 | FRMD4A | 0.0039 | !A,A: 1.37(1.13, 1.67) | 0.0007 | C: 1.34(1.13, 1.59) | 0.0839 | 0.0228 | 0.0443 | 0.0104 |
| RS6602180 | 10: 017232368 | DNMT2 | 0.0126 | C,C: 1.4(1.12, 1.76) | 0.0092 | C: 1.2(1.05, 1.36) | 0.0713 | 0.0222 | 0.0440 | 0.1448 |
| RS11015065 | 10: 018928375 | NSUN6 | 0.0011 | T,T: 1.58(1.2, 2.08) | 0.0003 | T: 1.61(1.24, 2.09) | 0.0106 | 0.0040 | 0.0737 | 0.0279 |
| RS4636555 | 10: 018943611 | NSUN6 | 0.0078 | !A,A: 1.36(1.12, 1.66) | 0.0044 | C: 1.22(1.06, 1.39) | 0.0479 | 0.0229 | 0.0212 | 0.0854 |
| RS7895709 | 10: 018945941 | NSUN6 | 0.0064 | !C,C: 1.37(1.13, 1.66) | 0.0047 | T: 1.22(1.06, 1.39) | 0.0477 | 0.0174 | 0.0247 | 0.1033 |
| RS7901343 | 10: 032029614 | | 0.0254 | A,G: 1.3(1.07, 1.57) | 0.2193 | A: 1.1(0.95, 1.27) | 0.1393 | 0.0436 | 0.0066 | 0.9189 |
| RS733394 | 10: 049618247 | C10ORF64 | 0.0004 | C,C: 1.5(1.22, 1.85) | 0.0002 | C: 1.29(1.13, 1.47) | 0.0601 | 0.0410 | 0.0057 | 0.0016 |
| RS4838650 | 10: 049664276 | C10ORF64 | 0.0362 | G,G: 2.04(1.08, 3.86) | 0.887 | G: 1.02(0.84, 1.22) | 0.0432 | 0.0606 | 0.0357 | 0.0589 |
| RS12264297 | 10: 049695610 | | 0.0012 | C,C: 2.83(1.39, 5.76) | 0.0007 | C: 1.41(1.16, 1.73) | 0.0357 | 0.0265 | 0.0286 | 0.0106 |
| RS3849150 | 10: 049779229 | WDFY4 | 0.0022 | !T,T: 1.42(1.15, 1.77) | 0.0004 | C: 1.42(1.17, 1.72) | 0.0212 | 0.0073 | 0.0486 | 0.0189 |
| RS2466756 | 10: 050046112 | C10ORF128 | 0.0041 | !T,T: 1.37(1.13, 1.65) | 0.0017 | G: 1.25(1.09, 1.44) | 0.0853 | 0.0365 | 0.0515 | 0.0211 |
| RS2454566 | 10: 053403551 | PRKG1 | 0.0282 | !G,T: 1.28(1.06, 1.55) | 0.1222 | G: 1.12(0.97, 1.28) | 0.0472 | 0.0156 | 0.0406 | 0.8832 |
| RS12414156 | 10: 056612592 | | 0.0007 | !A,G: 1.49(1.21, 1.84) | 0.0021 | A: 1.32(1.11, 1.56) | 0.0485 | 0.0143 | 0.0047 | 0.0676 |
| RS3851256 | 10: 060363423 | | 0.0753 | T,T: 1.22(1.01, 1.47) | 0.0228 | T: 1.19(1.03, 1.38) | 0.0075 | 0.0834 | 0.0209 | 0.1475 |
| RS2664328 | 10: 064440254 | | 0.0005 | !C,C: 3.53(1.76, 7.06) | 0.081 | T: 1.18(0.98, 1.41) | 0.0031 | 0.0044 | 0.0130 | 0.7501 |
| RS4746906 | 10: 071185736 | | 0.0044 | !C,C: 2.27(1.32, 3.91) | 0.0056 | T: 1.29(1.08, 1.53) | 0.0911 | 0.0289 | 0.0083 | 0.0897 |
| RS11593153 | 10: 071269048 | COL13A1 | 0.0004 | !A,A: 1.47(1.19, 1.8) | 7.13E−05 | C: 1.44(1.2, 1.73) | 0.0506 | 0.0224 | 0.0053 | 0.0009 |
| RS4747096 | 10: 072188015 | ADAMTS14 | 0.0128 | G,G: 2.38(1.31, 4.35) | 0.1126 | G: 1.16(0.97, 1.38) | 0.0038 | 0.8945 | 0.1094 | 0.0403 |
| RS7098277 | 10: 076100549 | ADK | 0.0309 | G,G: 1.29(1.07, 1.55) | 0.012 | G: 1.22(1.05, 1.42) | 0.0570 | 0.0451 | 0.0405 | 0.1312 |
| RS7097101 | 10: 078837535 | KCNMA1 | 0.0147 | !A,A: 1.34(1.07, 1.68) | 0.0046 | G: 1.34(1.1, 1.64) | 0.0536 | 0.0272 | 0.0237 | 0.1004 |
| RS2349157 | 10: 085169216 | | 0.0009 | !A,A: 4.06(1.71, 9.66) | 0.5048 | G: 1.07(0.88, 1.31) | 0.0156 | 0.2868 | 0.0473 | 1.0000 |
| RS7394286 | 10: 088739172 | KIAA1975, | 0.03 | A,A: 1.29(1.07, 1.56) | 0.0248 | A: 1.17(1.02, 1.35) | 0.0847 | 0.0410 | 0.0086 | 0.2170 |
| | | LOC643150 | | | | | | | | |
| RS816278 | 10: 096107550 | NOC3L | 0.0041 | A,G: 1.31(1.09, 1.58) | 0.6975 | G: 1.03(0.9, 1.18) | 0.0238 | 0.3826 | 0.0425 | 0.1721 |
| RS1035209 | 10: 101335356 | | 0.0002 | !C,T: 1.52(1.24, 1.86) | 0.0097 | T: 1.25(1.06, 1.48) | 0.0278 | 0.2275 | 0.0043 | 0.0217 |
| RS2273696 | 10: 102249696 | SEC31L2 | 0.0015 | T,T: 1.37(1.13, 1.65) | 0.0004 | T: 1.29(1.12, 1.49) | 0.0832 | 0.0296 | 0.0173 | 0.0051 |
| RS10509792 | 10: 107314115 | | 0.0096 | G,G: 1.93(1.09, 3.42) | 0.7515 | T: 1.03(0.86, 1.23) | 0.0146 | 0.0392 | 0.0014 | 0.0108 |
| RS2475176 | 10: 114610872 | | 0.0008 | !C,C: 1.39(1.15, 1.68) | 0.0002 | A: 1.35(1.15, 1.57) | 0.0701 | 0.0221 | 0.0070 | 0.0025 |
| RS1327694 | 10: 114627535 | | 0.0008 | A,A: 1.67(1.21, 2.32) | 0.0003 | A: 1.3(1.13, 1.5) | 0.0384 | 0.0366 | 0.0095 | 0.0034 |
| RS11595716 | 10: 114633926 | | 0.0003 | !T,T: 1.37(1.14, 1.66) | 7.67E−05 | C: 1.33(1.16, 1.54) | 0.0177 | 0.0106 | 0.0086 | 0.0029 |
| RS9328842 | 10: 124755847 | ZNFN1A5 | 0.0154 | !C,T: 1.32(1.09, 1.6) | 0.2293 | C: 1.09(0.95, 1.26) | 0.0985 | 0.0400 | 0.0081 | 0.7621 |
| RS10901639 | 10: 128263139 | | 0.0241 | A,A: 1.33(1.06, 1.68) | 0.0076 | A: 1.33(1.08, 1.65) | 0.0144 | 0.0122 | 0.0436 | 0.2149 |
| RS6482689 | 10: 130291187 | | 0.0101 | A,A: 4.73(1.49, 15.03) | 0.1394 | A: 1.19(0.95, 1.49) | 0.0028 | 1.0000 | 0.0977 | 0.0361 |
| RS9419165 | 10: 133708757 | | 0.0258 | A,A: 3.37(1.3, 8.77) | 0.273 | A: 1.12(0.92, 1.36) | 0.0584 | 0.0367 | 0.0396 | 0.6284 |
| RS4074794 | 11: 000399815 | SIGIRR | 0.0041 | T,T: 1.39(1.13, 1.71) | 0.001 | T: 1.36(1.13, 1.63) | 0.0901 | 0.0304 | 0.0548 | 0.0173 |
| RS3059 | 11: 000830319 | CD151, | 0.0032 | G,G: 1.68(1.23, 2.3) | 0.0047 | G: 1.23(1.07, 1.42) | 0.1057 | 0.0350 | 0.0088 | 0.0728 |
| | | POLR2L | | | | | | | | |
| RS10794350 | 11: 000936070 | AP2A2 | 1.14E−04 | G,G: 1.56(1.26, 1.92) | 7.66E−05 | G: 1.31(1.14, 1.49) | 0.0020 | 0.0101 | 0.0097 | 0.0026 |
| RS6421967 | 11: 000973966 | AP2A2 | 0.0002 | A,A: 1.49(1.23, 1.81) | 0.0003 | A: 1.29(1.12, 1.48) | 0.0155 | 0.0802 | 0.0024 | 0.0008 |
| RS4077293 | 11: 000986522 | AP2A2 | 0.0022 | !T,T: 1.33(1.1, 1.6) | 0.0006 | C: 1.28(1.11, 1.47) | 0.0240 | 0.0141 | 0.0537 | 0.0200 |
| RS4076950 | 11: 001005021 | LOC642724, | 0.0037 | !T,T: 1.33(1.1, 1.61) | 0.0009 | C: 1.27(1.1, 1.46) | 0.0715 | 0.0261 | 0.0505 | 0.0162 |
| | | MUC6 | | | | | | | | |
| RS11246386 | 11: 001034938 | | 0.0006 | C,C: 1.44(1.19, 1.74) | 0.0014 | C: 1.29(1.1, 1.5) | 0.0089 | 0.0127 | 0.0615 | 0.0462 |
| RS1386153 | 11: 010986908 | | 0.0019 | !C,T: 1.39(1.15, 1.67) | 0.0126 | T: 1.2(1.04, 1.38) | 0.0195 | 0.1607 | 0.0569 | 0.0388 |
| RS4757289 | 11: 015112898 | | 0.017 | !C,C: 1.31(1.07, 1.6) | 0.0873 | T: 1.13(0.98, 1.29) | 0.0097 | 0.8831 | 0.0825 | 0.0280 |
| RS6486334 | 11: 016972133 | PLEKHA7 | 0.003 | !T,T: 1.38(1.14, 1.66) | 0.0009 | C: 1.27(1.1, 1.46) | 0.0398 | 0.0106 | 0.0748 | 0.0350 |
| RS214097 | 11: 017248075 | | 0.0012 | C,C: 1.61(1.24, 2.07) | 0.005 | C: 1.21(1.06, 1.39) | 0.0039 | 0.0018 | 0.0476 | 0.3430 |
| RS2072231 | 11: 017475485 | USH1C | 0.0005 | !G,G: 6.79(1.79, 25.77) | 0.8675 | G: 1.02(0.82, 1.27) | 0.0237 | 0.6255 | 0.0228 | 0.8792 |
| RS7946133 | 11: 017748398 | KCNC1 | 0.0027 | T,T: 1.44(1.15, 1.8) | 0.0009 | T: 1.42(1.16, 1.74) | 0.0494 | 0.0167 | 0.0599 | 0.0238 |
| RS1015896 | 11: 017839962 | SERGEF | 0.0013 | C,C: 1.41(1.15, 1.73) | 0.0003 | C: 1.28(1.12, 1.46) | 0.0376 | 0.0125 | 0.0340 | 0.0126 |
| RS10833367 | 11: 020598084 | SLC6A5 | 0.0008 | !C,T: 1.38(1.15, 1.67) | 0.4714 | C: 1.05(0.92, 1.2) | 0.0319 | 0.9607 | 0.0191 | 0.3634 |
| RS1531005 | 11: 021283735 | NELL1 | 0.0363 | !G,G: 1.25(1.02, 1.53) | 0.0099 | A: 1.19(1.04, 1.36) | 0.0048 | 0.0222 | 0.0297 | 0.1851 |
| RS4335544 | 11: 021286758 | NELL1 | 0.0381 | !A,A: 1.28(1.05, 1.56) | 0.0127 | C: 1.19(1.04, 1.36) | 0.0476 | 0.0503 | 0.0178 | 0.1270 |
| RS1871953 | 11: 023149253 | | 0.0004 | C,C: 3.74(1.45, 9.64) | 0.3783 | T: 1.1(0.9, 1.35) | 0.0069 | 0.6090 | 0.0134 | 0.4631 |
| RS11027043 | 11: 023169469 | | 0.0016 | C,C: 3.9(1.38, 11.03) | 0.5708 | T: 1.07(0.85, 1.33) | 0.0401 | 0.7426 | 0.0194 | 0.6949 |
| RS1982099 | 11: 028993215 | | 0.0093 | !C,C: 1.32(1.1, 1.6) | 0.0024 | A: 1.26(1.08, 1.46) | 0.0875 | 0.0290 | 0.1189 | 0.0446 |
| RS262418 | 11: 035981694 | LDLRAD3 | 0.0003 | T,T: 27.06(1.61, 455.94) | 0.0157 | T: 1.36(1.06, 1.75) | 0.0316 | 0.3351 | 0.0096 | 0.0165 |
| RS2173553 | 11: 036048464 | LDLRAD3 | 0.0302 | C,C: 1.27(1.05, 1.53) | 0.0108 | C: 1.21(1.05, 1.4) | 0.0046 | 0.0148 | 0.0318 | 0.2572 |
| RS331455 | 11: 036497125 | | 0.0004 | !T,T: 2.02(1.41, 2.89) | 0.0113 | C: 1.21(1.04, 1.41) | 0.0382 | 0.2165 | 0.0082 | 0.0192 |
| RS4755854 | 11: 044605807 | | 3.57E−05 | !C,T: 1.54(1.27, 1.86) | 0.1065 | C: 1.12(0.98, 1.28) | 0.0194 | 0.2766 | 0.0015 | 0.2331 |
| RS7124245 | 11: 044900176 | TSPAN18 | 0.0003 | C,C: 3.76(1.75, 8.06) | 0.5577 | C: 1.06(0.88, 1.29) | 0.0059 | 0.9455 | 0.0396 | 0.4849 |
| RS10769258 | 11: 047347615 | SPI1 | 0.0054 | C,C: 1.32(1.1, 1.6) | 0.0013 | C: 1.26(1.1, 1.45) | 0.0942 | 0.0314 | 0.0534 | 0.0162 |
| RS755553 | 11: 047388879 | SLC39A13 | 0.0036 | C,C: 1.35(1.12, 1.63) | 0.0009 | C: 1.27(1.1, 1.46) | 0.0585 | 0.0184 | 0.0595 | 0.0186 |
| RS1015195 | 11: 056300593 | GENE_FOR_TAR92809 | 2.54E−05 | T,T: 2.37(1.56, 3.6) | 7.22E−05 | T: 1.36(1.17, 1.58) | 0.0146 | 0.0064 | 0.0013 | 0.0046 |
| RS518385 | 11: 056301321 | GENE_FOR_TAR92809 | 0.0182 | !T,T: 1.36(1.09, 1.7) | 0.0094 | C: 1.19(1.05, 1.36) | 0.0413 | 0.1454 | 0.0864 | 0.0308 |
| RS569798 | 11: 056308204 | | 0.0024 | !G,G: 1.57(1.21, 2.03) | 0.0039 | A: 1.22(1.07, 1.4) | 0.0484 | 0.2734 | 0.0146 | 0.0037 |
| RS1788998 | 11: 056344551 | GENE_FOR_TAR92812 | 0.0024 | !C,C: 1.57(1.21, 2.03) | 0.0038 | A: 1.22(1.07, 1.4) | 0.0291 | 0.2315 | 0.0197 | 0.0049 |
| RS1943624 | 11: 056349021 | | 6.89E−05 | !A,A: 1.61(1.29, 2.01) | 5.90E−05 | G: 1.31(1.15, 1.5) | 0.0133 | 0.0174 | 0.0042 | 0.0012 |
| RS764855 | 11: 056366988 | | 0.0009 | !C,C: 1.44(1.18, 1.76) | 0.0003 | A: 1.28(1.12, 1.46) | 0.0193 | 0.0109 | 0.0382 | 0.0104 |
| RS1947298 | 11: 057728736 | OR1S2, | 0.0284 | !A,A: 1.47(1.1, 1.97) | 0.022 | G: 1.18(1.02, 1.36) | 0.0153 | 0.2341 | 0.1292 | 0.0474 |
| | | GENE_FOR_TAR92825 | | | | | | | | |
| RS7109608 | 11: 061960452 | AHNAK | 0.0057 | C,T: 1.38(1.12, 1.7) | 0.1063 | C: 1.15(0.97, 1.37) | 0.0309 | 0.0091 | 0.0026 | 0.8061 |
| RS7122303 | 11: 068782158 | | 0.0028 | C,T: 1.44(1.17, 1.78) | 0.0121 | C: 1.26(1.05, 1.51) | 0.0361 | 0.2481 | 0.0262 | 0.0192 |
| RS1939469 | 11: 075913868 | C11ORF30 | 0.0276 | !C,C: 1.32(1.07, 1.62) | 0.0084 | T: 1.28(1.07, 1.54) | 0.0131 | 0.0283 | 0.0296 | 0.1225 |
| RS2372897 | 11: 077420474 | | 0.0002 | A,A: 1.43(1.17, 1.74) | 6.28E−05 | A: 1.32(1.15, 1.51) | 0.0036 | 0.0011 | 0.0436 | 0.0160 |
| RS230669 | 11: 077553034 | | 1.08E−05 | C,C: 1.57(1.29, 1.9) | 2.89E−06 | C: 1.39(1.21, 1.59) | 0.0005 | 0.0005 | 0.0071 | 0.0016 |
| RS1015197 | 11: 092490826 | | 0.0049 | T,T: 4.35(1.56, 12.19) | 0.0465 | T: 1.23(1, 1.52) | 0.0056 | 0.7549 | 0.0429 | 0.0162 |
| RS649944 | 11: 094006944 | | 0.0132 | !A,G: 1.28(1.07, 1.55) | 0.1772 | G: 1.1(0.96, 1.25) | 0.0656 | 0.0329 | 0.0149 | 0.9251 |
| RS528098 | 11: 095594860 | MAML2 | 0.0113 | !T,T: 1.3(1.06, 1.58) | 0.0052 | C: 1.28(1.08, 1.53) | 0.0228 | 0.0227 | 0.0066 | 0.1235 |
| RS562833 | 11: 095599955 | MAML2 | 0.0136 | !C,C: 1.33(1.09, 1.62) | 0.0048 | T: 1.29(1.08, 1.54) | 0.0187 | 0.0191 | 0.0441 | 0.1496 |
| RS682837 | 11: 102558888 | DYNC2H1 | 0.0061 | !T,T: 1.46(1.12, 1.91) | 0.0034 | C: 1.46(1.13, 1.88) | 0.0876 | 0.0364 | 0.0492 | 0.0502 |
| RS1791459 | 11: 105925316 | | 0.0014 | A,A: 1.46(1.18, 1.79) | 0.0003 | A: 1.39(1.16, 1.67) | 0.0629 | 0.0234 | 0.0229 | 0.0064 |
| RS585287 | 11: 113549649 | ZBTB16 | 0.0299 | !T,T: 1.29(1.07, 1.55) | 0.0141 | C: 1.2(1.04, 1.38) | 0.0493 | 0.0320 | 0.0286 | 0.1997 |
| RS560195 | 11: 113651461 | | 0.0201 | G,G: 1.47(1.11, 1.95) | 0.0088 | G: 1.43(1.1, 1.86) | 0.0261 | 0.0972 | 0.1255 | 0.0424 |
| RS12361635 | 11: 130186012 | | 0.0023 | A,G: 1.47(1.18, 1.82) | 0.0033 | A: 1.33(1.1, 1.6) | 0.0352 | 0.0967 | 0.0302 | 0.0114 |
| RS7943936 | 11: 130214277 | | 0.0003 | !T,T: 1.51(1.21, 1.87) | 6.85E−05 | C: 1.31(1.15, 1.5) | 0.0149 | 0.0057 | 0.0162 | 0.0042 |
| RS7943757 | 11: 130221367 | LOC399978 | 0.0003 | !C,C: 1.47(1.17, 1.86) | 7.60E−05 | T: 1.31(1.15, 1.49) | 0.0171 | 0.0056 | 0.0176 | 0.0047 |
| RS7119425 | 11: 130238395 | LOC399978 | 0.0008 | !C,C: 1.48(1.17, 1.88) | 0.0002 | T: 1.29(1.13, 1.48) | 0.0066 | 0.0017 | 0.0469 | 0.0296 |
| RS6590535 | 11: 130274243 | SNX19 | 0.0042 | !T,T: 1.39(1.14, 1.69) | 0.0013 | C: 1.33(1.12, 1.57) | 0.0385 | 0.0130 | 0.1238 | 0.0465 |
| RS574322 | 11: 131326210 | HNT | 0.0037 | T,T: 1.43(1.16, 1.78) | 0.0009 | T: 1.39(1.14, 1.68) | 0.0640 | 0.0386 | 0.0092 | 0.0114 |
| RS3099792 | 11: 131612962 | HNT | 0.005 | !C,T: 1.36(1.13, 1.64) | 0.1271 | T: 1.11(0.97, 1.27) | 0.0099 | 0.9208 | 0.0780 | 0.0422 |
| RS4482038 | 11: 132422822 | OPCML | 0.0006 | A,A: 1.62(1.26, 2.09) | 0.0004 | A: 1.53(1.21, 1.94) | 0.0165 | 0.0166 | 0.0288 | 0.0092 |
| RS7126587 | 11: 132433645 | OPCML | 1.28E−04 | G,G: 1.6(1.28, 2) | 2.82E−05 | G: 1.53(1.26, 1.87) | 0.0540 | 0.0247 | 0.0020 | 0.0004 |
| RS3920986 | 11: 132653665 | OPCML | 0.0335 | !T,T: 1.69(1.13, 2.53) | 0.0574 | G: 1.16(1, 1.36) | 0.0867 | 0.0314 | 0.0154 | 0.5686 |
| RS2041382 | 12: 004824812 | | 0.0031 | !A,A: 1.4(1.15, 1.7) | 0.0075 | G: 1.2(1.05, 1.38) | 0.0175 | 0.0935 | 0.0929 | 0.0401 |
| RS10492062 | 12: 004907076 | | 0.003 | A,G: 1.48(1.15, 1.92) | 0.0616 | G: 1.25(0.99, 1.57) | 0.0442 | 0.9357 | 0.0175 | 0.0111 |
| RS2969097 | 12: 008782794 | FAM80B | 0.0048 | C,C: 1.56(1.19, 2.04) | 0.014 | C: 1.19(1.04, 1.36) | 0.0015 | 0.0004 | 0.0487 | 0.8831 |
| RS1990479 | 12: 008802742 | FAM80B | 0.0132 | G,G: 1.34(1.1, 1.63) | 0.0084 | G: 1.2(1.05, 1.37) | 0.0065 | 0.0021 | 0.0386 | 0.5371 |
| RS7979266 | 12: 010124344 | CLEC1A | 0.0421 | !G,G: 1.72(1.11, 2.67) | 0.2939 | A: 1.09(0.93, 1.28) | 0.1110 | 0.0453 | 0.0183 | 0.6576 |
| RS2541128 | 12: 011708010 | ETV6 | 0.0187 | !A,A: 2.2(1.05, 4.61) | 0.6177 | A: 1.05(0.86, 1.28) | 0.0474 | 0.0174 | 0.0018 | 0.1105 |
| RS1861482 | 12: 011724925 | ETV6 | 0.0113 | !G,G: 2.19(1.08, 4.44) | 0.5861 | G: 1.06(0.87, 1.28) | 0.0173 | 0.0071 | 0.0011 | 0.0608 |
| RS7132921 | 12: 012632336 | | 0.0016 | !C,T: 1.39(1.15, 1.68) | 0.0753 | T: 1.13(0.99, 1.29) | 0.0278 | 0.1856 | 0.0488 | 0.2323 |
| RS10841260 | 12: 019597931 | | 0.0102 | !C,T: 1.32(1.09, 1.59) | 0.0777 | T: 1.13(0.99, 1.3) | 0.0064 | 0.0031 | 0.0338 | 0.7335 |
| RS952658 | 12: 020756568 | SLCO1C1 | 0.0015 | !T,T: 1.4(1.16, 1.7) | 0.0003 | C: 1.35(1.15, 1.58) | 0.0068 | 0.0013 | 0.1511 | 0.0497 |
| RS10842096 | 12: 023067472 | | 0.0027 | !T,T: 1.39(1.15, 1.68) | 0.0013 | G: 1.29(1.1, 1.5) | 0.0739 | 0.0427 | 0.0317 | 0.0121 |
| RS472100 | 12: 024370761 | SOX5 | 0.0081 | C,C: 1.3(1.06, 1.59) | 0.0051 | C: 1.29(1.08, 1.54) | 0.0615 | 0.0413 | 0.1025 | 0.0407 |
| RS2955479 | 12: 024583650 | SOX5 | 0.0032 | A,A: 2.41(1.37, 4.24) | 0.0056 | A: 1.28(1.08, 1.53) | 2.10E−04 | 3.97E−05 | 0.0426 | 1.0000 |
| RS1487657 | 12: 024613521 | FLJ32894 | 0.0017 | !T,T: 1.39(1.15, 1.68) | 0.0004 | G: 1.28(1.12, 1.47) | 0.0136 | 0.0034 | 0.0866 | 0.0400 |
| RS10505945 | 12: 024803300 | | 0.0027 | !G,G: 1.83(1.29, 2.61) | 0.0089 | A: 1.22(1.05, 1.42) | 0.0618 | 0.0224 | 0.0190 | 0.1536 |
| RS7304591 | 12: 044769621 | | 0.0388 | !G,G: 3.95(1.21, 12.85) | 0.218 | T: 1.17(0.92, 1.49) | 0.0568 | 0.0245 | 0.0274 | 0.6623 |
| RS10506265 | 12: 044802761 | | 0.0012 | T,T: 1.65(1.22, 2.23) | 0.0005 | T: 1.67(1.25, 2.23) | 0.0132 | 0.0087 | 0.0744 | 0.0274 |
| RS10875798 | 12: 047019151 | ZNF641 | 0.0129 | T,T: 1.32(1.1, 1.6) | 0.0111 | T: 1.21(1.05, 1.39) | 0.0075 | 0.3320 | 0.0414 | 0.0134 |
| RS2272306 | 12: 051840550 | CSAD | 0.0072 | !A,A: 1.31(1.06, 1.63) | 0.0046 | G: 1.32(1.09, 1.61) | 0.0852 | 0.0334 | 0.0242 | 0.0613 |
| RS4422358 | 12: 054155985 | | 0.0021 | A,A: 1.38(1.13, 1.67) | 0.0006 | A: 1.27(1.11, 1.45) | 0.0325 | 0.0129 | 0.0567 | 0.0196 |
| RS894010 | 12: 060670094 | FAM19A2 | 0.0022 | G,G: 2.64(1.29, 5.4) | 0.0014 | G: 1.37(1.13, 1.65) | 0.0082 | 0.0029 | 0.0120 | 0.1138 |
| RS1245653 | 12: 060886900 | | 0.0081 | G,G: 3.22(1.11, 9.31) | 0.4171 | A: 1.09(0.89, 1.35) | 0.0228 | 0.0620 | 0.0212 | 0.5134 |
| RS1565730 | 12: 065143477 | GRIP1 | 0.004 | A,G: 1.29(1.07, 1.57) | 0.9696 | A: 1(0.86, 1.17) | 0.0413 | 0.2801 | 0.0218 | 0.2377 |
| RS11615839 | 12: 069133885 | | 0.0312 | A,G: 1.32(1.07, 1.62) | 0.0884 | G: 1.16(0.98, 1.38) | 0.0331 | 0.0113 | 0.0103 | 1.0000 |
| RS11115162 | 12: 080950597 | | 0.0012 | !C,T: 1.43(1.18, 1.75) | 0.0036 | T: 1.27(1.08, 1.49) | 0.0357 | 0.0132 | 0.0146 | 0.1070 |
| RS10777084 | 12: 086904225 | C12ORF50 | 0.0018 | !C,C: 5.44(1.73, 17.12) | 0.0214 | T: 1.28(1.04, 1.58) | 0.0016 | 0.4792 | 0.0403 | 0.0159 |
| RS11107018 | 12: 092338033 | UBE2N | 0.0013 | !G,G: 2.76(1.5, 5.09) | 0.0062 | A: 1.27(1.07, 1.51) | 0.0417 | 0.0293 | 0.0263 | 0.0986 |
| RS1319893 | 12: 097960713 | ANKS1B | 0.0055 | !G,T: 1.34(1.11, 1.62) | 0.7777 | G: 1.02(0.89, 1.17) | 0.0168 | 0.0622 | 0.0180 | 0.1818 |
| RS10860593 | 12: 099351399 | | 0.0004 | !T,T: 1.59(1.26, 2) | 0.0002 | C: 1.49(1.2, 1.84) | 0.0999 | 0.0316 | 0.0006 | 0.0028 |
| RS11110385 | 12: 099376836 | | 0.0006 | T,T: 1.4(1.15, 1.69) | 1.20E−04 | T: 1.31(1.14, 1.5) | 0.0046 | 0.0010 | 0.0983 | 0.0331 |
| RS7969185 | 12: 100080857 | SLC5A8 | 0.0131 | A,G: 1.26(1.03, 1.56) | 0.6224 | G: 1.05(0.88, 1.25) | 0.0463 | 0.0320 | 0.0211 | 0.2278 |
| RS2133298 | 12: 101784764 | PAH | 0.0042 | A,A: 1.4(1.15, 1.72) | 0.0031 | A: 1.22(1.07, 1.4) | 0.0316 | 0.0145 | 0.0130 | 0.0888 |
| RS1718301 | 12: 101795323 | PAH | 0.0059 | G,G: 1.39(1.14, 1.7) | 0.0072 | G: 1.2(1.05, 1.37) | 0.0724 | 0.0312 | 0.0074 | 0.1074 |
| RS1863538 | 12: 106267905 | BTBD11 | 0.0058 | T,T: 1.39(1.13, 1.69) | 0.0038 | T: 1.22(1.07, 1.39) | 0.0181 | 0.0625 | 0.0980 | 0.0295 |
| RS1861586 | 12: 106438271 | BTBD11 | 0.0216 | !A,A: 1.5(1.12, 2.01) | 0.0281 | G: 1.17(1.02, 1.34) | 0.0062 | 0.3533 | 0.0967 | 0.0404 |
| RS4964598 | 12: 106460457 | BTBD11 | 0.0003 | !G,G: 2.39(1.53, 3.72) | 0.0093 | A: 1.23(1.05, 1.44) | 0.0023 | 0.1746 | 0.0437 | 0.0293 |
| RS6539407 | 12: 106944648 | | 0.0015 | C,C: 2.03(1.3, 3.17) | 0.5459 | C: 1.05(0.9, 1.23) | 0.0263 | 0.6000 | 0.0251 | 0.1804 |
| RS886476 | 12: 111803854 | RPH3A | 0.0055 | T,T: 1.43(1.12, 1.84) | 0.0014 | T: 1.25(1.09, 1.43) | 0.0916 | 0.0316 | 0.0473 | 0.0151 |
| RS7978582 | 12: 112035775 | RASAL1 | 0.0043 | G,G: 2.1(1.2, 3.69) | 0.0018 | G: 1.33(1.11, 1.59) | 0.0258 | 0.0126 | 0.0894 | 0.0411 |
| RS1878420 | 12: 116537177 | KSR2 | 0.0034 | !T,T: 1.43(1.15, 1.78) | 0.0017 | C: 1.24(1.08, 1.41) | 0.0348 | 0.1209 | 0.0125 | 0.0048 |
| RS4767658 | 12: 117003824 | FLJ20674 | 0.0014 | !T,T: 1.39(1.15, 1.69) | 0.0002 | C: 1.29(1.13, 1.48) | 0.0120 | 0.0079 | 0.0389 | 0.0137 |
| RS10849646 | 12: 118262051 | CCDC60 | 0.0003 | !C,T: 1.46(1.21, 1.76) | 0.5893 | C: 1.04(0.91, 1.18) | 0.0020 | 0.0077 | 0.0013 | 0.0664 |
| RS4760974 | 12: 126603427 | | 0.0065 | !T,T: 1.34(1.11, 1.63) | 0.0016 | C: 1.25(1.09, 1.43) | 0.0425 | 0.0323 | 0.0846 | 0.0298 |
| RS4411335 | 12: 128273807 | TMEM132D | 7.41E−05 | !T,T: 1.53(1.26, 1.86) | 0.0002 | C: 1.3(1.13, 1.49) | 0.0166 | 0.0098 | 0.0031 | 0.0060 |
| RS1482294 | 12: 128276744 | TMEM132D | 0.0002 | !G,G: 1.5(1.23, 1.83) | 6.50E−05 | A: 1.32(1.15, 1.5) | 0.0108 | 0.0037 | 0.0138 | 0.0052 |
| RS6486483 | 12: 128591932 | TMEM132D | 0.0008 | !G,G: 1.44(1.16, 1.79) | 0.0002 | A: 1.28(1.12, 1.46) | 0.0020 | 0.0026 | 0.0352 | 0.0307 |
| RS11831111 | 12: 131205371 | | 0.059 | !A,A: 1.25(1.02, 1.53) | 0.0208 | G: 1.17(1.03, 1.34) | 0.0008 | 0.0189 | 0.0278 | 0.3443 |
| RS6490774 | 13: 022529340 | | 0.0067 | C,T: 1.77(1.21, 2.59) | 0.0049 | C: 1.65(1.16, 2.35) | 0.0295 | 0.1217 | 0.0420 | 0.0261 |
| RS9512417 | 13: 026306722 | | 0.0114 | G,G: 1.31(1.08, 1.58) | 0.0027 | G: 1.26(1.09, 1.47) | 0.0660 | 0.0314 | 0.0915 | 0.0361 |
| RS12583235 | 13: 029655737 | | 0.059 | !A,A: 1.26(1.04, 1.52) | 0.0299 | G: 1.17(1.02, 1.34) | 0.0213 | 0.3461 | 0.0517 | 0.0284 |
| RS9508795 | 13: 030022651 | | 0.0044 | C,C: 1.32(1.09, 1.61) | 0.001 | C: 1.26(1.1, 1.44) | 0.0499 | 0.0139 | 0.0843 | 0.0274 |
| RS9529615 | 13: 034205066 | | 0.1139 | !A,A: 1.2(1, 1.46) | 0.0387 | C: 1.16(1.01, 1.33) | 0.0270 | 0.1209 | 0.0227 | 0.1699 |
| RS275966 | 13: 035184806 | | 0.01 | T,T: 1.33(1.09, 1.61) | 0.0031 | T: 1.23(1.07, 1.41) | 0.0752 | 0.0309 | 0.1048 | 0.0337 |
| RS9533366 | 13: 042527595 | DNAJC15 | 0.0094 | !A,A: 1.33(1.1, 1.6) | 0.0027 | G: 1.25(1.08, 1.45) | 0.0844 | 0.0279 | 0.0580 | 0.0471 |
| RS9533385 | 13: 042567737 | DNAJC15 | 0.0076 | !T,T: 1.34(1.11, 1.61) | 0.0024 | C: 1.26(1.09, 1.45) | 0.0980 | 0.0318 | 0.0484 | 0.0362 |
| RS9533819 | 13: 043796037 | | 0.0043 | !C,C: 1.64(1.18, 2.26) | 0.0024 | T: 1.25(1.08, 1.44) | 0.0052 | 0.0015 | 0.0291 | 0.2674 |
| RS9567459 | 13: 044145334 | | 0.0054 | A,A: 1.3(1.08, 1.57) | 0.0013 | A: 1.27(1.1, 1.48) | 0.0977 | 0.0314 | 0.0536 | 0.0215 |
| RS474863 | 13: 045958364 | | 0.0029 | !G,G: 1.99(1.23, 3.21) | 0.0011 | A: 1.32(1.12, 1.57) | 0.0305 | 0.0213 | 0.0843 | 0.0277 |
| RS842383 | 13: 046157226 | LRCH1 | 0.0062 | T,T: 1.84(1.24, 2.73) | 0.009 | T: 1.23(1.05, 1.43) | 0.0396 | 0.0108 | 0.0195 | 0.2728 |
| RS2273816 | 13: 049192057 | KPNA3 | 0.0208 | !G,G: 2.6(1.27, 5.35) | 0.311 | A: 1.1(0.91, 1.33) | 0.0025 | 0.4142 | 0.0573 | 0.0196 |
| RS9316484 | 13: 049543165 | DLEU2 | 0.0021 | !C,C: 2.2(1.32, 3.67) | 0.0024 | T: 1.29(1.09, 1.52) | 0.0888 | 0.0496 | 0.0272 | 0.0274 |
| RS4886077 | 13: 051896698 | VPS36 | 0.0017 | G,G: 1.41(1.16, 1.71) | 0.0068 | G: 1.21(1.05, 1.38) | 0.0126 | 0.0724 | 0.0788 | 0.0426 |
| RS6561671 | 13: 052080315 | LOC653891 | 0.0043 | G,G: 1.46(1.16, 1.84) | 0.0033 | G: 1.22(1.07, 1.4) | 0.0315 | 0.0720 | 0.0565 | 0.0187 |
| RS9536470 | 13: 053058723 | | 0.0455 | !A,C: 1.28(1.05, 1.56) | 0.149 | A: 1.13(0.96, 1.33) | 0.0436 | 0.0199 | 0.0473 | 0.8566 |
| RS4322543 | 13: 056500910 | | 8.70E−05 | !A,A: 2.14(1.43, 3.2) | 1.15E−04 | G: 1.35(1.16, 1.56) | 0.0255 | 0.0108 | 0.0033 | 0.0051 |
| RS577241 | 13: 056596687 | | 1.21E−04 | !A,A: 2.07(1.38, 3.11) | 1.14E−04 | G: 1.35(1.16, 1.56) | 0.0408 | 0.0151 | 0.0024 | 0.0036 |
| RS9570776 | 13: 062481608 | | 0.0417 | C,C: 1.31(1.06, 1.63) | 0.0986 | C: 1.12(0.98, 1.28) | 0.1219 | 0.0469 | 0.0146 | 0.7781 |
| RS9598990 | 13: 065001632 | | 0.0398 | A,C: 1.41(1.06, 1.87) | 0.0529 | C: 1.3(1, 1.69) | 0.0377 | 0.5791 | 0.0744 | 0.0337 |
| RS1325678 | 13: 066726347 | | 0.0007 | T,T: 1.56(1.24, 1.96) | 0.0018 | T: 1.24(1.08, 1.41) | 0.0281 | 0.0512 | 0.0205 | 0.0127 |
| RS7983142 | 13: 066740589 | | 0.0005 | T,T: 1.79(1.32, 2.42) | 0.0011 | T: 1.27(1.1, 1.46) | 0.0211 | 0.0168 | 0.0198 | 0.0255 |
| RS1146878 | 13: 075772974 | | 0.0041 | G,G: 1.45(1.15, 1.83) | 0.0016 | G: 1.24(1.08, 1.42) | 0.0511 | 0.0161 | 0.0498 | 0.0343 |
| RS9546644 | 13: 083751649 | | 0.0025 | !C,C: 1.38(1.11, 1.71) | 0.0005 | T: 1.26(1.11, 1.44) | 0.0039 | 0.0056 | 0.0076 | 0.0271 |
| RS3861162 | 13: 083801107 | | 0.0045 | !C,C: 1.39(1.12, 1.73) | 0.0011 | T: 1.25(1.09, 1.43) | 0.0387 | 0.0227 | 0.0037 | 0.0144 |
| RS9546953 | 13: 084412352 | | 0.0003 | !A,A: 1.86(1.36, 2.54) | 0.0199 | G: 1.18(1.03, 1.36) | 0.0215 | 0.2024 | 0.0111 | 0.0439 |
| RS4076203 | 13: 086794849 | | 0.0037 | !A,A: 2.44(1.34, 4.46) | 0.5739 | C: 1.06(0.88, 1.27) | 0.0412 | 0.3658 | 0.0360 | 0.1277 |
| RS2148229 | 13: 092281547 | GPC5 | 0.0004 | G,G: 1.52(1.21, 1.9) | 0.0426 | G: 1.15(1.01, 1.31) | 0.0081 | 0.1076 | 0.0410 | 0.1884 |
| RS9590101 | 13: 094341946 | | 0.0182 | !A,G: 1.33(1.09, 1.62) | 0.0239 | A: 1.21(1.03, 1.42) | 0.0324 | 0.3884 | 0.0887 | 0.0302 |
| RS1806507 | 13: 096836578 | MBNL2 | 0.0109 | !G,G: 1.29(1.06, 1.56) | 0.0027 | A: 1.23(1.07, 1.41) | 0.1061 | 0.0391 | 0.1107 | 0.0376 |
| RS1328657 | 13: 096981887 | | 0.0006 | !G,G: 1.4(1.16, 1.69) | 9.30E−05 | A: 1.34(1.16, 1.54) | 0.0224 | 0.0053 | 0.0282 | 0.0071 |
| RS9513260 | 13: 097079638 | | 0.0106 | !T,T: 1.3(1.08, 1.57) | 0.0021 | C: 1.25(1.08, 1.44) | 0.1405 | 0.0446 | 0.0832 | 0.0248 |
| RS4772260 | 13: 099443724 | | 0.0066 | !T,T: 1.35(1.1, 1.65) | 0.0022 | C: 1.23(1.08, 1.41) | 0.0086 | 0.0083 | 0.0219 | 0.0962 |
| RS8002928 | 13: 099743604 | PCCA | 0.0086 | G,G: 1.37(1.1, 1.69) | 0.003 | G: 1.22(1.07, 1.4) | 0.0865 | 0.0416 | 0.0882 | 0.0307 |
| RS12874523 | 13: 105487642 | | 0.0074 | !A,C: 1.39(1.13, 1.71) | 0.0127 | A: 1.25(1.05, 1.48) | 0.0498 | 0.0156 | 0.0470 | 0.2536 |
| RS9521112 | 13: 108414403 | RP11-54H7.1 | 0.0022 | !A,G: 1.36(1.07, 1.73) | 0.2765 | G: 1.13(0.91, 1.4) | 0.0062 | 0.0248 | 0.0099 | 0.5522 |
| RS9588148 | 13: 109819082 | COL4A2 | 0.0012 | T,T: 1.44(1.19, 1.76) | 0.0005 | T: 1.34(1.14, 1.59) | 0.0377 | 0.0268 | 0.0269 | 0.0089 |
| RS9521748 | 13: 109860681 | COL4A2 | 0.0008 | A,A: 1.71(1.27, 2.31) | 0.0008 | A: 1.27(1.11, 1.46) | 0.0037 | 0.0070 | 0.1040 | 0.0424 |
| RS11841484 | 13: 110771589 | C13ORF16 | 0.0021 | !T,T: 1.54(1.2, 1.98) | 0.0004 | C: 1.52(1.21, 1.92) | 0.0756 | 0.0255 | 0.0211 | 0.0064 |
| RS7140768 | 14: 019966697 | LOC123103, | 5.66E−05 | T,T: 1.81(1.39, 2.36) | 0.0009 | T: 1.26(1.1, 1.44) | 0.0017 | 0.0049 | 0.0187 | 0.0625 |
| | | LOC650481 | | | | | | | | |
| RS17277375 | 14: 019979312 | LOC441672 | 0.0029 | C,T: 1.38(1.15, 1.67) | 0.0911 | C: 1.13(0.98, 1.29) | 0.0857 | 0.0489 | 0.0044 | 0.6944 |
| RS433673 | 14: 022920742 | MYH6, | 0.0003 | !T,T: 7.21(1.91, 27.23) | 0.9571 | T: 1.01(0.82, 1.25) | 0.0068 | 0.6312 | 0.0277 | 0.5134 |
| | | CMTM5 | | | | | | | | |
| RS3825581 | 14: 023067853 | ZFHX2, | 0.0262 | G,G: 1.59(1.11, 2.26) | 0.0242 | G: 1.19(1.02, 1.38) | 0.1392 | 0.0473 | 0.0475 | 0.2267 |
| | | ZNF409 | | | | | | | | |
| RS3794485 | 14: 026129186 | NOVA1 | 0.0049 | T,T: 1.37(1.12, 1.67) | 0.0012 | T: 1.33(1.12, 1.58) | 0.1034 | 0.0464 | 0.0336 | 0.0119 |
| RS1955773 | 14: 029800649 | | 0.0017 | G,G: 1.41(1.17, 1.71) | 0.0013 | G: 1.26(1.09, 1.44) | 0.0380 | 0.0284 | 0.0444 | 0.0210 |
| RS1431062 | 14: 033752846 | LOC651341, | 0.001 | A,A: 2.04(1.38, 3.01) | 0.004 | A: 1.25(1.08, 1.46) | 0.0885 | 0.0472 | 0.0067 | 0.0478 |
| | | LOC644347 | | | | | | | | |
| RS1431063 | 14: 033753171 | LOC644347, | 0.0009 | G,G: 2.03(1.37, 3) | 0.0032 | G: 1.26(1.08, 1.47) | 0.0833 | 0.0369 | 0.0070 | 0.0423 |
| | | LOC651341 | | | | | | | | |
| RS2010608 | 14: 034289096 | | 0.0028 | A,A: 1.44(1.16, 1.8) | 0.0008 | A: 1.42(1.16, 1.73) | 0.0375 | 0.0263 | 0.0464 | 0.0180 |
| RS8015303 | 14: 034316223 | BAZ1A | 0.003 | A,A: 1.57(1.18, 2.09) | 0.0008 | A: 1.59(1.21, 2.08) | 0.0598 | 0.0443 | 0.0371 | 0.0100 |
| RS712332 | 14: 036336400 | SLC25A21 | 0.0307 | !C,T: 1.28(1.03, 1.6) | 0.2809 | C: 1.11(0.92, 1.35) | 0.0121 | 0.6289 | 0.1067 | 0.0432 |
| RS1266264 | 14: 045254345 | | 0.0177 | C,C: 1.56(1.14, 2.12) | 0.0489 | C: 1.15(1, 1.33) | 0.0994 | 0.0348 | 0.0242 | 0.4855 |
| RS981951 | 14: 045397270 | | 0.0059 | !C,C: 1.3(1.08, 1.57) | 0.0017 | T: 1.26(1.09, 1.46) | 0.0197 | 0.0144 | 0.0834 | 0.0393 |
| RS2933187 | 14: 046447470 | MAMDC1 | 0.0057 | C,C: 1.31(1.09, 1.58) | 0.0016 | C: 1.28(1.1, 1.49) | 0.0531 | 0.0163 | 0.1047 | 0.0386 |
| RS4901053 | 14: 050287324 | NIN | 0.0002 | !G,G: 1.51(1.21, 1.89) | 5.95E−05 | T: 1.31(1.15, 1.5) | 0.1025 | 0.0417 | 0.0011 | 0.0003 |
| RS7140492 | 14: 057590253 | C14ORF37 | 0.001 | !G,T: 1.8(1.3, 2.49) | 0.0008 | T: 1.65(1.23, 2.23) | 0.0315 | 0.5076 | 0.0005 | ##### |
| RS393170 | 14: 059688357 | DHRS7 | 0.0003 | T,T: 1.5(1.19, 1.89) | 1.00E−04 | T: 1.52(1.23, 1.88) | 0.0088 | 0.0025 | 0.0351 | 0.0155 |
| RS10483727 | 14: 060142628 | | 0.0163 | !C,C: 1.31(1.08, 1.6) | 0.0457 | T: 1.15(1, 1.31) | 0.0759 | 0.0299 | 0.0375 | 0.5630 |
| RS1270013 | 14: 061572437 | SYT16 | 0.0086 | !C,C: 1.32(1.09, 1.59) | 0.0024 | T: 1.27(1.09, 1.48) | 0.0835 | 0.0339 | 0.0981 | 0.0386 |
| RS4078408 | 14: 065342587 | | 0.0029 | A,G: 1.35(1.12, 1.64) | 0.5703 | G: 1.05(0.9, 1.21) | 0.0268 | 0.0615 | 0.0215 | 0.3472 |
| RS8013361 | 14: 065483143 | | 0.0002 | !C,C: 1.68(1.29, 2.2) | 8.61E−05 | T: 1.32(1.15, 1.51) | 0.0028 | 0.0009 | 0.0452 | 0.0248 |
| RS2238195 | 14: 071873833 | RGS6 | 0.0035 | C,C: 1.76(1.17, 2.65) | 0.0014 | C: 1.29(1.1, 1.51) | 0.0288 | 0.0123 | 0.1025 | 0.0429 |
| RS847330 | 14: 071899350 | RGS6 | 0.0036 | C,C: 1.66(1.18, 2.34) | 0.0015 | C: 1.27(1.1, 1.47) | 0.0460 | 0.0244 | 0.0727 | 0.0259 |
| RS1014087 | 14: 073035327 | MGC48595 | 3.57E−05 | G,G: 5.09(2.18, 11.87) | 0.2001 | G: 1.13(0.94, 1.38) | 0.0322 | 0.7797 | 0.0011 | 0.1444 |
| RS3784011 | 14: 075001425 | JDP2 | 0.0054 | !C,C: 1.33(1.1, 1.61) | 0.0014 | T: 1.26(1.09, 1.44) | 0.0337 | 0.0126 | 0.0981 | 0.0365 |
| RS11623206 | 14: 075819928 | | 0.0078 | !T,T: 2.21(1.31, 3.72) | 0.0483 | C: 1.18(1, 1.39) | 0.0267 | 0.6317 | 0.0526 | 0.0274 |
| RS850748 | 14: 076187303 | | 0.0202 | A,G: 1.31(1.08, 1.58) | 0.0962 | A: 1.13(0.98, 1.31) | 0.0294 | 0.0098 | 0.0284 | 0.8355 |
| RS1950265 | 14: 077664518 | | 0.019 | G,G: 1.37(1.08, 1.74) | 0.1852 | G: 1.1(0.96, 1.25) | 0.1003 | 0.0390 | 0.0102 | 0.9250 |
| RS8022732 | 14: 086360515 | | 0.0054 | C,C: 1.5(1.13, 2) | 0.0016 | C: 1.25(1.09, 1.44) | 0.0386 | 0.0168 | 0.0252 | 0.0388 |
| RS2254354 | 14: 086375280 | | 0.0058 | T,T: 1.43(1.12, 1.84) | 0.0017 | T: 1.24(1.08, 1.42) | 0.1130 | 0.0444 | 0.0300 | 0.0192 |
| RS10498611 | 14: 086598814 | | 0.0014 | !C,T: 1.43(1.17, 1.76) | 0.0951 | T: 1.15(0.98, 1.37) | 0.0435 | 0.1032 | 0.0248 | 0.5186 |
| RS1288709 | 14: 087291337 | | 0.0078 | !T,T: 1.94(1.19, 3.16) | 0.0045 | C: 1.27(1.08, 1.51) | 0.0232 | 0.2357 | 0.0121 | 0.0035 |
| RS737076 | 14: 095032380 | | 0.0067 | !G,G: 1.37(1.13, 1.67) | 0.0045 | A: 1.22(1.06, 1.39) | 0.0690 | 0.0199 | 0.0394 | 0.0718 |
| RS10498648 | 14: 095714790 | | 0.0068 | A,A: 1.32(1.1, 1.59) | 0.002 | A: 1.25(1.09, 1.45) | 0.0249 | 0.0150 | 0.0990 | 0.0469 |
| RS1109302 | 14: 095728883 | | 0.0018 | !T,T: 1.46(1.19, 1.81) | 0.0021 | C: 1.23(1.08, 1.41) | 0.1075 | 0.0418 | 0.0107 | 0.0213 |
| RS2400352 | 14: 098022578 | | 0.0012 | A,G: 1.58(1.22, 2.05) | 0.0061 | A: 1.39(1.1, 1.76) | 0.0458 | 0.0918 | 0.0143 | 0.0348 |
| RS7493924 | 14: 100110151 | | 0.0602 | !C,T: 1.32(1.05, 1.67) | 0.0367 | T: 1.25(1.02, 1.53) | 0.0225 | 0.4870 | 0.0722 | 0.0334 |
| RS2269318 | 14: 102478372 | CDC42BPB | 0.0005 | !A,A: 1.5(1.19, 1.9) | 0.0003 | G: 1.49(1.2, 1.86) | 0.0815 | 0.0311 | 0.0054 | 0.0040 |
| RS875014 | 14: 102748906 | | 0.0045 | A,A: 8.33(1.53, 45.44) | 0.5915 | A: 1.08(0.83, 1.4) | 0.0222 | 0.0272 | 0.0005 | 0.1157 |
| RS2841231 | 14: 104411688 | KIAA0284 | 0.009 | T,T: 1.47(1.11, 1.95) | 0.0032 | T: 1.23(1.07, 1.41) | 0.0237 | 0.0446 | 0.0722 | 0.0347 |
| RS12906657 | 15: 021878752 | | 0.0093 | !C,C: 1.36(1.11, 1.66) | 0.0188 | T: 1.17(1.03, 1.34) | 0.0373 | 0.3533 | 0.0528 | 0.0177 |
| RS3101683 | 15: 025554889 | | 0.0243 | A,A: 1.3(1.07, 1.58) | 0.011 | A: 1.2(1.04, 1.37) | 0.0648 | 0.0255 | 0.0468 | 0.1555 |
| RS1456212 | 15: 029211346 | | 0.0034 | A,A: 2.81(1.33, 5.92) | 0.0039 | A: 1.32(1.1, 1.6) | 0.0409 | 0.0740 | 0.0533 | 0.0228 |
| RS4780094 | 15: 031287357 | LOC649021 | 0.0006 | !A,A: 1.43(1.18, 1.74) | 1.01E−04 | G: 1.39(1.18, 1.65) | 0.1095 | 0.0398 | 0.0022 | 0.0006 |
| RS8037685 | 15: 035565656 | | 0.0244 | A,G: 1.36(1.08, 1.73) | 0.0889 | G: 1.2(0.98, 1.48) | 0.0350 | 0.0127 | 0.0391 | 0.9404 |
| RS10518743 | 15: 040017144 | EHD4 | 0.005 | !T,T: 1.39(1.1, 1.74) | 0.0018 | C: 1.39(1.13, 1.72) | 0.0419 | 0.0176 | 0.1114 | 0.0414 |
| RS11854484 | 15: 043332770 | SLC28A2 | 0.0077 | !C,C: 1.3(1.07, 1.57) | 0.0018 | T: 1.25(1.09, 1.43) | 0.0701 | 0.0280 | 0.0921 | 0.0377 |
| RS2444043 | 15: 053321634 | RAB27A | 0.0034 | C,C: 1.42(1.14, 1.78) | 0.0009 | C: 1.25(1.1, 1.43) | 0.0278 | 0.0075 | 0.0446 | 0.0387 |
| RS7175728 | 15: 053681850 | | 1.29E−04 | !C,C: 1.62(1.25, 2.1) | 3.52E−05 | T: 1.33(1.16, 1.53) | 0.0038 | 0.0020 | 0.0162 | 0.0042 |
| RS1509405 | 15: 054109146 | LOC440280 | 0.0384 | !A,A: 1.31(1.06, 1.61) | 0.0165 | G: 1.18(1.03, 1.34) | 0.1001 | 0.0466 | 0.0444 | 0.2031 |
| RS3191402 | 15: 057216452 | MYO1E | 0.0161 | !C,T: 1.3(1.08, 1.57) | 0.1378 | C: 1.11(0.97, 1.27) | 0.0080 | 1.0000 | 0.1076 | 0.0449 |
| RS2960807 | 15: 057957580 | | 0.0097 | G,T: 1.34(1.11, 1.63) | 0.1136 | T: 1.13(0.97, 1.31) | 0.0164 | 0.0142 | 0.0367 | 0.8315 |
| RS10519070 | 15: 058856045 | RORA | 0.0041 | !T,T: 1.44(1.16, 1.8) | 0.0013 | C: 1.39(1.14, 1.69) | 0.0664 | 0.0197 | 0.0435 | 0.0276 |
| RS4984247 | 15: 061545700 | | 0.0096 | C,T: 1.41(1.13, 1.76) | 0.0064 | C: 1.31(1.08, 1.59) | 0.0164 | 0.0906 | 0.0963 | 0.0366 |
| RS8041258 | 15: 067423676 | PAQR5 | 0.0017 | C,T: 1.84(1.25, 2.73) | 0.0178 | T: 1.55(1.08, 2.23) | 0.0160 | 0.3205 | 0.0352 | 0.0244 |
| RS4886630 | 15: 071882793 | | 0.003 | C,C: 9.01(1.66, 48.81) | 0.6167 | C: 1.06(0.85, 1.32) | 0.0443 | 0.2245 | 0.0434 | 0.5668 |
| RS950169 | 15: 082497465 | ADAMTSL3 | 4.04E−05 | G,G: 1.43(1.19, 1.73) | 1.29E−05 | G: 1.4(1.2, 1.63) | 0.0011 | 0.0006 | 0.0170 | 0.0046 |
| RS2135551 | 15: 082498855 | ADAMTSL3 | 2.32E−05 | A,A: 1.44(1.2, 1.74) | 6.83E−06 | A: 1.41(1.22, 1.64) | 0.0010 | 0.0004 | 0.0122 | 0.0033 |
| RS1911155 | 15: 082578639 | FAM42B | 9.97E−05 | A,A: 1.43(1.18, 1.72) | 2.20E−05 | A: 1.39(1.19, 1.61) | 0.0044 | 0.0015 | 0.0132 | 0.0033 |
| RS12910334 | 15: 082749322 | KIAA1920 | 0.0002 | A,A: 1.43(1.18, 1.72) | 3.58E−05 | A: 1.37(1.18, 1.59) | 0.0049 | 0.0016 | 0.0192 | 0.0049 |
| RS3743162 | 15: 083231973 | SLC28A1 | 0.016 | T,T: 1.28(1.05, 1.55) | 0.0045 | T: 1.27(1.08, 1.5) | 0.1254 | 0.0477 | 0.0855 | 0.0475 |
| RS2173091 | 15: 084598403 | | 0.0095 | !C,C: 1.38(1.12, 1.69) | 0.0047 | T: 1.3(1.09, 1.56) | 0.0693 | 0.0444 | 0.1192 | 0.0469 |
| RS11855600 | 15: 086764263 | | 0.0038 | C,C: 2.45(1.41, 4.25) | 0.0871 | C: 1.16(0.98, 1.37) | 0.0189 | 0.7077 | 0.0536 | 0.0403 |
| RS2589949 | 15: 088756019 | IQGAP1 | 0.0217 | !A,A: 1.53(1.11, 2.11) | 0.2281 | G: 1.09(0.95, 1.26) | 0.1088 | 0.0453 | 0.0178 | 0.8347 |
| RS2388814 | 15: 092874459 | | 0.0025 | !A,G: 1.32(1.08, 1.6) | 0.778 | A: 1.03(0.88, 1.2) | 0.0452 | 0.3760 | 0.0422 | 0.6595 |
| RS8044035 | 16: 003191523 | | 1.32E−05 | !A,G: 1.59(1.31, 1.93) | 0.0003 | A: 1.33(1.14, 1.56) | 0.0116 | 0.0061 | 0.0006 | 0.0169 |
| RS548927 | 16: 003197869 | | 0.0056 | A,A: 1.58(1.16, 2.15) | 0.0023 | A: 1.58(1.18, 2.13) | 0.0440 | 0.0306 | 0.0453 | 0.0377 |
| RS7201758 | 16: 008512159 | | 0.0042 | !A,C: 1.4(1.14, 1.71) | 0.0431 | A: 1.18(1.01, 1.39) | 0.0210 | 0.3769 | 0.0784 | 0.0493 |
| RS7187654 | 16: 010558868 | EMP2 | 0.0099 | A,A: 1.34(1.1, 1.64) | 0.0338 | A: 1.16(1.01, 1.32) | 0.0825 | 0.0396 | 0.0435 | 0.3379 |
| RS1548956 | 16: 010664693 | FLJ32871 | 0.038 | C,T: 1.23(1.02, 1.48) | 0.6989 | C: 1.03(0.9, 1.18) | 0.0208 | 0.0106 | 0.0028 | 0.0584 |
| RS3213646 | 16: 020717568 | EXOD1, | 0.0086 | !C,C: 1.29(1.06, 1.56) | 0.0022 | T: 1.24(1.08, 1.41) | 0.1325 | 0.0431 | 0.0611 | 0.0217 |
| | | ACSM3, | | | | | | | | |
| | | LOC653783 | | | | | | | | |
| RS11074709 | 16: 025640038 | HS3ST4 | 0.0005 | !A,C: 1.46(1.19, 1.79) | 0.0762 | C: 1.17(0.99, 1.38) | 0.0277 | 0.0527 | 0.0069 | 0.5561 |
| RS4238915 | 16: 025643984 | HS3ST4 | 0.0006 | !G,T: 1.45(1.19, 1.77) | 0.0751 | T: 1.16(0.99, 1.37) | 0.0265 | 0.0518 | 0.0075 | 0.5610 |
| RS4787948 | 16: 027248560 | IL4R | 0.0035 | !A,A: 1.35(1.12, 1.63) | 0.0007 | G: 1.29(1.11, 1.49) | 0.0140 | 0.0143 | 0.0395 | 0.0192 |
| RS772859 | 16: 027701027 | KIAA0556 | 0.0134 | C,T: 1.3(1.08, 1.57) | 0.0858 | C: 1.13(0.98, 1.29) | 0.1301 | 0.0477 | 0.0105 | 0.6672 |
| RS4788009 | 16: 027849046 | | 0.0032 | !A,A: 1.62(1.19, 2.21) | 0.0014 | G: 1.26(1.09, 1.45) | 0.0144 | 0.0384 | 0.0531 | 0.0149 |
| RS151230 | 16: 028490716 | CCDC101 | 0.0064 | !C,C: 1.35(1.08, 1.69) | 0.0024 | T: 1.37(1.12, 1.68) | 0.0417 | 0.0231 | 0.0103 | 0.0438 |
| RS1362567 | 16: 045782194 | ITFG1 | 0.0012 | C,C: 1.49(1.2, 1.85) | 0.0007 | C: 1.4(1.15, 1.7) | 0.0562 | 0.0346 | 0.0197 | 0.0076 |
| RS10521169 | 16: 047463980 | | 0.005 | !T,T: 1.36(1.13, 1.65) | 0.0015 | C: 1.29(1.11, 1.52) | 0.0214 | 0.0140 | 0.0124 | 0.0482 |
| RS12930072 | 16: 047510482 | | 0.0022 | T,T: 1.4(1.16, 1.69) | 0.0011 | T: 1.28(1.11, 1.49) | 0.0012 | 0.0324 | 0.0291 | 0.0157 |
| RS8053786 | 16: 047566891 | | 0.0053 | A,A: 1.3(1.07, 1.57) | 0.0013 | A: 1.29(1.11, 1.51) | 0.0911 | 0.0324 | 0.0124 | 0.0160 |
| RS2200537 | 16: 053769135 | | 0.0165 | !C,C: 1.71(1.12, 2.63) | 0.0088 | T: 1.24(1.06, 1.45) | 0.1613 | 0.0482 | 0.0431 | 0.0829 |
| RS728571 | 16: 053771594 | | 0.0005 | G,G: 1.48(1.21, 1.81) | 8.90E−05 | G: 1.42(1.19, 1.69) | 0.0134 | 0.0054 | 0.0105 | 0.0058 |
| RS1436413 | 16: 061775362 | | 0.0064 | G,G: 1.32(1.09, 1.59) | 0.0016 | G: 1.25(1.09, 1.44) | 0.0661 | 0.0287 | 0.0721 | 0.0296 |
| RS427576 | 16: 072386771 | | 0.0042 | A,A: 1.39(1.13, 1.71) | 0.0012 | A: 1.25(1.09, 1.42) | 0.1091 | 0.0413 | 0.0400 | 0.0129 |
| RS725710 | 16: 073431014 | | 0.0005 | C,C: 1.52(1.22, 1.88) | 1.01E−04 | C: 1.46(1.21, 1.77) | 0.0100 | 0.0047 | 0.0253 | 0.0091 |
| RS1390907 | 16: 075501858 | | 0.0002 | !A,G: 1.48(1.22, 1.78) | 0.0121 | A: 1.2(1.04, 1.37) | 0.0331 | 0.0247 | 0.0017 | 0.2033 |
| RS8059438 | 16: 081773975 | CDH13 | 0.0008 | !T,T: 1.5(1.18, 1.9) | 0.0003 | C: 1.5(1.2, 1.88) | 0.0080 | 0.0037 | 0.0656 | 0.0300 |
| RS3829514 | 16: 083652499 | KIAA0513 | 0.0008 | !C,T: 1.74(1.25, 2.42) | 0.0041 | T: 1.59(1.15, 2.18) | 0.0103 | 0.0121 | 0.0281 | 0.1339 |
| RS11117407 | 16: 084411812 | | 0.0029 | !C,C: 2.59(1.37, 4.89) | 0.0051 | C: 1.28(1.08, 1.53) | 0.0488 | 0.0205 | 0.0355 | 0.1148 |
| RS2965946 | 16: 088044113 | ANKRD11 | 0.0042 | T,T: 1.72(1.2, 2.47) | 0.4102 | T: 1.07(0.92, 1.23) | 0.0437 | 0.7851 | 0.0431 | 0.1352 |
| RS8060934 | 16: 088447526 | SPIRE2 | 0.0159 | T,T: 1.33(1.06, 1.66) | 0.0041 | T: 1.22(1.06, 1.39) | 0.1179 | 0.0411 | 0.1277 | 0.0433 |
| RS8072032 | 17: 004895930 | | 0.0009 | G,G: 1.61(1.23, 2.11) | 0.0003 | G: 1.61(1.24, 2.09) | 0.0568 | 0.0378 | 0.0027 | 0.0026 |
| RS4790743 | 17: 004940854 | ZFP3 | 0.0008 | A,A: 1.65(1.21, 2.27) | 0.0006 | A: 1.69(1.25, 2.29) | 0.0526 | 0.0416 | 0.0108 | 0.0078 |
| RS1619016 | 17: 007491278 | | 0.0041 | !A,A: 1.42(1.13, 1.78) | 0.0012 | G: 1.41(1.14, 1.73) | 0.0223 | 0.0097 | 0.1412 | 0.0476 |
| RS714407 | 17: 008801218 | | 0.0456 | A,G: 1.3(1.05, 1.61) | 0.1207 | A: 1.16(0.96, 1.4) | 0.0223 | 0.7768 | 0.0401 | 0.0141 |
| RS11653099 | 17: 009811124 | GAS7 | 0.0317 | !C,T: 1.27(1.05, 1.53) | 0.438 | T: 1.06(0.93, 1.21) | 0.0589 | 0.0289 | 0.0278 | 0.2801 |
| RS719470 | 17: 014147087 | HS3ST3B1 | 0.0109 | !T,T: 1.31(1.07, 1.59) | 0.003 | C: 1.23(1.07, 1.4) | 0.0814 | 0.0271 | 0.1185 | 0.0466 |
| RS2191176 | 17: 014745432 | LOC440402 | 0.0012 | !T,T: 1.36(1.12, 1.64) | 0.0003 | C: 1.32(1.14, 1.53) | 0.0316 | 0.0093 | 0.0371 | 0.0123 |
| RS230977 | 17: 015199094 | | 0.015 | A,G: 1.37(1.11, 1.7) | 0.0238 | G: 1.24(1.03, 1.49) | 0.0137 | 0.4963 | 0.0475 | 0.0158 |
| RS4613115 | 17: 027953003 | MYO1D | 0.0276 | A,G: 1.27(1.05, 1.53) | 0.2366 | A: 1.09(0.95, 1.24) | 0.0327 | 0.4092 | 0.0367 | 0.0142 |
| RS4796217 | 17: 031893304 | LOC441792 | 0.0074 | C,C: 1.34(1.11, 1.62) | 0.0103 | C: 1.21(1.05, 1.41) | 0.0344 | 0.1248 | 0.0867 | 0.0310 |
| RS713101 | 17: 040548347 | PLCD3 | 0.0005 | A,A: 1.61(1.26, 2.05) | 0.0071 | A: 1.2(1.05, 1.37) | 0.0102 | 0.0637 | 0.0487 | 0.0632 |
| RS9896945 | 17: 051713575 | ANKFN1 | 0.01 | A,A: 1.28(1.05, 1.55) | 0.0024 | A: 1.24(1.08, 1.41) | 0.0481 | 0.1132 | 0.0090 | 0.0070 |
| RS2270150 | 17: 056915467 | TBX4 | 0.0142 | !A,G: 1.29(1.04, 1.6) | 0.2991 | G: 1.1(0.92, 1.33) | 0.0237 | 0.5570 | 0.0525 | 0.0281 |
| RS8080666 | 17: 059840612 | | 0.0195 | G,G: 1.36(1.1, 1.7) | 0.0337 | G: 1.15(1.01, 1.32) | 0.0127 | 0.3827 | 0.1032 | 0.0347 |
| RS9303513 | 17: 062462327 | | 0.0023 | !A,A: 1.68(1.25, 2.25) | 0.0192 | G: 1.18(1.03, 1.36) | 0.0491 | 0.1517 | 0.0423 | 0.0685 |
| RS1540373 | 17: 066573542 | | 7.26E−05 | !A,A: 1.99(1.36, 2.91) | 0.6467 | G: 1.04(0.89, 1.21) | 0.0066 | 0.9569 | 0.0104 | 0.5165 |
| RS8064529 | 17: 071031008 | TSEN54 | 0.0015 | T,T: 1.77(1.28, 2.46) | 0.0852 | T: 1.14(0.98, 1.31) | 0.0083 | 0.0119 | 0.0449 | 0.9591 |
| RS7502321 | 17: 076608112 | | 0.0042 | T,T: 1.46(1.11, 1.91) | 0.0013 | T: 1.25(1.09, 1.43) | 0.0597 | 0.0232 | 0.0398 | 0.0281 |
| RS1662813 | 18: 003309915 | | 0.0009 | A,G: 1.49(1.21, 1.85) | 0.0022 | G: 1.33(1.11, 1.6) | 0.0239 | 0.0404 | 0.0344 | 0.0268 |
| RS2847108 | 18: 004582823 | | 0.0023 | G,G: 1.44(1.17, 1.77) | 0.0039 | G: 1.22(1.07, 1.39) | 0.0164 | 0.0325 | 0.0855 | 0.0411 |
| RS12455648 | 18: 008465207 | | 0.0015 | !T,T: 1.87(1.32, 2.64) | 0.0253 | C: 1.18(1.02, 1.37) | 0.0031 | 0.4186 | 0.0547 | 0.0231 |
| RS9962727 | 18: 010822851 | C18ORF58 | 0.0037 | T,T: 1.32(1.1, 1.6) | 0.0009 | T: 1.28(1.11, 1.48) | 0.0177 | 0.0070 | 0.1098 | 0.0442 |
| RS4796974 | 18: 013249774 | C18ORF1, | 0.0003 | C,C: 1.58(1.22, 2.04) | 0.1677 | C: 1.1(0.96, 1.26) | 0.0127 | 0.3207 | 0.0209 | 0.3378 |
| | | LOC651125 | | | | | | | | |
| RS7232054 | 18: 013261319 | C18ORF1 | 2.09E−05 | !A,G: 1.53(1.26, 1.84) | 0.6566 | A: 1.03(0.9, 1.18) | 0.0016 | 0.7308 | 0.0135 | 0.7752 |
| RS2042743 | 18: 013417800 | C18ORF1 | 0.0097 | !A,C: 1.32(1.1, 1.6) | 0.3805 | A: 1.06(0.93, 1.21) | 0.0423 | 0.0525 | 0.0425 | 0.5415 |
| RS1941611 | 18: 020830023 | | 0.0009 | G,G: 1.42(1.18, 1.72) | 0.0003 | G: 1.32(1.14, 1.53) | 0.0137 | 0.0099 | 0.0367 | 0.0114 |
| RS5022348 | 18: 020893235 | | 0.0003 | T,T: 1.4(1.15, 1.71) | 5.04E−05 | T: 1.32(1.15, 1.51) | 0.0501 | 0.0145 | 0.0041 | 0.0012 |
| RS12966353 | 18: 024297001 | | 0.0028 | A,A: 1.56(1.18, 2.07) | 0.0012 | A: 1.25(1.09, 1.44) | 0.0192 | 0.0075 | 0.0932 | 0.0433 |
| RS4799700 | 18: 025807829 | | 0.0074 | !C,C: 1.4(1.12, 1.74) | 0.0037 | T: 1.22(1.07, 1.39) | 0.0387 | 0.0589 | 0.0708 | 0.0243 |
| RS7244533 | 18: 025834901 | | 0.0366 | !T,T: 1.28(1.06, 1.54) | 0.0284 | C: 1.18(1.02, 1.38) | 0.0362 | 0.4779 | 0.0509 | 0.0198 |
| RS1260719 | 18: 026824023 | DSC3 | 0.0043 | G,T: 1.35(1.12, 1.63) | 0.0854 | G: 1.13(0.99, 1.29) | 0.0138 | 0.0070 | 0.0095 | 0.7717 |
| RS638252 | 18: 027628181 | | 0.0337 | G,G: 1.34(1.06, 1.7) | 0.1952 | G: 1.09(0.96, 1.25) | 0.0609 | 0.0215 | 0.0277 | 0.6690 |
| RS2391 | 18: 031222379 | | 0.0466 | G,G: 4.59(1.17, 18.08) | 0.2579 | G: 1.16(0.9, 1.48) | 0.0043 | 0.4083 | 0.0508 | 0.0157 |
| RS519309 | 18: 032638832 | C18ORF10 | 0.001 | C,C: 5.6(1.79, 17.55) | 0.6665 | C: 1.05(0.85, 1.3) | 0.0193 | 1.0000 | 0.0489 | 0.5321 |
| RS323285 | 18: 032989897 | KIAA1328 | 0.0009 | T,T: 5.61(1.79, 17.57) | 0.7045 | T: 1.05(0.85, 1.29) | 0.0257 | 0.9402 | 0.0399 | 0.6955 |
| RS11082000 | 18: 033171021 | BRUNOL4 | 0.0049 | A,A: 1.34(1.11, 1.62) | 0.0013 | A: 1.3(1.11, 1.53) | 0.0243 | 0.0373 | 0.0172 | 0.0141 |
| RS7242236 | 18: 034485243 | | 0.0059 | T,T: 1.4(1.14, 1.73) | 0.0045 | T: 1.21(1.06, 1.38) | 0.1121 | 0.0466 | 0.0404 | 0.0424 |
| RS1943413 | 18: 034491676 | | 0.0046 | A,A: 1.52(1.18, 1.97) | 0.0112 | A: 1.19(1.04, 1.36) | 0.0401 | 0.0194 | 0.0483 | 0.1844 |
| RS987380 | 18: 037702007 | | 0.0095 | !G,G: 3.08(1.17, 8.11) | 0.0058 | A: 1.38(1.1, 1.73) | 0.0227 | 0.0077 | 0.0251 | 0.2320 |
| RS872035 | 18: 041552646 | | 0.0013 | !G,G: 1.43(1.17, 1.74) | 0.0004 | A: 1.36(1.15, 1.62) | 0.0588 | 0.0203 | 0.0191 | 0.0085 |
| RS4624284 | 18: 042584602 | ST8SIA5 | 0.001 | T,T: 2.11(1.4, 3.17) | 0.0111 | T: 1.22(1.05, 1.42) | 0.0217 | 0.1528 | 0.0338 | 0.0357 |
| RS9953229 | 18: 044135485 | | 0.0013 | C,C: 1.39(1.13, 1.71) | 0.0002 | C: 1.28(1.12, 1.47) | 0.0529 | 0.0225 | 0.0167 | 0.0041 |
| RS745611 | 18: 044163038 | | 0.0004 | C,C: 1.42(1.17, 1.71) | 7.93E−05 | C: 1.34(1.16, 1.54) | 0.0010 | 0.0006 | 0.0625 | 0.0325 |
| RS1222938 | 18: 048195164 | DCC | 0.0013 | A,G: 1.39(1.15, 1.69) | 0.3958 | G: 1.07(0.92, 1.24) | 0.0415 | 1.0000 | 0.0230 | 0.2298 |
| RS4309482 | 18: 050901467 | | 0.0134 | A,A: 1.29(1.06, 1.57) | 0.0031 | A: 1.23(1.07, 1.41) | 0.1012 | 0.0334 | 0.1318 | 0.0398 |
| RS337718 | 18: 067925258 | | 0.0007 | !C,T: 1.39(1.15, 1.68) | 0.681 | C: 1.03(0.89, 1.19) | 0.0231 | 0.6710 | 0.0274 | 0.8745 |
| RS10871696 | 18: 068024582 | | 0.0005 | !A,C: 1.45(1.2, 1.75) | 0.0935 | C: 1.12(0.98, 1.29) | 0.0124 | 0.4245 | 0.0289 | 0.1171 |
| RS4536550 | 18: 069099471 | | 0.0152 | A,A: 1.32(1.09, 1.59) | 0.0103 | A: 1.21(1.05, 1.4) | 0.0232 | 0.1037 | 0.1540 | 0.0457 |
| RS999647 | 18: 070700538 | ZNF407 | 0.0032 | !T,T: 1.4(1.15, 1.7) | 0.002 | C: 1.24(1.08, 1.42) | 0.0167 | 0.0271 | 0.1135 | 0.0353 |
| RS9960454 | 18: 070798925 | | 0.0017 | !A,A: 1.42(1.17, 1.73) | 0.0012 | G: 1.25(1.09, 1.43) | 0.0094 | 0.0156 | 0.1092 | 0.0345 |
| RS906516 | 18: 070916956 | | 0.0233 | G,G: 1.4(1.1, 1.79) | 0.0202 | G: 1.17(1.03, 1.34) | 0.0272 | 0.7294 | 0.0107 | 0.0033 |
| RS2732239 | 18: 071762651 | | 0.1034 | !C,C: 1.24(1.02, 1.51) | 0.0398 | A: 1.2(1.01, 1.41) | 0.0394 | 0.0981 | 0.0217 | 0.2309 |
| RS2926622 | 18: 071764657 | | 0.0978 | !T,T: 1.24(1.02, 1.51) | 0.0358 | C: 1.2(1.01, 1.42) | 0.0403 | 0.0861 | 0.0217 | 0.2309 |
| RS2612377 | 18: 071771223 | | 0.1032 | !A,A: 1.24(1.02, 1.51) | 0.0396 | G: 1.2(1.01, 1.42) | 0.0398 | 0.0973 | 0.0217 | 0.2309 |
| RS689902 | 18: 072248957 | ZNF516 | 0.0066 | !C,C: 1.37(1.13, 1.67) | 0.004 | T: 1.22(1.07, 1.4) | 0.0572 | 0.0443 | 0.1113 | 0.0446 |
| RS1789107 | 18: 072993175 | | 0.0915 | G,G: 1.19(0.99, 1.44) | 0.0264 | G: 1.17(1.02, 1.35) | 0.0281 | 0.0790 | 0.0244 | 0.1799 |
| RS4541161 | 18: 073966360 | | 0.0312 | !C,T: 1.26(1.05, 1.53) | 0.8581 | C: 1.01(0.88, 1.17) | 0.0170 | 0.0389 | 0.0128 | 0.0256 |
| RS11878115 | 18: 074498350 | | 0.0785 | A,A: 1.21(1, 1.46) | 0.029 | A: 1.19(1.02, 1.39) | 0.0406 | 0.2884 | 0.0036 | 0.0439 |
| RS7227107 | 18: 075328464 | NFATC1 | 0.0057 | G,G: 1.66(1.19, 2.31) | 0.0057 | G: 1.22(1.06, 1.41) | 0.1217 | 0.0465 | 0.0286 | 0.0551 |
| RS160189 | 18: 075333697 | NFATC1 | 0.0003 | G,G: 1.89(1.37, 2.6) | 0.0021 | G: 1.25(1.09, 1.44) | 0.0239 | 0.0216 | 0.0118 | 0.0396 |
| RS8102902 | 19: 003720049 | RAXL1, | 0.0043 | !G,G: 2.16(1.25, 3.73) | 0.0037 | T: 1.28(1.08, 1.52) | 0.0159 | 0.0774 | 0.0619 | 0.0196 |
| | | MRPL54 | | | | | | | | |
| RS10518246 | 19: 015020981 | | 0.0057 | A,A: 1.38(1.13, 1.69) | 0.0057 | A: 1.28(1.08, 1.52) | 0.0211 | 0.0855 | 0.0999 | 0.0295 |
| RS3181163 | 19: 015027162 | CASP14 | 0.0077 | !C,T: 1.39(1.13, 1.71) | 0.0135 | T: 1.24(1.05, 1.47) | 0.0182 | 0.1472 | 0.1277 | 0.0426 |
| RS8108738 | 19: 018116359 | MAST3 | 0.0173 | A,G: 1.3(1.08, 1.57) | 0.6125 | G: 1.04(0.91, 1.18) | 0.0066 | 0.1734 | 0.0834 | 0.0433 |
| RS8103146 | 19: 020582566 | | 0.0068 | C,C: 1.42(1.14, 1.76) | 0.0031 | C: 1.35(1.11, 1.65) | 0.0501 | 0.0262 | 0.1089 | 0.0496 |
| RS10756 | 19: 035196656 | C19ORF2 | 0.0094 | !C,T: 1.33(1.1, 1.61) | 0.0369 | C: 1.17(1.01, 1.35) | 0.0560 | 0.0253 | 0.0463 | 0.4396 |
| RS10415219 | 19: 043842075 | ACTN4 | 0.0107 | !A,A: 1.37(1.11, 1.69) | 0.0259 | G: 1.16(1.02, 1.33) | 0.0312 | 0.3814 | 0.0687 | 0.0241 |
| RS2279144 | 19: 043893621 | ACTN4 | 0.0063 | !T,T: 1.4(1.13, 1.72) | 0.0198 | C: 1.17(1.03, 1.34) | 0.0270 | 0.4088 | 0.0410 | 0.0146 |
| RS2604879 | 19: 045887119 | NUMBL | 0.0046 | A,G: 1.4(1.14, 1.71) | 0.0268 | A: 1.21(1.02, 1.43) | 0.0390 | 0.0198 | 0.0484 | 0.4663 |
| RS659555 | 19: 054521637 | SLC6A16, | 0.0003 | G,G: 1.45(1.2, 1.75) | 0.0019 | G: 1.26(1.09, 1.45) | 0.0515 | 0.0396 | 0.0052 | 0.0223 |
| | | AK097351 | | | | | | | | |
| RS10403271 | 19: 055778845 | | 0.0107 | A,A: 1.28(1.06, 1.54) | 0.0029 | A: 1.26(1.08, 1.47) | 0.0941 | 0.0445 | 0.0252 | 0.0283 |
| RS9917107 | 19: 056306273 | | 1.25E−04 | !G,G: 4.24(1.99, 9.04) | 0.0755 | T: 1.19(0.98, 1.44) | 0.0152 | 0.6444 | 0.0028 | 0.0295 |
| RS2062655 | 19: 056495675 | | 0.0026 | A,C: 1.33(1.1, 1.6) | 0.2096 | C: 1.09(0.96, 1.25) | 0.0147 | 0.6965 | 0.0226 | 0.0300 |
| RS6509525 | 19: 056509963 | LOC402665 | 0.0341 | C,T: 1.28(1.06, 1.54) | 0.4989 | T: 1.05(0.92, 1.2) | 0.0394 | 0.2639 | 0.0509 | 0.0384 |
| RS3848559 | 19: 056526006 | LOC402665, | 0.0007 | !C,C: 1.5(1.2, 1.87) | 0.0365 | T: 1.15(1.01, 1.32) | 0.0197 | 0.0586 | 0.0285 | 0.2392 |
| | | LOC147645 | | | | | | | | |
| RS2288522 | 19: 060400287 | PTPRH | 0.0833 | A,A: 1.28(1.03, 1.59) | 0.0373 | A: 1.23(1.02, 1.5) | 0.0423 | 0.6180 | 0.0476 | 0.0158 |
| RS8106321 | 19: 061466373 | | 0.0008 | T,T: 1.45(1.17, 1.8) | 0.0686 | T: 1.13(0.99, 1.29) | 0.0107 | 0.0369 | 0.0456 | 0.6725 |
| RS6048226 | 20: 000233323 | | 0.0131 | !G,G: 1.44(1.12, 1.85) | 0.0065 | A: 1.21(1.06, 1.38) | 0.0751 | 0.0229 | 0.0362 | 0.1131 |
| RS6108123 | 20: 000821277 | ANGPT4 | 0.0062 | A,A: 1.36(1.12, 1.64) | 0.0022 | A: 1.24(1.08, 1.42) | 0.0541 | 0.0177 | 0.0793 | 0.0437 |
| RS7268029 | 20: 004466594 | | 1.13E−04 | A,G: 1.56(1.22, 2) | 0.0753 | G: 1.22(0.98, 1.52) | 0.0214 | 0.4916 | 0.0051 | 0.0748 |
| RS6053111 | 20: 005029937 | C20ORF30 | 0.01 | C,C: 1.32(1.09, 1.6) | 0.0026 | C: 1.23(1.08, 1.42) | 0.1392 | 0.0496 | 0.0735 | 0.0252 |
| RS6053268 | 20: 005226993 | | 0.0326 | !C,T: 1.27(1.05, 1.55) | 0.4308 | C: 1.07(0.91, 1.24) | 0.0239 | 0.0635 | 0.0061 | 0.0034 |
| RS2182712 | 20: 006316089 | | 0.0019 | !T,T: 1.39(1.15, 1.67) | 0.0006 | G: 1.29(1.12, 1.49) | 0.0388 | 0.0136 | 0.0476 | 0.0207 |
| RS10485749 | 20: 011374730 | | 0.0454 | A,G: 1.27(1.05, 1.54) | 0.1503 | A: 1.11(0.96, 1.29) | 0.0170 | 0.8735 | 0.0961 | 0.0312 |
| RS730183 | 20: 011390419 | | 0.0392 | C,T: 1.28(1.06, 1.55) | 0.14 | C: 1.12(0.97, 1.29) | 0.0170 | 0.8735 | 0.0872 | 0.0274 |
| RS4814019 | 20: 011456980 | | 2.49E−05 | !A,A: 1.51(1.25, 1.82) | 3.40E−06 | C: 1.4(1.22, 1.61) | 0.0248 | 0.0145 | 0.0003 | ##### |
| RS293569 | 20: 030568637 | | 0.0003 | !C,T: 1.7(1.28, 2.26) | 0.0014 | T: 1.56(1.19, 2.04) | 0.0091 | 0.0214 | 0.0169 | 0.0266 |
| RS6093318 | 20: 038337505 | | 0.0165 | A,G: 1.33(1.08, 1.64) | 0.1303 | G: 1.15(0.96, 1.38) | 0.1103 | 0.0424 | 0.0123 | 0.8515 |
| RS1014748 | 20: 038821628 | | 0.0021 | A,A: 3.51(1.63, 7.57) | 0.0577 | A: 1.21(1, 1.46) | 0.0295 | 0.3812 | 0.0447 | 0.0774 |
| RS2284271 | 20: 043038835 | STK4 | 0.0016 | !T,T: 1.48(1.15, 1.9) | 0.0009 | C: 1.49(1.18, 1.89) | 0.0352 | 0.0135 | 0.0561 | 0.0255 |
| RS2743312 | 20: 043369569 | MATN4, | 0.0159 | G,G: 1.32(1.08, 1.62) | 0.0036 | G: 1.22(1.07, 1.39) | 0.0046 | 0.0285 | 0.0475 | 0.0663 |
| | | RBPSUHL | | | | | | | | |
| RS231584 | 20: 048773002 | | 0.0026 | !C,C: 1.57(1.19, 2.08) | 0.0016 | T: 1.54(1.18, 2.01) | 0.0309 | 0.0186 | 0.0265 | 0.0250 |
| RS427034 | 20: 054901032 | | 0.0004 | G,G: 1.44(1.19, 1.74) | 1.06E−04 | G: 1.35(1.16, 1.58) | 0.0011 | 0.0022 | 0.0385 | 0.0209 |
| RS6064592 | 20: 055788953 | | 0.0023 | A,G: 1.41(1.14, 1.74) | 0.0692 | G: 1.18(0.99, 1.41) | 0.0071 | 0.6993 | 0.0636 | 0.0344 |
| RS183777 | 20: 057244560 | C20ORF174 | 0.0457 | C,C: 1.31(1.06, 1.62) | 0.0685 | C: 1.13(0.99, 1.29) | 0.1191 | 0.0414 | 0.0245 | 0.6384 |
| RS6089479 | 20: 059710358 | CDH4 | 0.002 | !A,G: 1.42(1.17, 1.72) | 0.0337 | A: 1.18(1.01, 1.38) | 0.0440 | 0.0411 | 0.0261 | 0.3533 |
| RS6062821 | 20: 061246930 | | 0.0012 | !A,G: 1.4(1.16, 1.69) | 0.2373 | A: 1.08(0.95, 1.24) | 0.0192 | 0.1201 | 0.0395 | 0.9251 |
| RS6090265 | 20: 061249931 | | 0.0005 | T,T: 1.54(1.2, 1.98) | 0.1825 | T: 1.1(0.96, 1.26) | 0.0194 | 0.1412 | 0.0168 | 0.7029 |
| RS2315656 | 20: 061888781 | BTBD4 | 0.0136 | G,G: 1.33(1.1, 1.61) | 0.0161 | G: 1.18(1.03, 1.36) | 0.0978 | 0.0366 | 0.0237 | 0.1863 |
| RS8130901 | 21: 013879844 | | 0.0017 | !G,G: 1.51(1.18, 1.92) | 0.0879 | A: 1.13(0.98, 1.29) | 0.0235 | 0.8444 | 0.0236 | 0.0290 |
| RS2205533 | 21: 015674652 | LOC388814 | 0.0096 | G,G: 1.38(1.12, 1.69) | 0.0254 | G: 1.17(1.02, 1.33) | 0.0137 | 0.4968 | 0.0393 | 0.0123 |
| RS2823252 | 21: 015694445 | | 0.0094 | A,A: 1.36(1.11, 1.67) | 0.0248 | A: 1.17(1.02, 1.33) | 0.0114 | 0.3295 | 0.0780 | 0.0291 |
| RS859164 | 21: 017530786 | | 0.0431 | !C,T: 1.29(1.06, 1.57) | 0.0746 | T: 1.16(0.99, 1.36) | 0.0686 | 0.0243 | 0.0473 | 0.7762 |
| RS2825682 | 21: 019912457 | | 0.0294 | !C,C: 2.54(1.19, 5.44) | 0.595 | A: 1.06(0.87, 1.28) | 0.0442 | 0.0180 | 0.0051 | 0.0998 |
| RS2826406 | 21: 020870513 | | 0.0119 | !T,T: 1.27(1.05, 1.55) | 0.0035 | C: 1.22(1.07, 1.4) | 0.1234 | 0.0400 | 0.1152 | 0.0473 |
| RS416707 | 21: 021048681 | | 0.0123 | G,T: 1.33(1.1, 1.62) | 0.1493 | T: 1.13(0.96, 1.32) | 0.0908 | 0.0379 | 0.0119 | 0.9557 |
| RS2831449 | 21: 028359620 | LOC284825 | 0.0109 | !T,T: 1.5(1.14, 1.98) | 0.0025 | G: 1.5(1.15, 1.94) | 0.1252 | 0.0446 | 0.0893 | 0.0295 |
| RS2832238 | 21: 029471232 | C21ORF7 | 0.0059 | !G,T: 1.32(1.08, 1.61) | 0.5407 | T: 1.05(0.9, 1.23) | 0.0054 | 0.0141 | 0.0145 | 0.1384 |
| RS2834732 | 21: 035308597 | RUNX1 | 0.0035 | T,T: 1.38(1.14, 1.66) | 0.0096 | T: 1.2(1.05, 1.38) | 0.1152 | 0.0438 | 0.0095 | 0.1179 |
| RS2835404 | 21: 036905003 | | 0.0193 | G,G: 3.16(1.3, 7.68) | 0.086 | G: 1.19(0.98, 1.46) | 0.1216 | 0.0408 | 0.0148 | 0.5878 |
| RS2409948 | 21: 038315359 | | 0.0008 | !T,T: 3.11(1.48, 6.51) | 0.0009 | C: 1.38(1.14, 1.67) | 0.0227 | 0.0148 | 0.0388 | 0.0344 |
| RS596958 | 21: 039945361 | | 0.0003 | !T,T: 1.71(1.27, 2.3) | 0.0003 | C: 1.71(1.28, 2.28) | 0.0176 | 0.0213 | 0.0060 | 0.0045 |
| RS2239385 | 21: 039949617 | B3GALT5 | 8.01E−05 | !G,G: 1.52(1.25, 1.83) | 3.54E−05 | A: 1.38(1.18, 1.61) | 0.0094 | 0.0058 | 0.0093 | 0.0028 |
| RS2254583 | 21: 040373134 | DSCAM | 0.0046 | !A,A: 1.4(1.11, 1.77) | 0.0014 | G: 1.42(1.15, 1.76) | 0.0703 | 0.0343 | 0.0679 | 0.0202 |
| RS3746889 | 21: 041573816 | | 0.0055 | !T,T: 1.83(1.22, 2.73) | 0.0021 | C: 1.82(1.24, 2.69) | 0.0477 | 0.0219 | 0.0239 | 0.0589 |
| RS233274 | 21: 044813916 | C21ORF29 | 0.0002 | G,T: 1.47(1.22, 1.77) | 0.8656 | G: 1.01(0.89, 1.16) | 0.0028 | 0.2845 | 0.0294 | 0.4503 |
| RS2238732 | 22: 017295347 | PRODH | 0.0031 | T,T: 1.64(1.17, 2.31) | 0.0029 | T: 1.66(1.19, 2.31) | 0.0498 | 0.0324 | 0.0458 | 0.0389 |
| RS5748218 | 22: 017831186 | UFD1L | 0.0489 | !A,C: 1.29(1.05, 1.59) | 0.1375 | C: 1.15(0.96, 1.37) | 0.1002 | 0.0332 | 0.0339 | 1.0000 |
| RS5752534 | 22: 026122500 | | 0.0009 | A,A: 1.42(1.18, 1.72) | 0.0003 | A: 1.33(1.14, 1.55) | 0.0659 | 0.0245 | 0.0138 | 0.0043 |
| RS12537 | 22: 028753460 | LOC400924, | 0.0027 | C,T: 1.39(1.15, 1.68) | 0.3307 | C: 1.07(0.94, 1.23) | 0.0479 | 0.0832 | 0.0101 | 0.6959 |
| | | MTMR3 | | | | | | | | |
| RS139998 | 22: 035192447 | TXN2 | 0.0072 | !T,T: 1.37(1.13, 1.68) | 0.002 | C: 1.31(1.1, 1.55) | 0.0809 | 0.0286 | 0.0361 | 0.0341 |
| RS5756219 | 22: 035221804 | FOXRED2 | 6.43E−05 | !C,C: 1.53(1.25, 1.88) | 1.44E−05 | A: 1.34(1.18, 1.53) | 0.0008 | 0.0002 | 0.0094 | 0.0162 |
| RS2076083 | 22: 035242670 | EIF3S7 | 0.0017 | C,T: 1.47(1.19, 1.82) | 0.0082 | C: 1.27(1.06, 1.51) | 0.0277 | 0.0142 | 0.0378 | 0.1805 |
| RS2009667 | 22: 035501557 | RABL4 | 0.0014 | A,A: 1.53(1.21, 1.93) | 0.0032 | A: 1.22(1.07, 1.4) | 0.0045 | 0.0016 | 0.0437 | 0.3209 |
| RS135720 | 22: 036993765 | KIAA1660, | 00014 | G,G: 1.36(1.12, 1.65) | 0.0002 | G: 1.29(1.13, 1.48) | 0.0424 | 0.0130 | 0.0289 | 0.0080 |
| | | C22ORF5 | | | | | | | | |
| RS132451 | 22: 043535767 | TIG48667, | 0.0038 | !G,G: 1.65(1.22, 2.23) | 0.0059 | A: 1.22(1.06, 1.4) | 0.0510 | 0.0183 | 0.0492 | 0.1216 |
| | | ARHGAP8, | | | | | | | | |
| | | LOC553158 | | | | | | | | |
| RS7410678 | 22: 043605937 | LOC553158, | 0.0082 | !C,C: 2.11(1.26, 3.52) | 0.0148 | T: 1.23(1.04, 1.45) | 0.0625 | 0.0199 | 0.0409 | 0.2726 |
| | | ARHGAP8, | | | | | | | | |
| | | TIG48667 | | | | | | | | |
| RS5765043 | 22: 043610547 | TIG48667, | 0.0012 | !C,C: 3.22(1.54, 6.73) | 0.0034 | T: 1.32(1.1, 1.59) | 0.0035 | 0.0009 | 0.0087 | 0.3897 |
| | | ARHGAP8, | | | | | | | | |
| | | LOC553158 | | | | | | | | |
| RS2285119 | 22: 043615277 | ARHGAP8, | 0.0108 | A,A: 1.36(1.06, 1.74) | 0.0087 | A: 1.38(1.09, 1.74) | 0.0122 | 0.0090 | 0.0293 | 0.2531 |
| | | LOC553158, | | | | | | | | |
| | | TIG48667 | | | | | | | | |
| RS132146 | 22: 046452725 | LOC284930 | 0.0127 | T,T: 1.59(1.17, 2.18) | 0.0348 | T: 1.17(1.01, 1.35) | 0.0796 | 0.0268 | 0.0124 | 0.4208 |
| RS916249 | 22: 047995693 | LOC150400 | 0.0004 | !A,C: 1.42(1.18, 1.72) | 0.3782 | A: 1.06(0.93, 1.21) | 0.0231 | 0.3055 | 0.0151 | 0.8504 |
| RS8140508 | 22: 047996263 | LOC150400 | 0.0002 | !C,T: 1.44(1.19, 1.73) | 0.3422 | C: 1.07(0.93, 1.22) | 0.0121 | 0.2616 | 0.0179 | 0.8135 |
| RS761878 | 22: 048635681 | ZBED4 | 0.0417 | !G,G: 1.26(1.01, 1.56) | 0.0213 | A: 1.26(1.04, 1.54) | 0.0162 | 0.0199 | 0.0307 | 0.2829 |
| RS138229 | 22: 048899534 | MOV10L1 | 1.40E−04 | A,A: 1.55(1.26, 1.91) | 1.01E−04 | A: 1.3(1.14, 1.49) | 0.0169 | 0.0127 | 0.0095 | 0.0042 |
| RS84504 | 22: 048901374 | MOV10L1 | 0.0007 | T,T: 1.55(1.23, 1.97) | 0.0005 | T: 1.27(1.11, 1.45) | 0.0450 | 0.0197 | 0.0177 | 0.0119 |
| RS5915682 | X: 003966940 | | 0.0262 | !A,G: 1.56(1.11, 2.19) | 0.3725 | A: 1.1(0.9, 1.35) | 0.0359 | 0.4698 | 0.1174 | 0.0326 |
| RS1552186 | X: 005708640 | | 0.0013 | !A,C: 1.67(1.22, 2.29) | 0.0193 | A: 1.21(1.03, 1.42) | 0.0061 | 0.0016 | 0.0310 | 0.9532 |
| RS996407 | X: 005709394 | | 0.0019 | !C,T: 1.65(1.21, 2.26) | 0.024 | T: 1.2(1.03, 1.42) | 0.0091 | 0.0023 | 0.0310 | 0.9532 |
| RS1016627 | X: 009791571 | APXL | 0.0557 | !A,A: 1.4(1.02, 1.92) | 0.0021 | G: 1.31(1.1, 1.55) | 0.0792 | 0.0243 | 0.5576 | 0.0411 |
| RS4830433 | X: 009906656 | XM_066971 | 0.0247 | A,A: 1.49(1.06, 2.1) | 0.3686 | A: 1.08(0.92, 1.27) | 0.0203 | 0.2068 | 0.0026 | 1.0000 |
| RS3027323 | X: 014471436 | GLRA2 | 0.2605 | A,A: 1.24(0.9, 1.7) | 0.0058 | A: 1.28(1.08, 1.52) | 0.1080 | 0.0199 | 0.0275 | 0.1186 |
| RS7391251 | X: 017130740 | | 0.0104 | G,G: 1.62(1.18, 2.23) | 0.0012 | G: 1.35(1.13, 1.61) | 0.0795 | 0.0388 | 0.0597 | 0.0140 |
| RS5955936 | X: 018046471 | | 0.0101 | G,G: 1.73(1.13, 2.65) | 0.0256 | G: 1.2(1.02, 1.42) | 0.0377 | 0.4851 | 0.1882 | 0.0159 |
| RS2285563 | X: 024936914 | ARX | 0.6635 | A,A: 1.28(0.7, 2.34) | 0.0008 | A: 1.37(1.14, 1.64) | 0.1146 | 0.0158 | 0.2219 | 0.0235 |
| RS7063687 | X: 024940207 | ARX | 0.4593 | C,T: 1.23(0.88, 1.73) | 0.0027 | C: 1.36(1.12, 1.67) | 0.4957 | 0.0035 | 0.0328 | 0.1908 |
| RS2765386 | X: 032975809 | DMD | 0.0012 | !C,C: 1.78(1.3, 2.45) | 0.0018 | T: 1.34(1.12, 1.61) | 0.0082 | 0.0196 | 0.1205 | 0.0389 |
| RS5972756 | X: 033049643 | DMD | 0.0018 | T,T: 5.19(1.28, 21.1) | 0.8011 | C: 1.03(0.81, 1.32) | 0.0201 | 0.3951 | 0.0446 | 0.6399 |
| RS5971730 | X: 033398410 | | 0.0005 | !T,T: 2.22(1.46, 3.36) | 0.0444 | C: 1.18(1.01, 1.39) | 0.0466 | 0.2056 | 0.0003 | 0.1281 |
| RS2168227 | X: 039308165 | | 0.0276 | !A,A: 2.47(1.21, 5.04) | 0.0018 | G: 1.34(1.11, 1.6) | 0.0170 | 0.0940 | 0.4433 | 0.0074 |
| RS4615740 | X: 040266364 | | 0.0019 | !T,T: 2.44(1.32, 4.51) | 0.0448 | C: 1.19(1.01, 1.42) | 0.0208 | 0.4243 | 0.0031 | 0.0415 |
| RS714472 | X: 042108199 | | 0.0384 | !G,T: 1.46(1.06, 2.02) | 0.1243 | T: 1.15(0.97, 1.36) | 0.0093 | 0.0003 | 0.0317 | 0.1431 |
| RS4335267 | X: 044940333 | CXORF36 | 0.0211 | !T,T: 1.56(1.14, 2.14) | 0.0157 | C: 1.23(1.04, 1.46) | 0.0176 | 0.0053 | 0.0224 | 0.5395 |
| RS2005947 | X: 048176609 | | 0.0273 | A,G: 1.54(1.12, 2.1) | 0.1383 | A: 1.13(0.96, 1.33) | 0.3799 | 0.0201 | 0.0204 | 0.8152 |
| RS880441 | X: 053360748 | | 0.001 | !C,C: 8.65(1.61, 46.38) | 0.0043 | A: 1.34(1.1, 1.64) | 0.0040 | 0.1603 | 0.0748 | 0.0077 |
| RS5914700 | X: 056577077 | | 0.0034 | C,T: 1.58(1.15, 2.18) | 0.0996 | T: 1.17(0.97, 1.41) | 0.0459 | 0.3394 | 0.0342 | 0.1826 |
| RS4826478 | X: 056594703 | | 0.0022 | G,T: 1.63(1.18, 2.24) | 0.0904 | T: 1.18(0.98, 1.42) | 0.0404 | 0.3093 | 0.0275 | 0.1815 |
| RS5914710 | X: 056616214 | | 0.0017 | A,C: 1.65(1.2, 2.27) | 0.0908 | A: 1.18(0.98, 1.41) | 0.0320 | 0.2813 | 0.0342 | 0.2050 |
| RS1925233 | X: 068529091 | | 0.0107 | T,T: 2.04(1.28, 3.26) | 0.0031 | T: 1.29(1.09, 1.52) | 0.0067 | 0.0374 | 0.5504 | 0.0408 |
| RS5937220 | X: 068557242 | | 0.0399 | A,A: 1.97(1.15, 3.36) | 0.0082 | A: 1.27(1.06, 1.5) | 0.0346 | 0.1051 | 0.6592 | 0.0341 |
| RS2411961 | X: 078395137 | | 0.0101 | G,G: 1.61(1.14, 2.29) | 0.0015 | G: 1.44(1.15, 1.81) | 0.0546 | 0.0159 | 0.1980 | 0.0383 |
| RS5940328 | X: 092860053 | | 0.0002 | !A,A: 2.15(1.47, 3.14) | 0.0309 | G: 1.32(1.03, 1.69) | 0.0199 | 0.1543 | 0.0022 | 0.1233 |
| RS5990454 | X: 095049095 | | 0.0059 | A,A: 1.74(1.23, 2.47) | 0.0109 | A: 1.34(1.07, 1.67) | 0.0157 | 0.1587 | 0.0136 | 0.0276 |
| RS5921266 | X: 096196914 | DIAPH2 | 0.0363 | A,A: 1.5(1.09, 2.07) | 0.1574 | A: 1.13(0.96, 1.33) | 0.0691 | 0.0419 | 0.0172 | 1.0000 |
| RS720403 | X: 099654701 | | 0.0525 | G,G: 1.47(1.07, 2.01) | 0.0162 | G: 1.24(1.04, 1.47) | 0.0939 | 0.0168 | 0.0488 | 0.3197 |
| RS12156914 | X: 100220184 | TMEM35 | 0.0004 | !A,A: 2.43(1.5, 3.93) | 0.0096 | G: 1.24(1.05, 1.46) | 0.0063 | 0.0093 | 0.0026 | 0.3146 |
| RS7058109 | X: 113114824 | | 0.0301 | !A,G: 1.53(1.11, 2.12) | 0.1584 | A: 1.14(0.95, 1.37) | 0.0134 | 0.3976 | 0.0321 | 0.0034 |
| RS7888180 | X: 113116314 | | 0.0042 | !G,T: 2.01(1.3, 3.12) | 0.0332 | T: 1.35(1.02, 1.77) | 0.0254 | 0.6320 | 0.0660 | 0.0136 |
| RS4621978 | X: 113134583 | | 0.0317 | !G,T: 1.51(1.08, 2.11) | 0.1761 | G: 1.15(0.95, 1.4) | 0.0028 | 0.3339 | 0.0463 | 0.0022 |
| RS543229 | X: 113727242 | LOC653062, | 0.0669 | C,C: 1.53(1.06, 2.2) | 0.006 | C: 1.37(1.09, 1.72) | 0.0420 | 0.0187 | 0.0223 | 0.1332 |
| | | HTR2C, | | | | | | | | |
| | | LOC653976 | | | | | | | | |
| RS5988087 | X: 113841112 | LOC653062, | 0.0415 | T,T: 1.59(1.1, 2.3) | 0.0014 | T: 1.46(1.16, 1.83) | 0.0247 | 0.0042 | 0.0208 | 0.1080 |
| | | HTR2C, | | | | | | | | |
| | | LOC653976 | | | | | | | | |
| RS11167436 | X: 113850316 | LOC653062, | 0.0415 | A,A: 1.59(1.1, 2.3) | 0.0011 | A: 1.47(1.17, 1.85) | 0.0247 | 0.0032 | 0.0208 | 0.1080 |
| | | HTR2C, | | | | | | | | |
| | | LOC653976 | | | | | | | | |
| RS5987947 | X: 114764437 | PLS3 | 0.002 | C,C: 2.24(1.39, 3.59) | 0.0967 | C: 1.15(0.98, 1.36) | 0.1069 | 0.0435 | 0.0069 | 0.8067 |
| RS7885278 | X: 120144651 | | 0.1254 | C,C: 1.56(0.98, 2.49) | 0.0013 | C: 1.32(1.12, 1.56) | 0.1938 | 0.0190 | 0.5595 | 0.0278 |
| RS3005849 | X: 122795009 | | 0.1782 | !C,C: 1.43(0.96, 2.14) | 0.0083 | T: 1.24(1.06, 1.46) | 0.0354 | 0.4179 | 0.3113 | 0.0039 |
| RS5958427 | X: 123192472 | | 0.0336 | C,C: 14.83(0.82, 269.42) | 0.0875 | C: 1.27(0.97, 1.66) | 0.0229 | 0.7754 | 0.0091 | 0.0311 |
| RS3810726 | X: 130050974 | RP13- | 0.0091 | !A,G: 1.67(1.11, 2.52) | 0.0209 | A: 1.36(1.05, 1.77) | 0.0522 | 0.0297 | 0.0498 | 0.2638 |
| | | 102H20.1 | | | | | | | | |
| RS5920535 | X: 143615817 | | 0.0565 | G,G: 1.84(1.07, 3.16) | 0.0324 | G: 1.21 (1.02, 1.44) | 0.0374 | 0.9028 | 0.1133 | 0.0027 |
| RS1029098 | X: 143994013 | | 0.0186 | !G,G: 1.58(1.13, 2.21) | 0.0628 | A: 1.17(0.99, 1.38) | 0.0458 | 0.0918 | 0.0189 | 0.3473 |

¹Significant Concordant Association was defined as having a genotypic and/or allelic exact p-value ≦0.05 in 1) the Munich collection, 2) Aberdeen collection and 3) the the combined set of all Munich + Aberdeen subjects.
²PolyName are RS identifiers.
³See Table 4 for a description of genes listed in this table.
⁴Key denoting strength of association
p-value ≦ 0.0005 in bold underline
0.0005 < p-value ≦ 0.005 in bold
0.005 < p-value ≦ 0.05 in italic
⁵For example, if a SNP had genotypes AA, AG and GG, 3 chi-square tests were performed contrasting cases and controls: 1) AA vs AG + GG, 2) AG vs AA + GG and 3) GG vs AA + AG. An odds ratio was then calculated for the test with the largest chi-square statistic. If the odds ratio was >1, this genotype was reported as the risk genotype. If the odds ratio was <1, then 1) the risk genotype was reported as “!” (“!” means “not”) this genotype and 2) a new odds ratio was calculated as the inverse of the original odds ratio. This new odds ratio was reported.

TABLE 4
Gene Description for Genes Listed in Tables 2 and 3.

| | | | Entrez | |
|---|---|---|---|---|
| Gene | Map | HUGO | Gene | |
| Symbol | Location | Gene¹ | ID² | Gene Description |
| ABCC5 | 3q27 | TRUE | 10057 | ATP-binding cassette, sub-family C (CFTR/MRP), member 5 |
| ABLIM2 | 4p16-p15 | TRUE | 84448 | actin binding LIM protein family, member 2 |
| ACOX3 | 4p15.3 | TRUE | 8310 | acyl-Coenzyme A oxidase 3, pristanoyl |
| ACOXL | 2q13 | TRUE | 55289 | acyl-Coenzyme A oxidase-like |
| ACSM3 | 16p13.11 | TRUE | 6296 | acyl-CoA synthetase medium-chain family member 3 |
| ACTN4 | 19q13 | TRUE | 81 | actinin, alpha 4 |
| ADAMTS14 | 10q2 | TRUE | 140766 | ADAM metallopeptidase with thrombospondin type 1 motif, 14 |
| ADAMTSL3 | 15q25.2 | TRUE | 57188 | ADAMTS-like 3 |
| ADARB2 | 10p15.3 | TRUE | 105 | adenosine deaminase, RNA-specific, B2 (RED2 homolog rat) |
| ADH4 | 4q21-q24\|4q22 | TRUE | 127 | alcohol dehydrogenase 4 (class II), pi polypeptide |
| ADK | 10q22\|10q11-q24 | TRUE | 132 | adenosine kinase |
| ADRA1B | 5q23-q32 | TRUE | 147 | adrenergic, alpha-1B-, receptor |
| AGPAT4 | 6q26 | TRUE | 56895 | 1-acylglycerol-3-phosphate O-acyltransferase 4 |
| | | | | (lysophosphatidic acid acyltransferase, delta) |
| AHI1 | 6q23.3 | TRUE | 54806 | Abelson helper integration site 1 |
| AHNAK | 11q12.2 | TRUE | 79026 | AHNAK nucleoprotein (desmoyokin) |
| AHSG | 3q27 | TRUE | 197 | alpha-2-HS-glycoprotein |
| AK024457 | | FALSE | | SPTREMBL Q9H7M1 KNOWN |
| AK097351 | | FALSE | | TIG47587 |
| ANGPT4 | 20p13 | TRUE | 51378 | angiopoietin 4 |
| ANKFN1 | 17q22 | TRUE | 162282 | ankyrin-repeat and fibronectin type III domain containing 1 |
| ANKRD11 | 16q24.3 | TRUE | 29123 | ankyrin repeat domain 11 |
| ANKS1B | 12q23.1 | TRUE | 56899 | ankyrin repeat and sterile alpha motif domain containing 1B |
| AP2A2 | 11p15.5 | TRUE | 161 | adaptor-related protein complex 2, alpha 2 subunit |
| APXL | Xp22.3 | TRUE | 357 | apical protein-like (Xenopus laevis) |
| ARHGAP8 | 22q13.31 | TRUE | 23779 | Rho GTPase activating protein 8 |
| ARL9 | 4q12 | TRUE | 132946 | ADP-ribosylation factor-like 9 |
| ARMC9 | 2q37.1 | TRUE | 80210 | armadillo repeat containing 9 |
| ARX | Xp22.1-p21.3 | TRUE | 170302 | aristaless related homeobox |
| ASTN2 | 9q33.1 | TRUE | 23245 | astrotactin 2 |
| ATP1A4 | 1q21-q23 | TRUE | 480 | ATPase, Na+/K+ transporting, alpha 4 polypeptide |
| ATP6V0E | 5q35.2 | TRUE | 8992 | ATPase, H+ transporting, lysosomal 9 kDa, V0 subunit e |
| ATXN7L1 | 7q22.1 | TRUE | 57485 | ataxin 7-like 1 |
| B3GALT5 | 21q22.3 | TRUE | 10317 | UDP-Gal:betaGlcNAc beta 1,3-galactosyltransferase, |
| | | | | polypeptide 5 |
| BAT1 | 6p21.3 | TRUE | 7919 | HLA-B associated transcript 1 |
| BAT2 | 6p21.3 | TRUE | 7916 | HLA-B associated transcript 2 |
| BAZ1A | 14q12-q13 | TRUE | 11177 | bromodomain adjacent to zinc finger domain, 1A |
| BAZ2B | 2q23-q24 | TRUE | 29994 | bromodomain adjacent to zinc finger domain, 2B |
| BCKDHB | 6q13-q15 | TRUE | 594 | branched chain keto acid dehydrogenase E1, beta |
| | | | | polypeptide (maple syrup urine disease) |
| BRUNOL4 | 18q12 | TRUE | 56853 | bruno-like 4, RNA binding protein (Drosophila) |
| BTBD11 | 12q23.3 | TRUE | 121551 | BTB (POZ) domain containing 11 |
| BTBD4 | 20q13.33 | TRUE | 140685 | BTB (POZ) domain containing 4 |
| C10ORF128 | 10q11.22 | TRUE | 170371 | chromosome 10 open reading frame 128 |
| C10ORF64 | 10q11.22 | TRUE | 159491 | chromosome 10 open reading frame 64 |
| C11ORF30 | 11q13.5 | TRUE | 56946 | chromosome 11 open reading frame 30 |
| C12ORF50 | 12q21.32 | TRUE | 160419 | chromosome 12 open reading frame 50 |
| C13ORF16 | 13q34 | TRUE | 121793 | chromosome 13 open reading frame 16 |
| C14ORF37 | 14q23.1 | TRUE | 145407 | chromosome 14 open reading frame 37 |
| C18ORF1 | 18p11.2 | TRUE | 753 | chromosome 18 open reading frame 1 |
| C18ORF10 | 18q12.2 | TRUE | 25941 | chromosome 18 open reading frame 10 |
| C18ORF58 | 18p11.22 | TRUE | 284222 | chromosome 18 open reading frame 58 |
| C19ORF2 | 19q12 | TRUE | 8725 | chromosome 19 open reading frame 2 |
| C1ORF96 | 1q42.13 | TRUE | 126731 | chromosome 1 open reading frame 96 |
| C2 | 6p21.3 | TRUE | 717 | complement component 2 |
| C20ORF174 | 20q13.32 | TRUE | 128611 | chromosome 20 open reading frame 174 |
| C20ORF30 | 20p12.3 | TRUE | 29058 | chromosome 20 open reading frame 30 |
| C21ORF29 | 21q22.3 | TRUE | 54084 | chromosome 21 open reading frame 29 |
| C21ORF7 | 21q22.3 | TRUE | 56911 | chromosome 21 open reading frame 7 |
| C22ORF5 | 22q12 | TRUE | 25829 | chromosome 22 open reading frame 5 |
| CACNB4 | 2q22-q23 | TRUE | 785 | calcium channel, voltage-dependent, beta 4 subunit |
| CAPN9 | 1q42.11-q42.3 | TRUE | 10753 | calpain 9 |
| CASP14 | 19p13.1 | TRUE | 23581 | caspase 14, apoptosis-related cysteine peptidase |
| CAST1 | 3p14.3 | FALSE | 26059 | CAZ-associated structural protein |
| CCDC101 | 16p11.2 | TRUE | 112869 | coiled-coil domain containing 101 |
| CCDC60 | 12q24.23 | TRUE | 160777 | coiled-coil domain containing 60 |
| CD151 | 11p15.5 | TRUE | 977 | CD151 molecule (Raph blood group) |
| CDC25C | 5q31 | TRUE | 995 | cell division cycle 25C |
| CDC42BPB | 14q32.3 | TRUE | 9578 | CDC42 binding protein kinase beta (DMPK-like) |
| CDH13 | 16q24.2-q24.3 | TRUE | 1012 | cadherin 13, H-cadherin (heart) |
| CDH4 | 20q13.3 | TRUE | 1002 | cadherin 4, type 1, R-cadherin (retinal) |
| CDH6 | 5p15.1-p14 | TRUE | 1004 | cadherin 6, type 2, K-cadherin (fetal kidney) |
| CLASP2 | 3p22.3 | TRUE | 23122 | cytoplasmic linker associated protein 2 |
| CLCA1 | 1p31-p22 | TRUE | 1179 | chloride channel, calcium activated, family member 1 |
| CLEC1A | 12p13.2 | TRUE | 51267 | C-type lectin domain family 1, member A |
| CMTM5 | 14q11.2 | TRUE | 116173 | CKLF-like MARVEL transmembrane domain containing 5 |
| CNGB3 | 8q21-q22 | TRUE | 54714 | cyclic nucleotide gated channel beta 3 |
| CNTNAP2 | 7q35-q36 | TRUE | 26047 | contactin associated protein-like 2 |
| COL13A1 | 10q22 | TRUE | 1305 | collagen, type XIII, alpha 1 |
| COL25A1 | 4q25 | TRUE | 84570 | collagen, type XXV, alpha 1 |
| COL4A2 | 13q34 | TRUE | 1284 | collagen, type IV, alpha 2 |
| CR2 | 1q32 | TRUE | 1380 | complement component (3d/Epstein Barr virus) receptor 2 |
| CSAD | 12q13.11-q14.3 | TRUE | 51380 | cysteine sulfinic acid decarboxylase |
| CTNNA2 | 2p12-p11.1 | TRUE | 1496 | catenin (cadherin-associated protein), alpha 2 |
| CXORF36 | | TRUE | 79742 | chromosome X open reading frame 36 |
| DCC | 18q21.3 | TRUE | 1630 | deleted in colorectal carcinoma |
| DENND1A | 9q33.2 | TRUE | 57706 | DENN/MADD domain containing 1A |
| DEPDC2 | 8q13.2 | TRUE | 80243 | DEP domain containing 2 |
| DGKI | 7q32.3-q33 | TRUE | 9162 | diacylglycerol kinase, iota |
| DHRS7 | 14q23.1 | TRUE | 51635 | dehydrogenase/reductase (SDR family) member 7 |
| DIAPH2 | Xq22 | TRUE | 1730 | diaphanous homolog 2 (Drosophila) |
| DIP2C | 10p15.3 | TRUE | 22982 | DIP2 disco-interacting protein 2 homolog C (Drosophila) |
| DKFZP686A01247 | 4p13 | FALSE | 22998 | hypothetical protein |
| DLC1 | 8p22 | TRUE | 10395 | deleted in liver cancer 1 |
| DLEU2 | 13q14.3 | TRUE | 8847 | deleted in lymphocytic leukemia, 2 |
| DMD | Xp21.2 | TRUE | 1756 | dystrophin (muscular dystrophy, Duchenne and Becker types) |
| DNAH8 | 6p21.31-p21.1 | TRUE | 1769 | dynein, axonemal, heavy polypeptide 8 |
| DNAJC15 | 13q14.1 | TRUE | 29103 | DnaJ (Hsp40) homolog, subfamily C, member 15 |
| DNMT2 | 10p15.1 | TRUE | 1787 | DNA (cytosine-5-)-methyltransferase 2 |
| DNMT3A | 2p23 | TRUE | 1788 | DNA (cytosine-5-)-methyltransferase 3 alpha |
| DOCK2 | 5q35.1 | TRUE | 1794 | dedicator of cytokinesis 2 |
| DPT | 1q12-q23 | TRUE | 1805 | dermatopontin |
| DPYSL3 | 5q32 | TRUE | 1809 | dihydropyrimidinase-like 3 |
| DSC3 | 18q12.1 | TRUE | 1825 | desmocollin 3 |
| DSCAM | 21q22.2 | TRUE | 1826 | Down syndrome cell adhesion molecule |
| DYNC1I1 | 7q21.3-q22.1 | TRUE | 1780 | dynein, cytoplasmic 1, intermediate chain 1 |
| DYNC2H1 | 11q21-q22.1 | TRUE | 79659 | dynein, cytoplasmic 2, heavy chain 1 |
| EDIL3 | 5q14 | TRUE | 10085 | EGF-like repeats and discoidin I-like domains 3 |
| EEF1D | 8q24.3 | TRUE | 1936 | eukaryotic translation elongation factor 1 delta (guanine |
| | | | | nucleotide exchange protein) |
| EEFSEC | 3q21.3 | TRUE | 60678 | eukaryotic elongation factor, selenocysteine-tRNA-specific |
| EHD4 | 15q11.1 | TRUE | 30844 | EH-domain containing 4 |
| EIF2C2 | 8q24 | TRUE | 27161 | eukaryotic translation initiation factor 2C, 2 |
| EIF3S2 | 1p34.1 | TRUE | 8668 | eukaryotic translation initiation factor 3, subunit 2 beta, 36 kDa |
| EIF3S7 | 22q13.1 | TRUE | 8664 | eukaryotic translation initiation factor 3, subunit 7 zeta, |
| | | | | 66/67 kDa |
| EIF4A2 | 3q28 | TRUE | 1974 | eukaryotic translation initiation factor 4A, isoform 2 |
| ELOVL7 | 5q12.1 | TRUE | 79993 | ELOVL family member 7, elongation of long chain fatty acids |
| | | | | (yeast) |
| EMP2 | 16p13.2 | TRUE | 2013 | epithelial membrane protein 2 |
| ERBB4 | 2q33.3-q34 | TRUE | 2066 | v-erb-a erythroblastic leukemia viral oncogene homolog 4 |
| | | | | (avian) |
| ETV6 | 12p13 | TRUE | 2120 | ets variant gene 6 (TEL oncogene) |
| EVC | 4p16 | TRUE | 2121 | Ellis van Creveld syndrome |
| EXOD1 | 16p12.2 | TRUE | 112479 | exonuclease domain containing 1 |
| FAM19A2 | 12q14.1 | TRUE | 338811 | family with sequence similarity 19 (chemokine (C—C motif)- |
| | | | | like), member A2 |
| FAM42B | 15q25.2 | TRUE | 388159 | family with sequence similarity 42, member B |
| FAM80B | 12p13.31 | TRUE | 57494 | family with sequence similarity 80, member B |
| FAM82A | 2p22.2 | TRUE | 151393 | family with sequence similarity 82, member A |
| FBXL21 | 5q31 | TRUE | 26223 | F-box and leucine-rich repeat protein 21 |
| FGF12 | 3q28 | TRUE | 2257 | fibroblast growth factor 12 |
| FHIT | 3p14.2 | TRUE | 2272 | fragile histidine triad gene |
| FIBCD1 | 9q34.12-q34.13 | TRUE | 84929 | fibrinogen C domain containing 1 |
| FLJ00049 | 7p22.2 | FALSE | 645372 | FLJ00049 protein |
| FLJ16124 | 2p14 | FALSE | 440867 | FLJ16124 protein |
| FLJ20674 | 12q24.23 | FALSE | 54621 | hypothetical protein FLJ20674 |
| FLJ30838 | 2p16.1 | FALSE | 400955 | hypothetical gene supported by AL832565 |
| FLJ32871 | 16p13.13 | FALSE | 146279 | hypothetical protein FLJ32871 |
| FLJ32894 | 12p12.1 | FALSE | 144360 | hypothetical protein LOC144360 |
| FLJ35024 | 9p24.2 | FALSE | 401491 | hypothetical LOC401491 |
| FLJ36748 | 5q33.1 | FALSE | 134265 | hypothetical protein FLJ36748 |
| FLJ39502 | 2q31.2 | FALSE | 285025 | hypothetical protein FLJ39502 |
| FLJ40411 | 2q37.3 | FALSE | 375316 | FLJ40411 protein |
| FLJ42342 | 9q22.2 | FALSE | 401539 | hypothetical gene supported by AK124333 |
| FNBP1 | 9q34 | TRUE | 23048 | formin binding protein 1 |
| FOXRED2 | 22q12.3 | TRUE | 80020 | FAD-dependent oxidoreductase domain containing 2 |
| FRMD3 | 9q21.32 | TRUE | 257019 | FERM domain containing 3 |
| FRMD4A | 10p13 | TRUE | 55691 | FERM domain containing 4A |
| FSTL4 | 5q31.1 | TRUE | 23105 | follistatin-like 4 |
| FUBP3 | 9q34.11-q34.12 | TRUE | 8939 | far upstream element (FUSE) binding protein 3 |
| GABBR1 | 6p21.31 | TRUE | 2550 | gamma-aminobutyric acid (GABA) B receptor, 1 |
| GABBR2 | 9q22.1-q22.3 | TRUE | 9568 | gamma-aminobutyric acid (GABA) B receptor, 2 |
| GABRB2 | 5q34 | TRUE | 2561 | gamma-aminobutyric acid (GABA) A receptor, beta 2 |
| GALNT14 | 2p23.1 | TRUE | 79623 | UDP-N-acetyl-alpha-D-galactosamine:polypeptide N- |
| | | | | acetylgalactosaminyltransferase 14 (GalNAc-T14) |
| GALNT17 | 4q34.1 | FALSE | 442117 | polypeptide N-acetylgalactosaminyltransferase 17 |
| GALT | 9p13 | TRUE | 2592 | galactose-1-phosphate uridylyltransferase |
| GAS7 | 17p13.1 | TRUE | 8522 | growth arrest-specific 7 |
| GATA4 | 8p23.1-p22 | TRUE | 2626 | GATA binding protein 4 |
| GENE_FOR_TAR92809 | | FALSE | | |
| GENE_FOR_TAR92812 | | FALSE | | |
| GENE_FOR_TAR92825 | | FALSE | | |
| GLIS3 | 9p24.2 | TRUE | 169792 | GLIS family zinc finger 3 |
| GLRA2 | Xp22.1-p21.3 | TRUE | 2742 | glycine receptor, alpha 2 |
| GMCL1 | 2p13.3 | TRUE | 64395 | germ cell-less homolog 1 (Drosophila) |
| GPC5 | 13q32 | TRUE | 2262 | glypican 5 |
| GRIP1 | 12q14.3 | TRUE | 23426 | glutamate receptor interacting protein 1 |
| GRM7 | 3p26.1-p25.1 | TRUE | 2917 | glutamate receptor, metabotropic 7 |
| GTF2IRD1 | 7q11.23 | TRUE | 9569 | GTF2I repeat domain containing 1 |
| HCG18 | 6p21.3 | TRUE | 414777 | HLA complex group 18 |
| HCG9 | 6p21.3 | TRUE | 10255 | HLA complex group 9 |
| HCP5 | 6p21.3 | TRUE | 10866 | HLA complex P5 |
| HDAC2 | 6q21 | TRUE | 3066 | histone deacetylase 2 |
| HIVEP3 | 1p34 | TRUE | 59269 | human immunodeficiency virus type I enhancer binding |
| | | | | protein 3 |
| HLA-A | 6p21.3 | TRUE | 3105 | major histocompatibility complex, class I, A |
| HLA-B | 6p21.3 | TRUE | 3106 | major histocompatibility complex, class I, B |
| HLA-C | 6p21.3 | TRUE | 3107 | major histocompatibility complex, class I, C |
| HLA-G | 6p21.3 | TRUE | 3135 | HLA-G histocompatibility antigen, class I, G |
| HNT | 11q25 | FALSE | 50863 | neurotrimin |
| HS3ST3B1 | 17p12-p11.2 | TRUE | 9953 | heparan sulfate (glucosamine) 3-O-sulfotransferase 3B1 |
| HS3ST4 | 16p11.2 | TRUE | 9951 | heparan sulfate (glucosamine) 3-O-sulfotransferase 4 |
| HTR2C | Xq24 | TRUE | 3358 | 5-hydroxytryptamine (serotonin) receptor 2C |
| IL4R | 16p11.2-12.1 | TRUE | 3566 | interleukin 4 receptor |
| IPPK | 9q21.33-q22.31 | TRUE | 64768 | inositol 1,3,4,5,6-pentakisphosphate 2-kinase |
| IQGAP1 | 15q26.1 | TRUE | 8826 | IQ motif containing GTPase activating protein 1 |
| IQGAP2 | 5q13.3 | TRUE | 10788 | IQ motif containing GTPase activating protein 2 |
| ITFG1 | 16q12.1 | TRUE | 81533 | integrin alpha FG-GAP repeat containing 1 |
| JDP2 | 14q24.3 | FALSE | 122953 | jun dimerization protein 2 |
| KALRN | 3q21.1-q21.2 | TRUE | 8997 | kalirin, RhoGEF kinase |
| KCNC1 | 11p15 | TRUE | 3746 | potassium voltage-gated channel, Shaw-related subfamily, |
| | | | | member 1 |
| KCNMA1 | 10q22.3 | TRUE | 3778 | potassium large conductance calcium-activated channel, |
| | | | | subfamily M, alpha member 1 |
| KCNMB2 | 3q26.2-q27.1 | TRUE | 10242 | potassium large conductance calcium-activated channel, |
| | | | | subfamily M, beta member 2 |
| KCTD20 | 6p21.31 | TRUE | 222658 | potassium channel tetramerisation domain containing 20 |
| KIAA0090 | 1p36.13 | TRUE | 23065 | KIAA0090 |
| KIAA0284 | 14q32.33 | TRUE | 283638 | KIAA0284 |
| KIAA0513 | 16q24.1 | TRUE | 9764 | KIAA0513 |
| KIAA0556 | 16p12.1-p11.2 | FALSE | 23247 | KIAA0556 protein |
| KIAA1026 | 1p36.21 | FALSE | 23254 | kazrin |
| KIAA1328 | 18q12.2 | TRUE | 57536 | KIAA1328 |
| KIAA1660 | | FALSE | 85374 | KIAA1660 protein |
| KIAA1679 | 2q21-q22 | FALSE | 80731 | KIAA1679 protein |
| KIAA1920 | 15q25.2 | FALSE | 114817 | KIAA1920 protein |
| KIAA1975 | 10q23.2 | FALSE | 119385 | KIAA1975 protein similar to MRIP2 |
| KPNA3 | 13q14.3 | TRUE | 3839 | karyopherin alpha 3 (importin alpha 4) |
| KSR2 | 12q24.22-q24.23 | TRUE | 283455 | kinase suppressor of ras 2 |
| LDLRAD3 | 11p13 | TRUE | 143458 | low density lipoprotein receptor class A domain containing 3 |
| LOC123103 | 14q11.2 | FALSE | 123103 | similar to kelch-like 9 |
| LOC130940 | 2q24.1 | FALSE | 130940 | hypothetical protein BC015395 |
| LOC147645 | 19q13.33 | FALSE | 147645 | hypothetical protein LOC147645 |
| LOC150400 | 22q13.33 | FALSE | 150400 | similar to 60S ribosomal protein L35 |
| LOC154860 | 7q31.33 | FALSE | 154860 | hypothetical protein LOC154860 |
| LOC255167 | 5p15.31 | FALSE | 255167 | hypothetical protein LOC255167 |
| LOC284825 | 21q21.3 | FALSE | 284825 | hypothetical protein LOC284825 |
| LOC284930 | 22q13.32 | FALSE | 284930 | hypothetical protein LOC284930 |
| LOC285692 | 5p15.31 | FALSE | 285692 | hypothetical protein LOC285692 |
| LOC339483 | 1p35.1 | FALSE | 339483 | hypothetical LOC339483 |
| LOC388814 | 21q21.1 | FALSE | 388814 | hypothetical LOC388814 |
| LOC389072 | 2q33.3 | FALSE | 389072 | hypothetical protein LOC389072 |
| LOC399978 | 11q24.3 | FALSE | 399978 | hypothetical gene supported by BC031979 |
| LOC400924 | 22q12.2 | FALSE | 400924 | hypothetical gene supported by AK056895 |
| LOC402665 | 19q13.33 | FALSE | 402665 | similar to opioid binding protein/cell adhesion molecule-like |
| LOC440173 | 14q12 | FALSE | 440173 | LOC440173 |
| LOC440280 | 15q21.3 | FALSE | 440280 | similar to CCR4-NOT transcription complex, subunit 6-like |
| | | | | isoform 1 |
| LOC440402 | 17p12 | FALSE | 440402 | hypothetical LOC440402 |
| LOC440704 | 1q31.1-q31.2 | FALSE | 440704 | hypothetical gene supported by BC042042 |
| LOC441031 | 4q22.3 | FALSE | 441031 | hypothetical LOC441031 |
| LOC441204 | 7p15.2 | FALSE | 441204 | hypothetical locus LOC441204 |
| LOC441672 | 14q11.2 | FALSE | 441672 | similar to solute carrier family 12, member 3 |
| LOC441792 | 17q12 | FALSE | 441792 | similar to Nitric oxide synthase, inducible (NOS type II) |
| | | | | (Inducible NOS) (iNOS) (Hepatocyte NOS) (HEP-NOS) |
| LOC51334 | 5q23.1 | FALSE | 51334 | mesenchymal stem cell protein DSC54 |
| LOC553158 | 22q13 | FALSE | 553158 | PRR5-ARHGAP8 fusion |
| LOC641906 | | FALSE | 641906 | hypothetical protein LOC641906 |
| LOC641939 | | FALSE | 641939 | hypothetical protein LOC641939 |
| LOC642132 | | FALSE | 642132 | similar to roundabout 1 isoform b |
| LOC642724 | 11p15.5 | FALSE | 642724 | similar to mucin 6, gastric |
| LOC643150 | 10q23.2 | FALSE | 643150 | similar to Ribosome biogenesis protein BMS1 homolog |
| LOC644094 | 1p12 | FALSE | 644094 | hypothetical protein LOC644094 |
| LOC644347 | 14q13.1 | FALSE | 644347 | hypothetical protein LOC644347 |
| LOC644536 | 5q34 | FALSE | 644536 | hypothetical protein LOC644536 |
| LOC645358 | 9q33.1 | FALSE | 645358 | hypothetical protein LOC645358 |
| LOC645521 | 2p24.2 | FALSE | 645521 | similar to Zinc finger FYVE domain-containing protein 9 |
| | | | | (Mothers against decapentaplegic homolog interacting |
| | | | | protein) (Madh-interacting protein) (Smad anchor for receptor |
| | | | | activation) (Receptor activation anchor) (hSARA) (Novel |
| | | | | serine protease) (NSP . . . |
| LOC645995 | 4q28.3 | FALSE | 645995 | hypothetical protein LOC645995 |
| LOC646099 | 6q25.1 | FALSE | 646099 | hypothetical protein LOC646099 |
| LOC646107 | 8q24.3 | FALSE | 646107 | hypothetical protein LOC646107 |
| LOC646324 | 2q35 | FALSE | 646324 | hypothetical protein LOC646324 |
| LOC647021 | 2p16.1 | FALSE | 647021 | hypothetical protein LOC647021 |
| LOC647029 | 2p16.1 | FALSE | 647029 | hypothetical protein LOC647029 |
| LOC647666 | | FALSE | 647666 | hypothetical protein LOC647666 |
| LOC648034 | | FALSE | 648034 | hypothetical protein LOC648034 |
| LOC649021 | | FALSE | 649021 | hypothetical protein LOC649021 |
| LOC650422 | | FALSE | 650422 | similar to RAS p21 protein activator 4 |
| LOC650481 | | FALSE | 650481 | similar to kelch-like 9 |
| LOC651125 | | FALSE | 651125 | similar to zinc finger protein 567 |
| LOC651341 | | FALSE | 651341 | hypothetical protein LOC651341 |
| LOC651586 | | FALSE | 651586 | hypothetical protein LOC651586 |
| LOC652160 | | FALSE | 652160 | hypothetical protein LOC652160 |
| LOC652202 | | FALSE | 652202 | hypothetical protein LOC652202 |
| LOC652214 | | FALSE | 652214 | hypothetical protein LOC652214 |
| LOC652408 | | FALSE | 652408 | similar to myosin light chain kinase isoform 1 |
| LOC653062 | Xq23 | FALSE | 653062 | similar to 5-hydroxytryptamine (serotonin) receptor 2C |
| LOC653175 | 7p12.1 | FALSE | 653175 | family with sequence similarity 29, member A pseudogene |
| LOC653783 | 16p12.2 | FALSE | 653783 | similar to similar to RIKEN cDNA 4933424N09 gene |
| LOC653796 | 2q37.1 | FALSE | 653796 | similar to SH2 containing inositol phosphatase isoform b |
| LOC653891 | | FALSE | 653891 | similar to heterogeneous nuclear ribonucleoprotein A1-like |
| LOC653976 | | FALSE | 653976 | similar to 5-hydroxytryptamine (serotonin) receptor 2C |
| LPA | 6q26 | TRUE | 4018 | lipoprotein, Lp(a) |
| LRBA | 4q31.3 | TRUE | 987 | LPS-responsive vesicle trafficking, beach and anchor |
| | | | | containing |
| LRCH1 | 13q14.13-q14.2 | TRUE | 23143 | leucine-rich repeats and calponin homology (CH) domain |
| | | | | containing 1 |
| LRP1B | 2q21.2 | TRUE | 53353 | low density lipoprotein-related protein 1B (deleted in tumors) |
| LRP2 | 2q24-q31 | TRUE | 4036 | low density lipoprotein-related protein 2 |
| LRRN1 | 3p26.2 | TRUE | 57633 | leucine rich repeat neuronal 1 |
| LTBP1 | 2p22-p21 | TRUE | 4052 | latent transforming growth factor beta binding protein 1 |
| LY75 | 2q24 | TRUE | 4065 | lymphocyte antigen 75 |
| LYPLAL1 | 1q41 | TRUE | 127018 | lysophospholipase-like 1 |
| MAD1L1 | 7p22 | TRUE | 8379 | MAD1 mitotic arrest deficient-like 1 (yeast) |
| MAGI1 | 3p14.1 | TRUE | 9223 | membrane associated guanylate kinase, WW and PDZ |
| | | | | domain containing 1 |
| MAGI2 | 7q21 | TRUE | 9863 | membrane associated guanylate kinase, WW and PDZ |
| | | | | domain containing 2 |
| MAMDC1 | 14q21.3 | TRUE | 161357 | MAM domain containing 1 |
| MAML2 | 11q21 | TRUE | 84441 | mastermind-like 2 (Drosophila) |
| MAML3 | 4q28 | TRUE | 55534 | mastermind-like 3 (Drosophila) |
| MAP1B | 5q13 | TRUE | 4131 | microtubule-associated protein 1B |
| MAP3K7IP2 | 6q25.1-q25.3 | TRUE | 23118 | mitogen-activated protein kinase kinase kinase 7 interacting |
| | | | | protein 2 |
| MARCO | 2q12-q13 | TRUE | 8685 | macrophage receptor with collagenous structure |
| MAST3 | 19p13.11 | TRUE | 23031 | microtubule associated serine/threonine kinase 3 |
| MATN4 | 20q13.1-q13.2 | TRUE | 8785 | matrilin 4 |
| MBNL2 | 13q32.1 | TRUE | 10150 | muscleblind-like 2 (Drosophila) |
| ME1 | 6q12 | TRUE | 4199 | malic enzyme 1, NADP(+)-dependent, cytosolic |
| MGC15875 | 5q35.3 | FALSE | 85007 | hypothetical protein LOC85007, isoform 2 |
| MGC34713 | 5q15 | FALSE | 285600 | hypothetical protein MGC34713 |
| MGC48595 | 14q24.3 | FALSE | 399671 | hypothetical MGC48595 |
| MICB | 6p21.3 | TRUE | 4277 | MHC class I polypeptide-related sequence B |
| MMP16 | 8q21 | TRUE | 4325 | matrix metallopeptidase 16 (membrane-inserted) |
| MOV10L1 | 22q13.33 | TRUE | 54456 | Mov10l1, Moloney leukemia virus 10-like 1, homolog (mouse) |
| MRPL1 | 4q21.1 | TRUE | 65008 | mitochondrial ribosomal protein L1 |
| MRPL54 | 19p13.3 | TRUE | 116541 | mitochondrial ribosomal protein L54 |
| MTHFD1L | 6q25.1 | TRUE | 25902 | methylenetetrahydrofolate dehydrogenase (NADP+ |
| | | | | dependent) 1-like |
| MTMR3 | 22q12.2 | TRUE | 8897 | myotubularin related protein 3 |
| MTNR1A | 4q35.1 | TRUE | 4543 | melatonin receptor 1A |
| MUC6 | 11p15.5-p15.4 | TRUE | 4588 | mucin 6, oligomeric mucus/gel-forming |
| MUSK | 9q31.3-q32 | TRUE | 4593 | muscle, skeletal, receptor tyrosine kinase |
| MXD1 | 2p13-p12 | TRUE | 4084 | MAX dimerization protein 1 |
| MYH6 | 14q12 | TRUE | 4624 | myosin, heavy polypeptide 6, cardiac muscle, alpha |
| | | | | (cardiomyopathy, hypertrophic 1) |
| MYO10 | 5p15.1-p14.3 | TRUE | 4651 | myosin X |
| MYO1D | 17q11-q12 | TRUE | 4642 | myosin ID |
| MYO1E | 15q21-q22 | TRUE | 4643 | myosin IE |
| MYRIP | 3p22.1 | TRUE | 25924 | myosin VIIA and Rab interacting protein |
| NAALADL2 | 3q26.31 | TRUE | 254827 | N-acetylated alpha-linked acidic dipeptidase-like 2 |
| NAP5 | 2q21.2 | FALSE | 344148 | Nck-associated protein 5 |
| NELL1 | 11p15.2-p15.1 | TRUE | 4745 | NEL-like 1 (chicken) |
| NFASC | 1q32.1 | TRUE | 23114 | neurofascin homolog (chicken) |
| NFATC1 | 18q23 | TRUE | 4772 | nuclear factor of activated T-cells, cytoplasmic, calcineurin- |
| | | | | dependent 1 |
| NIN | 14q22.1 | TRUE | 51199 | ninein (GSK3B interacting protein) |
| NLGN1 | 3q26.31 | TRUE | 22871 | neuroligin 1 |
| NOC3L | 10q23.33 | TRUE | 64318 | nucleolar complex associated 3 homolog (S. cerevisiae) |
| NOVA1 | 14q | TRUE | 4857 | neuro-oncological ventral antigen 1 |
| NPHP4 | 1p36.22 | TRUE | 261734 | nephronophthisis 4 |
| NRXN1 | 2p16.3 | TRUE | 9378 | neurexin 1 |
| NSUN6 | 10p12.33 | TRUE | 221078 | NOL1/NOP2/Sun domain family, member 6 |
| NTNG1 | 1p13.3 | TRUE | 22854 | netrin G1 |
| NUMBL | 19q13.13-q13.2 | TRUE | 9253 | numb homolog (Drosophila)-like |
| OPCML | 11q25 | TRUE | 4978 | opioid binding protein/cell adhesion molecule-like |
| OR1S2 | 11q12.1 | TRUE | 219958 | olfactory receptor, family 1, subfamily S, member 2 |
| PACRG | 6q26 | TRUE | 135138 | PARK2 co-regulated |
| PAH | 12q22-q24.2 | TRUE | 5053 | phenylalanine hydroxylase |
| PAP2D | 1p21.3 | FALSE | 163404 | phosphatidic acid phosphatase type 2 |
| PAQR5 | 15q23 | TRUE | 54852 | progestin and adipoQ receptor family member V |
| PARK2 | 6q25.2-q27 | TRUE | 5071 | Parkinson disease (autosomal recessive, juvenile) 2, parkin |
| PBX1 | 1q23 | TRUE | 5087 | pre-B-cell leukemia transcription factor 1 |
| PCCA | 13q32 | TRUE | 5095 | propionyl Coenzyme A carboxylase, alpha polypeptide |
| PCSK5 | 9q21.3 | TRUE | 5125 | proprotein convertase subtilisin/kexin type 5 |
| PCSK9 | 1p32.3 | TRUE | 255738 | proprotein convertase subtilisin/kexin type 9 |
| PDE8B | 5q14.1 | TRUE | 8622 | phosphodiesterase 8B |
| PDZD2 | 5p13.3 | TRUE | 23037 | PDZ domain containing 2 |
| PGM2 | 4p14 | TRUE | 55276 | phosphoglucomutase 2 |
| PHYH | 10pter-p11.2 | TRUE | 5264 | phytanoyl-CoA 2-hydroxylase |
| PKHD1 | 6p12.2 | TRUE | 5314 | polycystic kidney and hepatic disease 1 (autosomal |
| | | | | recessive) |
| PLCD3 | 17q21.31 | TRUE | 113026 | phospholipase C, delta 3 |
| PLEKHA7 | 11p15.1 | TRUE | 144100 | pleckstrin homology domain containing, family A member 7 |
| PLS3 | Xq23 | TRUE | 5358 | plastin 3 (T isoform) |
| PLXNA2 | 1q32.2 | TRUE | 5362 | plexin A2 |
| PLXNA4B | 7q32.3 | TRUE | 91584 | plexin A4, B |
| POLR2L | 11p15 | TRUE | 5441 | polymerase (RNA) II (DNA directed) polypeptide L, 7.6 kDa |
| PPP2R5A | 1q32.2-q32.3 | TRUE | 5525 | protein phosphatase 2, regulatory subunit B (B56), alpha |
| | | | | isoform |
| PRKG1 | 10q11.2 | TRUE | 5592 | protein kinase, cGMP-dependent, type I |
| PRODH | 22q11.21 | TRUE | 5625 | proline dehydrogenase (oxidase) 1 |
| PSD3 | 8pter-p23.3 | TRUE | 23362 | pleckstrin and Sec7 domain containing 3 |
| PSMB8 | 6p21.3 | TRUE | 5696 | proteasome (prosome, macropain) subunit, beta type, 8 |
| | | | | (large multifunctional peptidase 7) |
| PTCRA | 6p21.3 | TRUE | 171558 | pre T-cell antigen receptor alpha |
| PTD004 | 2q31.1 | FALSE | 29789 | GTP-binding protein PTD004 |
| PTN | 7q33-q34 | TRUE | 5764 | pleiotrophin (heparin binding growth factor 8, neurite growth- |
| | | | | promoting factor 1) |
| PTPN14 | 1q32.2 | TRUE | 5784 | protein tyrosine phosphatase, non-receptor type 14 |
| PTPRH | 19q13.4 | TRUE | 5794 | protein tyrosine phosphatase, receptor type, H |
| PTPRK | 6q22.2-23.1 | TRUE | 5796 | protein tyrosine phosphatase, receptor type, K |
| PYCRL | 8q24.3 | TRUE | 65263 | pyrroline-5-carboxylate reductase-like |
| RAB27A | 15q15-q21.1 | TRUE | 5873 | RAB27A, member RAS oncogene family |
| RABL4 | 22q13.1 | TRUE | 11020 | RAB, member of RAS oncogene family-like 4 |
| RASAL1 | 12q23-q24 | TRUE | 8437 | RAS protein activator like 1 (GAP1 like) |
| RAXL1 | 19p13.3 | TRUE | 84839 | retina and anterior neural fold homeobox like 1 |
| RBPSUHL | 20q12-q13.1 | TRUE | 11317 | recombining binding protein suppressor of hairless |
| | | | | (Drosophila)-like |
| RGS6 | 14q24.3 | TRUE | 9628 | regulator of G-protein signalling 6 |
| RNF39 | 6p21.3 | TRUE | 80352 | ring finger protein 39 |
| ROBO1 | 3p12 | TRUE | 6091 | roundabout, axon guidance receptor, homolog 1 (Drosophila) |
| RORA | 15q21-q22 | TRUE | 6095 | RAR-related orphan receptor A |
| RP11-54H7.1 | 13q33.3 | FALSE | 23026 | myosin heavy chain Myr 8 |
| RP13- | Xq26.1 | FALSE | 158763 | hypothetical protein FLJ30058 |
| 102H20.1 | | | | |
| RPH3A | 12q24.13 | TRUE | 22895 | rabphilin 3A homolog (mouse) |
| RUNX1 | 21q22.3 | TRUE | 861 | runt-related transcription factor 1 (acute myeloid leukemia 1; |
| | | | | aml1 oncogene) |
| RYR2 | 1q42.1-q43 | TRUE | 6262 | ryanodine receptor 2 (cardiac) |
| SCARB2 | 4q21.1 | TRUE | 950 | scavenger receptor class B, member 2 |
| SCD5 | 4q21.22 | TRUE | 79966 | stearoyl-CoA desaturase 5 |
| SCHIP1 | 3q25.33 | TRUE | 29970 | schwannomin interacting protein 1 |
| SDC2 | 8q22-q23 | TRUE | 6383 | syndecan 2 (heparan sulfate proteoglycan 1, cell surface- |
| | | | | associated, fibroglycan) |
| SEC31L2 | 10q24.31 | TRUE | 25956 | SEC31-like 2 (S. cerevisiae) |
| SEMA3D | 7q21.11 | TRUE | 223117 | sema domain, immunoglobulin domain (Ig), short basic |
| | | | | domain, secreted, (semaphorin) 3D |
| SEMA5A | 5p15.2 | TRUE | 9037 | sema domain, seven thrombospondin repeats (type 1 and |
| | | | | type 1-like), transmembrane domain (TM) and short |
| | | | | cytoplasmic domain, (semaphorin) 5A |
| SERGEF | 11p14.3 | TRUE | 26297 | secretion regulating guanine nucleotide exchange factor |
| SIGIRR | 11p15.5 | TRUE | 59307 | single immunoglobulin and toll-interleukin 1 receptor (TIR) |
| | | | | domain |
| SLC25A21 | 14q11.2 | TRUE | 89874 | solute carrier family 25 (mitochondrial oxodicarboxylate |
| | | | | carrier), member 21 |
| SLC28A1 | 15q25-26 | TRUE | 9154 | solute carrier family 28 (sodium-coupled nucleoside |
| | | | | transporter), member 1 |
| SLC28A2 | 15q15 | TRUE | 9153 | solute carrier family 28 (sodium-coupled nucleoside |
| | | | | transporter), member 2 |
| SLC2A9 | 4p16-p15.3 | TRUE | 56606 | solute carrier family 2 (facilitated glucose transporter), |
| | | | | member 9 |
| SLC39A13 | 11p11.2 | TRUE | 91252 | solute carrier family 39 (zinc transporter), member 13 |
| SLC5A7 | 2q12 | TRUE | 60482 | solute carrier family 5 (choline transporter), member 7 |
| SLC5A8 | 12q23.2 | TRUE | 160728 | solute carrier family 5 (iodide transporter), member 8 |
| SLC6A1 | 3p25-p24 | TRUE | 6529 | solute carrier family 6 (neurotransmitter transporter, GABA), |
| | | | | member 1 |
| SLC6A16 | 19q13.1-q13.4 | TRUE | 28968 | solute carrier family 6, member 16 |
| SLC6A5 | 11p15.2-p15.1 | TRUE | 9152 | solute carrier family 6 (neurotransmitter transporter, glycine), |
| | | | | member 5 |
| SLC9A9 | 3q24 | TRUE | 285195 | solute carrier family 9 (sodium/hydrogen exchanger), member 9 |
| SLCO1C1 | 12p12.2 | TRUE | 53919 | solute carrier organic anion transporter family, member 1C1 |
| SLIT3 | 5q35 | TRUE | 6586 | slit homolog 3 (Drosophila) |
| SMARCA2 | 9p22.3 | TRUE | 6595 | SWI/SNF related, matrix associated, actin dependent |
| | | | | regulator of chromatin, subfamily a, member 2 |
| SMPDL3B | 1p35.3 | TRUE | 27293 | sphingomyelin phosphodiesterase, acid-like 3B |
| SNX14 | 6q14.3 | TRUE | 57231 | sorting nexin 14 |
| SNX19 | 11q25 | TRUE | 399979 | sorting nexin 19 |
| SOX5 | 12p12.1 | TRUE | 6660 | SRY (sex determining region Y)-box 5 |
| SP100 | 2q37.1 | TRUE | 6672 | SP100 nuclear antigen |
| SPATA17 | 1q41 | TRUE | 128153 | spermatogenesis associated 17 |
| SPI1 | 11p11.2 | TRUE | 6688 | spleen focus forming virus (SFFV) proviral integration |
| | | | | oncogene spi1 |
| SPIRE2 | 16q24 | TRUE | 84501 | spire homolog 2 (Drosophila) |
| SPOCK1 | 5q31 | TRUE | 6695 | sparc/osteonectin, cwcv and kazal-like domains proteoglycan |
| | | | | (testican) 1 |
| SPP2 | 2q37-qter | TRUE | 6694 | secreted phosphoprotein 2, 24 kDa |
| SPSB1 | 1p36.22 | TRUE | 80176 | splA/ryanodine receptor domain and SOCS box containing 1 |
| SPSB4 | 3q23 | TRUE | 92369 | splA/ryanodine receptor domain and SOCS box containing 4 |
| ST7 | 7q31.1-q31.3 | TRUE | 7982 | suppression of tumorigenicity 7 |
| ST8SIA5 | 18q21.1 | TRUE | 29906 | ST8 alpha-N-acetyl-neuraminide alpha-2,8-sialyltransferase 5 |
| STK10 | 5q35.1 | TRUE | 6793 | serine/threonine kinase 10 |
| STK38 | 6p21 | TRUE | 11329 | serine/threonine kinase 38 |
| STK4 | 20q11.2-q13.2 | TRUE | 6789 | serine/threonine kinase 4 |
| SULF1 | 8q13.2-q13.3 | TRUE | 23213 | sulfatase 1 |
| SYT16 | 14q23.2 | TRUE | 83851 | synaptotagmin XVI |
| TAP1 | 6p21.3 | TRUE | 6890 | transporter 1, ATP-binding cassette, sub-family B (MDR/TAP) |
| TBX4 | 17q21-q22 | TRUE | 9496 | T-box 4 |
| TCAG7.981 | 7p15.2-p15.1 | FALSE | 221895 | juxtaposed with another zinc finger gene 1 |
| TCBA1 | 6q21 | TRUE | 154215 | T-cell lymphoma breakpoint associated target 1 |
| TCF19 | 6p21.3 | TRUE | 6941 | transcription factor 19 (SC1) |
| TGFBR3 | 1p33-p32 | TRUE | 7049 | transforming growth factor, beta receptor III (betaglycan, |
| | | | | 300 kDa) |
| THADA | 2p21 | TRUE | 63892 | thyroid adenoma associated |
| TIG44321 | | FALSE | | |
| TIG48667 | | FALSE | | LOC553158 |
| TLR5 | 1q41-q42 | TRUE | 7100 | toll-like receptor 5 |
| TMEM132D | 12q24.32-q24.33 | TRUE | 121256 | transmembrane protein 132D |
| TMEM163 | 2q21.3 | TRUE | 81615 | transmembrane protein 163 |
| TMEM35 | Xq22.1 | TRUE | 59353 | transmembrane protein 35 |
| TNR | 1q24 | TRUE | 7143 | tenascin R (restrictin, janusin) |
| TNS1 | 2q35-q36 | TRUE | 7145 | tensin 1 |
| TSEN54 | 17q25.1 | TRUE | 283989 | tRNA splicing endonuclease 54 homolog (S. cerevisiae) |
| TSPAN18 | 11p11.2 | TRUE | 90139 | tetraspanin 18 |
| TTC27 | 2p22.3 | TRUE | 55622 | tetratricopeptide repeat domain 27 |
| TXN2 | 22q13.1 | TRUE | 25828 | thioredoxin 2 |
| UBE2N | 12q22 | TRUE | 7334 | ubiquitin-conjugating enzyme E2N (UBC13 homolog, yeast) |
| UFD1L | 22q11.21 | TRUE | 7353 | ubiquitin fusion degradation 1 like (yeast) |
| URG4 | 7p13 | FALSE | 55665 | up-regulated gene 4 |
| USH1C | 11p15.1-p14 | TRUE | 10083 | Usher syndrome 1C (autosomal recessive, severe) |
| USH2A | 1q41 | TRUE | 7399 | Usher syndrome 2A (autosomal recessive, mild) |
| VPS36 | 13q14.3 | TRUE | 51028 | vacuolar protein sorting 36 (yeast) |
| WBSCR17 | 7q11.23 | TRUE | 64409 | Williams-Beuren syndrome chromosome region 17 |
| WDFY4 | 10q11.23 | TRUE | 57705 | WDFY family member 4 |
| WIPI2 | 7p22.1 | TRUE | 26100 | WD repeat domain, phosphoinositide interacting 2 |
| WNT2 | 7q31 | TRUE | 7472 | wingless-type MMTV integration site family member 2 |
| XKR4 | 8q12.1 | TRUE | 114786 | XK, Kell blood group complex subunit-related family, member 4 |
| XM_066971 | | FALSE | | |
| ZBED4 | 22q13.33 | TRUE | 9889 | zinc finger, BED-type containing 4 |
| ZBTB16 | 11q23.1 | TRUE | 7704 | zinc finger and BTB domain containing 16 |
| ZFHX2 | 14q11.2 | TRUE | 85446 | zinc finger homeobox 2 |
| ZFP3 | 17p13.2 | TRUE | 124961 | zinc finger protein 3 homolog (mouse) |
| ZNF390 | 6p22.1 | TRUE | 222696 | zinc finger protein 390 |
| ZNF407 | 18q23 | TRUE | 55628 | zinc finger protein 407 |
| ZNF409 | 14q11.2 | TRUE | 22830 | zinc finger protein 409 |
| ZNF516 | 18q23 | TRUE | 9658 | zinc finger protein 516 |
| ZNF533 | 2q31.2-q31.3 | TRUE | 151126 | zinc finger protein 533 |
| ZNF641 | 12q13.11 | TRUE | 121274 | zinc finger protein 641 |
| ZNFN1A5 | 10q26 | TRUE | 64376 | zinc finger protein, subfamily 1A, 5 |
| ZNRD1 | 6p21.3 | TRUE | 30834 | zinc ribbon domain containing 1 |
| ZUBR1 | 1p36.13 | TRUE | 23352 | zinc finger, UBR1 type 1 |

¹HUGO gene = TRUE indicates the gene symbol is an official, unique Human Genome name. http://www.gene.ucl.ac.uk/nomenclature/index.html
²ENTREZ Gene ID is the gene ID assigned by NCBI http://www.ncbi.nlm.nih.gov/Database/index.html

### REFERENCES

Allison D B, Faith M S, and Nathan J S. (1996) Risch's Lambda Values for Human Schizophrenia. International Journal of Schizophrenia Related Metabolic Disorders 20 (11):990-999.
American Psychiatric Association (1994): Diagnostic and Statistical Manual of Mental Disorders (DSM-IV); 4^{th} Edition, Washington D.C., APA.
Barbato A. Schizophrenia and public health WHO 1998
Baskin M L et al (2005) Prevalence of Schizophrenia in the United States. Schizophrenia Review 6 (1):5-7.
Cardno A G, Gottesman II. Twin studies of schizophrenia: From bow-and-arrow concordances to Star Wars Mx and functional genomics. Am J Med Genet. 2000 January; 97(1):12-17.
Cardno A G, Marshall E J, Coid B, Macdonald A M, Ribchester T R, Davies N J, Venturi P, Jones L A, Lewis S W, Sham P C, Goffesman T I, Farmer A E, McGuffin P, Reveley A M, Murray R M. Heritability estimates for psychotic disorders: the Maudsley twin psychosis series. Arch Gen Psychiatry. 1999 February; 56(2):162-8
Comuzzie A G and Allison D B. (1998) The search for human Schizophrenia genes. Science 280 (5368):1374-1377.
Crandall, M A. The US Market for Schizophrenia Treatment and Weight Management. Theta Report. 2001.
Devlin B. Roeder K. Wassernan L. Genomic control, a new approach to genetic-based association studies. Theoretical Population Biology. 60(3):155-66, 2001 Nov.
Fleiss J, Levin B., Paik M C. (2003) Statistical Methods for Rates and Proportions. 3^{rd} Edition. Wiley.
The Genetics of Schizophrenia. 1994. Bouchard C (ed). Boca Raton: CRC Press.
McGue M, Gottesman II. A single dominant gene still cannot account for the transmission of schizophrenia. Arch Gen Psychiatry. 1989 May; 46(5):478-80.
Meyer J M SAJ (1994) Twin Studies of Human Schizophrenia. The Genetics of Schizophrenia. Boca Raton: CRC Press.
Ref Type: Generic
Mehta, C. and Patel, N. (1983) A Network Algorithm for Performing Fisher's Exact Test in rXc contingency tables. Journal of the American Statistical Association 78:427-434.
Meng, Z. et al. (2003) Selection of Genetic Markers for Assoication Analyses, Using Linkage Disequilbrium and Haplotypes. American Journal of Human Genetics 71(1): 115-130.
National Institutes of Health. Clinical (1998) Guidelines on the Identification, Evaluation, and Treatment of Overweight and Schizophrenia in Adults. National Heart, Lung and Blood Institute in cooperation with The National Institute of Diabetes and Digestive and Kidney Diseases. NIH Publication No. 98-4083.
Ref Type: Generic
Riley B, McGuffin P. Linkage and associated studies of schizophrenia. Am J Med Genet (Semin. Med. Genet.) 2000, 97:23-44
Risch N. Linkage strategies for genetically complex traits. I. Multilocus models. Am J Hum Genet. 1990 February; 46(2):222-8
Roses A D., Burns D K., Chissoe S., Middleton L., St Jean P., (2005) Disease-specific target selection: A Critical First Step Down the Right Road. Drug Discovery Today 10: 177-189.
Sorensen TIA SA (1994) Overview of the adoption studies. The Genetics of Schizophrenia. Boca Raton: CRC Press.
Stunkard A J, Foch T T, and Hrubec Z (1986) A twin study of human Schizophrenia. JAMA 256 (1):51-54.
Taylor J D., Briley D., Nguyen Q., Long K., Iannone M A., Li M S., Ye F., Afshari A., Lai E., Wagner M., Chen J., Weiner M P. (2001) Flow cytometric platform for high-throughput single nucleotide polymorphism analysis. [Journal Article] Biotechniques. 30(3): 661-6, 668-9, March.
Thaker G K, Carpenter WT Jr. Advances in schizophrenia. Nat Med. 2001 June; 7(6):667-71
Weir, B S. (1996) Genetic Data Analysis II. Sinauer Associates, Inc., Sunderland, Mass., pp. 109-1110.
Zaykin D V, Zhivotovsky L A, Weir BS (1995) Exact tests for association between alleles at arbitrary numbers of loci. Genetica 96:169-178.

1. A method of screening a small molecule compound for use in treating schizophrenia, comprising screening a test compound against a target selected from the group consisting of the gene products encoded by the genes of Table 3, where activity against said target indicates the test compound has potential use in treating schizophrenia.
